(19) United States
(12) Patent Application Publication
(10) Pub. No.: US 2025/0155447 A1
(43) Pub. Date: May 15, 2025

(54) METHODS AND SYSTEMS FOR PROCESSING POLYMERIC ANALYTES

(71) Applicant: Glyphic Biotechnologies, Inc., New York, NY (US)

(72) Inventors: David DODD, San Francisco, CA (US); YashoNandini SINGH, Alameda, CA (US); Daniel Masao Estandian, Antioch, CA (US); Andrew John PRICE, San Mateo, CA (US); Asmamaw WASSIE, New York, NY (US); Boyang HUA, Oakland, CA (US); Jai PRAKASH, Fremont, CA (US); Joshua Young Cynming YANG, Richardson, TX (US)

(21) Appl. No.: 18/907,854

(22) Filed: Oct. 7, 2024

Related U.S. Application Data
(63) Continuation of application No. PCT/US23/17954, filed on Apr. 7, 2023.
(60) Provisional application No. 63/328,981, filed on Apr. 8, 2022.
(60) Provisional application No. 63/423,602, filed on Nov. 8, 2022.

(51) Int. Cl. G01N 33/68; G01N 33/543

(52) U.S. Cl. CPC G01N 33/6818; G01N 33/54333; G01N 33/6848; G01N 2333/9015; G01N 2458/10

(57) ABSTRACT

Novel approaches for processing polymeric analytes, such as biopolymers, are provided herein. A method for processing a polymeric analyte may comprise coupling a monomer of the polymeric analyte to a capture moiety, cleaving the monomer from the polymeric analyte, and analyzing the cleaved monomer, e.g., using a binding agent. The methods described herein may employ the use of activating agents.

### CROSS REFERENCE

This application is a continuation application of International Application PCT/US23/17954, filed on Apr. 7, 2023, which claims priority to United States Provisional Patent Application Nos. 63/328,981, filed Apr. 8, 2022, and 63/423,602, filed on Nov. 8, 2022, each of which applications is incorporated by reference herein in its entirety.

### STATEMENT REGARDING FEDERALLY SPONSORED RESEARCH OR DEVELOPMENT

This invention was made with U.S. government support under Grant Number HG012563, awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jan. 24, 2025, is named 60652-703_301_SL.xml and is 37,342 bytes in size.

### BACKGROUND

Analysis of polymeric analytes, both naturally occurring and synthetic, is important for characterizing systems, understanding biology and pathology, and development of therapeutics. Biological polymers, such as proteins, drive cellular processes including differentiation, migration, and disease. As such, direct measurement and characterization of biological polymers is necessary for disease modeling and medicine.

While technological advancements have made significant strides in characterizing some types of biological polymers (e.g., nucleic acid molecules), challenges remain in selectively measuring and analyzing proteins in a high-throughput manner. Current approaches for studying proteins can be limited in selectivity, sensitivity, throughput, or require a priori knowledge. As such, new approaches for characterizing and analyzing proteins is needed.

### SUMMARY

Recognized herein is a need for novel approaches for characterizing and analyzing polymeric analytes, such as proteins, in an accurate and high-throughput manner. Provided herein are methods, systems, and compositions for analyzing (e.g., via sequencing) polymeric analytes that address the abovementioned needs. A method of the present disclosure may comprise providing a polymeric analyte comprising a plurality of monomers, cleaving a monomer of the plurality of monomers, and analyzing the cleaved monomer. The analysis of the cleaved monomer may comprise contacting the cleaved monomer with a binding agent that recognizes the cleaved monomer, or a portion thereof. The methods disclosed herein may also employ local tethering of polymerizable molecules. In some instances, the cleaved monomer may be coupled to a capture moiety, and the binding agent may couple to the cleaved monomer. A first polymerizable molecule comprising identifying information of the monomer may couple to a second polymerizable molecule which is adjacent to the capture moiety. Subsequently, the process may be repeated to analyze all or a subset of the monomers of the polymeric analyte. Further characterization of the polymerizable molecules may be performed, which may aid in the identification of the individual monomers of the polymeric analyte.

In an aspect, provided herein is a method for sequencing a peptide with single-molecule sensitivity, comprising: (a) providing the peptide, wherein the peptide comprises a plurality of amino acids; and (b) sequencing the peptide to determine an identity and order of at least a subset of the plurality of amino acids, wherein an individual read accuracy of an amino acid is greater than 50% for at least 5 different amino acids.

In some embodiments, at least one amino acid of the plurality of amino acids comprises a post-translational modification. In some embodiments, (a) comprises providing a plurality of peptides including the peptide. In some embodiments, the individual read accuracy of the amino acid is greater than 50%, wherein the individual read accuracy is not affected by a side chain of an adjacent amino acid. In some embodiments, the amino acid comprises one or more amino acids. In some embodiments, the one or more amino acids is fewer than 6 amino acids. In some embodiments, the amino acid comprises only one amino acid.

In some embodiments, (b) comprises: (i) cleaving the amino acid from the peptide; (ii) coupling the amino acid to a capture moiety to generate an amino acid-capture moiety complex; (iii) contacting the amino acid-capture moiety complex with a binding agent; (iv) providing a first polymerizable molecule; and (v) coupling the first polymerizable molecule to a second polymerizable molecule. In some embodiments, (ii) occurs prior to (i). In some embodiments, (ii) occurs during (i). In some embodiments, (ii) occurs subsequent to (i). In some embodiments, (iv) occurs prior to (iii). In some embodiments, (iv) occurs during (iii). In some embodiments, (iv) occurs subsequent to (iii). In some embodiments, during (iv), the binding agent is not coupled to the first polymerizable molecule. In some embodiments, the binding agent comprises an activating agent, wherein the activating agent facilitates coupling of the first polymerizable molecule to the second polymerizable molecule. In some embodiments, the activating agent comprises an activating polymerizable molecule, and further comprising, subsequent to (iv), coupling the first polymerizable molecule to the activating polymerizable molecule. In some embodiments, the coupling is mediated using a splint molecule comprising a sequence complementary to at least a portion of (i) the first polymerizable molecule, (ii) the second polymerizable molecule and (iii) the activating polymerizable molecule. In some embodiments, the first polymerizable molecule and the activating polymerizable molecules are nucleic acid molecules and wherein the coupling of the first polymerizable molecule to the activating polymerizable molecule occurs via hybridization chain reaction (HCR). In some embodiments, the activating agent comprises an enzyme. In some embodiments, the enzyme comprises a peroxidase, a Cas protein, a ligase, a kinase, or a restriction enzyme. In some embodiments, the enzyme comprises a kinase, wherein the kinase is configured to phosphorylate the second polymerizable molecule, thereby allowing attachment of the first polymerizable molecule to the second polymerizable molecule. In some embodiments, the binding agent is coupled indirectly to the first polymerizable molecule. In some embodiments, the binding agent comprises an activating polymerizable molecule, and wherein the first polymerizable molecule is coupled to the activating polymerizable molecule. In some embodiments, the binding agent comprises an activating agent, wherein the activating agent is configured to facilitate coupling of the first polymerizable molecule to the second polymerizable molecule. In some embodiments, the activating agent comprises an enzyme or a nucleic acid molecule. In some embodiments, the activating agent comprises an anchoring nucleic acid molecule, wherein the anchoring nucleic acid molecule is configured to couple to an additional second polymerizable molecule adjacent to the second polymerizable molecule. In some embodiments, the activating agent comprises an enzyme selected from the group consisting of: a peroxidase, a Cas protein, a ligase, a kinase, and a restriction enzyme. In some embodiments, the enzyme is a kinase, and further comprising, using the kinase to phosphorylate the second polymerizable molecule, thereby allowing coupling of the first polymerizable to the second polymerizable molecule. In some embodiments, the enzyme is a horseradish peroxidase (HRP) or an ascorbate peroxidase (APEX), wherein the HRP or the APEX activates the first polymerizable molecule or the second polymerizable molecule, thereby coupling the first polymerizable molecule to the second polymerizable molecule. In some embodiments, the enzyme is a restriction enzyme and wherein the second polymerizable molecule comprises a restriction recognition site, and further comprising, cleaving the second polymerizable molecule with the restriction enzyme prior to the coupling of the first polymerizable molecule to the second polymerizable molecule. In some embodiments, the binding agent comprises an activating nucleic acid molecule and wherein the first polymerizable molecule is a nucleic acid molecule, and further comprising, prior to (iii), coupling the first polymerizable molecule to the activating nucleic acid sequence. In some embodiments, the method further comprises generating the first polymerizable molecule via hybridization chain reaction (HCR). In some embodiments, the generating comprises (A) providing a first hairpin molecule and a second hairpin molecule; (B) coupling the first hairpin molecule to the activating nucleic acid sequence; and (C) coupling the second hairpin molecule to the first hairpin molecule, thereby generating the first polymerizable molecule coupled to the activating nucleic acid molecule. In some embodiments, subsequent to (C), the first polymerizable molecule comprises a flap sequence, and further comprising, using the flap sequence to couple the first polymerizable molecule to the second polymerizable molecule. In some embodiments, the method further comprises repeating (i)-(v) at least once. In some embodiments, the capture moiety comprises a third polymerizable molecule. In some embodiments, the first polymerizable molecule, the second polymerizable molecule or the third polymerizable molecule comprises a nucleic acid molecule. In some embodiments, the nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof. In some embodiments, the nucleic acid molecule comprises a hexonucleic acid (HNA). In some embodiments, greater than 70% of nucleotides of the nucleic acid molecule are thymines or cytosines. In some embodiments, the third polymerizable comprises a DNA molecule comprising a priming site, a sequencing site, a unique molecular identifier, a barcode sequence, a cleavage site, or a combination thereof. In some embodiments, the first polymerizable molecule comprises a first nucleic acid molecule and wherein the third polymerizable molecule comprises a second nucleic acid molecule. In some embodiments, the coupling in (iv) comprises hybridization of at least a portion of the first nucleic acid molecule to at least a portion of the second nucleic acid molecule. In some embodiments, the method further comprises performing a nucleic acid extension reaction. In some embodiments, the cleaving in (i) comprises cleaving one or more amino acids from the peptide. In some embodiments, the first polymerizable molecule comprises a first nucleic acid molecule and the second polymerizable molecule comprises a second nucleic acid molecule. In some embodiments, the coupling in (ii) comprises ligation or hybridization of the first nucleic acid molecule to the second nucleic acid molecule. In some embodiments, the coupling in (ii) is mediated by a splint molecule. In some embodiments, the first polymerizable molecule comprises a first reactive moiety and the second polymerizable molecule comprises a second reactive moiety capable of reacting with the first reactive moiety. In some embodiments, the coupling in (ii) comprises reacting the first moiety and the second moiety. In some embodiments, the first reactive moiety or the second reactive moiety comprises a click chemistry moiety. In some embodiments, the method further comprises decoupling the amino acid from the capture moiety. In some embodiments, the method further comprises, repeating (b). In some embodiments, the method further comprises identifying at least a portion of the first polymerizable molecule or derivative thereof. In some embodiments, the identifying comprises sequencing the first polymerizable molecule or derivative thereof. In some embodiments, the sequencing is performed using next generation DNA sequencing. In some embodiments, the identifying comprises contacting the first polymerizable molecule or derivative thereof with a probe. In some embodiments, the coupling of (ii) is mediated using a linker. In some embodiments, the linker is a bifunctional linker. In some embodiments, the linker comprises a first reactive group and a second reactive group, wherein the first reactive group is capable of coupling to the amino acid and wherein the second reactive group is capable of coupling to the capture moiety. In some embodiments, the amino acid is a terminal amino acid. In some embodiments, the first reactive group comprises a thiocyanate, aldehyde group, dansyl chloride, or fluorobenzene. In some embodiments, the fluorobenzene is dinitrofluorobenzene. In some embodiments, the thiocyanate comprises a phenylisothiocyanate (PITC) moiety and wherein the coupling of (ii) generates a phenylthiocarbamoyl (PTC) derivative of the amino acid. In some embodiments, the method further comprises, subsequent to (e), derivatizing the phenylthiocarbamoyl (PTC) derivative to generate a thiazolinone (ATZ) derivative or a phenylthiohydantoin (PTH) derivative. In some embodiments, the method further comprises, derivatizing the ATZ derivative or the PTH derivative to another PTC derivative. In some embodiments, the cleaving in (i) is performed by applying a stimulus. In some embodiments, the stimulus comprises a change in pH. In some embodiments, the change in pH comprises the use of an acid. In some embodiments, the change in pH comprises the use of a base. In some embodiments, the stimulus comprises the use of a Lewis acid. In some embodiments, the Lewis acid comprises boron triflate (BF₃), boron trifluoride etherate, boron trichloride, boron tribromide, boron triiodide, or scandium triflate. In some embodiments, the stimulus comprises electromagnetic radiation. In some embodiments, the electromagnetic radiation is applied using a microwave. In some embodiments, the capture moiety comprises a third reactive group that is capable of reacting with the second reactive group. In some embodiments, the first reactive group, the second reactive group, or the third reactive group comprises a click chemistry moiety. In some embodiments, the second reactive group and the third reactive group comprise a click chemistry pair. In some embodiments, the linker comprises an amino acid reactive group and an additional polymerizable molecule, wherein the additional polymerizable molecule is configured to couple to the capture moiety. In some embodiments, the amino acid reactive group is capable of cleaving the amino acid. In some embodiments, the additional polymerizable molecule comprises an enzyme recognition site. In some embodiments, the enzyme recognition site is recognized by a nuclease. In some embodiments, the method further comprises subsequent to (iv), cleaving the additional polymerizable molecule, thereby releasing the amino acid from the capture moiety. In some embodiments, the nuclease is a restriction enzyme, a Cas protein, or an Argonaut nuclease. In some embodiments, the cleaving in (i) comprises applying a stimulus. In some embodiments, the stimulus comprises a change in pH. In some embodiments, the stimulus comprises the use of a Lewis acid. In some embodiments, the Lewis acid comprises boron triflate (BF₃), boron trifluoride etherate, boron trichloride, boron tribromide, boron triiodide, or scandium triflate. In some embodiments, the stimulus comprises electromagnetic radiation. In some embodiments, the electromagnetic radiation is applied using a microwave. In some embodiments, the stimulus comprises a biological stimulus. In some embodiments, the biological stimulus comprises use of an enzyme. In some embodiments, the enzyme is a metalloprotease, aminopeptidase, or exopeptidase. In some embodiments, the method further comprises providing a metal catalyst. In some embodiments, the amino acid comprises a modification, and wherein the enzyme recognizes the modification. In some embodiments, the first polymerizable molecule comprises a moiety that identifies the binding agent or the amino acid. In some embodiments, the first polymerizable molecule comprises a nucleic acid molecule comprising a barcode sequence that identifies the binding agent or the amino acid. In some embodiments, the method further comprises subjecting the amino acid-capture moiety complex to conditions sufficient to inhibit binding of an additional binding agent to the amino acid-capture moiety complex. In some embodiments, the conditions comprise a chemical or enzymatic treatment. In some embodiments, the method further comprises blocking a carboxyl group or an amine group of the peptide. In some embodiments, the binding agent comprises an antibody, antibody fragment, aptamer, scFv, nanobody, anticalin, tRNA-acyl synthetase, peptide, peptide mimetic, a polysaccharide, or derivative thereof. In some embodiments, the binding agent comprises an enzyme. In some embodiments, the enzyme comprises a metalloprotease, aminopeptidase, or exopeptidase. In some embodiments, the peptide, the capture moiety, or the second polymerizable molecule is coupled to a substrate. In some embodiments, the substrate is a solid support. In some embodiments, the substrate is substantially planar. In some embodiments, the substrate is a bead or particle. In some embodiments, the peptide, the capture moiety, or the second polymerizable molecule is coupled to the substrate via a click chemistry moiety. In some embodiments, the peptide, the capture moiety, or the second polymerizable molecule is coupled to the support via a functional group. In some embodiments, the functional group is added to the peptide, the capture moiety, or the second polymerizable molecule using an enzyme. In some embodiments, the enzyme comprises an amidase. In some embodiments, the peptide, the capture moiety, or the second polymerizable molecule is coupled to the substrate using a linker molecule. In some embodiments, the substrate is functionalized with a functional group, and further comprising, attaching the peptide, the capture moiety, or the second polymerizable molecule to the functional group. In some embodiments, the peptide, the capture moiety, and the second polymerizable molecule are coupled to the substrate. In some embodiments, the method further comprises passivating the substrate. In some embodiments, the passivating decreases nonspecific binding of the binding agent. In some embodiments, at least one amino acid of the plurality of amino acids comprises a non-naturally occurring modification. In some embodiments, the method further comprises generating the peptide comprising the non-naturally occurring modification. In some embodiments, the generating comprises alkylating or acetylating the at least one amino acid, beta-elimination of a phosphate group, or use of PITC or acetic anhydride. In some embodiments, the generating comprises converting a cysteine residue to cysteic acid. In some embodiments, the generating comprises use of an oxidizing or reducing agent.

In some embodiments, the peptide is derived from a biological sample.

In some embodiments, (b) comprises: (i) cleaving the amino acid from the peptide; (ii) coupling the amino acid to a capture moiety to generate an amino acid-capture moiety complex; (iii) contacting the amino acid-capture moiety complex with a binding agent, wherein the binding agent comprises an activating agent; and (iv) detecting a product from the activating agent. In some embodiments, the activating agent comprises an enzyme that generates the product. In some embodiments, the enzyme is a horseradish peroxidase or ascorbate peroxidase.

In another aspect, provided herein is a method for processing a polymeric analyte, comprising: (a) providing the polymeric analyte and a capture moiety, wherein the polymeric analyte comprises a plurality of monomers; (b) cleaving a monomer of the plurality of monomers from the polymeric analyte; (c) coupling the monomer to the capture moiety to generate a monomer-capture moiety complex; (d) subsequent to (b) and (c), contacting the monomer-capture moiety complex with a binding agent comprising an activating agent; (e) providing a first polymerizable molecule; and (f) using the activating agent to couple the first polymerizable molecule to a second polymerizable molecule.

In some embodiments, (e) occurs prior to (d). In some embodiments, (e) occurs during (d). In some embodiments, (e) occurs subsequent to (d). In some embodiments, during (e), the binding agent is not coupled to the first polymerizable molecule. In some embodiments, the binding agent is coupled indirectly to the first polymerizable molecule. In some embodiments, the activating agent comprises an activating polymerizable molecule, and further comprising, coupling the first polymerizable molecule to the activating polymerizable molecule. In some embodiments, the first polymerizable molecule, the second polymerizable molecule, and the activating polymerizable molecule are nucleic acid molecules, and wherein the coupling is mediated using a splint molecule comprising a sequence complementary to at least a portion of (i) the first polymerizable molecule, (ii) the second polymerizable molecule and (iii) the activating polymerizable molecule. In some embodiments, the first polymerizable molecule comprises a nucleic acid molecule, and further comprising, generating the first polymerizable molecule via hybridization chain reaction (HCR). In some embodiments, the generating comprises (A) providing a first hairpin molecule and a second hairpin molecule; (B) coupling the first hairpin molecule to the activating polymerizable molecule; and (C) coupling the second hairpin molecule to the first hairpin molecule, thereby generating the first polymerizable molecule coupled to the activating polymerizable molecule. In some embodiments, subsequent to (C), the first polymerizable molecule comprises a flap sequence, and further comprising, using the flap sequence to couple the first polymerizable molecule to the second polymerizable molecule. In some embodiments, the method further comprises coupling an additional first hairpin molecule to the first polymerizable molecule and an additional second hairpin molecule to the additional first hairpin molecule, thereby generating an additional first polymerizable molecule that is coupled to the first polymerizable molecule. In some embodiments, the method further comprises coupling the additional first polymerizable molecule to an additional second polymerizable molecule. In some embodiments, the method further comprises repeating (b)-(f), wherein the repeating comprises generating additional first polymerizable molecules by repeating (A)-(C). In some embodiments, the repeating of (A)-(C) comprises providing additional hairpin molecules, wherein at least a subset of the additional hairpin molecules comprises a different sequence than the first hairpin molecule or the second hairpin molecule. In some embodiments, the subset of the additional hairpin molecules comprises a sequence that is configured to couple to at least a portion of the first polymerizable molecule. In some embodiments, the method further comprises cleaving a portion of the first polymerizable molecule. In some embodiments, the activating agent comprises an anchoring nucleic acid molecule, wherein the anchoring nucleic acid molecule is configured to couple to an additional second polymerizable molecule adjacent to the second polymerizable molecule. In some embodiments, the first polymerizable molecule is coupled to the binding agent. In some embodiments, the activating agent comprises an enzyme selected from the group consisting of: a peroxidase, a Cas protein, a ligase, a kinase, and a restriction enzyme. In some embodiments, the enzyme is a kinase, and further comprising, using the kinase to phosphorylate the second polymerizable molecule, thereby allowing coupling of the first polymerizable to the second polymerizable molecule. In some embodiments, the enzyme is a horseradish peroxidase (HRP) or an ascorbate peroxidase (APEX), wherein the HRP or the APEX activates the first polymerizable molecule or the second polymerizable molecule, thereby coupling the first polymerizable molecule to the second polymerizable molecule. In some embodiments, the enzyme is a restriction enzyme and wherein the second polymerizable molecule comprises a restriction recognition site, and further comprising, cleaving the second polymerizable molecule with the restriction enzyme prior to the coupling of the first polymerizable molecule to the second polymerizable molecule. In some embodiments, (c) is performed prior to (b). In some embodiments, (c) is performed during (b). In some embodiments, (c) is performed subsequent to (b). In some embodiments, the cleaving in (b) comprises cleaving one or more monomers of the plurality of monomers. In some embodiments, the capture moiety comprises a third polymerizable molecule. In some embodiments, the first polymerizable molecule, the second polymerizable molecule or the third polymerizable molecule comprises a nucleic acid molecule. In some embodiments, the nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof. In some embodiments, the nucleic acid molecule comprises a hexonucleic acid (HNA). In some embodiments, greater than 70% of nucleotides of the nucleic acid molecule are thymines or cytosines. In some embodiments, the third polymerizable comprises a DNA molecule comprising a priming site, a sequencing site, a unique molecular identifier, a barcode sequence, a cleavage site, or a combination thereof. In some embodiments, the first polymerizable molecule comprises a first nucleic acid molecule and wherein the third polymerizable molecule comprises a second nucleic acid molecule. In some embodiments, the coupling in (e) comprises hybridization of at least a portion of the first nucleic acid molecule to at least a portion of the second nucleic acid molecule. In some embodiments, the method further comprises performing a nucleic acid extension reaction. In some embodiments, the first polymerizable molecule comprises a first nucleic acid molecule and the second polymerizable molecule comprises a second nucleic acid molecule. In some embodiments, the coupling in (c) comprises ligation or hybridization of the first nucleic acid molecule to the second nucleic acid molecule. In some embodiments, the coupling in (c) is mediated by a splint molecule. In some embodiments, the first polymerizable molecule comprises a first reactive moiety and the second polymerizable molecule comprises a reactive moiety capable of reacting with the first reactive moiety. In some embodiments, the coupling in (c) comprises reacting the first moiety and the second moiety. In some embodiments, the first reactive moiety or the second reactive moiety comprises a click chemistry moiety. In some embodiments, the method further comprises decoupling the monomer from the capture moiety. In some embodiments, the method further comprises repeating (b)-(e). In some embodiments, the method further comprises identifying at least a portion of the first polymerizable molecule or derivative thereof. In some embodiments, the identifying comprises sequencing the first polymerizable molecule or derivative thereof. In some embodiments, the sequencing is performed using next generation DNA sequencing. In some embodiments, the identifying comprises using hybridization of probes. In some embodiments, the coupling of (c) is mediated using a linker. In some embodiments, the linker is a bifunctional linker. In some embodiments, the linker comprises a first reactive group and a second reactive group, wherein the first reactive group is capable of coupling to the monomer and wherein the second reactive group is capable of coupling to the capture moiety. In some embodiments, the polymeric analyte is a peptide comprising a plurality of amino acids and the first reactive group is capable of coupling to an amino acid. In some embodiments, the amino acid is a terminal amino acid. In some embodiments, at least one amino acid of the plurality of amino acids comprises a post-translational modification. In some embodiments, the first reactive group comprises a thiocyanate, aldehyde group, dansyl chloride, or fluorobenzene. In some embodiments, the thiocyanate comprises a phenylisothiocyanate (PITC) moiety and wherein the coupling of (c) generates a phenylthiocarbamoyl derivative of the amino acid. In some embodiments, the method further comprises, subsequent to (e), derivatizing the phenylthiocarbamoyl derivative to generate a thiazolinone (ATZ) derivative or a phenylthiohydantoin (PTH)-amino acid derivative. In some embodiments, the cleaving in (b) is performed by applying a stimulus. In some embodiments, the stimulus comprises a change in pH. In some embodiments, the change in pH comprises the use of an acid. In some embodiments, the change in pH comprises the use of a base. In some embodiments, the stimulus comprises the use of a Lewis acid. In some embodiments, the Lewis acid comprises boron triflate (BF₃), boron trifluoride etherate, boron trichloride, boron tribromide, boron triiodide, or scandium triflate. In some embodiments, the stimulus comprises electromagnetic radiation. In some embodiments, the electromagnetic radiation is applied using a microwave. In some embodiments, the capture moiety comprises a third reactive group that is capable of reacting with the second reactive group. In some embodiments, the first reactive group, the second reactive group, or the third reactive group comprises a click chemistry moiety. In some embodiments, the second reactive group and the third reactive group comprise a click chemistry pair. In some embodiments, the linker comprises an amino acid reactive group and an additional polymerizable molecule, wherein the additional polymerizable molecule is configured to couple to the capture moiety. In some embodiments, the additional polymerizable molecule comprises a nucleic acid molecule comprising an enzyme recognition site. In some embodiments, the enzyme recognition site is recognized by a nuclease. In some embodiments, the method further comprises subsequent to (e), cleaving the additional polymerizable molecule, thereby releasing the one or more monomers from the capture moiety. In some embodiments, the nuclease is a restriction enzyme, a Cas protein, or an Argonaut nuclease. In some embodiments, the polymeric analyte comprises a macromolecule. In some embodiments, the macromolecule comprises a peptide comprising a plurality of amino acids, wherein the monomer is an amino acid of the plurality of amino acids. In some embodiments, the amino acid is a terminal amino acid. In some embodiments, at least one amino acid of the plurality of amino acids comprises a post-translational modification. In some embodiments, at least one amino acid of the plurality of amino acids comprises a non-naturally occurring modification. In some embodiments, the method further comprises generating the peptide comprising the non-naturally occurring modification. In some embodiments, the generating comprises alkylating or acetylating the at least one amino acid, beta-elimination of a phosphate group, or use of PITC or acetic anhydride. In some embodiments, the generating comprises converting a cysteine residue to cysteic acid. In some embodiments, the generating comprises use of an oxidizing or reducing agent. In some embodiments, the non-naturally occurring modification is located on the amino acid. In some embodiments, the macromolecule is derived from a biological sample. In some embodiments, the polymeric analyte comprises a peptoid. In some embodiments, the binding agent comprises an antibody, antibody fragment, aptamer, scFv, nanobody, anticalin, tRNA-acyl synthetase, peptide, peptide mimetic, a polysaccharide, or derivative thereof. In some embodiments, the binding agent comprises an enzyme. In some embodiments, the enzyme comprises a metalloprotease, aminopeptidase, or exopeptidase. In some embodiments, the polymeric analyte, the capture moiety, or the second polymerizable molecule is coupled to a substrate. In some embodiments, the substrate is a solid support. In some embodiments, the substrate is substantially planar. In some embodiments, the substrate is a bead or particle. In some embodiments, the polymeric analyte, the capture moiety, or the second polymerizable molecules is coupled to the substrate via a click chemistry moiety. In some embodiments, the polymeric analyte, the capture moiety, or the second polymerizable molecules is coupled to the substrate via a functional group. In some embodiments, the functional group is added to the polymeric analyte, the capture moiety, or the second polymerizable molecule using an enzyme. In some embodiments, the enzyme comprises an amidase. In some embodiments, the polymeric analyte, the capture moiety, or the second polymerizable molecules is coupled to the substrate using a linker molecule. In some embodiments, the substrate is functionalized with a functional group, and further comprising, attaching the polymeric analyte, the capture moiety, or the second polymerizable molecule to the functional group. In some embodiments, the polymeric analyte, the capture moiety, and the second polymerizable molecule are coupled to the substrate. In some embodiments, the method further comprises passivating the substrate. In some embodiments, the passivating decreases nonspecific binding of the binding agent. In some embodiments, the cleaving in (b) comprises applying a stimulus. In some embodiments, the stimulus comprises a change in pH. In some embodiments, the stimulus comprises the use of a Lewis acid. In some embodiments, the Lewis acid comprises boron triflate (BF₃), boron trifluoride etherate, boron trichloride, boron tribromide, boron triiodide, or scandium triflate. In some embodiments, the stimulus comprises a biological stimulus. In some embodiments, the biological stimulus comprises use of an enzyme. In some embodiments, the enzyme is a metalloprotease, aminopeptidase, or exopeptidase. In some embodiments, the method further comprises providing a metal catalyst. In some embodiments, the monomer comprises a modification, and wherein the enzyme recognizes the modification. In some embodiments, the first polymerizable molecule comprises a moiety that identifies the binding agent or the monomer. In some embodiments, the first polymerizable molecule comprises a nucleic acid molecule comprising a sequence that identifies the binding agent or the monomer. In some embodiments, the method further comprises subjecting the monomer-capture moiety complex to conditions sufficient to inhibit binding of an additional binding agent to the monomer-capture moiety complex. In some embodiments, the conditions comprise a chemical or enzymatic treatment. In some embodiments, the monomer is <10 nm in size. In some embodiments, the monomer has a molecular mass of less than 210 daltons.

In yet another aspect, disclosed herein is a method for processing an analyte, comprising: (a) providing (i) the analyte, (ii) a binding agent comprising an activating agent, (iii) a first polymerizable molecule, and (iv) a second polymerizable molecule, wherein the analyte is <10 nm in size; (b) contacting the analyte with the binding agent; and (c) using the activating agent to couple the first polymerizable molecule to the second polymerizable molecule.

In some embodiments, the binding agent is not coupled to the first polymerizable molecule. In some embodiments, the binding agent is coupled indirectly to the first polymerizable molecule. In some embodiments, the activating agent comprises an activating polymerizable molecule, and further comprising, coupling the first polymerizable molecule to the activating polymerizable molecule. In some embodiments, the first polymerizable molecule, the second polymerizable molecule, and the activating polymerizable molecule are nucleic acid molecules, and wherein the coupling is mediated using a splint molecule comprising a sequence complementary to at least a portion of (i) the first polymerizable molecule, (ii) the second polymerizable molecule and (iii) the activating polymerizable molecule. In some embodiments, the first polymerizable molecule comprises a nucleic acid molecule, and further comprising, generating the first polymerizable molecule via hybridization chain reaction (HCR). In some embodiments, the generating comprises (A) providing a first hairpin molecule and a second hairpin molecule; (B) coupling the first hairpin molecule to the activating polymerizable molecule; and (C) coupling the second hairpin molecule to the first hairpin molecule, thereby generating the first polymerizable molecule coupled to the activating polymerizable molecule. In some embodiments, subsequent to (C), the first polymerizable molecule comprises a flap sequence, and further comprising, using the flap sequence to couple the first polymerizable molecule to the second polymerizable molecule. In some embodiments, the activating agent comprises an anchoring nucleic acid molecule, wherein the anchoring nucleic acid molecule is configured to couple to an additional second polymerizable molecule adjacent to the second polymerizable molecule. In some embodiments, the first polymerizable molecule is coupled to the binding agent. In some embodiments, the activating agent comprises an enzyme selected from the group consisting of: a peroxidase, a Cas protein, a ligase, a kinase, and a restriction enzyme. In some embodiments, the enzyme is a kinase, and further comprising, using the kinase to phosphorylate the second polymerizable molecule, thereby allowing coupling of the first polymerizable to the second polymerizable molecule. In some embodiments, the enzyme is a horseradish peroxidase (HRP) or an ascorbate peroxidase (APEX), wherein the HRP or the APEX activates the first polymerizable molecule or the second polymerizable molecule, thereby coupling the first polymerizable molecule to the second polymerizable molecule. In some embodiments, the enzyme is a restriction enzyme and wherein the second polymerizable molecule comprises a restriction recognition site, and further comprising, cleaving the second polymerizable molecule with the restriction enzyme prior to the coupling of the first polymerizable molecule to the second polymerizable molecule. In some embodiments, the analyte comprises a monomeric unit of a polymer. In some embodiments, the polymer is a peptide and the monomeric unit comprises an amino acid. In some embodiments, at least one amino acid of the peptide comprises a post-translational modification. In some embodiments, the method further comprises, prior to (a), providing a polymer comprising a plurality of monomers, coupling a monomer of the plurality of monomers to a substrate, and cleaving the monomer from the polymer, thereby yielding the analyte coupled to the substrate. In some embodiments, the polymer comprises a peptide and the monomer comprises an amino acid or modified amino acid. In some embodiments, the coupling is mediated by a linker. In some embodiments, the linker comprises a first reactive moiety that is capable of coupling to the monomer and a second reactive moiety that is capable of coupling to the substrate.

In yet another aspect, provided herein is a method for processing an analyte, comprising: (a) providing (i) the analyte, (ii) a binding agent, and (iii) a first polymerizable molecule, wherein the first polymerizable molecule is not coupled to the binding agent; and (iv) a second polymerizable molecule, wherein the analyte is <10 nm in size; (b) contacting the analyte with the binding agent; and (c) coupling the first polymerizable molecule to the second polymerizable molecule.

In some embodiments, the binding agent comprises an activating polymerizable molecule, and further comprising, coupling the first polymerizable molecule to the activating polymerizable molecule. In some embodiments, the first polymerizable molecule, the second polymerizable molecule, and the activating polymerizable molecule are nucleic acid molecules, and wherein the coupling is mediated using a splint molecule comprising a sequence complementary to at least a portion of (i) the first polymerizable molecule, (ii) the second polymerizable molecule and (iii) the activating polymerizable molecule. In some embodiments, the first polymerizable molecule comprises a nucleic acid molecule, and further comprising, generating the first polymerizable molecule via hybridization chain reaction (HCR). In some embodiments, the generating comprises (A) providing a first hairpin molecule and a second hairpin molecule; (B) coupling the first hairpin molecule to the activating polymerizable molecule; and (C) coupling the second hairpin molecule to the first hairpin molecule, thereby generating the first polymerizable molecule coupled to the activating polymerizable molecule. In some embodiments, subsequent to (C), the first polymerizable molecule comprises a flap sequence, and further comprising, using the flap sequence to couple the first polymerizable molecule to the second polymerizable molecule. In some embodiments, the binding agent comprises an anchoring nucleic acid molecule, wherein the anchoring nucleic acid molecule is configured to couple to an additional second polymerizable molecule adjacent to the second polymerizable molecule. In some embodiments, the binding agent comprises an enzyme coupled thereto, wherein the enzyme is selected from the group consisting of: a peroxidase, a Cas protein, a ligase, a kinase, and a restriction enzyme. In some embodiments, the enzyme is a kinase, and further comprising, using the kinase to phosphorylate the second polymerizable molecule, thereby allowing coupling of the first polymerizable to the second polymerizable molecule. In some embodiments, the enzyme is a horseradish peroxidase (HRP) or an ascorbate peroxidase (APEX), wherein the HRP or the APEX activates the first polymerizable molecule or the second polymerizable molecule, thereby coupling the first polymerizable molecule to the second polymerizable molecule. In some embodiments, the enzyme is a restriction enzyme and wherein the second polymerizable molecule comprises a restriction recognition site, and further comprising, cleaving the second polymerizable molecule with the restriction enzyme prior to the coupling of the first polymerizable molecule to the second polymerizable molecule. In some embodiments, the analyte comprises a monomeric unit of a polymer. In some embodiments, the polymer is a peptide and the monomeric unit comprises an amino acid. In some embodiments, at least one amino acid of the peptide comprises a post-translational modification. In some embodiments, the method further comprises, prior to (a), providing a polymer comprising a plurality of monomers, coupling a monomer of the plurality of monomers to a substrate, and cleaving the monomer from the polymer, thereby yielding the analyte coupled to the substrate. In some embodiments, the polymer comprises a peptide and the monomer comprises an amino acid or modified amino acid. In some embodiments, the coupling is mediated by a linker. In some embodiments, the linker comprises a first reactive moiety that is capable of coupling to the monomer and a second reactive moiety that is capable of coupling to the substrate.

In another aspect of the present disclosure, provided herein is a method, comprising: (a) providing (i) a peptide comprising a plurality of amino acids, wherein an amino acid of the plurality of amino acids comprises a non-naturally occurring modification of the plurality of amino acids, (ii) a substrate coupled to the peptide, and (iii) a linker; (b) using the linker to couple the amino acid or an additional amino acid of the plurality of amino acids to the substrate to yield a substrate-coupled amino acid; and (c) subsequent to (b), cleaving the substrate-coupled amino acid from the peptide, thereby yielding (i) a removed substrate-coupled amino acid and (ii) a remainder of the peptide. In some embodiments, the method further comprises subsequent to (c), contacting the removed substrate-coupled amino acid with a binding agent. In some embodiments, the binding agent comprises a first polymerizable molecule, and wherein the substrate comprises a second polymerizable molecule. In some embodiments, the first polymerizable molecule comprises a moiety that identifies the binding agent or the amino acid. In some embodiments, the first polymerizable molecule comprises a nucleic acid molecule comprising a barcode sequence that identifies the binding agent or the amino acid. In some embodiments, the method further comprises coupling the first polymerizable molecule to the second polymerizable molecule. In some embodiments, the method further comprises sequencing the first polymerizable molecule. In some embodiments, the sequencing is performed using next generation DNA sequencing. In some embodiments, the first polymerizable molecule or the second polymerizable molecule comprises a nucleic acid molecule. In some embodiments, the nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof. In some embodiments, the nucleic acid molecule comprises a hexonucleic acid (HNA). In some embodiments, greater than 70% of nucleotides of the nucleic acid molecule are thymines or cytosines. In some embodiments, the first polymerizable molecule comprises a first nucleic acid molecule and the second polymerizable molecule comprises a second nucleic acid molecule. In some embodiments, the coupling of the first polymerizable molecule and the second polymerizable molecule comprises ligation or hybridization of the first nucleic acid molecule to the second nucleic acid molecule. In some embodiments, the coupling of the first polymerizable molecule and the second polymerizable molecule is mediated by a splint molecule. In some embodiments, the coupling comprises hybridization of at least a portion of the first polymerizable molecule to at least a portion of the second polymerizable molecule. In some embodiments, the method further comprises performing a nucleic acid extension reaction. In some embodiments, the first polymerizable molecule comprises a first reactive moiety and the second polymerizable molecule comprises a second reactive moiety capable of reacting with the first reactive moiety. In some embodiments, the coupling in (ii) comprises reacting the first moiety and the second moiety. In some embodiments, the first reactive moiety or the second reactive moiety comprises a click chemistry moiety. In some embodiments, the binding agent comprises an antibody, antibody fragment, aptamer, nanobody, peptide, peptide mimetic, a polysaccharide, or derivative thereof. In some embodiments, the binding agent comprises an enzyme. In some embodiments, the enzyme comprises a metalloprotease, aminopeptidase, or exopeptidase. In some embodiments, the cleaving in (c) comprises applying a stimulus. In some embodiments, the stimulus comprises a change in pH. In some embodiments, the stimulus comprises the use of a Lewis acid. In some embodiments, the Lewis acid comprises boron triflate (BF₃), boron trifluoride etherate, boron trichloride, boron tribromide, boron triiodide, or scandium triflate. In some embodiments, the stimulus comprises electromagnetic radiation. In some embodiments, the electromagnetic radiation is applied using a microwave. In some embodiments, the stimulus is a biological stimulus. In some embodiments, the biological stimulus comprises use of an enzyme. In some embodiments, the enzyme is a metalloprotease, aminopeptidase, or exopeptidase. In some embodiments, the method further comprises providing a metal catalyst. In some embodiments, the amino acid comprises a modification, and wherein the enzyme recognizes the modification. In some embodiments, the method further comprises subjecting the removed substrate-coupled amino acid to conditions sufficient to inhibit binding of an additional binding agent to the removed substrate-coupled amino acid. In some embodiments, the conditions comprise a chemical or enzymatic treatment. In some embodiments, the method further comprises blocking a carboxyl group or an amine group of the peptide. In some embodiments, the cleaving in (c) comprises cleaving one or more amino acids from the peptide. In some embodiments, the method further comprises removing the amino acid of the substrate-coupled amino acid from the substrate. In some embodiments, the substrate comprises a capture moiety, wherein the capture moiety is coupled to the amino acid or the additional amino acid via the linker. In some embodiments, the capture moiety comprises a polymerizable molecule. In some embodiments, the polymerizable molecule is a nucleic acid molecule. In some embodiments, the nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof. In some embodiments, the nucleic acid molecule comprises a hexonucleic acid (HNA). In some embodiments, greater than 70% of nucleotides of the nucleic acid molecule are thymines or cytosines. In some embodiments, the nucleic acid molecule comprises a priming site, a sequencing site, a unique molecular identifier, a barcode sequence, a cleavage site, or a combination thereof. In some embodiments, the method further comprises, prior to (a), generating the peptide comprising the non-naturally occurring modification. In some embodiments, the generating comprises alkylating or acetylating an amino acid residue of the peptide. In some embodiments, the generating comprises beta-elimination of a phosphate group and addition of a thiol group. In some embodiments, the generating comprises using phenylisothiocyanate or acetic anhydride. In some embodiments, the non-naturally occurring modification is on an N-terminal or C-terminal amino acid of the peptide. In some embodiments, (c) is performed using a protease that recognizes the non-naturally occurring modification. In some embodiments, the coupling in (b) comprises coupling a C-terminal amino acid to the substrate. In some embodiments, the method further comprises, generating the peptide comprising the non-naturally occurring modification. In some embodiments, the generating comprises subjecting the peptide to conditions sufficient to block all carboxyl groups. In some embodiments, the coupling of the C-terminal amino acid is performed using a photoredox reaction or an enzymatic reaction. In some embodiments, the enzymatic reaction comprises carboxypeptidase or amidase. In some embodiments, the coupling in (b) comprises coupling a N-terminal amino acid to the substrate. In some embodiments, the linker is a bifunctional linker. In some embodiments, the linker comprises a first reactive group and a second reactive group, wherein the first reactive group is capable of coupling to the amino acid and wherein the second reactive group is capable of coupling to the substrate. In some embodiments, the amino acid is a terminal amino acid. In some embodiments, the first reactive group comprises a thiocyanate, aldehyde group, dansyl chloride, or fluorobenzene. In some embodiments, the thiocyanate comprises a phenylisothiocyanate (PITC) moiety and wherein the coupling of (b) comprises coupling the PITC moiety to the amino acid to generate a phenylthiocarbamoyl (PTC) derivative of the amino acid. In some embodiments, the method further comprises, subsequent to (c), derivatizing the PTC derivative to generate a thiazolinone (ATZ) derivative or a phenylthiohydantoin (PTH)-amino acid derivative. In some embodiments, the cleaving in (c) is performed by applying a stimulus. In some embodiments, the stimulus comprises a change in pH. In some embodiments, the change in pH comprises the use of an acid. In some embodiments, the change in pH comprises the use of a base. In some embodiments, the stimulus comprises the use of a Lewis acid. In some embodiments, the Lewis acid comprises boron triflate (BF₃), boron trifluoride etherate, boron trichloride, boron tribromide, boron triiodide, or scandium triflate. In some embodiments, the stimulus comprises electromagnetic radiation. In some embodiments, the electromagnetic radiation is applied using a microwave. In some embodiments, the substrate comprises a capture moiety that comprises a third reactive group that is capable of reacting with the second reactive group. In some embodiments, the first reactive group, the second reactive group, or the third reactive group comprises a click chemistry moiety. In some embodiments, the second reactive group and the third reactive group comprise a click chemistry pair. In some embodiments, the linker comprises an amino acid reactive group and a polymerizable molecule, wherein the polymerizable molecule is configured to couple to the substrate. In some embodiments, the polymerizable molecule comprises an enzyme recognition site. In some embodiments, the enzyme recognition site is recognized by a nuclease. In some embodiments, the nuclease is a restriction enzyme, a Cas protein, or an Argonaut nuclease. In some embodiments, the method further comprises contacting the removed substrate-coupled amino acid with a binding agent. In some embodiments, the binding agent comprises an antibody, antibody fragment, aptamer, scFv, nanobody, anticalin, tRNA-acyl synthetase, peptide, peptide mimetic, a polysaccharide, or derivative thereof. In some embodiments, the binding agent comprises an enzyme. In some embodiments, the enzyme comprises a metalloprotease, aminopeptidase, or exopeptidase. In some embodiments, the substrate is a solid support. In some embodiments, the substrate is substantially planar. In some embodiments, the substrate is a bead or particle. In some embodiments, the peptide is coupled to the support via a click chemistry moiety. In some embodiments, the peptide is coupled to the support via a functional group. In some embodiments, the functional group is added to the peptide, the capture moiety, or the second polymerizable molecule using an enzyme. In some embodiments, the enzyme comprises an amidase. In some embodiments, the peptide is coupled to the support via a linker molecule. In some embodiments, the substrate is functionalized with a functional group, and further comprising, attaching the peptide to the functional group. In some embodiments, the method further comprises passivating the substrate. In some embodiments, the cleaving in (c) comprises applying a stimulus. In some embodiments, the stimulus comprises a change in pH. In some embodiments, the stimulus comprises the use of a Lewis acid. In some embodiments, the Lewis acid comprises boron triflate (BF₃), boron trifluoride etherate, boron trichloride, boron tribromide, boron triiodide, or scandium triflate. In some embodiments, the stimulus comprises electromagnetic radiation. In some embodiments, the electromagnetic radiation is applied using a microwave. In some embodiments, the stimulus is a biological stimulus. In some embodiments, the biological stimulus comprises use of an enzyme. In some embodiments, the enzyme is a metalloprotease, aminopeptidase, or exopeptidase. In some embodiments, the method further comprises providing a metal catalyst. In some embodiments, the amino acid comprises a modification, and wherein the enzyme recognizes the modification. In some embodiments, the method further comprises blocking a carboxyl group or an amine group of the peptide. In some embodiments, the method further comprises generating the peptide comprising the non-naturally occurring modification. In some embodiments, the generating comprises alkylating or acetylating the at least one amino acid, beta-elimination of a phosphate group, or use of PITC or acetic anhydride. In some embodiments, the generating comprises converting a cysteine residue to cysteic acid. In some embodiments, the generating comprises use of an oxidizing or reducing agent. In some embodiments, the peptide is derived from a biological sample.

In another aspect, provided herein is a method for sequencing a peptide, comprising: (a) providing a substrate comprising the peptide and a nucleic acid molecule; (b) providing a linker, wherein the linker comprises an amino acid reactive group and a substrate-tethering moiety; (c) coupling the linker to a terminal amino acid of the peptide and to the substrate; (d) cleaving the terminal amino acid, thereby providing a cleaved amino acid-linker complex coupled to the substrate; (e) contacting the cleaved amino acid-linker complex with a binding agent comprising an activating agent; (f) providing a nucleic acid barcode molecule; and (g) using the activating agent to couple the nucleic acid barcode molecule to the nucleic acid molecule.

In some embodiments, the substrate comprises a capture moiety, wherein the substrate-tethering moiety is configured to couple to the capture moiety. In some embodiments, the capture moiety comprises an additional nucleic acid molecule, wherein the linker comprises a linking nucleic acid molecule, and wherein, in (c), the linking nucleic acid molecule couples to the additional nucleic acid molecule. In some embodiments, the capture moiety comprises a first click chemistry moiety and wherein the linker comprises a second click chemistry moiety, and wherein the coupling in (c) comprises reacting the first click chemistry moiety with the second click chemistry moiety. In some embodiments, the activating agent comprises an activating nucleic acid molecule, and further comprising, coupling the nucleic acid barcode molecule to the activating polymerizable molecule. In some embodiments, the coupling is mediated using a splint molecule comprising a sequence complementary to at least a portion of (i) the nucleic acid barcode molecule, (ii) the nucleic acid molecule and (iii) the activating nucleic acid molecule. In some embodiments, the method further comprises generating the nucleic acid barcode molecule via hybridization chain reaction (HCR). In some embodiments, the generating comprises (A) providing a first hairpin molecule and a second hairpin molecule; (B) coupling the first hairpin molecule to the activating agent; and (C) coupling the second hairpin molecule to the first hairpin molecule, thereby generating the nucleic acid barcode molecule. In some embodiments, the activating agent comprises an activating nucleic acid molecule. In some embodiments, subsequent to (C), the nucleic acid barcode molecule comprises a flap sequence, and wherein (g) is mediated using the flap sequence to couple the nucleic acid barcode molecule to the nucleic acid molecule. In some embodiments, the nucleic acid barcode molecule comprises the first hairpin molecule or the second hairpin molecule. In some embodiments, the activating agent comprises an anchoring nucleic acid molecule, wherein the anchoring nucleic acid molecule is configured to couple to an additional nucleic acid molecule adjacent to the nucleic acid molecule. In some embodiments, the nucleic acid barcode molecule is coupled to the binding agent. In some embodiments, the activating agent comprises an enzyme selected from the group consisting of a peroxidase, a Cas protein, a ligase, a kinase, and a restriction enzyme. In some embodiments, the enzyme is a kinase, and further comprising, using the kinase to phosphorylate the nucleic acid molecule, thereby allowing coupling of the nucleic acid barcode molecule to the nucleic acid molecule. In some embodiments, the enzyme is a horseradish peroxidase (HRP) or an ascorbate peroxidase (APEX), wherein the HRP or the APEX activates the nucleic acid barcode molecule or the nucleic acid molecule, thereby coupling the nucleic acid barcode molecule to the nucleic acid molecule. In some embodiments, the enzyme is a restriction enzyme and wherein the nucleic acid molecule comprises a restriction recognition site, and further comprising, cleaving the nucleic acid molecule with the restriction enzyme prior to the coupling of the nucleic acid barcode molecule to the nucleic acid molecule. In some embodiments, at least one amino acid of the peptide comprises a post-translational modification. In some embodiments, the coupling in (c) is mediated by a linker.

In another aspect, provided herein is a method for characterizing a plurality of analytes, comprising: (a) providing a first analyte, a second analyte, a first binding agent capable of coupling to the first analyte and not the second analyte, and a second binding agent capable of coupling to both the first analyte and the second analyte; (b) contacting the first analyte and the second analyte with the first binding agent; (c) subsequent to (b), contacting the first analyte and the second analyte with the second binding agent; (d) identifying the first binding agent and the second binding agent; and (e) using the first binding agent and the second binding agent identified in (d) to identify the first analyte and the second analyte.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A schematically shows an example workflow for processing and analyzing a polymeric analyte in a highly accurate manner. FIG. 1B schematically shows another example workflow for analyzing a polymeric analyte using an activating polymerizable molecule. FIG. 1C schematically shows another example workflow for analyzing a polymeric analyte using an activating polymerizable molecule. FIG. 1D schematically shows another example workflow for analyzing a plurality of monomers of a polymeric analyte. FIG. 1E schematically shows an example activating agent for coupling of polymerizable molecules for analyzing a polymeric analyte. FIG. 1F schematically shows another example of an activating agent for coupling of polymerizable molecules for analyzing a polymeric analyte.

FIG. 2 shows an example of a linker that can be used to couple to a polymerizable molecule and a monomer of a polymeric analyte, which can be used in a workflow for sequencing a polymeric analyte, as described herein.

FIG. 3 shows an example workflow of using a library of binding agents having varying monomeric specificities.

FIG. 4 schematically shows an example of modifying a monomer of a polymeric analyte.

FIG. 5 schematically shows examples of modifying cleaved monomers to prevent coupling of a binding agent.

FIG. 6 schematically shows an example workflow for modifying a cleaved monomer to prevent coupling of a binding agent.

FIG. 7 schematically shows another example of modifications to cleaved monomers to prevent coupling of a binding agent.

FIG. 8 schematically shows example approaches for controlling the distance between polymerizable molecules as described herein.

FIG. 9 schematically shows approaches for coupling amino acid residues to polymerizable molecules.

FIG. 10 schematically shows an approach for barcoding peptides using a bridge amplification approach.

FIG. 11 schematically shows an example workflow for processing a sample comprising cells by partitioning the cells into individual partitions, releasing the proteins contained within the cells, and barcoding the proteins.

FIG. 12 schematically shows an example workflow for partitioning cells and barcoding the proteins or peptides of the cell.

FIG. 13 schematically shows an example workflow for spatial barcoding of proteins within tissue samples.

FIG. 14 schematically shows another example workflow for spatial barcoding of proteins within tissue samples.

FIG. 15 schematically shows another example workflow for spatial barcoding of proteins within tissue samples.

FIG. 16A schematically shows an example model system using nucleic acid molecules for demonstrating coupling of a monomer to a capture moiety. FIG. 16B shows example data of coupling of a first nucleic acid molecule to a second nucleic acid molecule, representative of coupling of a monomer to a capture moiety, across varying substrate types. FIG. 16C schematically shows a substrate comprising linkers for coupling nucleic acid molecules and example data of coupling of a first nucleic acid molecule to a second nucleic acid molecule, representative of coupling of a monomer to a capture moiety across the substrates. FIG. 16D shows example data of a cleavage event of the first nucleic acid molecule from the second nucleic acid molecule, representative of cleaving of a monomer from a capture moiety.

FIG. 17A shows an example experimental workflow to investigate the effects of Edman degradation treatment on a cleavage event of a first nucleic acid molecule from a second nucleic acid molecule. FIG. 17B shows example data of the experimental workflow of FIG. 17A.

FIG. 18A shows an example approach for coupling a linker with an amino acid of a peptide to generate a linker-peptide complex. FIG. 18B shows an example alternative Edman degradation approach for cleaving a terminal amino acid from a peptide and the efficiency of such an approach. FIG. 18C shows an additional example alternative Edman degradation approach for cleaving a terminal amino acid from a peptide and the efficiency of such an approach. FIG. 18D shows an example approach for cleaving a terminal amino acid from a peptide and a different quantitation of the efficiency of such an approach. FIG. 18E shows cleavage efficiency of peptides cleaved with an Edman degradation approach. FIG. 18F shows another example approach for cleaving a terminal amino acid from a peptide. FIG. 18G shows the cleavage efficiency of peptides cleaved with an Edman degradation approach and two non-standard Edman degradation approaches.

FIG. 19 shows example data of the stability of nucleic acid molecules in an alternative Edman degradation approach.

FIG. 20 schematically shows an example of a sequencing approach to analyze or characterize polymeric analytes. FIG. 20 discloses SEQ ID NO: 19.

FIG. 21 shows a computer system that is programmed or otherwise configured to implement methods provided herein.

FIG. 22A shows a schematic of a substrate-polymeric analyte-linker complex (e.g., bead-peptide-linking nucleic acid molecule) used as a model input for validation studies of local tethering via coupling of a monomer to a capture moiety, cleavage, and detection of the monomer (e.g., N-terminal amino acid). FIG. 22B shows example data of local tethering efficiency. FIG. 22C shows example data of cleavage efficiency. FIG. 22D shows a schematic of detection of monomers using a binding agent across two rounds of local tethering, cleavage, and detection. FIG. 22E shows example data of a first round of detection. FIG. 22F shows example data of a second round of detection. FIG. 22A discloses SEQ ID NOS 20-22, 17 and 29, respectively, in order of appearance.

FIG. 23 shows example data of the stacking efficiency of a second nucleic acid barcode molecule onto a first nucleic acid barcode molecule.

FIG. 24 schematically shows a linker comprising a linking nucleic acid molecule that comprises a self-splinting region.

FIG. 25A shows example sequences for generating a polymerizable molecule using hybridization chain reaction. FIG. 25A discloses SEQ ID NOS 12-15 and 27-28, respectively, in order of appearance. FIG. 25B shows another example of generating a polymerizable molecule comprising a flap sequence using hybridization chain reaction. FIG. 25B discloses SEQ ID NOS 24-26, 24-26, 24-26, 23, 12, 12 and 12, respectively, in order of appearance. FIG. 25C shows example data of the hybridization chain reaction. FIG. 25D shows another example of generating a polymerizable molecule comprising a flap sequence using hybridization chain reaction. FIG. 25D discloses SEQ ID NOS 16, 18, 16, 18, 16, 18, 23, 12, 12 and 12, respectively, in order of appearance.

FIG. 25E shows example data of the hybridization chain reaction. FIG. 25F show an example of coupling of a polymerizable molecule generated from hybridization chain reaction to a substrate-bound capture moiety. FIG. 25F discloses SEQ ID NOS 17, 17, 17, 30, 17, 16, 18, 16, 18, 16, 18, 23, 12, 12 and 12, respectively, in order of appearance. FIG. 25G shows example data of the coupling of the polymerizable molecule to the substrate-bound capture moiety.

FIG. 26A schematically shows a workflow for detecting a monomeric analyte using a binding agent comprising an enzyme activating agent. FIG. 26B schematically shows an example monomeric analyte. FIG. 26C shows example data of the coupling efficiency of an enzyme substrate using the enzyme activating agent. FIG. 26D shows example data of absorbance as a measure of the coupling efficiency. FIG. 26E shows example data of fluorescence as a measure of the coupling efficiency. FIG. 26F shows additional example data of fluorescence as a measure of the coupling efficiency.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

As used herein, the term “protein” generally refers to a molecule comprising two or more amino acids joined by a peptide bond. A protein may also be referred to as a “polypeptide”, “oligopeptide”, or “peptide”. A protein can be a naturally occurring molecule, or a synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications or by chemical modification. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “peptide” may refer to any short, single peptide chain. A peptide may be no more than about 100, 95, 90, 85, 80, 75, 70, 65, 60, 55, 50, 45, 40, 35, 30, 25, 20, 15, 10, 5, or less than about 5 amino acids in length. A peptide may have a known or unknown biological function or activity. Peptides can include natural, synthetic, modified, or degraded proteins or peptides, or a combination thereof.

As used herein, “polypeptide” refers to two or more amino acids linked together by a peptide bond. The term “polypeptide” includes proteins that have a C-terminal end and an N-terminal end as generally known in the art and may be synthetic in origin or naturally occurring. As used herein “at least a portion of the polypeptide” refers to 2 or more amino acids of the polypeptide. A polypeptide may comprise one or more peptides. Optionally, a portion of the polypeptide includes at least: 1, 5, 10, 20, 30 or 50 amino acids, either consecutive or with gaps, of the complete amino acid sequence of the polypeptide, or the full amino acid sequence of the polypeptide.

As used herein, the term “sample” refers to a collected substance or material that comprises or is suspected to comprise one or more analytes of interest (e.g., biomolecules, e.g., polypeptides). A sample may be modified for purposes such as storage or stability. A sample may be naturally occurring or synthetic. A sample may be processed to separate or remove unwanted fractions or impurities from the analyte(s) of interest. A sample may be enriched or purified. For example, a sample may comprise a fraction of a separation process (e.g., chromatography, fractionation, electrophoresis, etc.). Alternatively, a sample may not be subjected to processing that separates or removes any unwanted fractions or impurities from the analyte(s) of interest. A sample may be obtained from any suitable source or location, including from organisms, cells, tissues, cell preparations, cell-free compositions, the environment (e.g., air, water, dirt, soil, agriculture, soil, dust, sewage). A sample may be obtained from an organism or part of an organism, such as from a fluid, tissue, or cell. A sample may include biological and/or non-biological components. As used herein, the terms “biological sample” or “biological source” refer to a sample that is derived from a predominantly biological system or organism, such as one or more viral particles, cells (e.g. individualized cells), organelles (e.g. individualized organelles), tissues, bodily fluids, bone, cartilage, and exoskeleton. A biological sample may comprise a majority of biological material on a mass basis, excluding the weight of fluid within the sample. Biological samples may comprise one or more proteins, referred to herein as protein samples. Biological samples can be acquired from various sources, e.g., from a clinical patient sample, such as blood, serum, plasma, Cerebral Spinal Fluid (CSF), saliva, mucosal secretions, sputum, urine, lymph, perspiration, vaginal fluid, semen, fecal matter, amniotic fluid, perspiration, synovial fluid, etc. A biological sample may be processed to purify and retain one or more biomolecules (e.g., proteins, nucleic acids, carbohydrates, lipids, glycoproteins, lipoproteins, metabolites, etc.) from the biological sample. A biological sample (e.g., a protein sample) may be derived from cultured cells, which may be treated or untreated. A biological sample (e.g., a protein sample) can also result from tissue specimens, such as biopsy samples, which may optionally be processed to liberate biomolecules (e.g., proteins) contained therein. Tissue samples may also be derived from in vivo specimens, including fresh, frozen, acute, and fixed tissues.

As used herein, the terms “antibody” and “immunoglobulin” may generally refer to proteins that can recognize and bind to a specific antigen. An antibody or immunoglobulin may refer to an antibody isotype, fragments of antibodies including, but not limited to, Fab, Fv, scFv, and Fd fragments, chimeric antibodies, humanized antibodies, single-chain antibodies, and fusion proteins including an antigen-binding portion of an antibody and a non-antibody protein. The antibodies may be detectably labeled, e.g., with a fluorophore, radioisotope, enzyme (e.g., a peroxidase) or ribozyme or DNAzyme which generates a detectable product, fluorescent protein, nucleic acid barcode sequence, and the like. The antibodies may be further conjugated to other moieties, such as members of specific binding pairs, e.g., biotin (member of biotin-avidin specific binding pair), and the like. Also encompassed by the terms are nanobodies, Fab′, Fv, F(ab′)2, scFv, and other antibody fragments that retain specific binding to antigen. Antibodies may exist in a variety of other forms including, for example, Fv, Fab, and (Fab)2, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., Immunology, Benjamin, N.Y., 2^{nd} ed. (1984), and Hunkapiller and Hood, Nature, 323, 15-16 (1986), which are herein incorporated by reference). Naturally occurring immunoglobulins or antibody types include immunoglobulin A, immunoglobulin G, immunoglobulin D, immunoglobulin E, immunoglobulin M, or other immunoreactive components.

“Binding” or “coupling” as used herein generally refers to a covalent or non-covalent interaction between two molecules (referred to herein as “binding partners”, e.g., a substrate and an enzyme or an antibody and an epitope). Binding between binding partners may be specific or non-specific. Binding or coupling of a molecule to another molecule may occur through hydrogen bonding, electrostatic interaction, ionic bonding, van der Waals forces, metallic bonds, molecular bonds, or any useful interaction.

As used herein, “specifically binds” or “binds specifically” generally refers to an interaction between binding partners (e.g., a binding partner and a cognate molecule) such that the binding partners bind to one another, but do not bind to other molecules that may be present in the environment (e.g., in a biological sample, in tissue, in an in vitro assay) under a set of conditions. A specific binding interaction may entail a binding partner that binds to a cognate molecule. The specific binding interaction may entail the binding of the binding partner to its cognate molecule at a significantly or substantially higher level or with greater affinity as compared to the binding of the binding partner to a non-cognate molecule. A specific binding interaction may entail a first binding partner that has greater selectivity of binding to the cognate molecule as compared to a non-cognate molecule.

The terms “nucleic acid”, “nucleic acid molecule”, “oligonucleotide” and “polynucleotide” may be used interchangeably herein and generally refer to a polymeric form of naturally occurring or synthetic nucleotides, or analogs thereof, of any length. A nucleic acid molecule may comprise one or more deoxyribonucleotides, deoxynucleotide triphosphates, dideoxynucleotide triphosphates, ribonucleotides, hexitol nucleotides, cyclohexane nucleotides, or analogs or combinations thereof. A nucleic acid molecule may comprise, e.g., DNA, RNA, HNA, CeNA, and modified forms thereof. A nucleic acid molecule may comprise nucleotides that are linked by phosphodiester bonds. A nucleic acid molecule may have any two- or three-dimensional structure, and may perform any function, known or unknown. A nucleic acid molecule may be single stranded, double stranded, or partially double stranded. Non-limiting examples of polynucleotides include a gene, a gene fragment, exons, introns, messenger RNA (mRNA), transfer RNA, ribosomal RNA, noncoding RNA, small interfering RNA, short hairpin RNA, micro RNA, scaRNA, ribozymes, riboswitches, viral RNA, complementary DNA (cDNA), cosmid DNA, mitochondrial DNA, chromosomal or genomic DNA, viral DNA, recombinant polynucleotides, branched polynucleotides, plasmids, vectors, isolated DNA of any sequence, control regions, isolated RNA of any sequence, nucleic acid probes, nucleic acid adapters, and primers. The nucleic acid molecule may be linear, circular, or any other geometry. Examples of polynucleotide analogs include but are not limited to xeno nucleic acid (XNA), bridged nucleic acid (BNA), glycol nucleic acid (GNA), hexitol nucleic acid (HNA), cyclohexane nucleic acid (CeNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. A nucleic acid molecule according to the present invention may include any polymer or oligomer of nucleotides such as pyrimidine and purine bases, such as cytosine, thymine, and uracil, and adenine and guanine, respectively and combinations thereof. The nucleic acid molecule may comprise any deoxyribonucleotide, ribonucleotide, hexitol-nucleotide, cyclohexane-nucleotide, peptide nucleic acid component, and any chemical variants thereof, such as methylated, 7-deaza purine analogs, 8-halopurine analogs, hydroxymethylated or glycosylated forms of these bases, and the like. The polymers or oligomers may be heterogeneous or homogenous in composition and may be isolated from naturally occurring sources or may be artificially or synthetically produced. A nucleic acid molecule may comprise DNA, RNA, HNA, CeNA or a mixture thereof, and may exist permanently or transitionally in single-stranded or double-stranded form, including homoduplex, heteroduplex, and hybrid states.

As used herein, the term “amino acid” generally refers to an organic compound that combines to form a protein or peptide. An amino acid generally comprises an amine group, a carboxylic acid group, and a side-chain specific to each amino acid, which serve as a monomeric subunit of a peptide. An amino acid may include the 20 standard, naturally occurring or canonical amino acids as well as non-standard amino acids. The standard, naturally-occurring or canonical amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, (3-amino acids, Homo-amino acids, Proline and Pyruvic acid derivatives, 3-substituted alanine derivatives, glycine derivatives, ring-substituted phenylalanine and tyrosine derivatives, linear core amino acids, and N-methyl amino acids.

As used herein, the term “post-translational modification” refers to modifications that occur on a peptide subsequent to translation. A post-translational modification may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, transglutamination, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, sumoylation, disulfide bond formation, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C1-C4 alkyl). A post-translational modification also includes modifications, such as but not limited to those described above, of amino acids falling between the amino and carboxy termini. The term post-translational modification can also include peptide modifications that include one or more detectable labels. A post-translational modification may be naturally occurring or synthetic.

As used herein, the term “binding agent” refers to a molecule, e.g., a nucleic acid molecule, a peptide, a polypeptide, a protein, carbohydrate, a synthetic molecule, or a small molecule that binds to, associates with, unites with, recognizes, or combines with another molecule. The binding agent may bind to a macromolecule or a component or feature of a macromolecule. A binding agent may form a covalent association or non-covalent association with a molecule, a macromolecule, or a component or feature of a macromolecule. A binding agent may also be a chimeric binding agent, composed of two or more types of molecules, such as a nucleic acid molecule-peptide chimeric binding agent, a carbohydrate-peptide chimeric binding agent, or a lipid-peptide chimeric binding agent. A binding agent may be a naturally occurring, synthetically produced, or recombinantly expressed molecule. A binding agent may bind to a single monomer or subunit of a polymeric analyte, such as a macromolecule (e.g., a single amino acid of a peptide) or bind to a plurality of linked subunits of a macromolecule (e.g., a di-peptide, tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypeptide, or protein, or bind to a conformational peptide, polypeptide, or protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferentially bind to a chemically modified or labeled amino acid over a non-modified or unlabeled amino acid. For example, a binding agent may preferentially bind to an amino acid that has been modified with an acetyl moiety, guanyl moiety, dansyl moiety, isothiocyanate moiety, 2,4-dinitrophenol (DNP) moiety, SNP moiety, etc., over an amino acid that does not possess such a moiety. A binding agent may bind to a post-translational modification, either naturally occurring or synthetic, of a peptide molecule. A binding agent may exhibit selective binding to a component or feature of a macromolecule (e.g., a binding agent may selectively bind to one of the 20 possible natural amino acid residues and with bind with very low affinity or not at all to the other 19 natural amino acid residues). A binding agent may exhibit less selective binding, where the binding agent is capable of binding a plurality of components or features of a macromolecule (e.g., a binding agent may bind with similar affinity to two or more different amino acid residues). A binding agent may comprise a tag, which may be coupled to the binding agent via a linker.

As used herein, the term “linker” generally refers to a molecule or moiety that is involved in joining two or more molecules. A linker may facilitate a covalent or noncovalent interaction of two or more molecules. A linker may be a crosslinker. The linker can be unifunctional, bifunctional, trifunctional, quadrifunctional, or polyfunctional. A linker can be or comprise a nucleotide, a nucleotide analog, an amino acid, a peptide, a polypeptide, or a non-nucleotide chemical moiety, such as an organic or inorganic compound. A linker may comprise a polymer, such as a polyethylene glycol (PEG), poly-L-lysine (PLL), poly (DL-lactic acid) (PLA), poly (DL-lactide-co-glycoside) (PLGA), polyornithine, polyarginine, etc. A linker may comprise one or more reactive ends, e.g., an amine-reactive group, a carboxyl-reactive group, a sulfhydryl-reactive group, a hydroxyl-reactive group, etc. In some examples, a linker may be used to join different molecule types, e.g., different biomolecule types such as a peptide with a nucleic acid molecule, a lipid with a peptide, a carbohydrate with a peptide, etc.; non-biomolecule types; or a biomolecule to a non-biomolecule. For example, a linker may be used to join a binding agent with a tag, a tag with a macromolecule (e.g., peptide, nucleic acid molecule), a macromolecule with a solid support, a tag with a solid support, etc. A linker may join two molecules via enzymatic reaction or chemistry reaction. A linker may comprise one or more click chemistry moieties. A linker may join more than two molecules, e.g., via enzymatic or chemical reactions.

The term “conjugated” as used herein generally refers to a covalent or ionic interaction between two entities, e.g., molecules, compounds, or combinations thereof.

As used herein, the term “tag” generally refers to a molecule or moiety that is conjugated to a molecule. A tag may comprise a detectable label, e.g., a fluorophore or fluorescent protein, a radioactive isotope, an enzyme (e.g., a chromogenic or fluorescent protein, proteins that can catalyze chromogenic substrates) or ribozyme or DNAzyme, a mass tag, a metal, a hapten (e.g., biotin, digoxigenin, urushiol, fluorescein), a vibrational or FTIR tag (e.g., alkyne group). A tag may comprise a biomolecule, such as a nucleic acid molecule, a protein, a lipid, a carbohydrate, or a combination thereof. A tag may comprise one or more nucleic acid molecules, which may optionally encode information regarding the tag or the molecule onto which a tag is conjugated (e.g., a binding agent, such as an antibody). For example, a tag may comprise a nucleic acid barcode molecule. A tag may comprise an organic compound or an inorganic compound.

As used herein, the term “barcode” generally refers to an identifying feature that may be used to distinguish similar items. A barcode may comprise a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 bases), which may provide a unique identifier tag or origin information for a molecule (e.g., protein, polypeptide, peptide), a binding agent, a set of binding agents from a binding cycle, a sample molecule, a set of samples, molecules within a compartment (e.g., droplet, bead, partition or separated location), macromolecules within a set of compartments, a fraction of macromolecules, a set of macromolecule fractions, a spatial region or set of spatial regions, a library of macromolecules, or a library of binding agents. A barcode can be an artificial sequence or a naturally occurring sequence including peptides, proteins, protein complexes, carbohydrates, and synthetic polymeric materials. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. A population of barcodes may comprise error correcting barcodes. Barcodes can be used to computationally deconvolute sequence reads derived from an individual molecule, sample, library, etc. Barcodes may comprise multiplexed information, e.g., arising from different samples, compartments, individual molecules, etc. A barcode can also be used for deconvolution of a collection of molecules that have been distributed into small compartments for enhanced mapping. For example, rather than mapping a peptide back to the proteome, the peptide can be mapped back to its originating protein molecule or protein complex. A barcode may comprise any useful structure moiety or motif, e.g., hairpins, loop sequences, or spacers. Barcodes can comprise artificial or modified nucleic acids, e.g., locked nucleic acids (LNA), protein nucleic acids (PNA), hexitol nucleic acids (HNA), cyclohexane nucleic acids (CeNA), or a combination thereof. Barcodes may comprise or be generated using a protein, e.g., Tal effector, Cas protein (e.g., Cas9), Argonaut, or coiled coils.

As used herein, a “sample barcode”, also referred to as “sample tag” generally refers to a barcode molecule comprising identifying information of a sample from which a barcoded molecule derives.

As used herein, a “spatial barcode” generally refers to a barcode molecule comprising identifying information of a region of a 2-D or 3-D sample (e.g., a tissue section) from which a molecule originates or is derived. Spatial barcodes may be used for molecular pathology on tissue sections. A spatial barcode may allow for multiplex sequencing of a plurality of samples or libraries from tissue section(s).

As used herein, a “temporal barcode” generally refers to a barcode molecule comprising time-based information relating to the barcoded molecule. The types of time-based data encoded in a temporal barcode can include information such as a life-time of a barcoded molecule, a time of collection of a sample, a time or duration since the beginning of an experiment or induction with a stimulus, information on the age of a cell or tissue, a sequence of interactions between molecules, a cycle number among an iterative process, among others. It is possible for different types of barcodes (e.g., spatial, temporal, cell-specific) to be combined in one multiplexed barcode.

As used herein, the term “nucleic acid sequence” or “oligonucleotide sequence” generally refers to a contiguous string of nucleotide bases and may refer to the particular placement of nucleotide bases in relation to each other as they appear in an oligonucleotide. Similarly, the term “polypeptide sequence” or “amino acid sequence” refers to a contiguous string of amino acids and may refer to the particular placement of amino acids in relation to each other as they appear in a polypeptide.

The terms “complementary” or “complementarity” refer to polynucleotides (i.e., a sequence of nucleotides) related by base-pairing rules. For example, the sequence “5′-AGT-3′,” is complementary to the sequence “5′-ACT-3′”. Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, GC content, stringency of the conditions involved, and the melting temperature of the formed hybrid. Hybridization methods involve the annealing of one nucleic acid to another, complementary nucleic acid, e.g., based on Watson-Crick base pairing.

As used herein, the term “proteomics” generally refers to quantitative and/or qualitative analysis of the proteome within a sample, such as biological sample, e.g., from cells, tissues, or bodily fluids. Proteomics may include the analysis of spatial distributions of proteins within a sample (e.g., cell and/or tissues). Proteomics may include studies of the dynamic state of the proteome, e.g., how one or more proteins change in time. A proteome may comprise multiple “-omes”, e.g., a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof.

The terminal amino acid at one end of the peptide chain that has a free amino group may be referred to herein as the “N-terminal amino acid” (NTAA). The terminal amino acid at the other end of the chain that has a free carboxyl group may be referred to herein as the “C-terminal amino acid” (CTAA). The amino acids making up a peptide may be numbered in order, with the peptide being “n” amino acids in length. As used herein, in some instances, NTAA may be considered the nth amino acid (also referred to herein as the “n NTAA”). In such cases, the next amino acid is the n-1 amino acid, then the n-2 amino acid, and so on down the length of the peptide from the N-terminal end to C-terminal end. Alternatively, CTAA may be considered the nth amino acid (also referred to herein as the “n CTAA”). In such cases, the next amino acid is the n-1, then the n-2 amino acid, and so on down the length of the peptide from the C-terminal end to N-terminal end. An NTAA, CTAA, or both may be modified or labeled with a chemical moiety.

As used herein, the terms “determining,” “measuring,” “assessing,” and “assaying” are used interchangeably and include both quantitative and qualitative determinations.

As used herein, the term “unique molecular identifier” or “UMI” generally refers to a molecule barcode comprising indexing information. A UMI may comprise a nucleic acid molecule of about 3 to about 150 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 bases) in length. A UMI may provide a unique identifier tag for each molecule (e.g., peptide, binding agent, a nucleic acid molecule) that comprises or is coupled to a UMI. A UMI may comprise a random sequence (e.g. a random N-mer). A UMI may be used to count the number of molecules in a given cell, partition, sample, etc., e.g., in situations where the given cell, partition, etc. comprises a common barcode sequence. For example, a partition may comprise a plurality of barcode molecules comprising a common barcode sequence, where each barcode molecule has a unique molecular identifier sequence. The common barcode sequence can be used to attribute all the barcoded molecules to the same given cell, partition, sample, etc., whereas the unique molecular identifier may be used to analyze the number of each originating molecules (that are barcoded) present in the cell, partition, etc.

As used herein, a “derivative” of a nucleic acid molecule generally refers to a nucleic acid molecule that is derived from an originating nucleic acid molecule. The derivative may have the same or substantially the same nucleotide sequence as the originating nucleic acid molecule, or the derivative may comprise a complement, partial complement, reverse complement, or partial reverse complement as the originating nucleic acid molecule. A derivative may be the same type of nucleic acid (e.g., DNA or RNA) as the originating nucleic acid molecule, or the derivative may be a different type of nucleic acid (e.g., cDNA generated from an RNA molecule). A nucleic acid molecule derivative may display sequence identity or complementarity as the originating nucleic acid molecule. The derivative nucleic acid molecule may also be subjected to additional processing from the originating nucleic acid molecule, e.g., chemical or enzymatic modification, splicing, ligation, polymerization, amplification, fragmentation, tagmentation (e.g., using a transposase), digestion, etc.

A derivative polypeptide or peptide may be derived from an originating polypeptide (or peptide). A derivative may comprise the same amino acid sequence as the originating polypeptide, or the sequence may be different. The derivative polypeptide may result from or be subjected to additional processing from the originating polypeptide, e.g., chemical or enzymatic modification. The derivative polypeptide may comprise one or more tags, nucleic acid molecules, barcode molecules, labels (e.g., detectable labels), fluorophores, probes, linkers, post-translational modifications, chemical protecting groups, or other chemical moieties.

As used herein, the term “compartment” or “partition” generally refers to a physical area or volume that separates or isolates a subset of molecules from a sample of molecules. For example, a compartment may separate an individual cell from other cells, or a subset of a sample's proteome from the rest of the sample's proteome. A compartment may be an aqueous compartment (e.g., microfluidic droplet), a solid compartment (e.g., picotiter well or microtiter well on a plate, tube, vial, gel bead), or a separated region on a surface. A compartment may comprise one or more beads to which macromolecules may be immobilized.

In some embodiments, the partitions as described herein are droplets. The terms “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g., an aqueous phase fluid (e.g., water). In some embodiments, the second fluid phase will be an immiscible phase carrier fluid. Thus, droplets according to the present disclosure may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, ribozymes, DNAzymes, reagents, and a variety of other components. The term droplet may be used to refer to a droplet produced in, on, or by a microfluidic device and/or flowed from or applied by a microfluidic device.

As used herein, the term “carrier fluid” refers to a fluid configured or selected to contain one or more discrete entities, e.g., droplets, as described herein. A carrier fluid may include one or more substances and may have one or more properties, e.g., viscosity, which allow it to be flowed through a microfluidic device or a portion thereof, such as a delivery orifice. In some embodiments, carrier fluids include, for example: oil or water, and may be in a liquid or gas phase.

As used herein, the term “solid support”, “solid surface”, or “solid substrate” or “substrate” refers to any solid material, including porous and non-porous materials, to which a molecule can be associated directly or indirectly. The molecule may be associated with the substrate by covalent or non-covalent interactions, or a combination thereof. A substrate may be two-dimensional (e.g., planar surface) or three-dimensional (e.g., gel matrix or bead). A solid support may comprise, in non-limiting examples, a bead, a microbead, an array, a glass surface, a silicon surface, a plastic surface, a filter, a membrane, nylon or other polymer, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, silica, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, polystyrene bead, a polymer bead, a methylstyrene bead, an agarose bead, an acrylamide bead, a solid core bead, a porous bead, a magnetic or paramagnetic bead, a glass bead, or a controlled pore bead. A bead may be spherical or an irregularly shaped. A bead's size may range from nanometers, e.g., 100 nm, to millimeters, e.g., 1 mm. In certain embodiments, beads range in size from about 0.2 micron to about 200 microns, or from about 0.5 micron to about 5 microns. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μm in diameter. In certain embodiments, “a bead” solid support may refer to an individual bead or a plurality of beads.

As used herein, “sequencing” generally refers to determining the order of: (A) nucleotides (base sequences) in a nucleic acid sample, e.g., DNA or RNA; or determining the order of (B) amino acids in all or part of a polymer, such as a protein, peptide, or other multimeric molecule. Many techniques are available, such as Sanger sequencing or High Throughput Sequencing technologies (HTS). Sanger sequencing may involve sequencing via detection through (capillary) electrophoresis, in which up to 384 capillaries may be sequence analyzed in one run. High throughput sequencing involves the parallel sequencing of thousands or millions or more sequences at once. HTS can be defined as Next Generation sequencing (NGS), i.e. techniques based on solid phase pyrosequencing or as Next-Next Generation sequencing based on single nucleotide real time sequencing (SMRT). HTS technologies are available such as offered by Roche, lllumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, nanopore sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, ThermoFisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “analyzing” a molecule generally refers to quantifying, characterizing, distinguishing, or a combination thereof, all or a portion of the components of a molecule (e.g., a macromolecule, a biological molecule such as a protein, amino acid, nucleic acid molecule, etc.). For example, analyzing a peptide, polypeptide, or protein may comprise determining all or a portion of the amino acid sequence (contiguous or non-continuous) of the peptide. Analyzing a macromolecule may include partial identification of a component of the macromolecule. For example, partial identification of amino acids in a protein sequence can identify an amino acid in the protein as belonging to a subset of possible amino acids. Analysis may be performed sequentially, e.g., beginning with analysis of the n NTAA, and then proceeding to the next amino acid of the peptide (i.e., n-1, n-2, n-3, and so forth). In such instances, sequencing may be performed by cleavage of the n NTAA, thereby converting the n-1 amino acid of the peptide to an N-terminal amino acid (referred to herein as the “n-1 NTAA”). Similarly, analysis of a peptide may begin from C-terminus towards the N-terminus with each round of cleavage from the C-terminus creating a new CTAA. Cleavage of the n CTAA converts the n-1 amino acid of the peptide to a C-terminal amino acid, referred to herein as an “n-1 CTAA”. Analyzing the peptide may also include determining a presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “analyte” generally refers to a substance that is of interest to be further identified, characterized, or measured. An analyte can be, in non-limiting examples, an ion, chemical, compound, small molecule, element, particle, metal, biomolecule (e.g., protein, lipid, carbohydrate, nucleic acid molecules that are existing in nature), macromolecule, metabolite, lipid, carbohydrate, peptide or protein, nucleic acid molecule, organelle, or cell. An analyte may be naturally occurring or synthetic. The analyte may be a solid, semi-solid, liquid, semi-liquid, gas, or plasma. The analyte may be characterized qualitatively or quantitatively. A portion of an analyte may be analyzed. For example, an analyte may be a peptide and the constituent amino acids may be analyzed. The analyte may comprise a polymer, also referred to herein as “polymeric analyte”, which generally refers to an analyte of interest that comprises one or more monomers. A polymeric analyte can be, in non-limiting examples, a group of ions, chemicals, compounds, small molecules, elements, particles, metals, or a biomolecule, macromolecule, metabolite, lipid, carbohydrate, peptide or protein, nucleic acid molecule, organelle, or cell.

As used herein, the term “array” generally refers to a population of molecules that is attached to one or more solid supports such that the molecules at one address can be distinguished from molecules at other addresses. An array can include different molecules that are each located at different addresses on a solid support. Alternatively, an array can include separate solid supports each functioning as an address that bears a different molecule, wherein the different molecules can be identified according to the locations of the solid supports on a surface to which the solid supports are attached, or according to the locations of the solid supports in a liquid such as a fluid stream. The molecules of the array can be, for example, nucleic acids such as SNAPs, polypeptides, proteins, peptides, oligopeptides, enzymes, ribozymes, DNAzymes, ligands, or receptors such as antibodies, functional fragments of antibodies or aptamers. The addresses of an array can optionally be optically observable and, in some configurations, adjacent addresses can be optically distinguishable when detected using a method or apparatus set forth herein.

As used herein, the term “functionalized” refers to any material or substance that has been modified to include a functional group. A functionalized material or substance may be naturally or synthetically functionalized. For example, a polypeptide can be naturally functionalized with a phosphate group, oligosaccharide (e.g., glycosyl, glycosylphosphatidylinositol or phosphoglycosyl), nitrosyl, methyl, acetyl, lipid (e.g., glycosyl phosphatidylinositol, myristoyl or prenyl), ubiquitin or other naturally occurring post-translational modification. A functionalized material or substance may be functionalized for any given purpose, including altering chemical properties (e.g., altering hydrophobicity or changing surface charge density) or altering reactivity (e.g., capable of reacting with a moiety or reagent to form a covalent bond to the moiety or reagent), adding new functions, features, capabilities, or properties of the surface or material. Functionalization of a surface or substrate may be achieved by using, adding, or depositing a linker, as is described elsewhere herein.

As used herein, the term “anchoring group” refers to a molecule or particle that serves as an intermediary attaching a protein or peptide to a surface (e.g., a solid support or a microbead). An anchoring group may be covalently or non-covalently attached to a surface and/or a polypeptide. An anchoring group may be a biomolecule, polymer, particle, nanoparticle, or any other entity that can attach to a surface or polypeptide. In some cases, an anchoring group may be a structured nucleic acid particle.

As used herein, the term “click reaction,” “click chemistry,” “click chemistry reaction,” or “bioorthogonal reaction” refers to single-step, thermodynamically favorable conjugation reaction utilizing biocompatible reagents. A click reaction may utilize no toxic or biologically incompatible reagents (e.g., acids, bases, heavy metals) or generate no toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kilojoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Exemplary bioorthogonal and click reactions are described in detail in WO 2019/195633A1, which is herein incorporated by reference in its entirety. Exemplary click reactions may include metal-catalyzed azide-alkyne cycloaddition, strain-promoted azide-alkyne cycloaddition, strain-promoted azide-nitrone cycloaddition, strained alkene reactions, thiolene reaction, Diels-Alder reaction, inverse electron demand Diels-Alder reaction, [3+2]cycloaddition, [4+1]cycloaddition, nucleophilic substitution, dihydroxylation, thiolyne reaction, photoclick, nitrone dipole cycloaddition, norbornene cycloaddition, oxanobomadiene cycloaddition, tetrazine ligation, and tetrazole photoclick reactions. Exemplary functional groups or reactive handles utilized to perform click reactions may include alkenes, alkynes (including cycloalkynes), azides, epoxides, amines, thiols, nitrones, isonitriles, isocyanides, aziridines, activated esters, and tetrazines.

As used herein, the terms “group” and “moiety” are intended to be synonymous when used in reference to the structure of a molecule. The terms refer to a component or part of the molecule. The terms do not necessarily denote the relative size of the component or part compared to the molecule, unless indicated otherwise. The terms do not necessarily denote the relative size of the component or part compared to any other component or part of the molecule, unless indicated otherwise. A group or moiety can contain one or more atoms.

As used herein, “primers” generally refer to nucleic acid molecules which can prime the synthesis of a nucleic acid molecule (e.g., DNA or RNA). A primer may be single stranded. A primer may comprise one or more recognition sites for a protein (e.g., a polymerizing enzyme, a restriction enzyme, a cleaving enzyme, a nuclease, etc.) to bind to the primer or a primer hybridized to a template strand. A primer may comprise DNA, RNA, or other nucleic acid analogs or noncanonical bases (e.g., spacer moieties, uracils, abasic sites). A primer may optionally comprise any number of functional sequences such as sequencing primer sequences (e.g., P5 or P7 sequences), sequencing primer-binding sequences, read sequences (e.g., R1 or R2 sequences), restriction sites, nuclease-recognition sites, abasic sites, cleavage sites, transposition sites, a barcode sequence, a unique molecular identifier (UMI), etc.

“Amplification” or “amplifying” generally refers to a polynucleotide amplification reaction, namely, a population of polynucleotides that are replicated from one or more starting sequences. Amplifying may refer to a variety of amplification reactions, including but not limited to polymerase chain reaction (PCR), linear polymerase reactions, nucleic acid sequence-based amplification, rolling circle amplification, loop-mediated isothermal amplification, helicase-dependent amplification, multiple displacement amplification, strand invasion based amplification, strand displacement amplification, recombinase polymerase amplification, nicking enzyme amplification reaction, nucleic acid sequence-based amplification, gp32-based amplification, and similar reactions. An amplification reaction may generate an amplicon. An amplification reaction may be performed isothermally or may require temperature changes.

An “adapter” as referred to herein, generally refers to a short nucleic acid molecule (e.g., about 10 to about 100 base pairs in length). An adapter may comprise a short double-stranded DNA molecule. An adapter may be attached, e.g., via polymerization or ligation, to an end of a DNA fragments or amplicons. Adapters may comprise synthetic oligonucleotides, e.g., oligonucleotides that have nucleotide sequences which are at least partially complementary to each other. An adapter may have blunt ends, may have staggered ends (also referred to herein as a 3′ or 5′ “overhang sequence” or “sticky end”, or a blunt end and a staggered end. Adapters may be attached (e.g., via ligation) to fragments to provide an adapter-ligated fragment; the adapter-ligated fragment may serve as a starting point for subsequent manipulation e.g., for amplification or sequencing. An adapter may be functionalized, e.g., conjugated with a tag, probe, detectable label, affinity capture reagent (e.g., biotin or streptavidin).

The term “capture moiety” as used herein generally refers to a molecule that is able to be coupled to another moiety or molecule. A capture moiety can be a biomolecule, e.g., a lipid, carbohydrate, sugar, amino acid, peptide or protein, nucleotide, nucleic acid molecule, metabolite, or a combination thereof (e.g., glycoproteins, lipoproteins, glycosaminoglycans, etc.). A capture moiety can be a small molecule, organic compound, inorganic compound, metal, polymer, ion, or other molecule or molecular compound. A capture moiety may comprise a macromolecule. A capture moiety may comprise an enzyme, ribozyme, DNAzyme, antibody, antibody fragment, nanobody, aptamer, biotin, streptavidin, avidin, neutravidin, or analogs or derivatives thereof. A capture moiety may comprise more than one molecule, e.g., a dimer, trimer, tetramer, pentamer, hexamer, heptamer, octamer, etc. A capture moiety can be a solid substrate or part of a solid substrate, or the capture moiety can be separate from a substrate, e.g., in a fluidic medium (e.g., air, in a liquid solution). A capture moiety may have specificity to a binding partner or a plurality of binding partners. A capture moiety may be able to bind to one molecule or moiety (univalent), or a plurality of molecules or moieties (multivalent). A capture moiety may be or comprise a reactive moiety or group, e.g., a click chemistry moiety.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R). References to these amino acids are also in the form of “[amino acid][residues/residues]” (e.g., lysine residue, lysine residues, leucine residue, leucine residues, etc.).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

### Analysis of Polymeric Analytes Via Local Tethering

The present disclosure provides for novel approaches for processing and analyzing polymeric analytes in a highly-parallelized and accurate manner. Systems and methods of the present disclosure may pertain to molecular analysis and identification of individual monomers of a polymeric analyte, e.g., with single-molecule sensitivity. The systems and methods of the present disclosure may comprise cleaving a monomer from the polymeric analyte, coupling (e.g., via local tethering) of the monomer to a capture moiety (e.g., located adjacent to the polymeric analyte), and contacting the monomer with a binding agent. The binding agent may bind specifically or semi-specifically to the monomer or monomer type and may comprise or be coupled to a polymerizable molecule with encoded information (e.g., on the identity of the binding agent or its cognate molecule), which may be transferred via coupling to and optional copying of the encoded information onto an additional polymerizable molecule. Alternatively, the polymerizable molecule with encoded information may be provided separately from the binding agent (e.g., not coupled to the binding agent), and, in some instances, the binding agent may comprise an activating agent that facilitates the coupling of the polymerizable molecule to the additional polymerizable molecule. The operations may be iterated or repeated any number of times to obtain information on all or a subset of the monomers of the polymeric analyte and optionally, the sequence of the monomers relative to the polymeric analyte. The information may be read out from the polymerizable molecules using, for example, next-generation sequencing approaches. The systems and methods provided herein enable novel approaches to sequencing polymeric analytes, such as peptides, to determine an identity and order of at least a subset of the amino acids of the peptide at a high accuracy for a given number of amino acids. In additional aspects, provided herein are systems and methods for processing an analyte that is less than 10 nanometers (nm) in size, comprising providing the analyte, a binding agent, a first polymerizable molecule, and a second polymerizable molecule, contacting the analyte with the binding agent, and coupling the first polymerizable molecule and the second polymerizable molecule. The first polymerizable may comprise identifying information of the analyte, and further analysis (e.g., sequencing) of the first polymerizable molecule may identify the analyte. In further additional aspects, also provided herein are systems and methods for processing a peptide comprising an amino acid comprising a non-naturally occurring modification; such a method may comprise providing the peptide and a substrate, coupling the amino acid or an additional amino acid to a substrate, and cleaving the substrate-coupled amino acid from the peptide to yield a removed substrate-coupled amino acid and a remainder of the peptide. In further additional aspects, disclosed herein are methods for characterizing a plurality of analytes using an ordered binding approach.

Beneficially, some of the methods disclosed herein comprise the identification of cleaved monomers, thereby obviating the “local environment” problem, in which the ability or specificity of a binding agent to bind to a specific monomer is affected by the adjacent monomers. By first cleaving the monomer from the polymeric analyte, the monomer is removed from the adjacent monomers, and binding agents can bind to individual monomers without the influence of the adjacent surrounding monomers. As such, the methods disclosed herein enable more accurate molecular identification and polymeric analyte sequencing, which has applications in diagnosing disease, monitoring protein dynamics or protein interactions, single-cell proteomics, developing or characterizing therapeutics, and more.

As described further herein, one or more methods of the present disclosure may employ the use of a linker that is capable of coupling to (i) a monomer of the polymeric analyte and (ii) a capture moiety, which may be used to locally tether the monomer adjacent to the polymeric analyte. The methods and systems disclosed herein may additionally comprise a substrate for the local tethering; the substrate may be coupled, for example, to the polymeric analyte, the capture moiety, and additional polymerizable molecules which can be encoded with information by copying or transfer of the information of the encoded polymerizable molecules to the additional polymerizable molecules. Further analysis of the encoded additional polymerizable molecules subsequent to copying or transfer of information may be performed, e.g., using NGS sequencing approaches, which can identify the individual monomers and the sequence (or position) in which they occur in the polymeric analyte.

The methods and systems disclosed herein have applications in studying polymeric analytes. A polymeric analyte may comprise a biological molecule, such as a protein or peptide, nucleic acid molecule, lipid, carbohydrate, or combination thereof. The methods and systems may also be useful in studying non-biological or synthetic polymers, such as peptoids, plastics, polyesters, etc. Accordingly, the methods and systems disclosed herein may be useful in interrogating and characterizing analytes across a wide range of fields, ranging from proteomics, genomics, transcriptomics, metabolomics, and polymer science. The methods and systems disclosed herein may provide for analysis of the polymeric analytes at single-molecule level sensitivity in a high-throughput format.

In one aspect of the present disclosure, provided herein is a method for processing a polymeric analyte comprising a plurality of monomers. Such a method may comprise cleaving of a monomer of the polymeric analyte and coupling the monomer to a capture moiety for subsequent analysis. The method may comprise providing (i) the polymeric analyte comprising a plurality of monomers and (ii) a capture moiety; cleaving a monomer of the plurality of monomers; coupling the monomer to the capture moiety to generate a monomer-capture moiety complex; contacting the monomer-capture moiety complex with a binding agent and a first polymerizable molecule; and coupling the first polymerizable molecule to a second polymerizable molecule. The first polymerizable molecule may comprise information about the binding agent, such as the identity of the binding agent or its cognate molecule, or the identity of the monomer. In some instances, the binding agent comprises an activating agent, and the activating agent facilitates coupling of the first polymerizable molecule to the second polymerizable molecule. The information of the first polymerizable molecule may be transferred to the second polymerizable molecule. Subsequent readout or identification of the first polymerizable molecule may yield information on the identity of the binding agent or the cleaved monomer. One or more operations may be iterated for any number of cycles to identify the sequence and identity of all or a subset of the monomers of the plurality of monomers.

Polymeric Analytes: A polymeric analyte may be a biomolecule, macromolecule, or synthetic molecule. The polymeric analyte may be a biomolecule or other biological molecule (e.g., derived from a sample) that comprises one or more monomers. Non-limiting examples of polymeric biomolecules include nucleic acid molecules (e.g., DNA molecule, RNA molecule, DNA:RNA hybrids, aptamers), peptides and proteins, polysaccharides, lipid polymers (e.g., diglycerides, triglycerides and other fatty acids). The polymeric analyte may be a synthetic molecule, e.g., a peptoid or synthetic polymer, or a peptidomimetic (e.g., a peptoid, a beta-peptide, a D-peptide peptidomimetic). Non-limiting examples of synthetic polymers include acrylics, nylons, silicones, viscose, rayon, polyesters, polycarboxylic acids, polyvinyl acetate, polyacrylamide, polyacrylate, polyethylene glycol, polyurethane, polylactic acid, silica, polystyrene, polyacrylonitrile, polybutadiene, polycarbonate, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride), or a combination thereof. The polymeric analytes may comprise a single polymer type (e.g., a homopolymer) or more than one polymer type (e.g., a copolymer) and may comprise random or arranged monomers. The polymeric analytes may be a block polymer, alternating copolymer, periodic copolymer, statistical copolymer, stereoblock copolymer, gradient copolymer, branched copolymer, graft copolymer, etc.

The polymeric analytes may be any size or comprise a range of sizes. The polymeric analyte may be about 1 nanometer (nm), about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (μm), about 10 μm, about 100 μm, about 1 millimeter mm in size or greater. A plurality of polymeric analytes may comprise polymeric analytes of similar size or within a range of sizes, e.g., between about 10 nm to about 100 nm, between about 50 nm to about 1 μm. Similarly, the polymeric analytes may have any molecular weight or range of molecular weights. The polymeric analytes may be about 10 daltons (Da), 100 Da, 500 Da, 1 kilodalton (kDa), 10 kDa, 100 kDa, 1,000 kDa, 10,000 kDa, 100,000 kDa, or greater. The polymeric analytes may comprise polymeric analytes of similar molecular weight or within a range of molecular weights.

The monomers of the polymeric analytes may comprise any size or range of sizes that is less than that of the entire polymeric analyte. A monomer may be about 0.1 nanometer (nm), about 0.5 nm, 1 about 1 nm, about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (μm), about 10 μm, about 100 μm, about 1 millimeter mm in size or greater. The monomers may have any molecular weight or range of molecular weights. The monomers may be about 1 dalton (Da), 10 Da, 100 Da, 500 Da, 1 kilodalton (kDa), 10 kDa, 100 kDa, 1,000 kDa, 10,000 kDa, 100,000 kDa, or greater. The monomers or polymeric analytes may range in size of molecular weight; for example, a polymeric analyte may comprise a peptide comprising amino acid monomers, which may vary in molecular weight from 75 Da (glycine) to 204 Da (tryptophan).

The polymeric analytes may comprise any number of monomers. The polymeric analytes may comprise about 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000 or more monomers. The polymeric analytes may comprise at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 500, at least about 1,000, at least about 5,000, at least about 10,000, at least about 50,000 at least about 100,000 or greater monomers. Alternatively, the polymeric analytes may comprise at most about 100,000, at most about 50,000, at most about 10,000, at most about 5,000, at most about 1,000, at most about 500, at most about 100, at most about 50, at most about 10, at most about 5, or fewer monomers. The polymeric analytes may comprise a range of monomers; for example, a polymeric analyte may comprise about 5 monomers whereas another polymeric analyte may comprise about 500 monomers.

In some instances, the polymeric analyte comprises a peptide comprising amino acid monomers. The peptide may be naturally occurring or synthetic and may be a part of or derived from a sample. The peptide may comprise any number of amino acids. The amino acids may be one of the 20 proteinogenic amino acids and may comprise any number of post-translational modifications. The peptides or any of the constituent amino acids may be processed to comprise a non-naturally occurring modification, e.g., contacted with protecting groups, alkylated, beta-elimination of phosphate groups, etc., as is described elsewhere herein. In some instances, the peptides are derived from larger peptides or proteins and are fragmented.

Substrates: One or more operations described herein may be performed using a substrate. For example, one or more molecules described herein (e.g., polymeric analyte, capture moiety, polymerizable molecule) may be coupled to a substrate. In some instances, the polymeric analyte, capture moiety, and one or more polymerizable molecules (e.g., the first or second polymerizable molecule), or a combination thereof may be provided coupled to one or more substrates. In one example, the polymeric analyte, capture moiety, and a polymerizable molecule (e.g., the second polymerizable molecule that couples to the first polymerizable molecule comprising encoded information) are coupled to a substrate. In some instances, more than one substrate may be used. In such cases, the substrates may comprise the same material or different material.

The substrate may be made from any suitable material, e.g., glass, silica, silicon, gel, polymer, etc., as is described elsewhere herein. In some instances, the substrate may be a particle, bead or a gel bead (e.g., polyacrylamide, agarose, polyethylene glycol, or TentaGel® bead). The substrate may be functionalized. One or more molecules, e.g., a capture moiety and the polymeric analyte (e.g., a peptide) may be coupled to the substrate via a covalent or non-covalent interaction. The coupling or attachment may be direct or indirect. The capture moiety and polymeric analyte (e.g., peptide) can be coupled to the substrate using any suitable chemistry, e.g., click chemistry moieties (e.g., alkyne-azide coupling), photoreactive groups (e.g., benzophenone, phenyldiazirine, phenylazide, 3-cyanovinylcarbazole phosphoramidite (CNVK)), 1-ethyl-3-(3-dimethylaminopropyl)carbodiimide hydrochloride (EDC), N-hydroxysulfosuccinimide (NHS), Sulfo-NHS, or NHS-esters, maleimides, thiols, biotin-avidin interactions, cystamine, glutaraldehyde, formaldehyde, succinimidyl 4-(N-maleimidomethyl)cyclohexame-1-carboxylate (SMCC), Sulfo-SMCC, 4-(4,6-Dimethoxy-1,3,5-triazin-2-yl)-4-methylmorpholinium chloride (DMTMM), silane (e.g., amino silanes), hydrazine, hydroxyl amine, etc., or a combination thereof.

In some instances, the substrate may be functionalized to comprise a coupling chemistry to couple the polymeric analyte or the capture moiety. In one non-limiting example, a substrate (e.g., bead or surface) may comprise an alkyne such as dibenzocyclooctyne (DBCO, e.g., DBCO-alcohol, DBCO-Boc, DBCO-NHS, DBCO-silane), which may be configured to react to an amine, a carboyxl or carbonyl, a sulfhydryl, etc. A DBCO-functionalized substrate may conjugate to a polymerizable molecule, e.g., an azide-functionalized polymerizable molecule (e.g., nucleic acid, protein) or polymeric analyte via a click chemistry reaction. In other examples, linkers such as bifunctional linkers may be used to attach a molecule to a substrate; such bifunctional linkers may comprise the same reactive moiety on both ends or a different moiety at each end (e.g., heterobifunctional linker). Additional examples of linkers are described elsewhere herein.

In some instances, a molecule (e.g., polymeric analytes, capture moieties, polymerizable molecules) may be coupled to the substrate using an enzymatic approach, e.g., as described elsewhere herein. For example, a chemical linker or moiety such as a click chemistry moiety may be attached to a polymeric analyte (e.g., peptide) using an enzyme. The chemical linker or moiety may be able to react with another chemical linker or moiety (e.g., click chemistry moiety) of a substrate, capture moiety, or polymerizable molecule.

The substrates may be coupled to any useful number of molecules (e.g., polymeric analytes, capture moieties, polymerizable molecules). In some instances, a substrate may comprise a plurality of polymeric analytes, a plurality of capture moieties, and/or a plurality of polymerizable molecules, which may be provided at any useful ratio or density. For example, the ratio of polymeric analytes to capture moieties or polymerizable molecules may be about 1:1, 1:5, 1:10, 1:20, 1:100, 1:1000, 1:10,000, 1:100,000, 1:1,000,000 or lower. In some instances, the ratio of polymeric analytes to capture moieties or polymerizable molecules may be at most about 1:1, at most about 1:5, at most about 1:10, at most about 1:20, at most about 1:100, at most about 1:1000, at most about 1:10,000, at most about 1:100,000, at most about 1:1,000,000 or lower.

Similarly, the molecules (e.g., polymeric analytes, capture moieties, or polymerizable molecules) may be coupled to the substrate at any useful density, for example about 1 molecule/square micron (μm²), about 10 molecules/μm², about 100 molecules/μm², about 1,000 molecules/μm², about 10,000 molecules/μm², about 100,000 molecules/μm², about 1,000,000 molecules/μm², about 10,000,000 molecules/μm², about 100,000,000 molecules/μm², about 1,000,000,000 molecules/μm², about 10,000,000,000 molecules/μm², about 100,000,000,000 molecules/μm², or greater. The polymeric analytes, capture moieties, and polymerizable molecules may be coupled to the substrate at a range of densities, e.g., from about 100 to about 10,000 molecules/μm², or from about 10 to about 1,000 molecules/μm². The density of the polymeric analytes, capture moieties, and polymerizable molecules may be the same or different. For example, the density of the polymerizable molecules may be 1-fold, 2-fold, 3-fold, 4-fold, 5-fold, 6-fold, 7-fold, 8-fold, 9-fold, 10-fold, 100-fold, 1000-fold, 10,000-fold, 100,000-fold, 1,000,000-fold or greater-fold lower than that of the polymeric analyte.

In some instances, the molecules (e.g., polymeric analytes, capture moieties, or polymerizable molecules) coupled to the substrate may be spaced apart at a designated or controlled distance. For example, the average spacing or distance between the polymerizable molecules coupled to the substrate may be about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 μm or greater. In some instances, the spacing between the polymerizable molecules coupled to the substrate may be at most about 1 μm, at most about 500 nm, at most about 100 nm, at most about 90 nm, at most about 80 nm, at most about 70 nm, at most about 60 nm, at most about 50 nm, at most about 40 nm, at most about 30 nm, at most about 20 nm, at most about 10 nm, at most about 5 nm, or less. Similarly, the spacing or distance between a polymeric analyte and a polymerizable molecule or capture moiety may be about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 μm or greater. In some instances, the average spacing between the polymerizable molecule and the polymeric analyte coupled to the substrate may be at most about 1 μm, at most about 500 nm, at most about 100 nm, at most about 90 nm, at most about 80 nm, at most about 70 nm, at most about 60 nm, at most about 50 nm, at most about 40 nm, at most about 30 nm, at most about 20 nm, at most about 10 nm, at most about 5 nm, or less. A range of average distances between the polymerizable molecules from one another or from the polymeric analytes may be used, e.g., from about 1 nm to about 40 nm, from about 2 nm to about 10 nm, etc.

The concentration or density of the molecules attached to the substrate may be modulated using one or more suitable approaches, including patterning or random deposition approaches. Examples of methods to control the concentration or density of the molecules attached to the substrate include limited dilution, addition of chaotropes (e.g., guanidine, formamide, urea), using metal organic compounds, etc. The molecules may be attached to the substrate in a patterned fashion, e.g., using self-assembling monolayers, photopatterning, lithography, etching, or a combination thereof, or the molecules may be randomly arranged.

The substrate may comprise any useful size or dimension (e.g., length, width, height, diameter, radius), surface area, volume, or ratio or combination thereof. The substrate may comprise a bead or particle that may comprise a diameter of about 1 nanometer (nm), about 2 nm, about 3 nm, about 4 nm, about 5 nm, about 6 nm, about 8 nm, about 9 nm, about 10 nm, about 20 nm, about 30 nm, about 40 nm, about 50 nm, about 60 nm, about 70 nm, about 80 nm, about 90 nm, about 100 nm, about 500 nm, about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1 millimeter (mm) or greater. The substrate may comprise a surface area of about 1 square nanometer (nm²), about 10 nm², about 100 nm², about 1,000 nm², about 10,000 nm², about 100,000 nm², about 1 μm², about 10 μm², about 100 μm², about 1,000 μm², about 10,000 μm², about 100,000 μm², about 1 mm², about 10 mm², about 100 mm², about 1,000 mm², about 10,000 mm², about 100,000 mm², about 1,000,000 mm² or greater.

The substrate may comprise a substantially planar substrate. Non-limiting examples of substantially planar substrates include microscope slides, microfluidic devices, flow cells, and multi-well plates. In some instances, the substrate may comprise a commercially available substrate, such as a DNA bead, chip or flow cell, e.g., Illumina® HiSeq, iSeq, MiniSeq, NextSeq, NovaSeq flow cells. In such instances, the molecules (e.g., polymeric analytes, capture moieties, or polymerizable molecules) coupled to the substrate may be designed to couple to the pre-provided DNA molecules (e.g., P5 or P7 sequences), or the pre-provided DNA molecules may be used as the capture moieties or polymerizable molecules, or for coupling of the polymeric analytes.

The molecules (e.g., polymeric analytes, capture moieties, or polymerizable molecules) may be coupled to the substrate in an ordered or random arrangement. In ordered arrangements, the molecules may be patterned using any conventional approach such as lithography (e.g., soft lithography, photolithography), etching (e.g., ion etching, photo etching), or other patterning approach. In some instances, a linker (e.g., bifunctional linker) may be used to facilitate the coupling of the molecules (e.g., polymeric analytes, polymerizable molecules, capture moieties) to the substrate; such linkers may be patterned using any useful technique such as self-assembling monolayers, photopatterning, lithography, etching. In some instances, the molecules may be coupled to the substrate in a random arrangement. For example, the molecules may be provided at a stoichiometric ratio or controlled concentration to couple the molecules at any useful ratio or density. Additional examples of substrate types, substrate functionalization and conjugation, and attachment of molecules to substrates are provided elsewhere herein.

Polymerizable Molecules: The polymerizable molecules described herein may be any useful type of polymerizable molecule. The polymerizable molecules may by naturally occurring, such as biological polymers (e.g., nucleic acid molecules, peptides, polysaccharides, fatty acids), or other naturally occurring polymers, e.g., rubber, cellulose, starches, polyhydroxyalkanoates, chitosan, dextran, structural proteins (e.g., collagen, hyaluronic acid, glycosaminoglycans), agarose, carrageenan, isphagula, acacia, agar, gelatin, shellac, xanthan gum, guar gum, alginate, etc. The polymerizable molecules may be synthetic, e.g., acrylics, nylons, silicones, viscose, rayon, polyesters, polycarboxylic acids, polyvinyl acetate, polyacrylamide, polyacrylate, polyethylene glycol, polyurethane, polylactic acid, silica, polystyrene, polyacrylonitrile, polybutadiene, polycarbonate, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and combinations thereof. The polymerizable molecules may comprise one or more reactive moieties (e.g., radical groups) to initiate polymerization or may be polymerized via contacting of an initiating agent (e.g., ammonium persulfate, peroxide, or other radicalizing agent). The polymerizable molecules may be polymerizable via contacting of an enzyme (e.g., polymerizing enzyme such as polymerases), ribozyme or DNAzyme. Alternatively or in addition to, the polymerizable molecules may be polymerizable via self-assembly. The polymerizable molecules may comprise a single polymer type (e.g., a homopolymer) or more than one polymer type (e.g., a copolymer) and may comprise random or arranged monomers. The polymerizable molecules may be a block polymer, alternating copolymer, periodic copolymer, statistical copolymer, stereoblock copolymer, gradient copolymer, branched copolymer, graft copolymer, etc.

The same or different types of polymerizable molecules may be used in the methods described herein. For example, the first polymerizable molecule (e.g., encoding polymerizable molecule) may be a nucleic acid molecule, and the second polymerizable molecule (e.g., for attachment and transfer of information of the first polymerizable molecule) may be a peptide. In another example, both the first polymerizable molecule and the second polymerizable molecule are nucleic acid molecules. In such an example, the first polymerizable molecule may be coupled to the second polymerizable molecule via ligation or hybridization. For instance, the first polymerizable molecule may comprise a first nucleic acid sequence and the second polymerizable molecule may comprise a second nucleic acid sequence. The first nucleic acid sequence may be complementary or partially complementary to the second nucleic acid sequence, and the coupling may comprise hybridizing the first nucleic acid sequence or portion thereof to the second nucleic acid sequence or portion thereof. Alternatively, the first nucleic acid sequence and the nucleic acid sequence may be complementary to two sequences of a splint or bridge oligonucleotide, and coupling may be mediated via hybridization to the splint oligo. The first nucleic acid sequence may be ligated to the second nucleic acid sequence, either chemically (e.g., via click chemistry approaches in which the first polymerizable molecule and the second polymerizable molecule both comprise one or more members of a click chemistry pair) or enzymatically (e.g., using a ligase or polymerase).

The polymerizable molecules may comprise functional portions. For example, the polymerizable molecules may comprise a member of a binding pair (e.g., biotin-streptavidin), a reactive moiety, a functional site, etc. A polymerizable molecule may comprise a nucleic acid molecule comprising a functional sequence, such as a primer sequence (e.g., universal priming site), a sequencing sequence (e.g., Illumina P5 or P7 sequences), a read sequence (e.g., R1 or R2 sequences), a unique molecular identifier (UMI), a barcode sequence, a cleavage site or sequence (e.g., a uracil or abasic site, a restriction site, a Cas-binding sequence), a transposition sequence (e.g., a mosaic end sequence), or a combination thereof.

The polymerizable molecules may be any useful size. The polymerizable molecules may be about 1 angstrom, about 2 angstrom, about 3 angstrom, about 4 angstrom, about 5 angstrom, about 6 angstrom, about 7 angstrom, about 8 angstrom, about 9 angstrom, about 10 angstrom, about 20 angstrom, about 30 angstrom, about 40 angstrom, about 50 angstrom, about 60 angstrom, about 70 angstrom, about 80 angstrom, about 90 angstrom, about 100 angstrom, about 200 angstrom, about 300 angstrom, about 400 angstrom, bout 500 angstrom, about 600 angstrom, about 700 angstrom, about 800 angstrom, about 900 angstrom, about 1000 angstrom, about 10,000 angstrom, about 100,000 angstrom or greater in size, length, or another dimension. In some instances, the polymerizable molecule (e.g., the first polymerizable molecule or the second polymerizable molecule) comprises a nucleic acid molecule comprising one or more nucleotide bases. The polymerizable molecule may comprise any useful number of nucleotide bases, e.g., about 1 base, about 2 bases, about 3 bases, about 4 bases, about 5 bases, about 6 bases, about 7 bases, about 8 bases, about 9 bases, about 10 bases, about 20 bases, about 30 bases, about 40 bases, about 50 bases, about 60 bases, about 70 bases, about 80 bases, about 90 bases, about 100 bases, about 200 bases, about 300 bases, about 400 bases, about 500 bases, about 600 bases, about 700 bases, about 800 bases, about 900 bases, about 1000 bases, or a greater number of bases.

The polymerizable molecules may comprise a nucleic acid molecule, which may comprise a modified nucleotide or non-canonical base. For instance, the polymerizable molecules may comprise a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof. In some instances, a polymerizable molecule may comprise a hexitol nucleic acid (HNA) or a cyclohexyl nucleic acid (CeNA), which may be useful in rendering the polymerizable molecule more resistant to acid degradation (e.g., as used in conventional Edman degradation). Alternatively or in addition to, a polymerizable molecule may comprise naturally occurring bases that are more resistant to acid degradation, e.g., be composed of primarily pyrimidines, e.g., thymine or cytosine. For example, a nucleic acid molecule may comprise at least 50%, at least 60%, at least 70%, at least 80%, at least 90%, or 100% thymines or cytosines, which can render the nucleic acid molecule more acid resistant as compared to a nucleic acid molecule comprising purines (adenines or guanines). Additional examples of modifying a polymerizable molecule to render it more resistant to certain reaction conditions are described elsewhere herein.

In some instances, a polymerizable molecule is an identical molecule as the capture moiety. For example, a substrate may comprise two identical nucleic acid molecules, one of which may be used as a capture moiety for coupling to the monomer of the polymeric analyte, and another which may be used as the polymerizable molecule for coupling to a polymerizable molecule of a binding agent. Accordingly, a substrate may comprise a plurality of polymerizable molecules (e.g., nucleic acid molecules), which can serve altogether as tetherable molecules for coupling to monomers and additional polymerizable molecules (e.g., encoding polymerizable molecules).

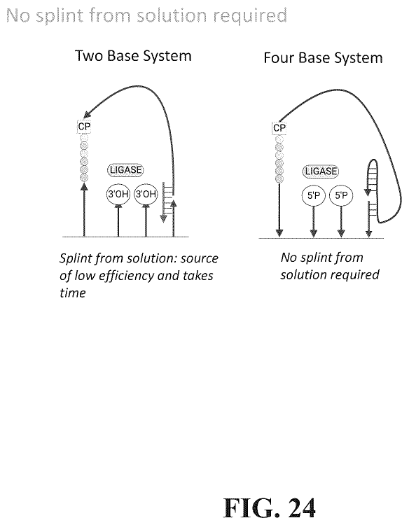

Linkers: One or more operations of the method may be mediated using a linker. In some instances, the coupling of the monomer to the capture moiety to generate a monomer-capture moiety complex is mediated using a linker. The coupling of the linker to the monomer or capture moiety may be covalent or noncovalent. In an example, a linker may comprise a first reactive group that is able to couple to a monomer of the polymeric analyte (e.g., an amino acid of a peptide) and optionally, cleave the amino acid from a peptide. For example, the first reactive group may be a thiocyanate conjugate, e.g., an isothiocyanate (ITC) such as phenyl isothiocyanate (PITC), 3-pyridyl isothiocyanate (PYITC), 2-piperidinoethyl isothiocyanate (PEITC), 3-(4-morpholino) propyl isothiocyanate (MPITC), 3-(diethylamino)propyl isothiocyanate (DEPTIC) or naphthylisothiocyanate (NITC), fluorescein isothiocyanate (FITC), ammonium thiocyanate, potassium thiocyanate, trimethylsilyl isothiocyanate (TMS-ITC), phenyl phosphoroisothiocyanatidate, acetyl isothiocyanate (AITC), or an aldehyde group, e.g., ortho-phthalaldehyde (OPA), 2,3-naphthalenedicarboxyaldehyde (NDA), 2-pyridinecarboxyaldehyde, which can react with an N-terminal amino acid (NTAA). The linker may additionally comprise a second reactive group that is capable of coupling, either directly or indirectly, to the capture moiety. In an example of direct coupling, the capture moiety may comprise a click chemistry moiety (e.g., alkyne), and the second reactive group of the linker may comprise an additional click chemistry moiety (e.g., azide) that can react with the click chemistry moiety of the capture moiety. Alternatively, the linker may be coupled indirectly to the capture moiety, e.g., via noncovalent interaction or via an intermediate linking molecule. In some instances, the intermediate linking molecule may comprise a third polymerizable molecule (e.g., a polymer or nucleic acid molecule) that can couple the linker to the capture moiety. In one such example, the intermediate linking molecule comprises a linking nucleic acid molecule, which may comprise (i) a third reactive group that is capable of coupling to the second reactive group (e.g., via alkyne-azide click chemistry) of the linker and (ii) a moiety that can couple to the capture moiety (e.g., another orthogonal click chemistry reaction, avidin-biotin interaction, nucleic acid coupling or hybridization). In some instances, the third intermediate linking molecule comprises a nucleic acid molecule that comprises (i) a click chemistry moiety (e.g., alkyne) that can conjugate to the first reactive group (e.g., azide) of the linker and (ii) a nucleic acid sequence that can couple to the capture moiety, e.g., via ligation, splint ligation, or hybridization. In some instances, the linker comprises a linking nucleic acid molecule that comprises a self-splinting moiety (e.g., see FIG. 24).

When applicable, the click chemistry moieties of the linker, polymerizable molecules, capture moiety, or intermediate linking molecule may comprise any suitable bioorthogonal moieties, as described elsewhere herein, e.g., alkenes, alkynes (e.g., cyclooctynes or derivatives thereof, e.g., aza-dimethoxycyclooctyne (DIMAC), symmetrical pyrrolocyclooctyne (SYPCO), pyrrolocyclooctyne (PYRROC), difluorocyclooctyne (DIFO), α,α-bis(trifluoromethyl)pyrrolocyclooctyne (TRIPCO), bicyclo[6.1.0]nonyne (BCN), dibenzocyclooctyne (DIBO), difluorobenzocyclooctyne (DIFBO), dibenzoazacyclo-octyne (DBCO), difluoro-aza-dibenzocyclooctyne (F2-DIBAC), biaryl-azacyclooctynone (BARAC), difluorodimethoxydibenzocyclooctynol (FMDIBO), difluorodimethoxydibenzocyclooctynone (keto-FMDIBO), and 3,3,6,6-tetramethylthiacycloheptyne (TMTH)), azides, epoxides, amines, thiols, nitrones, isonitriles, isocyanides, aziridines, activated esters, and tetrazines, and combinations, variations, or derivatives thereof. The click chemistry moieties may be subjected to conditions sufficient to react the first click chemistry moiety to the second click chemistry moiety, e.g., provision of metal catalysts, appropriate solvents, pH, temperature, ionic concentration, or light/energy, for any useful duration of time.

The first reactive group of the linker may be an amino acid-reactive moiety. The amino acid-reactive moiety of the linker may be any useful moiety that enables the reactive moiety to conjugate to and optionally cleave an amino acid. In some examples, the first reactive moiety can react with a terminal amino acid (e.g., NTAA or CTAA). In such examples, the first reactive moiety may comprise any primary amine or carboxylic group reactive group, including but not limited to isocyanates, acyl azides, NHS esters, sulfonyl chlorides, aldehydes, glyoxals, epoxides, oxiranes, carbonates, aryl halides, imidoesters, carbodiimides, anhydrides, phenyl esters, isothiocyanates (e.g., phenyl isothiocyanate, sodium isothiocyanate, ammonium isothiocyanates (e.g., tetrabutylammonium isothiocyanate, tetrabutylammonium isothiocyanate), diphenylphosphoryl isothiocyanate), acetyl chloride, cyanogen bromide, carboxypeptidases, azide, alkyne, DBCO, maleimide, succinimide, thiol-thiol disulfide bonds, tetrazine, TCO, vinyl, methylcyclopropene, acryloyl, allyl, among others. Additional examples of amino acid reactive groups are provided in U.S. Pat. No. 11,499,979, which is incorporated by reference herein in its entirety.

The linker may comprise any additional useful moiety. For example, the linker may comprise a releasable or cleavable moiety, which may facilitate removal of the monomer from the polymeric analyte, or portion thereof, or from the substrate. Such a releasable or cleavable moiety may comprise, for example, a disulfide bond, which may be releasable by contacting with a reducing agent (e.g., DTT, TCEP) or a nucleic acid molecule comprising a cleavage or restriction site. In some examples, the linker may couple to an intermediate linking molecule via the releasable or cleavable moiety, alternatively or in addition to the coupling via click chemistry moieties. As such, the coupling between the intermediate linking molecule and the linker may be reversible. The linker may additionally or alternatively comprise any number of spacing moieties, e.g., polymers (e.g., PEG, PVA, polyacrylamide), aminohexanoic acid, nucleic acids, alkyl chains, etc. Such spacing moieties may increase the distance between any other moieties of the linker, e.g., the amino acid-reactive group and the polymerizable molecule-reactive group.

The linker may comprise or be coupled to a detectable moiety, e.g., a fluorophore, radioisotope, mass tag, nucleic acid molecule (which can also act as a releasable or cleavable moiety), or other detectable moiety. In some examples, the linker comprises a fluorophore, which can enable localization visualization of the linker using single-molecule imaging. In another example, the monomer may be labeled with a first fluorophore and the linker may comprise a second fluorophore to enable localization visualization of the linker and the monomer (e.g., using two-channel imaging or FRET).

Use of a linker comprising two reactive groups may allow for coupling of the linker to (i) the monomer of the polymeric analyte and (ii) the intermediate linking molecule (e.g., third polymerizable molecule) or (iii) the capture moiety. In some instances, when an intermediate linking molecule is used, the linker may be pre-coupled to the intermediate linking molecule. For example, a precursor linker may comprise a monomer-binding group (e.g., PITC) and a click chemistry moiety (e.g., azide), which may be reacted with an intermediate linking molecule (e.g., linking nucleic acid molecule) comprising complementary click chemistry moiety (e.g., alkyne, such as DBCO) to generate a linker that comprises the linking nucleic acid molecule and is capable of coupling to the monomer and the capture moiety (e.g., another oligonucleotide). In some instances, the linker may be provided pre-coupled to or comprising the intermediate linking molecule.

In some instances, the polymeric analyte comprises a peptide that comprises amino acid monomers, and the coupling of the linker to an amino acid (e.g., NTAA or CTAA) can change the chemical structure of the amino acid. For example, if using a linker comprising an isothiocyanate moiety, the amino acid may be derivatized to a thiocarbamyl group (e.g., under mildly alkaline conditions) during or subsequent to contact with the isothiocyanate moiety. One or more further derivatizations may be performed. For instance, the amino acid or amino acid derivative (e.g., thiocarbamyl-derivatized amino acid) may be further derivatized to a thiazolone group (e.g., under acid conditions such as Edman degradation or cleavage), a thiohydantoin group, or other chemical moiety. Similarly, a thiazolone group or thiohydantoin group may be further derivatized to a thiocarbamyl group.

Capture Moieties: The capture moieties may couple to a monomer of the polymeric analyte via any suitable mechanism. The coupling of the monomer to the capture moiety may comprise a covalent interaction or a noncovalent interaction. The coupling may occur by interaction of binding pairs, e.g., biotin and avidin (or streptavidin), antigen or epitope and antibody or antibody fragment, cyclodextrins and small hydrophobic molecules (e.g., alkanes, benzene, polycyclics), cucurbiturils and adamantaneammonium or trimethylammoniomethyl ferrocene, cyclophane (e.g., calixarenes, cavitands, pillararenes, tetralactams), etc. In some embodiments, the coupling of the monomer to the capture moiety occurs through coupling of nucleic acid molecules (e.g., hybridization to one another or to a splint molecule).

In some instances, the capture moiety comprises an additional polymerizable molecule (e.g., a nucleic acid molecule or peptide). The monomer may be coupled with a complementary polymerizable molecule (e.g., to generate a monomer-oligonucleotide conjugate) and tethered to the capture moiety, e.g., via complementary base pairing directly or via a splint molecule. Alternatively, the monomer may be coupled to the capture moiety via a linker, as described above. In some examples, the linker comprises a monomer-coupling group (e.g., PITC, which can couple to or react with an amino acid of a peptide) and a nucleic acid molecule. The capture moiety may comprise an additional nucleic acid molecule, which may be coupled to the nucleic acid molecule of the linker via hybridization (directly or via a splint), ligation, or both.

The nucleic acid molecule of the capture moiety can comprise any naturally occurring, non-naturally occurring or engineered nucleotide base. For example, the nucleic acid molecule may comprise a pseudo-complementary base, a bridged nucleic acid, a xenonucleic acid, a locked nucleic acid, a peptide nucleic acid (PNA), a gamma-PNA, a morpholino, etc., as is described elsewhere herein. The capture moiety may comprise one or more functional sequences, including, but not limited to a priming sequence, sequencing sequence (e.g., P5 or P7 sequence), sequencing read sequence (e.g., R1 or R2 sequence), a mosaic end sequence, a transposase recognition sequence, a cleavage site (e.g., restriction site), a UMI, a blocking group, a spacer sequence, a barcode sequence, or other functional sequence. In some instances, the capture moiety comprises a cleavable or releasable moiety, e.g., a restriction enzyme recognition site, an abasic site, a uracil which can be cleaved using USER® or uracil DNA glycosylase, a disulfide bond that can be releasable upon addition of a reducing agent, etc.

In some instances, the capture moiety and a polymerizable molecule are provided coupled to a substrate. In one example, the substrate comprises, coupled thereto, a first nucleic acid molecule, a second nucleic acid molecule, and a capture moiety, which may be a third nucleic acid molecule. In some instances, a substrate may comprise identical nucleic acid molecules or sets of identical nucleic acid molecules across the substrate; these identical nucleic acid molecules may act as both a capture moiety and a polymerizable molecule to which additional polymerizable molecules (e.g., comprising encoded information) may couple. In some examples, a bead, flow cell, or chip comprising a plurality of nucleic acid molecules may be provided, and a subset of the plurality of nucleic acid molecules may act as capture moieties and another subset of the plurality of nucleic acid molecules may act as the polymerizable molecules to which additional polymerizable molecules comprising encoded information may couple. In some instances, commercially available beads (e.g., DNA beads or barcoded beads), flow cells, or chips, e.g., Illumina® HiSeq, iSeq, MiniSeq, NextSeq, NovaSeq, etc. may be used as the substrates described herein. In some instances, the substrate comprises a plurality of sequencing primer sequences (e.g., P5 or P7 sequences) or read sequences (e.g., R1 or R2), which can be used as capture moieties or polymerizable molecules to which additional polymerizable molecules comprising encoded information may couple.

The capture moiety may be coupled to a substrate. In such instances, the capture moiety may comprise a substrate-tethering group or linker or additional functional group. In some examples, the capture moiety comprises a nucleic acid molecule that comprises a substrate-tethering group, e.g., biotin, a click chemistry moiety such as an azide, that can couple to a substrate, e.g., a substrate comprising streptavidin or a complementary click chemistry moiety that can react with that of the substrate-tethering group. The capture moiety may additionally comprise a binding sequence, to which another nucleic acid molecule (e.g., a linking nucleic acid molecule, a linker-monomer complex. or a nucleic acid barcode molecule) can couple, e.g., via hybridization, ligation, or both. In some instances, the capture moiety comprises a single-stranded oligonucleotide or a single-stranded region in which a complementary oligonucleotide can hybridize. The complementary oligonucleotide may comprise a detectable label (e.g., fluorophore) that allows for detection of the capture moiety.

Cleaving: The cleaving of the monomer from the polymeric analyte may be achieved using any suitable mechanism, such as via application of a stimulus. The stimulus can be, for example, a chemical stimulus, a biological stimulus, a thermal stimulus (e.g., application of heat), a photo-stimulus, a physical or mechanical stimulus, or other type of stimulus or a combination of stimuli. In some instances, the stimulus comprises a chemical stimulus, e.g., a change in pH, application of an acid or base, addition of a lytic agent, initiating agent, radical-generating agent, reducing agent, etc. In some instances, the chemical stimulus comprises application of a Lewis acid (e.g., boron triflate, boron trifluoride etherate, boron trichloride, boron tribromide, boron triiodide, or scandium triflate). In some instances, the stimulus comprises a biological stimulus, e.g., enzyme (e.g., Edmanase, protease, endonuclease) or ribozyme or DNAzyme that can cleave or catalyze cleavage of the monomer from the polymeric analyte.

In some examples, the polymeric analyte comprises a peptide and the monomer comprises an amino acid (e.g., an NTAA, CTAA, or internal amino acid). The method may comprise using a linker comprising an amino acid reactive group (e.g., PITC) by coupling the amino acid reactive group of the linker with the amino acid and cleaving the amino acid from the peptide using a stimulus (e.g., change in pH, temperature). In an example, PITC may couple to an NTAA under mildly alkaline conditions to generate a phenylthiocarbamoyl (PTC) derivative of the NTAA, and cleavage of the NTAA from the peptide may be achieved using an Edman degradation reaction (e.g., application of an acid such as trifluoroacetic acid or boron triflate, optionally with heat), to generate a thiazolinone (ATZ) derivative or a phenylthiohydantoin (PTH) derivative. As described elsewhere herein, the linker may comprise a moiety or molecule (e.g., nucleic acid molecule or polymerizable molecule) that can also couple to the capture moiety such that the amino acid may be coupled to the capture moiety.

In some instances, more than one monomer may be cleaved from the polymeric analyte per cleavage event. The cleaving may comprise cleaving 2 monomers, 3 monomers, 4 monomers, 5 monomers, 6 monomers, 7 monomers, 8 monomers, 9 monomers, 10 monomers, or more. For example, the polymeric analyte may comprise a peptide comprising a plurality of amino acid monomers, and single amino acids, di-peptides, tri-peptides, quadri-peptides, or larger may be cleaved in the methods described herein. In some instances, at most about 10 monomers, at most about 9 monomers, at most about 8 monomers, at most about 7 monomers, at most about 6 monomers, at most about 5 monomers, at most about 4 monomers, at most about 3 monomers, or fewer monomers may be cleaved in a given cleavage event. In some instances, cleavage of greater than one monomer (e.g., amino acid) may be mediated using an enzyme (e.g., Edmanase, protease) or ribozyme or DNAzyme that is capable of recognizing or cleaving more than a single amino acid.

Cleavage of the monomer (or plurality of monomers) may be conducted using a biological stimulus, such as an enzyme or ribozyme or DNAzyme. The enzyme can be any useful cleaving enzyme, e.g., a protease, such as an Edmanase, cruzain, a cleaving protein (e.g., ClpS, ClpX), Proteinase K, exopeptidase, aminopeptidase, diaminopeptidase, serine protease, cysteine protease, threonine protease, aspartic protease, aspartic protease, glutamic protease, metalloprotease, asparagine peptide lyase, pepsin, trypsin, pancreatin, Lys-C, Glu-C, Asp-N, chymotrypsin, carboxypeptidase (e.g., carboxypeptidase A, carboxypeptidase B, carboxypeptidase Y), SUMO protease, elastase, papain, endoproteinase, proteinase, TrypZean®, bromelain, collagenase, hyaluronase, thermolysin, ficin, keratinase, tryptase, fibroblast activation, enterokinase, chymotrypsinogen, chymase, clostripain, calpain, alpha-lytic protease, proline specific endopeptidase, furin, thrombin, subtilisin, genenase, PCSK9, cathepsin, prolidase, methionine aminopeptidase, cathepsin C, 1-cyclohexen-1-yl-boronic acid pinacol ester, pyroglutamate aminopeptidase, renin, kininogen, kallikrein, DPPIV/CD26, thimet oligopeptidase, prolyl oligopeptidase, leucine aminopeptidase, dipeptidylpeptidase, or other enzyme or protease, or a combination or variation (e.g., engineered mutant or variant) thereof. In some instances, the cleaving enzyme or ribozyme or DNAzyme may be configured or engineered to cleave a terminal monomer or plurality of monomers; alternatively, the cleaving enzyme or ribozyme or DNAzyme may be configured or engineered to cleave off-site at a non-terminal location of the polymeric analyte, e.g., at an internal monomer within the polymeric analyte, at an n-1, n-2, n-3, n-4, n-5, n-6, n-7, n-8, n-9, n-10, etc. position, where n is the number of monomers in the polymeric analyte.

In the instances of enzymatic cleavage, additional reagents may be provided to catalyze or induce the cleavage. For instance, metalloproteases, aminopeptidases, or exopeptidases may facilitate cleavage of an amino acid or plurality of amino acids in the presence of a catalyst, e.g., metal or metal ion (e.g., cobalt). Accordingly, a catalyst may be provided in order to facilitate the binding of the enzyme to an amino acid or the subsequent cleavage of the amino acid from the peptide. In some examples, cleavage may be mediated by an apo-enzyme, which is inactive in the absence of a metal catalyst of cofactor, and cleavage may be controlled by addition of metal or metal ions.

Other examples of cleaving stimuli include: a photo stimulus (e.g., application of UV, X-rays, gamma rays, or other wavelength of light), mechanical stimulus (e.g., sonication, high pressure, electromagnetic energy), thermal stimulus (e.g., application of heat), or chemical stimulus. In some instances, the polymeric analyte may comprise or be altered to comprise a cleavable or labile bond that can be cleaved upon application of the appropriate stimulus, e.g., disulfide bonds (e.g., cleavable upon application of a chemical stimulus such as a reducing agent), ester linkages (e.g., cleavable with a change of pH), a vicinal-diol linkage (e.g., cleavable with sodium periodate), a Diels-Alder linkage (e.g., cleavable upon application of heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNase)).

Modifications of Monomers: In some instances, one or more monomers of the polymeric analyte may be modified. Modifications may be naturally-occurring (e.g., post translational modifications) or non-naturally occurring, such as by labeling or tagging, e.g., with an amino acid- or amine-reactive agent such as an isothiocyanate (e.g., PITC, NITC), 1-fluoro-2,-4-dinitrobenzene (DNFB), dansyl chloride, 4-sulfonyl-2-nitrobfluorobenzene (SNFB), an acetylating agent, an acylating agent, an alkylating agent, a guanidination agent, a thioacetylation agent, a thioacylation agent, a thiobenzoylation agent, or a derivative or combination thereof. Alternatively, or in addition to, the one or more monomers may be modified to comprise any useful moiety such as an adduct (e.g., a polymer such as PEG, a polymerizable molecule such as a nucleic acid molecule, a nanoparticle or nanotube, a peptide or protein), a lipid, a carbohydrate, a metabolite, a fluorophore, a hapten, a quencher, a tag (e.g., a fluorescent tag, a magnetic tag, a radioactive tag), a barcode, or other moiety. In some instances, a monomer of the polymeric analyte may be modified to facilitate recruitment of an enzyme (or ribozyme or DNAzyme) to recognize or cleave a terminal monomer (e.g., a NTAA or CTAA of a peptide, the 5′ or 3′ nucleotide of a nucleic acid molecule, or the first or last monomer of a polymer) or set of monomers. For example, a terminal amino acid of a peptide analyte may be modified with a saccharide in order to recruit a lectin or lectin-bound protease. In another example, one or more monomers of a polymeric analyte may comprise or be coupled to a nucleic acid molecule having a first sequence that is complementary to a second sequence comprised by an oligo-bound protease. Hybridization of the first sequence to the second sequence may facilitate local recruitment of the protease to the monomer to be cleaved. In yet another example, a peptide analyte may be modified with PITC, which may allow for recruitment and cleavage by an Edmanase. In some examples, modifications to monomers of a polymeric analyte may include epitope tags, which can facilitate binding of a binding agent to the monomer. Examples of such epitope tags include fluorophores, nucleic acid molecules, peptides, haptens, polymers, chemical moieties, or other adduct molecule. Additional examples of modifications to polymeric analytes are described elsewhere herein.

The polymeric analyte may comprise one or more modified monomers. The modification of the monomers may be naturally occurring, or synthetic. Synthetic modifications may be performed prior to, during, or subsequent to cleavage of a monomer from the polymeric analyte and may be advantageous in preserving the identity of the monomer. For instance, during standard Edman degradation reactions to cleave a terminal amino acid (monomer) from the peptide, some amino acid residues may be altered or rendered undetectable by the reaction conditions. In an example, the conditions of Edman degradation may cause oxidation of cysteine residues, dehydration or destruction of the phenylthiohydantoin (PTH) forms of serine or threonine, react with and modify lysine residues, or render some post-translational modifications undetectable. As such, it modifying a peptide prior to processing, e.g., to protect some of the amino acid residues or post-translational modifications may be useful in more accurately identifying each of the amino acid residues. In some examples, a peptide or portion thereof may be alkylated, e.g., to alkylate the cysteine residues (e.g., using 4-vinylpyridine, iodoacetamide, iodoacetate, chloroacetate); acetylated, e.g., to react serine or threonine residues form an ester (e.g., using acetyl chloride) or using acetic anhydride; oxidized, e.g., to convert cysteine residues to cysteic acid; reduced (e.g., using a reducing agent such as dithiothreitol, β-mercaptoethanol, or TCEP); contacted with a protecting group, e.g., phosphorylated residues may be protected (e.g., using a β-elimination of a phosphate group, with an optional Michael addition of a thiol group, e.g., as described in Knight, et al. Nat. Biotechnology. 21, 1047-1054 (2003), which is incorporated by reference herein in its entirety), etc. The polymeric analyte or monomer may be modified with a protecting group or moiety, such as a methyl, formyl, ethyl, acetyl, t-butyl, anisyl, benzyl, tifluoroacetyl, N-hydroxysuccinimide, t-butyloxycarbonyl (Boc), benzoyl, 4-methyl benzyl, thioanizyl, thiocresyl, benzyloxymethyl, 4-nitrophenyl, benzyloxycarbonyl, 2-nitrobenzoyl, 2-nitrophenylsulphenyl, 4-toluenesulphonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl (FMOC), triphenylmethyl, or 2,2,5,7,8-pentamethyl-chroman-6-sulphonyl group. The polymeric analyte or monomer may be treated with a protecting agent, e.g., carboxyethyl methanethiosulfonate (CEMTS), thiazolidine, mercaptophenyl acetic acid, cyanobenzothiazole (e.g., for lipidation of N-terminal cysteines), acetamidomethyl, 2-methylsulfonylethyl-oxycarbonyl, etc. In some instances, the lysine residues may be blocked (e.g., the primary amines of lysine residues may be reacted) using an isothiocyanate (e.g., PITC); optionally, an additional round of Edman degradation may be performed to generate a new N-terminal exposed end.

In some instances, a monomer of the polymeric analyte may be modified to facilitate cleavage of the monomer from the polymeric analyte. For example, an amino acid monomer of a peptide polymeric analytic may be modified such that it is recognized by an enzyme or ribozyme or DNAzyme. In some examples, such a modification may comprise acetylation of an amino acid, which can facilitate acyl peptide hydrolase cleavage of the acetylated amino acid. Additional or alternative modifications to the monomers, such as those described herein, may also facilitate recognition by or interaction with an engineered cleaving enzyme, ribozyme, or DNAzyme.

In some instances, a monomer comprising a naturally-occurring modification may be treated to remove or alter the naturally-occurring modification to render the polymeric analyte or monomer more amenable to the processing operations disclosed herein. For example, acetylation, formylation, methylation, and pyrrolidone carboxylic acid post-translational modifications may be removed prior to sequencing. Acetylation modifications may be removed with acyl peptide hydrolase or acid treatment (e.g., using TN HCl). Methylation may be removed, e.g., using aminopeptidases. Formylation modifications may be removed, for example, using acid treatment (e.g., 0.6M HCl treatment). Pyrrolidone carboxylic acid (PCA) may be removed with pyroglutamate aminopeptidase. Examples of C-terminal modifications may include amidation and methylation, both of which may be removed using carboxypeptidases.

Binding agents: The binding agent may be contacted with the monomer, e.g., subsequent to coupling of the monomer to the capture moiety and cleavage. The binding agent may be any useful molecule that can couple to the monomer or monomer-capture moiety complex. For example, a binding agent may be or comprise a protein or peptide (e.g., an antibody, antibody fragment, single chain variant fragment (scFv), nanobody, anticalin, tRNA synthetase or tRNA-acyl synthetase, a fibronectin domain), a peptide mimetic, a peptidomimetic (e.g., a peptoid, a beta-peptide, a D-peptide peptidomimetic), a polysaccharide, a nucleic acid molecule (e.g., aptamer), a somamer, a polymer, an inorganic compound, an organic compound, a small molecule, or derivatives (e.g., engineered variants) or combinations thereof. In instances where the polymeric analyte comprises a peptide, the binding agent may be able to bind to a modified amino acid (e.g., an amino acid coupled to a linker) or portion thereof. The binding agent may comprise a recognition site that specifically recognizes an amino acid, modified amino acid (e.g., an amino acid bound to a linker comprising a PITC moiety), or a derivatized, and optionally modified, amino acid. For example, the binding agent may be configured to recognize or have binding specificity to a moiety of a modified amino acid, such as a specific amino acid residue, the residue-linker complex, or derivatized amino acid (e.g., a thiocarbamoyl-derivatized residue, a thiazolone-derivatized residue, a thiohydantoin-derivatized residue, etc.), or a portion of a modified amino acid. In some instances, the binding agent may recognize and bind a cleaved or modified amino acid but not recognize or bind an amino acid when present in a peptide. In some instances, the binding agent may be derived or engineered from a naturally-occurring enzyme or protein, e.g., an aminopeptidase, exopeptidase, metalloprotease, antibody, anticalin, N-recognin protein, Clp protease, endoprotease (e.g. trypsin), or tRNA synthetase. In some examples, a binding agent may be or comprise a cleaving enzyme (e.g., trypsin, endoprotease) that has been modified to remove the peptidase activity. The binding agent may also recognize a terminal amino acid that is attached to a substrate; for example, after all but the final monomer of a polymeric analyte has been coupled to the capture moiety or capture moieties and cleaved, the final monomer may remain coupled to a substrate. Accordingly, the binding agent may recognize and bind the surface-coupled monomer.

The binding agents may be contacted with and specifically bind to cleaved monomers, monomer-linker complex, monomer-linker-capture moiety, or monomer-capture moiety complexes (altogether referred to herein as “monomeric analytes”). For example, a monomeric analyte may fall in any size or range of sizes that is less than that of the entire polymeric analyte. A monomeric analyte complex may be about 0.1 nanometer (nm), about 0.5 nm, 1 about 1 nm, about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1 micrometer (μm), about 10 μm, about 100 μm, about 1 millimeter mm in size or greater. The monomeric analyte may have any molecular weight or range of molecular weights. The monomeric analyte may be about 1 dalton (Da), 10 Da, 100 Da, 500 Da, 1 kilodalton (kDa), 10 kDa, 100 kDa, 1,000 kDa, 10,000 kDa, 100,000 kDa, or greater. The monomeric analyte may vary in molecular weight or length, e.g., depending on the amino acid residue.

Activating agents: The binding agent may comprise or be coupled (e.g., directly or indirectly) to an activating agent. The activating agent may facilitate coupling of polymerizable molecules, e.g., the first polymerizable molecule, which may comprise identifying information of the binding agent or the monomer (e.g., monomer type such as the amino acid type), to the second polymerizable molecule. Activating agents may comprise any useful chemical or biological moiety, e.g., a peptide or protein (e.g., enzyme), a ribozyme, a DNAzyme, a nucleic acid molecule, a lipid, a carbohydrate, a free radical or radical-generating agent, a catalyst, an ion, a small molecule, a mineral, a metal, a salt, a nucleophile, an electrophile, an oxidizing reagent, a reducing reagent, an acid, a base, or other reagent.

In some instances, the activating agent comprises a polymerizable molecule. The activating agent may comprise any useful type of polymerizable molecule (also referred to herein as “activating polymerizable molecule”), e.g., nucleic acid molecules, peptides or proteins (e.g., enzymes). The activating polymerizable molecule may be the same type of molecule as the binding agent (e.g., both peptides, both nucleic acid molecules, etc.), or they may be different. In some instances, the binding agent comprises a peptide (e.g., antibody or antibody fragment) and the activating polymerizable molecule comprises a nucleic acid molecule. The activating polymerizable molecule may be conjugated to the binding agent via a chemical conjugation approach, e.g., using linkers such as SMCC, (N-e-maleimidocaproyloxy)succinimide ester (EMCS), succinimidyl-4-(p-maleimidophenyl)butryate (SMPB), succinimidyl-(N-maleimidopropionamido-ethyleneglycol) ester (SMPEG), Succinimidyl (NHS) esters, succinimidyl-4-formylbenzamide (S-4FB), succinidmidyl-6-hydrazino-nicotinamide (S-HyNic), 4-Phenyl-3H-1,2,4-triazoline-3.5(4H)diones (PTAD) or other diazonium, 1-ethyl-3-3-dimethylaminoproyl carbodiimide hydrochloride (EDC), etc. Synthesis of the binding agent-activating polymerizable molecule conjugate (e.g., peptide-nucleic acid molecule conjugate) may also be carried out using solid-phase synthesis, fragment conjugation (e.g., using heterobifunctional crosslinkers such as those comprising an aliphatic chain and a maleimide group on one end and NHS on the other), click chemistry (e.g., strain-promoted azide alkyne cycloaddition, inverse-electron-demand Diels-Alder reactions), or combinations of approaches or chemistries. In some instances, the activating polymerizable molecule may be conjugated to the binding agent using an enzymatic approach. For example, a DNA-protein conjugate may be generated using a truncated a nuclease (e.g., Cas protein such as Cas9), a relaxase (e.g., VirD2), or other enzyme, ribozyme, or DNAzyme. In some instances, the activating polymerizable molecule may be conjugated to the binding agent using a SpyTag and SpyCatcher interaction, a biotin-avidin interaction, a SNAP-tag, or other interaction. Optional purification may be performed, e.g., using ion-exchange chromatography, affinity chromatography, high performance liquid chromatography, electrophoresis, electrofocusing, or other purification or separation technique.

In some instances, the activating polymerizable molecule is a nucleic acid molecule, which may facilitate coupling of one or more other nucleic acid molecules. In an example, the first polymerizable molecule, the second polymerizable molecule, and the activating polymerizable molecule may all comprise nucleic acid molecules. The first polymerizable molecule may comprise a nucleic acid barcode molecule that encodes for or comprises identifying information on the binding agent or the monomer. The activating polymerizable molecule may be a splint oligonucleotide to which the first polymerizable molecule and the second polymerizable molecule can hybridize. In such examples, the activating polymerizable molecule comprises a sequence complementary to at least a portion of the first polymerizable molecule and the second polymerizable molecule. In another example, the first polymerizable molecule is not complementary or otherwise coupled to the activating polymerizable molecule; in such instances, an additional splint oligonucleotide may be required to enable coupling of the activating polymerizable molecule to the first polymerizable molecule, e.g., via provision of a splint oligonucleotide that comprises a sequence complementary to at least a portion of the activating agent, the first polymerizable molecule, and optionally, the second polymerizable molecule.

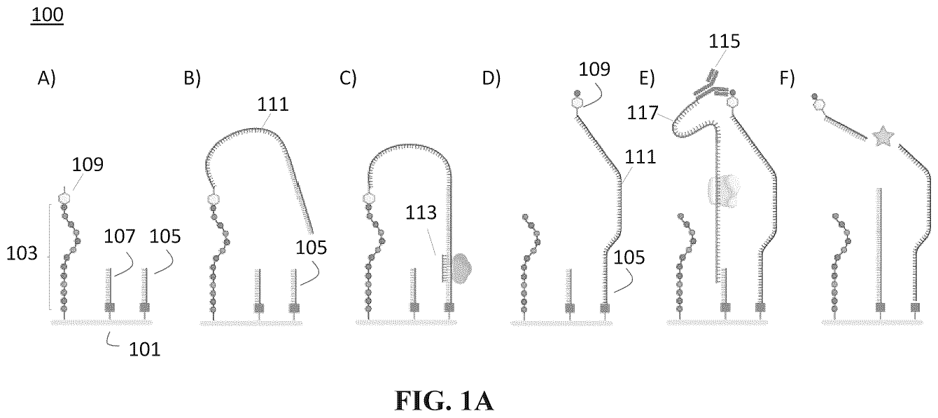

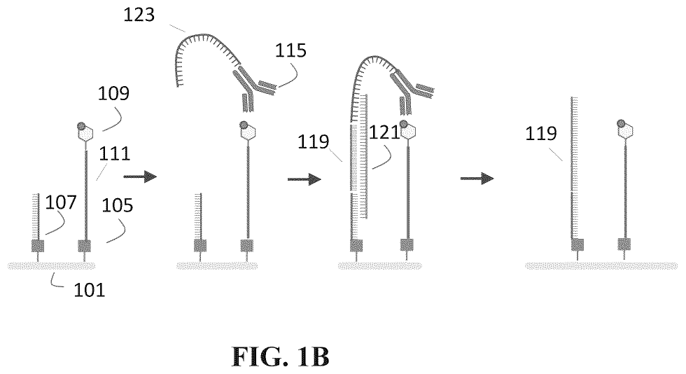

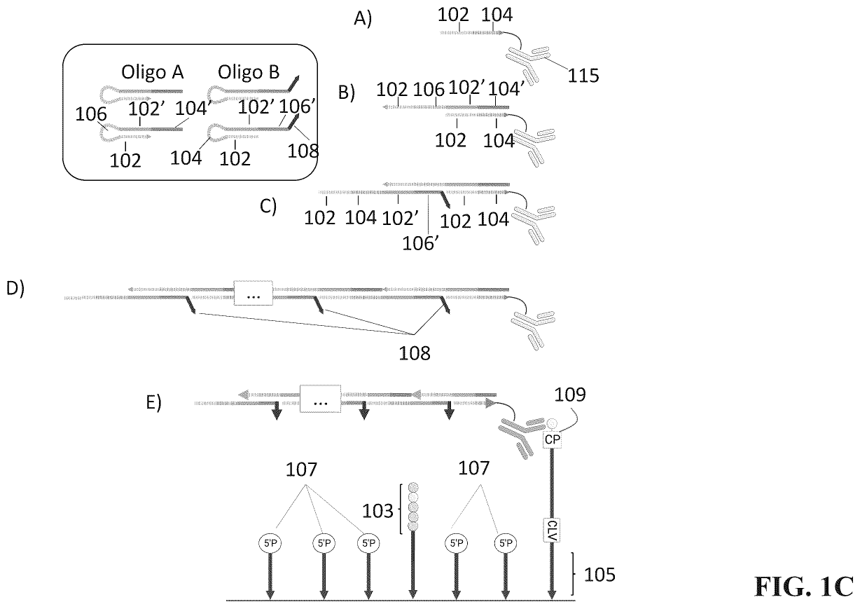

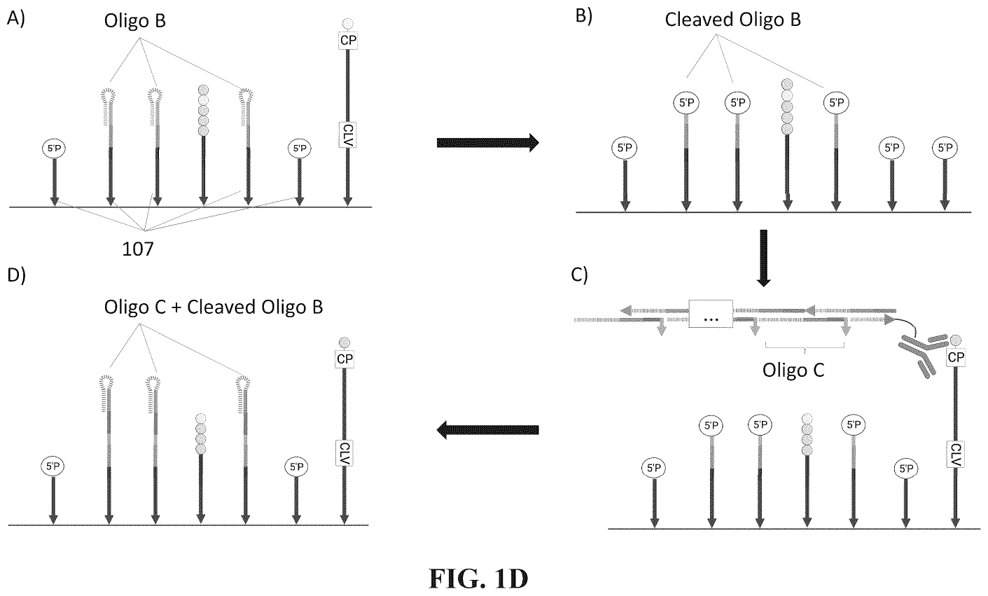

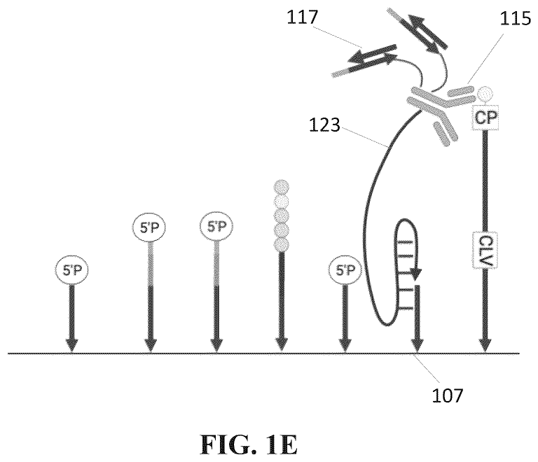

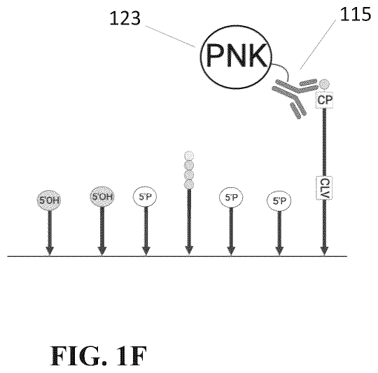

In some instances, the first polymerizable molecule comprises a nucleic acid barcode molecule, and the methods described herein may comprise generating the nucleic acid barcode molecule. The nucleic acid barcode molecule may be generated using the activating agent (e.g., activating polymerizable molecule) as an initiating nucleic acid molecule. In one such example, the activating agent comprises a nucleic acid molecule, and subsequent barcode sequence segments may be added to the activating agent, e.g., using hybridization chain reaction (HCR, e.g., as shown in FIG. 1C and described further elsewhere herein), thereby generating the first polymerizable molecule. One or more of the barcode sequence segments may comprise identifying information about the binding agent or its cognate binding molecule (e.g., a type of monomer, an amino acid type, etc.). The first polymerizable molecule may comprise any useful number of barcode sequence segments and may comprise additional functional sequences, e.g., primer sites, sequencing sites, restriction or cleavage sites, spacers, noncanonical nucleic acids, HNAs, PNAs, etc. In some instances, the binding agent may comprise an HCR component (e.g., a first hairpin molecule), which can be couple to an activating agent that is provided separately. Accordingly, the activating agent may be coupled to the binding agent via the HCR reaction. In some instances, the first polymerizable molecule may comprise a flap sequence, which can facilitate coupling of the first polymerizable molecule to the second polymerizable molecule or other polymerizable molecules.

In some instances, the activating agent may comprise a nucleic acid molecule that indirectly facilitates coupling of the first polymerizable molecule to the second polymerizable molecule. In an example, the binding agent may be coupled to a first polymerizable molecule (e.g., a nucleic acid barcode molecule), and an activating nucleic acid molecule. The activating nucleic acid molecule may act as an anchoring nucleic acid molecule and couple to an additional polymerizable molecule (e.g., on a substrate comprising multiple identical polymerizable molecules, including the second polymerizable molecule). The coupling of the activating nucleic acid molecule to the additional polymerizable molecule may aid in anchoring the binding agent to the monomer, thereby facilitating transfer or coupling of the first polymerizable molecule to the second polymerizable molecule (see, e.g., FIG. 1E). In some instances, the anchoring nucleic acid molecule increases the residence time of the binding agent on the monomer, thereby increasing the probability that the first polymerizable molecule couples to the second polymerizable molecule (or other polymerizable molecules).

Alternatively, or in addition to, the activating agent may comprise a polymerizable molecule that comprises a peptide or protein, e.g., an enzyme. The enzyme may facilitate coupling of the first polymerizable molecule to the second polymerizable molecule, directly or indirectly. The enzyme may be a ligating enzyme (e.g., a ligase, such as T4 or T7 DNA ligase, Circligase, Taq ligase, Splint® Ligase, etc.), a polymerizing enzyme (e.g., a DNA or RNA polymerase, reverse transcriptase), a kinase (e.g., protein kinases, lipid kinases, nucleic acid kinases such as polynucleotide kinase (PNK)), a phosphatase (e.g., alkaline phosphatase), a radical-generating or oxidizing enzyme such as a horse radish peroxidase (HRP) or ascorbate peroxidase (APEX), a nuclease (e.g., an endonuclease, restriction enzyme, a Cas enzyme), a phosphorylase, an ATPase, or other enzyme or combination of enzymes. In some instances, the enzyme is a cleaving enzyme, e.g., glycosylases (e.g., uracil glycosylase), restriction endonucleases, micrococcal nucleases, transposases, Cas proteins (e.g., Cas9), Argonaut endonucleases. In one such example, the second polymerizable molecule may comprise a cleavage or restriction site, and coupling of the first polymerizable molecule may occur only if the cleavage or restriction site is cleaved or removed.

The binding agent may be coupled to the activating agent (e.g., polymerizable molecule such as a nucleic acid molecule or enzyme) via a noncovalent interaction. For instance, the binding agent may comprise an avidin or streptavidin tag, to which biotin-conjugated polymerizable molecules can bind. Alternatively, the binding agent may comprise a biotin tag to which an avidin or streptavidin-conjugated polymerizable molecule can bind. Alternatively, the binding agent may be coupled to the activating agent covalently, e.g., using the chemical conjugation approaches described herein or via protein engineering approaches, e.g., generating a fusion protein comprising the polymerizable molecule (e.g., enzyme) and the binding agent (e.g., antibody, antibody fragment, nanobody), SpyTag and SpyCatcher interaction, SNAP-tag, or other attachment or coupling strategy.

In some instances, the binding agent comprises the first polymerizable molecule comprising identifying information of the binding agent. For example, the first polymerizable molecule may comprise a nucleic acid barcode molecule comprising a barcode sequence. The barcode sequence may encode for the identity of the binding agent or the cognate molecule. For example, a monomer (e.g., amino acid) of a polymeric analyte (e.g. peptide comprising a plurality of amino acids) may be cleaved and coupled to the capture moiety (e.g., on a substrate) and may be contacted with a binding agent (e.g., antibody, antibody fragment, nanobody). The binding agent may specifically recognize the amino acid residue or derivative thereof (e.g., a PTH, PTC, ATZ derivatized form) over other amino acid residues or derivatives thereof. The nucleic acid barcode molecule may comprise information that identifies the binding agent, which, due to the specificity of the binding agent to its target, may also identify the particular amino acid residue (or derivative). Alternatively, the binding agent may not comprise the first polymerizable molecule that comprises identifying information of the binding agent or the cognate molecule (e.g., the monomer).

More than one binding agent may be used for a single coupling event of the first and second polymerizable molecules. In some instances, multiple binding agents (e.g., a plurality of monoclonal antibodies or antibody fragments, a plurality of polyclonal antibodies or antibody fragments) may bind to the monomeric analyte. In some instances, a primary binding agent and a secondary binding agent that binds to the primary binding agent are employed. In some examples, subsequent to the binding of the binding agent to the monomer-capture moiety complex, an additional molecule (e.g., a secondary binding agent) comprising a detectable label, e.g., fluorophore, radioisotope, mass tag, an activating agent, or an identifying polymerizable molecule (e.g., nucleic acid barcode molecule) may be contacted with and bind to the binding agent that is bound to the monomer-capture moiety complex. In some examples, the additional molecule comprises an identifying polymerizable molecule, and the identifying polymerizable molecule may be coupled to or transferred to the second polymerizable molecule. In one non-limiting example, the binding agent comprises a primary antibody or antibody fragment (e.g., scFv or nanobody) that recognizes the monomeric analyte, e.g., the monomer-capture moiety complex (e.g., a terminal amino acid-linker-capture moiety complex) or portion thereof (e.g., the terminal amino acid, or the terminal amino acid-linker complex); subsequent to binding of the primary antibody or antibody fragment to the monomer-capture moiety complex or portion thereof, a secondary antibody or antibody fragment comprising or coupled to a polymerizable molecule (e.g., nucleic acid barcode molecule) is coupled to the primary antibody. The polymerizable molecule of the secondary antibody or antibody fragment may comprise information on the secondary antibody or antibody fragment or cognate molecule, the primary antibody or antibody fragment or cognate molecule, or other information. Transfer or coupling of the polymerizable molecule of the secondary antibody or antibody fragment to the additional polymerizable molecule can be mediated by any suitable technique, e.g., hybridization of nucleic acid molecules optionally mediated by a splint molecule, click chemistry, or association of high-affinity molecules (e.g., streptavidin and biotin). Alternatively, or in addition to, the primary or secondary binding agent may comprise an activating agent, e.g., an activating polymerizable molecule such as a nucleic acid molecule or an enzyme, as described elsewhere herein.

A binding agent may comprise or be coupled to any useful number of polymerizable molecules or activating agents. A binding agent may comprise or be coupled to 1, 2, 3, 4, 5, 6, 7, 8, 9, 10, 20, 30, 40, 50, 60, 70, 80, 90, 100 or more polymerizable molecules or activating agents. A population of binding agents may comprise or be coupled to on average, about 1, about 2, about 3, about 4, about 5, about 6, about 7, about 8, about 9, about 10, about 20, about 30, about 40, about 50, about 60, about 70, about 80, about 90, about 100, or more polymerizable molecules or activating agents per binding agent. A binding agent may comprise or be coupled to a numerical range of polymerizable molecules or activating agents, e.g., between 1 and 15 polymerizable molecules, between about 3 and 6 polymerizable molecules, between about 15 and 50 polymerizable molecules, etc.

In some instances, the binding agent comprises an activating polymerizable molecule that comprises additional multiplexed information. For example, the activating polymerizable molecule, e.g., a nucleic acid molecule, may comprise sequences that encode cycle or other temporal information or spatial information. In one such example, an array of peptides and capture moieties may be provided on a substrate. The array may comprise a plurality of individually addressable units, in which each (or a subset of) individually addressable units of the array comprises a peptide to be analyzed and a capture moiety. The binding agents, and the activating polymerizable molecules comprised therein or coupled thereto, may comprise spatial information (e.g., spatial barcode sequences) which uniquely identify the individually addressable units and thus the location of the array. The activating polymerizable molecules of the binding agents may additionally comprise temporal information, e.g., a cycle barcode that indicates the round or iteration in which the binding agent or activating polymerizable molecule is provided. Subsequent sequencing of the activating polymerizable molecule may be used to reveal the spatial information (e.g., the originating location in the array of a peptide or amino acid). In some instances, the activating polymerizable molecule may comprise a unique molecular identifier (UMI), which may be used to determine the quantity of a given binding agent or monomer (e.g., amino acid) for a given peptide, substrate, array, or sample.

Alternatively, or in addition to, the first polymerizable molecule comprising encoded information on the binding agent or the monomer type may comprise multiplexed information. For example, the first polymerizable molecule may comprise sequences that encode cycle or other temporal information or spatial information (e.g., spatial barcode sequences) which uniquely identify a location or individually addressable unit (e.g., of an array). The first polymerizable molecule may additionally comprise temporal information, e.g., a cycle barcode that indicates the round or iteration in which the binding agent or activating polymerizable molecule is provided. Subsequent sequencing of the first polymerizable molecule may be used to reveal the spatial information (e.g., the originating location in the array of a peptide or amino acid) or temporal information. In some instances, the first polymerizable molecule may comprise a unique molecular identifier (UMI), which may be used to determine the quantity of a given binding agent or monomer (e.g., amino acid) for a given peptide, substrate, array, or sample. The first polymerizable molecule may comprise any additional useful functional sequences, e.g., spacers, noncanonical nucleic acids, restriction sites, sequencing primers, read primers, primer sites or primer-binding sites, transposase sites, etc., as described elsewhere herein.

In some instances, the method may comprise contacting the monomer-capture moiety complex with a library of binding agents. The library of binding agents may comprise a plurality of binding agents that have specificity to different analytes. For example, the library of binding agents may comprise a plurality of binding agents that recognize different amino acids or derivatives thereof (e.g., derivatized amino acids such as the PTH, PTC, or ATZ forms), clusters of amino acids (e.g., dipeptides, tripeptides, etc.), or combinations of amino acids (e.g., amino acids with similar side chain groups). In one such example, a given binding agent may recognize and bind to more than one amino acid, optionally with different affinities or binding kinetics. The given binding agent may recognize and bind to a single amino acid, two different amino acids, three different amino acids, four different amino acids, etc. For instance, a given binding agent may bind to amino acids with similar residues, e.g., amino acids with positively-charged side chains (e.g., arginine, histidine, lysine), negatively-charged side chains (aspartic acid, glutamic acid), amino acids with polar uncharged side chains (e.g., serine, threonine, asparagine, glutamine), amino acids with hydrophobic side chains (e.g., alanine, valine, isoleucine, leucine, methionine, phenylalanine, tyrosine, trytophan), aliphatic side chains (e.g., glycine, alanine, valine, leucine, isoleucine), hydroxyl or sulfur or selenium-containing side chains (e.g., serine, cysteine, selenocysteine, threonine, methionine), aromatic side chains (e.g., phenylalanine, tyrosine, tryptophan), basic side chains (e.g., histidine, lysine, arginine), acidic side chains (e.g., aspartate, glutamate, asparagine, glutamine), or a combination thereof. Altogether, the library of binding agents may specifically recognize or bind to any number of different amino acids; for example, the library of binding agents may be configured to specifically bind to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different proteinogenic amino acids or derivatives thereof.

The library of binding agents may comprise any useful number of binding agents, each of which can have different binding specificities. For example, a first binding agent may recognize and one amino acid, and a second binding agent may recognize two amino acids, and a third binding agent may recognize three amino acids. In another example, a first binding agent may recognize one amino acid, a second binding agent may recognize a different amino acid, and a third binding agent may recognize a plurality of amino acids. It will be appreciated that any number of binding agents may be provided, and that each binding agent may have specificity to one or more amino acids. Altogether, the library of binding agents may bind to all 20 proteinogenic amino acids or derivatives thereof, or a subset (e.g., 10 or more, 15 or more) of the amino acids. In instances where primary and secondary binding agents are used (e.g., secondary binding agents that bind to primary binding agents), the primary binding agents may be derived from different species e.g., rabbit, mouse, goat, chicken, llama, etc., to which the secondary binding agents may selectively bind (e.g., anti-rabbit, anti-mouse, anti-goat, anti-chicken, anti-llama antibodies).

In some instances, passivation of a binding agent or of a substrate may be performed prior to or during contact with the cleaved monomer. Passivation may be achieved using a blocking agent or solution, such as milk proteins (e.g., lactoglobulin, lactalbumin, lactoferrin, casein, whey, immunoglobulin, insulin, growth factors, osteopontin), albumin (e.g., bovine serum albumin), Tween 20, commercially available blocking solutions, or a combination thereof. Alternatively, or in addition to, passivation may be performed using a polymer (e.g., polyethylene glycol), organic compound (e.g., oil, lipids), sugar, nanoparticle, inorganic compound, ion, etc.

Coupling of Polymerizable Molecules: The polymerizable molecules (e.g., the first polymerizable molecule and the second polymerizable molecule) may be coupled to one another using any useful approach. Such coupling may comprise a covalent interaction or a noncovalent interaction (e.g., ionic interaction, hydrophobic interaction, van der Waals forces, etc.). In some instances, the first polymerizable molecule and the second polymerizable molecule comprise nucleic acid molecules and may be coupled via hybridization, ligation, or both. For instance, the first polymerizable molecule may comprise a first sequence that is complementary to a second sequence of the second polymerizable molecule, and the coupling may occur via hybridization of the first sequence to the second sequence. Alternatively, the first sequence and the second sequence may not be complementary to one another but may be complementary to a third sequence and a fourth sequence, respectively, of a splint or bridge oligonucleotide. Accordingly, coupling of the first polymerizable molecule to the second polymerizable molecule may be mediated by hybridization of the first and second sequences to the third and fourth sequences, respectively, of the splint or bridge oligonucleotide.

In some instances, a nucleic acid reaction may be performed as part of or in addition to the coupling of the first polymerizable molecule to the second polymerizable molecule. For example, the first sequence of the first polymerizable molecule may hybridize to the second sequence of the second polymerizable molecule, and a nucleic acid extension reaction (e.g., using a polymerase) may be performed. Such an extension reaction may allow for transfer of the encoded information of one of the polymerizable molecules (e.g., the first polymerizable molecule) to another polymerizable molecule (e.g., the second polymerizable molecule). In another example, the first sequence of the first polymerizable molecule may be ligated to the second sequence of the second polymerizable molecule to provide a first polymerizable molecule covalently coupled to the second polymerizable molecule. In one such example, the first sequence may hybridize to a first splint sequence of a splint oligonucleotide, and the second sequence may hybridize to a second splint sequence of the splint oligonucleotide. A ligation may be performed to covalently link the first sequence to the second sequence. In some instances, an extension reaction and a ligation reaction may be performed to couple or transfer information from the first polymerizable molecule to the second polymerizable molecule. Optionally, the polymerizable molecules may be decoupled, e.g., via denaturation or cleavage at a cleavage site, subsequent to the coupling.

The polymerizable molecules may be coupled chemically, either covalently or noncovalently. In some instances, the first polymerizable molecule may be chemically linked to the second polymerizable molecule. For example, the first polymerizable molecule may comprise a first reactive moiety, and the second polymerizable molecule may comprise a second reactive moiety that is capable of reacting with the first reactive moiety. The first reactive moiety may be contacted with the second reactive moiety and be subjected to conditions sufficient to link the first reactive moiety to the second reactive moiety, e.g., via click chemistry. In other instances, the first polymerizable molecule may be coupled to the second polymerizable molecule via a noncovalent or indirect interaction, e.g., biotin-streptavidin.

In some instances, the first polymerizable molecule, e.g., comprising identifying information of the monomer (e.g., monomer identity such as the amino acid type) or the binding agent can be coupled to additional polymerizable molecules. For example, a substrate may comprise the polymeric analyte and capture moiety coupled thereto, along with a plurality of additional polymerizable molecules. Subsequent to coupling of the monomer to the capture moiety and cleavage of the monomer from the polymeric analyte, the monomer may be contacted with the same or different binding agents any number of times. The first polymerizable molecule of a binding agent may contact and be coupled to any number of the additional polymerizable molecules iteratively for repeated interrogation; for instance, the first polymerizable molecule may couple to a first additional polymerizable molecule, as described herein, and then subsequently cleaved or removed (e.g., via dehybridization, enzymatic cleavage) and contacted and coupled to a second additional polymerizable molecule. Such an approach may be advantageous in transferring several copies of the first polymerizable molecule to the substrate, e.g., to improve sensitivity. Alternatively, or in addition to, the first polymerizable molecule may iteratively be coupled to the same first additional polymerizable molecule, e.g., to generate a stacked polymerizable molecule comprising multiple copies of the polymerizable molecule of the binding agent.

Alternatively, or in addition to, a plurality of first polymerizable molecules may be provided and coupled to the additional polymerizable molecules. The plurality of first polymerizable molecules may comprise, for example, a plurality of nucleic acid barcode molecules comprising identical sequences. The plurality of first polymerizable molecules may be coupled to the additional polymerizable molecules, which can improve sensitivity of monomer detection (e.g., by having multiple detectable copies of the first polymerizable molecule). In some instances, the plurality of first polymerizable molecules comprise a plurality of nucleic acid barcode molecules with different sequences. In such instances, the different sequences may represent different monomer types (e.g., the 20 proteinogenic amino acids, post-translational modifications, etc.). The different sequences may additionally comprise a unique priming region, e.g., for annealing or coupling to an activating agent (e.g., coupled to a binding agent) that also comprises a unique priming sequence. For instance, each binding agent type that is specific to one or more monomers may comprise an activating agent with a unique priming sequence; upon provision of the plurality of first polymerizable molecules with the different sequences, the plurality of first polymerizable molecules may selectively bind to the different binding agents based on matching or complementarity of the unique priming sequences.

Decoupling Monomers, Binding Agents: In some instances, subsequent to the coupling of the first polymerizable molecule to the second polymerizable molecule, the monomer may be decoupled from the monomer-capture moiety complex or the substrate. The decoupling may be performed chemically, mechanically, or enzymatically. For example, the monomer may be coupled to the capture moiety via a linking nucleic acid molecule (e.g., a linker comprising a monomer reactive group and a linking nucleic acid molecule). The linking nucleic acid molecule may comprise a cleavage site, e.g., restriction site, and the decoupling may be performed by enzymatic cleavage at the cleavage site, using, for example, a restriction endonuclease. Alternatively, or in addition to, the capture moiety or any of the polymerizable molecules may comprise a cleavage site which may allow for decoupling of the monomer from a portion of the capture moiety. Examples of cleaving enzyme include, in non-limiting examples, glycosylases (e.g., uracil glycosylase), restriction endonucleases, micrococcal nucleases, transposases, Cas proteins (e.g., Cas9), Argonaut endonucleases, etc. Beneficially, the removal of the monomer from the monomer-capture moiety complex can allow the capture moiety to be available for subsequent reactions or iterations or to prevent additional binding agents (e.g., within a single cycle or in subsequent iterations) from binding to the capture moiety, which may help reduce erroneous or duplicative coupling of the polymerizable molecule of the additional binding agents to the capture moieties. Alternatively, or in addition to, the decoupling may occur using a stimulus, e.g., a photo-stimulus (such as UV, gamma, X-ray irradiation), thermal stimulus, chemical stimulus, etc. In some instances, the linker may comprise a cleavable group and application of the appropriate stimulus may result in cleaving of the linker, as described elsewhere herein.

Alternatively, or in addition to, the monomer may be altered such that it is rendered undetectable by the binding agent, e.g., to prevent binding of the binding agent to the cleaved monomer in subsequent iterations of cleaving, coupling to the capture moiety, and contacting with additional binding agents. For example, the monomer may be contacted with a blocking agent or derivatized such that the binding agent no longer recognizes the derivatized form. Such blocking strategies may be useful in eliminating the need to remove cleaved monomers following detection or transfer of information from the binding agent-coupled polymerizable molecules. Additional strategies for inhibiting binding of binding agents to cleaved monomers are described elsewhere herein.

Similarly, in some instances, the binding agent may be removed from the monomer-capture moiety complex at any useful or convenient operation, e.g., subsequent to coupling of the polymerizable molecules. Removal of the binding agent may be performed using chemical or enzymatic approaches, e.g., using chemical denaturants, detergents, acidic or alkaline conditions, heat, or proteases. Alternatively, or in addition to, if a polymerizable molecule is coupled to the binding agent, the polymerizable molecule may be removed from the binding agent, e.g., via a cleavage or restriction site and use of a cleaving enzyme (e.g., UDG, restriction enzyme), chemical cleavage, photolysis, or other approach. In some instances, the polymerizable molecule is coupled to the binding agent via a noncovalent interaction, e.g., desthiobiotin-avidin; accordingly, decoupling of the polymerizable molecule from the binding agent may be achieved by use of a competition agent, e.g., a higher-affinity biotin to competitively replace the desthiobiotin.

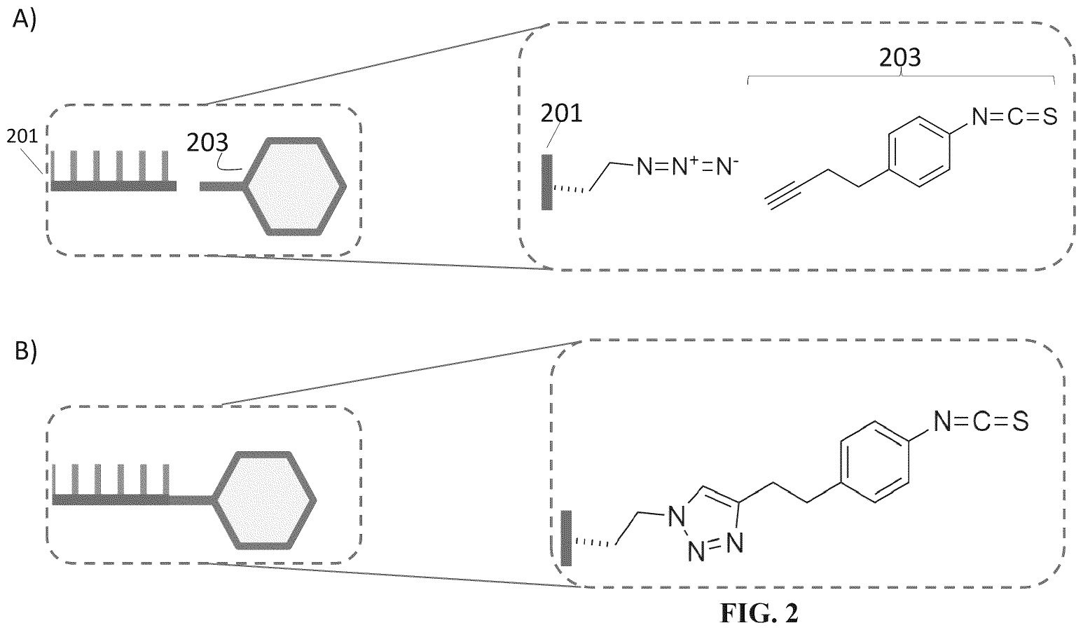

FIG. 1A shows an example workflow of analyzing a polymeric analyte. In workflow 100, a polymeric analyte 103 is provided, sequentially disassembled into individual monomers via contacting with capture moieties and cleavage, and the individual cleaved monomers are contacted with binding agents comprising polymerizable molecules that identify or encode for the binding agents. The polymerizable molecules are coupled to an additional polymerizable molecule, and the monomer is cleaved from the capture moiety. In FIG. 1A Panel A, a substrate 101 is coupled to a polymeric analyte 103 (e.g., a peptide to be sequenced), a capture moiety 105 (e.g., a nucleic acid primer), and an additional polymerizable molecule 107 (e.g., an additional nucleic acid primer). In some instances, the capture moiety 105 and the additional polymerizable molecule 107 are identical molecules (e.g., comprise the same sequence). The polymeric analyte may be contacted with a bifunctional linker 109, which comprises an amino acid coupling group (e.g., PITC) and a click chemistry moiety (e.g., azide). In some instances, the bifunctional linker 109 couples to a terminal monomer (e.g., terminal amino acid such as the N-terminal amino acid (NTAA)). Such coupling may be performed using a chemical reaction, e.g., of PITC with the terminal amino acid to generate a phenylthiocarbamoyl-derivatized amino acid. In FIG. 1A Panel B, a linking nucleic acid molecule 111 comprising a click chemistry moiety (e.g., alkyne such as DBCO) is reacted with the bifunctional linker 109 and covalently linked. In some instances, the linking nucleic acid molecule 111 and the bifunctional linker 109 are provided pre-coupled (see, e.g., FIG. 2). In FIG. 1A Panel C, the linking nucleic acid molecule 111 is coupled to the capture moiety 105, which may be mediated by hybridization of the linking nucleic acid molecule 111 to the capture moiety 105 (hybridization not shown), or by using a splint oligonucleotide 113 comprising sequences complementary to a sequence of the linking nucleic acid molecule 111 and the capture moiety 105. In some instances (not shown), the linking nucleic acid molecule 111 comprises a self-splinting sequence, such that the linking nucleic acid molecule 111 may couple to the capture moiety 105 in the absence of a separate splint molecule (see FIG. 24). A ligase may be used to covalently link the linking nucleic acid molecule 111 to the capture moiety 105. Alternatively, the linking nucleic acid molecule 111 may comprise a first reactive moiety (not shown) that can react with a second reactive moiety (not shown) of the capture moiety 105. In FIG. 1A Panel D, the system is subjected to conditions sufficient to cleave the terminal monomer (e.g., amino acid) from the polymeric analyte 103 (e.g., peptide). The conditions may include performing an Edman degradation reaction, e.g., using an acid and heat to cleave the terminal amino acid. The cleavage of the monomer from the polymeric analyte results in a cleaved monomer that is coupled to the bifunctional linker 109, linking nucleic acid molecule 111, and capture moiety 105. In FIG. 1A Panel E, a binding agent 115 (e.g., antibody, antibody fragment, nanobody, scFv) comprising another polymerizable molecule 117 (e.g., nucleic acid molecule) is provided. The binding agent 115 may bind specifically to the monomer (e.g., to a single amino acid), the monomer-linker complex (e.g., PITC-amino acid), or the monomer-linker-capture moiety complex. The polymerizable molecule 117 of the binding agent may comprise information, e.g., a barcode sequence, on the identity of the binding agent or the specific monomer (e.g., single amino acid) to which the binding agent binds. The polymerizable molecule 117 of the binding agent may comprise additional sequences, such as a UMI, restriction site, transposition site, a sequence to represent a cycle or iteration number, or other functional sequence, e.g., sequencing sites or sequences (e.g., P5 or P7 sequences), primer-binding sequences, read sequences (e.g., R1 or R2 sequences). The polymerizable molecule 117 of the binding agent may couple to the additional polymerizable molecule 107 that is coupled to the substrate 101. In some instances, an extension reaction may be performed (e.g., using a polymerase), to copy the sequence of the polymerizable molecule 117 of the binding agent to the additional polymerizable molecule 107 that is coupled to the substrate 101. Alternatively, the polymerizable molecule 117 of the binding agent may be ligated to the additional polymerizable molecule 107, either chemically (e.g., via click chemistry) or enzymatically (e.g., using a ligating enzyme or enzyme-analog such as a ribozyme or DNAzyme). In FIG. 1A Panel F, the monomer may be decoupled (e.g., removed or cleaved) from the capture moiety 105. For example, the monomer, bifunctional linker 109, and all or a portion of the linking nucleic acid molecule 111 may be cleaved (depicted as a star). The cleavage may be performed chemically, mechanically, or enzymatically. In an example of enzymatic cleavage, the linking nucleic acid molecule 111 may comprise a restriction site or other cleavage site (e.g., a uracil), and cleavage occurs by introduction of a restriction enzyme or cleaving enzyme (e.g., uracil DNA glycosylase) to cleave the restriction/cleavage site. The workflow 100 may then be iterated or repeated to sequence all or a portion of the polymeric analyte 103.

FIG. 1B schematically shows an example workflow of analyzing a polymeric analyte using a binding agent comprising an activating agent (e.g., polymerizable molecule). Part of the workflow as shown in FIG. 1A Panels A-D may be performed, thereby generating a cleaved monomeric analyte that is coupled to a bifunctional linker 109, a capture moiety 105, and in some instances, a linking nucleic acid molecule 111. A binding agent 115 comprising an activating polymerizable molecule 123 (e.g., activating nucleic acid molecule) is contacted with the monomeric analyte and may bind specifically to the monomeric analyte (e.g., to a single amino acid) or portion thereof, or the binding agent 115 may bind semi-specifically to a plurality of different monomers or portions thereof. A polymerizable molecule 119 (e.g., a nucleic acid barcode molecule), which may comprise identifying information of the monomer (e.g., the amino acid type) may be provided and coupled to the activating polymerizable molecule 123 via hybridization (not shown), or using a splint molecule 121, which comprises sequences complementary to a portion of the activating polymerizable molecule 123, the polymerizable molecule 119, and optionally, the additional polymerizable molecule 107. The polymerizable molecule 119 is optionally covalently coupled to the additional polymerizable molecule 107, e.g., via ligation, and the binding agent 115 and activating polymerizable molecule 123 may be removed to repeat the workflow for additional monomers of the polymeric analyte.

In some instances, the activating polymerizable molecule 123 comprises a unique priming sequence, and the splint molecule 121 comprises a unique priming complement sequence that can anneal to the unique priming sequence of the polymerizable molecule 123. Accordingly, multiplexing of multiple monomer types using multiple binding agents is achievable by designing the unique sequences of the activating polymerizable molecule 123, the splint molecule 121, and the polymerizable molecule 119 comprising the identifying information.

FIG. 1C-1D schematically show another example workflow for analyzing a polymeric analyte using hybridization chain reaction (HCR) to generate a polymerizable molecule comprising encoded information. In FIG. 1C Panel A, a binding agent 115 is provided coupled to an activating polymerizable molecule (e.g., nucleic acid molecule) that comprises a first sequence 104 and a second sequence 102. In FIG. 1C Panel B, two hairpin oligonucleotides (Oligo A and Oligo B) are provided. A first hairpin molecule, Oligo A, comprises sequences 104′ (complement to the first sequence 104), 102′ (complement to the second sequence 102), 106, and 102, which anneals to 102′. A second hairpin molecule, Oligo B, comprises a flap sequence 108, 106′, 102′, 104, and 102, which anneals to 102′. In some instances, Oligo A does not bind or hybridize to Oligo B in the absence of an activating sequence. Oligo A hybridizes to the activating polymerizable molecule by complementary base pairing to generate an Oligo A-activating polymerizable complex, which can prime hybridization of Oligo B. In FIG. 1C Panel C, Oligo B anneals to the Oligo A-activating polymerizable complex. In some instances, Oligo A, Oligo B, or both hairpin molecules comprise a barcode sequence segment. Subsequent to hybridization, the flap sequence 108 of Oligo B comprises a single-stranded overhang. In FIG. 1C Panel D, multiple rounds of HCR may be performed to anneal additional copies of Oligo A and Oligo B to generate a concatenated sequence of Oligo A and Oligo B molecules. In FIG. 1C Panel E, the binding agent 115 may be contacted with a monomeric analyte (e.g., as shown in workflow 100 and FIG. 1A Panels A-D) that is coupled to a capture moiety 105 on a substrate that also comprises a plurality of additional polymerizable molecules 107. The flap sequences 108 of the concatenated sequence can couple, e.g., via hybridization or a splint, to one or more of the additional polymerizable molecules 107. Alternatively, or in addition to, an extension reaction may be performed to couple Oligo B to an additional polymerizable molecule 107.

As described in the context of FIG. 1C, a first polymerizable molecule (e.g., comprising encoding information of the binding agent or the monomer type) may refer to any single or combination of the first hairpin molecule (Oligo A), the second hairpin molecule (Oligo B), or additional polymerizable molecules. For example, the first polymerizable molecule may comprise the first hairpin molecule, or the first polymerizable molecule may comprise the second hairpin molecule. In another example, the first polymerizable molecule may comprise a concatenated sequence of the first hairpin molecule and the second hairpin molecule. Any number of different hairpin molecules may be used for HCR, e.g., at least 2, at least 3, at least 4, at least 5, at least 6, at least 7, at least 8, at least 9, at least 10, or greater number of hairpin molecules. Similarly, the concatenated sequence of hairpin molecules may comprise any useful number of hairpin molecules, e.g., at least 2, at least 5, at least 10, at least 20, at least 50, at least 100, or greater number of hairpin molecules.

As described elsewhere herein, it will be appreciated that the order of operations may be altered. For example, the binding agent may be contacted with the monomeric analyte prior to conducting the HCR reaction. In another example, the HCR reaction may be conducted prior to contacting the binding agent with the monomeric analyte, e.g., to generate a barcoded library of binding agents prior to contacting with the monomeric analytes.

FIG. 1D schematically shows iteration of the workflow described in FIG. 1C. In FIG. 1D Panel A, a plurality of Oligo B molecules is coupled to the additional polymerizable molecules 107 via the flap sequence (108 of FIG. 1C). In FIG. 1D Panel B, cleavage or removal of a portion of the Oligo B molecules (e.g., sequences 102 and 106) may be performed. Optionally, the monomeric analyte may also be removed from the substrate, e.g., via cleavage of the linking nucleic acid molecule or the capture moiety. In FIG. 1D Panel C, the next (e.g., n-1) monomer is locally tethered to the substrate via a capture moiety, a bifunctional linker, and a linking nucleic acid molecule, as described in FIG. 1A. An additional binding agent is provided which can recognize the n-1 monomer, which may be coupled to an additional concatenated sequence. The additional concatenated sequence may comprise a different sequence than that of the (first) binding agent. In some examples, the additional concatenated sequence may be generated using the same activating polymerizable molecule comprising sequences 104 and 102, Oligo A, and Oligo C, which may comprise a different barcode sequence segment as Oligo B. Oligo C, like Oligo B, also comprises a flap sequence that enables coupling of Oligo C to the cleaved Oligo B molecules. In FIG. 1D Panel D, the flap sequences of the additional concatenated sequence are coupled, e.g., via hybridization or a splint or an extension reaction, to one or more of the cleaved Oligo B molecules, thereby generating a stacked polymerizable molecule comprising a first barcode sequence segment (Oligo B) that comprises the identity of the first (terminal) monomer and a second barcode sequence segment (Oligo C) that comprises the identity of the next (n-1) monomer. Additional iterations of the workflow may be performed, e.g., until all of the monomers have been locally tethered, cleaved, and contacted with binding agents.

In some instances, the activating agent comprises an anchoring nucleic acid molecule which can indirectly facilitate coupling of the first polymerizable molecule and the second polymerizable molecule. FIG. 1E schematically shows an example of a binding agent comprising a polymerizable molecule and an activating agent. The binding agent 115 may comprise one or more polymerizable molecules 117 that identifies the binding agent or its cognate molecule (e.g., a monomer type), and an activating agent 123 that comprises an anchoring nucleic acid molecule. The anchoring nucleic acid molecule may comprise a region that can couple to a substrate-bound polymerizable molecule 107. In some instances, the anchoring nucleic acid molecule comprises a self-splinting region that can partially hybridize to the substrate-bound polymerizable molecule 107. Optional ligation may be performed to covalently link the activating agent 123 to the substrate-bound polymerizable molecule 107. Prior to, during, or subsequent to the coupling of the activating agent 123 to the substrate-bound polymerizable molecule 107, a polymerizable molecule 117 coupled to the binding agent may couple to an additional substrate-bound polymerizable molecule. Alternatively, or in addition, polymerizable molecule 117 may couple to a substrate-bound polymerizable molecule comprising an encoded polymerizable molecule from a previous round or cycle or iteration, thereby generating a stacked polymerizable molecule comprising encoded information from multiple rounds or cycles.

FIG. 1F schematically shows an example activating agent comprising an enzyme. The binding agent 115 may be coupled to the activating agent 123, which may comprise any useful enzyme, e.g., a peroxidase, a ligase, a kinase, a phosphatase, a phosphorylase, or a nuclease, e.g., a Cas protein, a restriction enzyme. In some examples, the enzyme comprises a peroxidase (e.g., horseradish peroxidase or APEX) or other chromogenic or fluorogenic enzyme, which can generate a direct readout product (e.g., chemiluminescence, luminescence, fluorescence, etc.). The product may be detectable using standard imaging or fluorescence or luminescence detection methods. Alternatively, or in addition to, the enzyme may facilitate coupling of polymerizable molecules, e.g., coupling an encoding (first) polymerizable molecule such as a nucleic acid barcode molecule that identifies the binding agent or the monomer type to a second polymerizable molecule, e.g., a substrate-bound nucleic acid molecule. For example, a peroxidase may be used to locally couple a click chemistry-functionalized tyramide (e.g., azide-tyramide) to the substrate (e.g., comprising a tyrosine or radical acceptor) or to a substrate-bound (second) polymerizable molecule (e.g., a fluorescein-conjugated nucleic acid molecule, as described in Wilbanks et al. 2021. Chembiochem. 22(8):1400-1404, incorporated by reference herein, or a tyrosine-containing polymerizable molecule) in proximity to the monomeric analyte; subsequently, an encoding (first) polymerizable molecule comprising a complementary click chemistry moiety (e.g., DBCO) may conjugate to the tyramide-modified substrate-bound (second) polymerizable molecule. Alternatively, or in addition to, a tyramide-conjugated encoding (first) polymerizable molecule (e.g., nucleic acid barcode molecule) may be directly coupled to the substrate-bound (second) polymerizable molecule (e.g., a fluorescein-conjugated nucleic acid molecule or a tyrosine-containing polymerizable molecule) via the peroxidase coupled to the binding agent.

In another example, the activating agent comprises a ligase that may be used to ligate the encoding (first) polymerizable molecule to the substrate-bound (second) polymerizable molecule, optionally using a splint molecule. In yet other examples, the enzyme may be used to process or activate the substrate-bound (second) polymerizable molecule or the first polymerizable molecule to thereby enable coupling. For example, the substrate-bound (second) polymerizable molecule may comprise a restriction site or cleavage site that can be cleaved, e.g., using a restriction enzyme or other cleaving enzyme, thereby revealing a coupling site to which the encoding (first) polymerizable molecule can couple, e.g., via hybridization with one another or with a splint molecule. In some instances, the first and second polymerizable molecules may not be able to couple to another in the absence of the activating (e.g., cleaving) operation. In other examples, the enzyme may comprise a kinase, e.g., T7 polynucleotide kinase, which can phosphorylate the first or second polymerizable molecule, thereby allowing for ligation of the first polymerizable molecule to the second polymerizable molecule.

FIG. 2 schematically shows an example linker that may be used in sequencing polymeric analytes such as peptides. FIG. 2 Panel A shows a bifunctional linker 203 (e.g., 1-(but-3-yn-1-yl)-4-isothiocyanatobenzene, also referred to herein as alkyne-PITC or PITC-alkyne) comprising an amino acid reactive moiety (e.g., PITC) and an alkyne click chemistry moiety, which may be reacted with a polymerizable molecule 201 (e.g., a linking nucleic acid molecule) comprising a complementary azide click chemistry moiety. The bifunctional linker may also comprise a spacer moiety, e.g., an alkyl chain (an ethyl group is depicted) of any length, a polymer (e.g., PEG) of any length, etc. The spacer moiety may be located between the amino acid reactive moiety and the click chemistry moiety. FIG. 2 Panel B shows the product of a click chemistry cycloaddition reaction between the azide and alkyne groups to generate a linker molecule comprising the polymerizable molecule and the amino acid reactive moiety. The conjugation of the polymerizable molecule 201 to the bifunctional linker 203 may occur at any useful or convenient step. In alternative examples (not shown), the bifunctional linker 203 may comprise an azide group, e.g., 1-(2-azidoethyl)-4-isothiocyanatobenzene (also referred to herein as azide-PITC or PITC-azide), which can be reacted to a polymerizable molecule 201 comprising an alkyne moiety.

Iteration: In some instances, one or more of the operations described herein may be iterated or repeated. Iteration of the operations may allow for sequential processing, analysis, or identification of the individual monomers of the polymeric analyte, which can allow for reconstruction of the entire polymeric analyte. For example, referring to FIG. 1, the operations of workflow 100 may be conducted to encode the identity (e.g., via the polymerizable molecule 117 of the binding agent) of a terminal amino acid (e.g., NTAA) onto an additional polymerizable molecule 107. The operations of workflow 100 may then be repeated to encode the identities of the n-1 terminal amino acid, the n-2 terminal amino acid, the n-3 terminal amino acid, etc., until the entire or portion of the peptide is sequenced. The encoding may occur on the same (additional) polymerizable molecule 107, e.g., to generate a stacked polymerizable molecule comprising multiple polymerizable molecules from multiple binding agents, or the encoding may occur on additional polymerizable molecules (not shown) present on the substrate. In the former situation, in some instances, the polymerizable molecule of the second (or third, fourth, fifth, . . . nth) cycle may be configured to only couple to the first (or second, third, fourth, . . . n-lth) polymerizable molecule. For example, the first cycle binding agent polymerizable molecule may comprise a unique binding sequence that is absent on the additional polymerizable (or capture) molecules of the substrate, and to which the second cycle binding agent polymerizable molecule can bind. Accordingly, the second cycle binding agent polymerizable molecule can only bind to the first cycle binding agent polymerizable molecule and not to any of the additional polymerizable (or capture) molecules of the substrate. In the event that no binding occurs (a “null” event), a bridging polymerizable molecule may be provided that encodes for a null binding event but comprises the unique binding sequence, such that subsequent rounds may continue, even if a binding agent does not bind the cleaved monomer.

In instances where one or multiple additional polymerizable molecules are used, the polymerizable molecules 117 of the binding agents may additionally comprise temporal information on the cycle or iteration number, such that the order of the individual monomers may be determined. For example, for a given peptide, the terminal amino acid may be coupled to a capture moiety and cleaved, then contacted with a binding agent comprising a barcode sequence that identifies (i) the identity of the amino acid (e.g., any one of twenty proteinogenic amino acids) and (ii) the cycle number (e.g., cycle 1). The information encoded by the barcode sequence may be transferred, via coupling and optional copying, to an adjacent (additional) polymerizable molecule 107. Following cleavage of the monomer from the capture moiety (e.g., as shown in FIG. 1A Panel F), the workflow 100 may be repeated for the n-1 terminal amino acid, which may again be coupled to a capture moiety, cleaved, and contacted with a binding agent comprising an additional barcode sequence that identifies (i) the identity of the amino acid (e.g., any one of twenty proteinogenic amino acids) and (ii) the cycle number (e.g., cycle 2). The information encoded by the additional barcode sequence may be transferred to the same (additional) polymerizable molecule 107, or an additional polymerizable molecule (not shown) present on the substrate. In the former situation, the polymerizable molecule may then comprise information on the (i) the identity of the terminal amino acid, (ii) the cycle number of the terminal amino acid (cycle 1), (iii) the identity of the n-1 terminal amino acid, and (iv) the cycle number of the n-1 terminal amino acid (cycle 2), and so forth.

Alternatively, temporal information may be provided separately. For example, prior to, during, or subsequent to coupling of the first polymerizable molecule to the second polymerizable molecule, a temporal barcode may be provided that can couple to the first polymerizable molecule, the second polymerizable molecule, or both. The temporal barcode may comprise any useful agent, including a nucleic acid molecule, a peptide, a lipid, a carbohydrate, an enzyme (e.g., a chromogenic or fluorogenic enzyme) or a ribozyme or DNAzyme, a fluorophore, a dye, an intercalating agent, a dideoxynucleotide, a fluorescent nucleic acid molecule or nucleotide, a radioisotope, a mass tag, or other detectable label that can indicate the time or cycle (or iteration) number in which it is provided. In some instances, the temporal barcode comprises a cycle-specific nucleic acid barcode molecule, which can couple to the first polymerizable molecule (comprising the identity of the monomer) or to a terminal polymerizable molecule of a stacked polymerizable molecule comprising polymerizable molecules from multiple rounds or iterations. The temporal barcode may comprise any additional useful functional sequences, e.g., primer sites, sequencing sites, restriction sites, abasic or cleavable sites, etc. In some instances, the temporal barcode may comprise an amplification site that allows for bridge amplification of the temporal barcode and optionally, the coupled polymerizable molecules, to other capture or polymerizable molecules.

One or more operations may be repeated within a given workflow. For example, referring to workflow 100, any of the processes of FIG. 1A Panels A-F may be iterated or repeated. In some instances, multiple rounds of binding-agent coupling may be performed (e.g., as shown in FIG. 1A Panel E), optionally with wash operations, which can result in (i) multiple polymerizable molecules (e.g., 107) comprising the same encoded information of the polymerizable molecules 117 of the binding agent, (ii) the same polymerizable molecule (e.g., 107) comprising multiple copies of the same polymerizable molecule 117 of the binding agent, or (iii) both. This multiple-cycle encoding approach may be useful, for example in improving sensitivity or accuracy of detecting individual monomers.

Identification of polymerizable molecules: The polymerizable molecules may be subjected to sequencing to determine the identity of the individual monomers (e.g., amino acids). For example, following cleavage of the monomer from the capture moiety (e.g., as shown in FIG. 1A Panel F), or any number of iterations of workflow 100, the polymerizable molecules comprising information of the binding agents, and thus the identity of the monomers, may be removed from the substrate and prepared for sequencing (e.g., DNA sequencing, NGS). Removal of the polymerizable molecules may be accomplished using any useful approach, e.g., chemical or enzymatic cleavage. In some instances, any excess or uncoupled polymerizable molecules or capture moieties may be removed, e.g., prior to removal of the polymerizable molecules comprising monomer information. For example, referring again to FIG. 1A Panel B and FIG. 1A Panel E, the coupling events may generate double-stranded or partially-double-stranded molecules; accordingly the single-stranded polymerizable molecules or capture moieties that do not comprise a monomer or additional polymerizable molecules (e.g., from the binding agents) may be digested using an enzyme such as type II restriction endonucleases, S1 endonucleases.

Alternatively, or in addition to, the polymerizable molecules may be amplified (e.g., using nucleic acid amplification approaches such as polymerase chain reaction (PCR), isothermal amplification, ligation-mediated amplification, transcription-based amplification, etc.) to generate amplicons for sequencing. Amplification may be performed, for example, using the capture moieties or polymerizable molecules as primer binding sites. Alternatively, or in addition to, an adapter sequence comprising a primer binding site may be added to the polymerizable molecules. Any number of useful preparation operations may be performed, such as purification or enrichment, cleanup, nucleic acid reactions (e.g., ligation, extension, amplification, tagmentation, restriction enzyme cleavage, enzymatic treatment (e.g., using exonucleases, RNase, CRISPR, Argonaut, terminal transferase)), fragmenting, barcoding, addition of adapters, enzymatic treatment, etc. In some instances, the polymerizable molecules, or the substrates comprising the polymerizable molecules, may be filtered based on any useful characteristic or properties. Filtering based on a characteristic or property may achieve higher accuracy or less noise by removing poor quality molecules or enriching for higher quality polymerizable molecules prior to sequencing. For example, polymerizable molecules or substrates (e.g., beads or particles) containing the polymerizable molecules may be filtered by size or length, quantity, presence of particular sequences (e.g., primer sequences, sequences of interest), GC content, polarity, polarization, birefringence, fluorescence (or other optical property), anisotropy, charge, secondary structure (e.g., hairpins), or other useful metric, characteristic, or property or combinations thereof. Such filtration or enrichment may be performed using any suitable approach, e.g., affinity or hybridization approaches (e.g., bead-based affinity sequences or hybridization assays, which can enrich particular sequences), chromatography, size-based filtration, electrophoresis, electrofocusing, optoelectronics, digital fluidics, magnetic activated sorting, fluorescence activated sorting, flow cytometry, or other suitable technique.

Sequencing may be performed using a commercially available nanopore system, e.g., Oxford Nanopore Technologies, Genia Technologies, NobleGen, or Quantum Biosystem, or other sequencing and next generation sequencing systems, e.g., Illumina, BGI, Qiagen, ThermoFisher, PacBio, and Roche, including formats such as parallel bead arrays, sequencing by synthesis, sequencing by ligation (e.g., SOLiD), capillary electrophoresis, electronic microchips, “biochips,” microarrays, parallel microchips, single-molecule arrays, and Sanger sequencing, as is described elsewhere herein.

Sequencing may output the identity of the polymerizable molecules or sequences of polymerizable molecules that are coupled together. For example, referring again to FIG. 1, subsequent to one or more iterations of workflow 100, the polymerizable molecule 107 may comprise a nucleic acid sequence of the polymerizable molecule 117 of the binding agent (or complement thereof), or stacks of polymerizable molecules obtained from multiple rounds of binding agents binding to their target monomer or monomer-capture moiety complex (or complements thereof). Sequencing of the polymerizable molecule 107 may therefore yield sequencing reads that identify the nucleic acid sequence of the polymerizable molecule 117 of the binding agent and the information encoded therein, e.g., the cycle number and the identity of the binding agent or monomer (e.g., one of the 20 proteinogenic amino acids). In instances where the polymerizable molecule 117 of the binding agent comprises a nucleic acid molecule that encodes additional information (e.g., comprises barcode sequences, UMIs, cycle information, spatial information etc.), multiple types of information may be revealed from the nucleic acid sequencing of the polymerizable molecule 107.

Sequencing reads may be assembled using a de novo approach to identify the peptide or protein. For instance, fragmented peptides arising from a common parent protein may be labeled with a common barcode sequence, as described elsewhere herein. Putative peptide reads can thus be assembled based on the common barcode sequence, amino acid identity, and if applicable, cycle number. Erroneous reads may be identified through probabilistic modeling of accuracy of reads, resulting in reconstructed, fragmentary, peptide sequences (contigs) with possible gaps for missed or unidentified rounds/amino acid. An alternative option for de novo read reconstruction may employ end-to-end, unsupervised machine learning based reconstruction of peptide reads. This option may employ a Machine Learning Algorithm, such as a deep-learning based model that takes as its input NGS sequencing reads associated with a parent protein/peptide barcode, and outputs the likely reconstruction of peptide reads (contigs). Training of the model can be conducted with protein sequencing runs using known protein/peptide standards. The de novo reconstruction may output reconstructed, fragmentary, peptide sequences (contigs) with a probability assigned to each amino acid as well as the assembled peptide sequence. In some instances, a k-mer or De Brujin approach may be used for peptide sequence reconstruction. For example, reads arising from each polymerizable molecule may be broken down into shorter k-mer sequences. The k-mer sequences from the pool of reads may be assembled into longer contig sequences. A De Brujin graph may be generated, e.g., to represent splice variants, post-translational modifications, or other proteoforms. The isoforms may be assembled, and the expression level may be determined using a Bayesian approach. The assembled isoforms of proteins may be subjected to evaluation and error correction, e.g., by comparison with standard proteins that are spiked in samples, and assessing for missing segments of sequences, incorrect or redundant assembly, uniform coverage, etc.

Alternatively, the identity of the polymerizable molecules may be obtained without use of a sequencing approach. For instance, probes may be used to couple to particular regions of a polymerizable molecule. The probes may comprise nucleic acid probes with probe sequences that can be used to specifically detect a type of monomer. In one such example, the polymeric analyte comprises a peptide, and an individual amino acid (monomeric unit) may be coupled to a capture moiety and cleaved from the peptide. The monomer-capture moiety complex may be contacted with a binding agent (e.g., antibody, nanobody, scFv) comprising a nucleic acid barcode molecule (polymerizable molecule) that identifies the binding agent. The binding agent may be specific to one amino acid (e.g., of the 20 proteinogenic amino acids) and as such, the nucleic acid barcode molecule encodes for one specific amino acid. Accordingly, a nucleic acid probe having a complementary sequence to the nucleic acid barcode molecule of the binding agent may be used to identify the presence of the binding agent (e.g., via in situ hybridization). In some instances, the probes may comprise detectable labels or moieties, e.g., a fluorophore, radioisotope, mass tag, etc. For example, hybridization-based assays such as SeqFISH or Nanostring may be performed to probe or assay particular regions of a polymerizable molecule to determine its identity. In other examples, an amplification-based approach may be used to determine the presence and identity of a polymerizable molecule. For example, PCR or nested PCR approaches may be used to selectively probe for a particular sequence of a polymerizable molecule.

Alternatively or in addition to, the binding agent may comprise a detectable label or moiety. For example, the binding agent may comprise a fluorophore, radioisotope, mass tag, chromogenic enzyme (e.g., horse radish peroxidase), etc., which may be detectable using the appropriate imaging technique. Different binding agents (e.g., binding agents that recognize different monomers or amino acids) may be labeled with distinct labels, e.g., different fluorophores, which can be used to identify the presence of the monomer or amino acid. In some examples, fluorophore-labelled binding agents can be detected using single molecule imaging (e.g., total internal reflection, confocal, wide-field, or super resolution microscopy (e.g., PALM, STORM, STED)).

In some instances, the substrates comprising the polymerizable molecules (e.g., following one or more iterations of workflows depicted in FIG. 1A-1F) may be provided on an array for sequencing. For example, a plurality of beads comprising polymerizable molecules that encode for amino acids of a plurality of peptides may be provided on an array for sequencing. In one such example, the plurality of beads may be directly or indirectly coupled to an additional substrate (e.g., planar substrate, such as microscope slides or multi-well plates), and sequencing may be performed using image-based sequencing approaches (e.g., using sequencing by synthesis or in situ hybridization probes and a single-molecule resolution imaging system), amplification-based sequencing, or both. The plurality of beads may be coupled to the additional substrate using any suitable technique such as nucleic acid attachment using the polymerizable molecules or capture molecules, magnetic attachment (using a magnetic field and magnetic beads), optoelectronics, digital microfluidics, application of an electric field, gravity settling, centrifugation, capillary force, hydrogen bonding, electrostatic interactions or other suitable approach.

Advantageously, the methods and systems described herein may be particularly useful in achieving highly accurate sequencing, or identification of individual monomers (e.g., amino acids), of polymeric analytes (e.g., peptides). As described herein, the sequencing approaches disclosed herein provide for local tethering and cleavage of monomers, coupling of binding agents comprising polymerizable molecules with encoded information to the cleaved monomers, and coupling the polymerizable molecules to other local polymerizable molecules for transfer of the encoded information, which can then be used to identify (e.g., via sequencing) each monomer. As the methods outlined herein comprise removing the monomers and tethering them locally, followed by coupling of binding agents, the local environment problem (variability of binding agent specificity caused by different adjacent amino acids) is avoided, which can thus achieve highly accurate reads of single monomers (e.g., amino acids) with single-molecule sensitivity.

For example, the methods outlined herein may achieve an individual read accuracy, e.g., the probability of correctly identifying a single monomer (e.g., an amino acid) of greater than 10%, greater than 20%, greater than 30%, greater than 40%, greater than 50%, greater than 60%, greater than 70%, greater than 80%, greater than 90%, or higher. Similarly, the individual read accuracy may remain relatively constant as multiple iterations of the operations are performed. In other words, the individual read accuracy may not vary substantially depending on how many rounds or iterations of sequencing is performed. For example, a high individual read accuracy may be obtained for at least 3, at least 4, at least 5, at least 6, at least 7, at least 8, at least 9, at least 10, at least 20, at least 30, at least 40, at least 50, at least 60, at least 70, at least 80, at least 90, at least 100, at least 500, at least 1000 or greater number of iterations of sequencing for identification of individual monomers (e.g., amino acids) of a given polymeric analyte. For peptides, the methods provided herein may allow for distinction of individual amino acids from other amino acids with high specificity. For instance, using the methods described herein, at least one single amino acid residue may be distinguishable from the other 19 proteinogenic amino acid residues, or, in some cases, amino acids comprising post-translational modifications or unnatural amino acids. The methods described herein may allow for specific identification of 1, 2, 3, 4, 5, 6, 7, 8, 9, 10, 11, 12, 13, 14, 15, 16, 17, 18, 19 or all 20 proteinogenic amino acids.

The individual read accuracy may, in some instances, be defined by a probability function. For instance, the individual read accuracy may represent the probability of correctly identifying all or a subset of amino acids in a peptide. Accordingly, the individual read accuracy, in some contexts, may be described as P (amino acid A, position X)=Number of correct identifications/(Number of correct identifications+number of incorrect or failed identifications). The number of incorrect or failed identifications can include, for example, an incorrect amino acid identification (incorrect A), an insertion (incorrect X), or a deletion (incorrect X), or a combination thereof. In one such example, an indel in a given cycle (“cycle N”) may result in one failed or incorrect read, but subsequent cycles may be accurate, such that P=(N−1)/N.

In some instances, an individual read accuracy may be characterized by the probability of correctly identifying a class or group of amino acids. A class or group of amino acids may be classified, for example, based on physical, chemical, biological, physicochemical, or other properties. For example, aliphatic side chains (e.g., G, A, V, L, I), hydroxyl side chains or sulfur/selenium containing side chains (e.g., S, C, U, T, M), aromatic side chains (e.g., F, Y, W), basic side chains (e.g., H, K, R), acidic side chains (e.g., D, E, N, Q) may each constitute a class of amino acids. In another example, positively-charged side chains (e.g., arginine, histidine, lysine), negatively-charged side chains (aspartic acid, glutamic acid), polar uncharged side chains (e.g., serine, threonine, asparagine, glutamine), hydrophobic side chains (e.g., alanine, valine, isoleucine, leucine, methionine, phenylalanine, tyrosine, trytophan) may each constitute a class of amino acids. In some examples, certain types of post-translational modifications may constitute a class of amino acids. A class of amino acids may comprise 1, 2, 3, 4, 5, 6, 7, 8, 9, 10, or more members, and any number of classes comprising the same or different numbers of members may be used to characterize individual read accuracy. For example, a first class of amino acids may comprise 2 amino acid types (e.g., leucine and isoleucine), a second class of amino acids may comprise 3 amino acid types (e.g., tryptophan, phenylalanine, and tyrosine). In such an example, if a read outputs leucine when the actual residue is isoleucine, the first read is still counted as a correct identification. Similarly, if a read outputs tryptophan when the actual residue is phenylalanine or tyrosine, the read is also counted as a correct identification.

A relative (e.g., positional) individual read accuracy may also be ascribed to the individual monomers or to the polymeric analyte. The relative individual read accuracy may be the accuracy of relative positioning of each monomer in a sequence, but not the absolute position. For instance, for a peptide comprising the sequence A-G-N, if A is first correctly identified, G is not identified, and N is then correctly identified, then the relative individual read accuracy, or the correct identification of the order of a subset of amino acids (A and N) may be assigned (e.g., 100%) even though not every single amino acid is identified. In such an example, the relative individual read accuracy relies on the correct identification of the order or relative positioning of a subset of amino acids of a peptide, but not necessarily the absolute position of each of those identified amino acids.

Fingerprinting: The methods described herein may be useful in complete de novo protein or peptide sequencing (e.g., the identification of each amino acid in a peptide), or for fingerprinting a protein (e.g., identifying only a subset of amino acid types in a peptide and inferring from, or mapping the identified amino acids to, a reference database, to identify the peptide or protein). For fingerprinting, a subset of amino acids may be identified, e.g., using the approaches described herein, without the need of binding agents that are specific to all 20 proteinogenic amino acids. For example, 1, 2, 3, 4, 5, 6, 7, 8, 9, 10, 11, 12, 13, 14, 15, 16, 17, 18, or 19 different binding agents with single-amino acid or multi-amino acid specificity may be sufficient to determine the identity of a protein or peptide. For known proteome databases, reference-based reconstruction may be performed by simulating NGS reads that would be generated from the set of possible peptides in the workflow. For each possible peptide, a simulation can produce NGS reads mimicking the output of this protein sequence system. Next, the real (experimental) NGS reads from a run can be matched to simulated reads from candidate peptides from a database based on likelihood. This results in reconstructed, fragmentary, peptide sequences (contigs) with probability assigned to the assembled peptide sequence.

In some fingerprinting approaches, not all amino acids are required to be identified to accurately identify the protein. The mapping accuracy, or the accuracy of correctly identifying a protein (e.g., using a protein database) may be, for example, at least 50%, at least 60%, at least 70%, at least 80%, at least 90%, or greater, by correctly identifying only a subset of amino acids. For instance, only 1, 2, 3, 4, 5, 6, 7, 8, 9, 10, 11, 12, 13, 14, 15, 16, 17, 18, or 19 amino acids may need to be identified in order to identify the protein. In some instances, the individual read accuracy for single amino acids need not be highly accurate in order to identify a protein with high accuracy. For example, the individual read accuracy of a set of individual amino acids may be on average around 50%, around 60%, around 70%, around 80%, around 90%, etc. in order to yield a correct identification of the protein or peptide with at least 50%, at least 60%, at least 70%, at least 80%, at least 90%, or greater accuracy or confidence.

In some instances, the individual read accuracy for a single amino acid need not be above a threshold percentage in order to accurately identify a protein. For instance, a binding agent may have off-target binding to chemically similar residues (e.g., a binding agent against leucine may have off-target binding to isoleucine), which would result in poor individual read accuracy but still allow for correct identification of a peptide. In such examples, the exact amino acid (e.g., leucine) may not need to be identified, but the position (e.g., “X” position) or relative position can be denoted as one of a number of amino acids (e.g., either leucine or isoleucine). Contextual clues and homology may then be used against a reference protein database to identify the protein, knowing that the X-position amino acid is one of a number of amino acids (e.g., either leucine or isoleucine). For example, knowing the exact NTAA, the exact n-1 NTAA, and the X position amino acid (as one of a number of amino acids) may be sufficient to correctly identify the protein. Accordingly, the accuracy of correctly identifying a peptide may be high, even if the individual read accuracy is not.

High Throughput Sequencing/Parallelization: The methods described herein may be conducted in a parallelized, high-throughput format. Such parallelization may be achieved by having substrates comprising multiple polymeric analytes coupled thereto and performing the operations (e.g., coupling a monomer to the capture moiety, cleaving, contacting with a binding agent or library of binding agents, coupling of polymerizable molecules, optional cleavage of the monomer from the capture moiety) iteratively, across the substrate. The methods described herein may allow for parallel analysis of 1, 10, 100, 1,000, 10,000, 100,000, 1,000,000, 10,000,000, 100,000,000, 1,000,000,000 or greater number of polymeric analytes. In some instances, a library of binding agents may be used to recognize different monomer types (e.g., different amino acids of a peptide analyte or derivatized amino acids), such that different polymeric analytes (e.g., different peptides) may be processed on a single substrate, thereby increasing multiplexing or detection of multiple different analytes.

A library of binding agents may be used to recognize different monomers and to facilitate high-throughput readout. As described herein, the library of binding agents may comprise binding agents that can recognize a single monomer (e.g., a single cleaved amino acid) or multiple monomers (e.g., multiple cleaved amino acids). In some instances, binding agents with varying levels of specificity may be used in a sequence or order, which may help render a less-specific binding agent to be more specific, simply based on the sequence in which it is provided. For example, a first binding agent may be capable of specifically binding to a first monomeric analyte, and a second binding agent may be capable of binding to both the first monomeric analyte and a second monomeric analyte. The first binding agent may be provided and contacted with the first monomeric analyte and second monomeric analyte. Since the first binding agent is specific to the first monomeric analyte, the first binding agent will bind exclusively to the first monomeric analyte. Subsequently, the second binding agent may be provided; however, since the first monomeric analyte is bound to the first binding agent, the first monomeric analyte may be inaccessible (e.g., sterically blocked) to the second binding agent. As such, the second binding agent may bind only to the second monomeric analyte. Accordingly, identification of the first binding agent and second binding agent (e.g., through detection of a label/tag or through sequencing of polymerizable molecules coupled to the binding agents and optionally transferred to a substrate), may allow for identification of the first monomeric analyte and the second monomeric analyte.

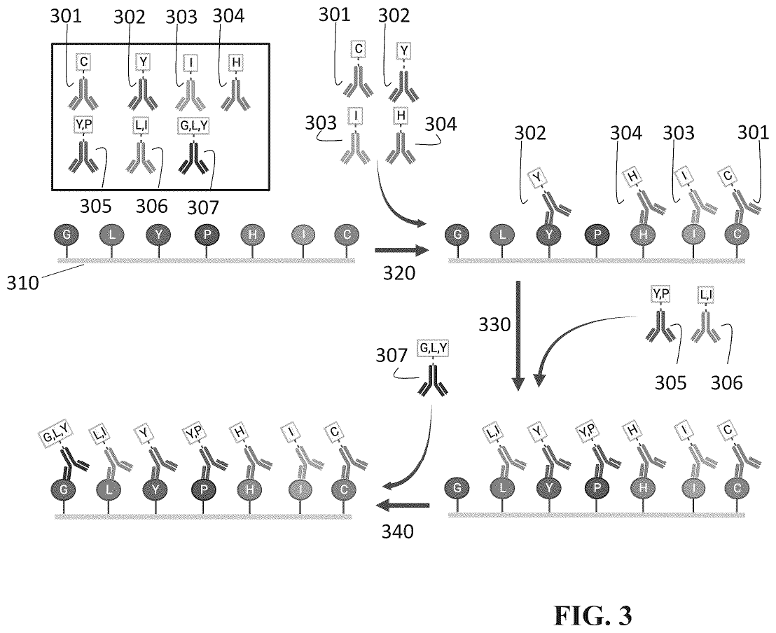

FIG. 3 schematically shows an order-dependent binding scheme of a library of binding agents and an example of how binding agents with less specificity (e.g., binding agents that can bind to multiple monomer types) can be optimized and rendered more specific based on the order of binding of each of the binding agents in the library. A substrate 310 is provided which has coupled thereto, a plurality of cleaved monomers (e.g., terminal amino acids cleaved from individual peptides). In this example, the substrate comprises, from left to right, a cleaved glycine (G), a cleaved leucine (L), a cleaved tyrosine (Y), a cleaved proline (P), a cleaved histidine (H), a cleaved isoleucine (I), and a cleaved cysteine (C). A library of binding agents (box) may comprise binding agents (e.g., antibodies) that have varying specificities. Binding agent 301 is specific to cysteine (C), binding agent 302 is specific to tyrosine (Y), binding agent 303 is specific to isoleucine (I), and binding agent 304 is specific to histidine (H). Binding agent 305 can bind to Y and P, binding agent 306 can bind to L and I, and binding agent 307 can bind to G, L, and Y.

In process 320, binding agents with single specificity (301, 302, 303, and 304) are provided and allowed to bind to the specific cleaved monomer to which each has specificity. Subsequently, in process 330, binding agents 305 and 306 with lower specificity (e.g., bind to greater than one cleaved monomer) are provided. Binding agent 305, which binds Y and P, binds to P, since the Y residues are already bound and unavailable due to inaccessibility caused by binding agent 302. Similarly, binding agent 306, which binds L and I, binds to L, since the I is already bound and unavailable due to binding agent 303. In process 340, binding agent 307 is provided, which can bind G, L, and Y. However, as all the G, L, and Y residues are bound by other binding agents and thus unavailable, the binding agent 307 specifically binds to G. Subsequent downstream analysis, e.g., identification of each binding agent (e.g., via imaging or sequencing of the binding agent polymerizable molecules coupled to the substrate, not shown) can reveal the identity of each of the binding agents and thus the monomeric analyte. For example, even though binding agent 307 can bind to three different monomeric analytes (G, L, Y), because of the ordered/sequential approach of the provision of the binding agents (e.g., blocking via binding of L and Y, thus leaving only G), the identity of G may be determined by the presence of binding agent 307.

In some instances, it may be useful to barcode the polymeric analytes prior to processing. Barcode sequences may be attached to the polymeric analytes at a single location (e.g., at a terminus), multiple locations, adjacent to the polymeric analyte (e.g., on a substrate), etc. as is described elsewhere herein. For example, a peptide may be labeled at the N-terminus, C-terminus, or an internal amino acid with a nucleic acid barcode molecule. The nucleic acid barcode sequence may comprise information or be unique to a partition or compartment, sample, peptide, etc. such that each unique barcode sequence can be traced back (e.g., subsequent to nucleic acid sequencing or other detection method) to the originating partition or compartment, sample, peptide, etc.

Alternatively, or in addition to, the capture moieties or polymerizable molecules may comprise a barcode sequence. The barcode sequence may be specific to a particular partition, sample, or spatial location. For example, a substrate may comprise a plurality of individually addressable units. The polymerizable units or capture moieties of each individually addressable unit may comprise a unique barcode specific to the individually addressable unit (e.g., a spatial barcode). The polymeric analytes may be coupled to the substrate such that each individually addressable unit comprises, on average, no more than one polymeric analyte. Such a distribution of polymeric analytes may be obtained, for example, using a limited dilution approach (e.g., diluting the polymeric analytes to reduce the number of polymeric analytes that may attach to a given individually addressable unit), or by introduction of chaotropic agents (e.g., guanidine, formamide, urea). The polymeric analytes may be distributed across the individually addressable units according to a Poisson distribution. Thus, for a given substrate, about 6%, 10%, 18%, 20%, 30%, 36%, 40%, or 50% of the individually addressable units may comprise one or fewer polymeric analytes.

Order of Operations: It will be appreciated that the operations presented in the methods described herein may be performed in any useful or convenient order and that some operations, in some instances, may be optional. For example, in some instances, the coupling of the monomer to the capture moiety may occur prior to, during, or subsequent to the cleaving of the monomer from the polymeric analyte. In another example, the substrate may be provided with the cleaved monomers coupled thereto, such that cleavage of the monomer from the polymeric analyte is obviated. In yet another example, in instances where a linker is used to couple to the monomer (e.g., amino acid) and the capture moiety or substrate, the linker may comprise a monomer-coupling group and subsequently be reacted with a substrate-binding group (e.g., an oligonucleotide); alternatively, the linker may be provided with the substrate-binding group as part of the linker (e.g., pre-conjugated to the substrate-binding group).

Additional operations may be performed at any useful or convenient step, e.g., prior to provision of the polymeric analyte (e.g., peptide) coupled to the substrate or subsequent to one or more of the processing operations (e.g., subsequent to coupling of the polymerizable molecules).

### Modification of Polymeric Analytes and Polymerizable Molecules

The present disclosure also provides approaches for modifying polymeric analytes or monomers of the polymeric analytes (e.g., amino acids of a peptide), as well as polymerizable molecules described herein. Such modifications may useful, for example, in rendering a monomer more resistant to certain reaction conditions (e.g., Edman degradation), to increase or decrease binding affinity of a binding agent to the modified monomer, to assist in docking or interfacing of the modified monomer to an enzyme (e.g., a protease, cleaving enzyme, binding agent, or enzyme analog, such as a ribozyme or DNAzyme), or other purpose.

In some instances, a polymeric analyte such as a peptide may be modified in order to render the peptide or a constituent amino acid more resistant to the reaction conditions for cleaving the amino acid from the peptide. As described elsewhere herein, Edman degradation reactions may be used to cleave terminal amino acids from peptides; such reaction conditions may comprise the use of strong acids (e.g., trifluoroacetic acid) or elevated temperatures, which can result in alterations of amino acid residues or post-translational modifications (PTMs). As such, peptides may be pre-processed in order to protect amino acid residues or PTMs. For example, the peptide may be subjected to alkylation, e.g., using 4-vinylpyridine, iodoacetamide, which may be useful in preventing oxidation of cysteine residues. The peptide may be subjected to acetylation, e.g., O-acetylation to form an ester such as acetyl chloride, which may be useful in preventing dehydration, racemization, or destruction of a derivatized (e.g., PTH form) of serine or threonine. The peptide may be subjected to β-elimination of a phosphate, followed by a Michael addition of a thiol group, (e.g., as described in Knight et al. 2003. Nature Biotechnology 21, 1047-1054, which is incorporated by reference herein) to detect phosphorylation events. The peptide may be contacted with phenyl isothiocyanate, acetic anhydride, or other amine-reactive group to protect lysine residues. Additional examples of peptide processing for Edman degradation can be found in Tarr, Methods of Protein Microcharacterization. pp 155-194, which is incorporated by reference herein.

A polymeric analyte or monomer may be modified to influence the interaction of a binding agent with the polymeric analyte or monomer. Controlling the interaction of the binding agent with the polymeric analyte or monomer (e.g., cleaved monomer) may allow for modifying or controlling the identification of individual monomers (e.g., amino acids of a peptide). For example, binding agents comprising polymerizable molecules with identifying information (e.g., nucleic acid barcode sequences) may be blocked or inhibited from recognizing or binding to a monomer (e.g., cleaved monomer); as such, the identifying information of the polymerizable molecules may not be transferred to an adjacent polymerizable molecule and thus the monomer would not be identified as being present. Such approaches to block or inhibit binding of the binding agents may be beneficial in sequential analysis of individual monomers of a polymeric analyte, e.g., to remove signal or detectability of a monomer after it has already been detected and to prevent presence of false positives in subsequent iterations.

In some instances, blocking of the binding agents may be performed by altering the chemical composition of a cleaved monomer (e.g., amino acid), thereby preventing recognition and coupling of the binding agents. Such chemical composition changes may be performed by derivatizing the cleaved monomer, adding of chemical groups to the cleaved monomer, or other chemical processing (e.g., addition or removal of groups) from the cleaved monomer. For example, as described herein, a peptide may be contacted with a linker comprising (i) an amino acid reactive moiety (e.g., PITC) and (ii) a capture moiety-coupling group, which may allow for simultaneous or sequential cleavage of a terminal amino acid (e.g., NTAA) from the peptide and tethering of the terminal amino acid to the capture moiety (e.g., coupled to a substrate). The resultant monomer-capture moiety complex may comprise a derivatized amino acid, e.g., a PTC-derivatized amino acid, to which a binding agent may couple. Blocking of the binding agent may be achieved by appending a blocking agent, e.g., a chemical group or adduct, to the monomer-capture moiety complex; for example, the monomer may be conjugated to a synthetic polymer (e.g., PEG), nucleic acid molecule, fluorophores, quenchers, nanotube, nanoparticle, small molecules, polypeptide or protein, fatty acid chain, or other large, sterically-hindering molecules. The blocking agents may be appended to the monomer using a chemical approach (e.g., reacting with an amino acid, e.g., via a photo-reaction) or enzymatically, e.g., using methyltransferases, tRNA synthetases, acetyltransferases, etc. The blocking agent may be able to react to the derivatized amino acid (e.g., PTC-derivatized amino acid) or a derivative thereof, e.g., ATZ, PTH or other derivatized forms of the amino acid. Alternatively, or in addition to, the blocking agent may be able to react another moiety of the monomer or to the capture moiety.

Conjugation of the blocking agent to the cleaved monomer may be achieved using chemical or enzymatic approaches. Chemical approaches may involve the use of one or more linkers (e.g., heterobifunctional linkers) to link the blocking agent to the cleaved monomer. Alternatively, or in addition to, enzymes such as engineered tRNA synthetases, methyltransferases, acetyltransferases, and the like may be used to add a moiety to the cleaved monomer.

In some instances, blocking of the binding agent from coupling to the monomer may be achieved by derivatizing the cleaved monomer. For example, a binding agent may have specificity and may bind to the PTC-derivatized form of an amino acid but not to ATZ- or PTH-derivatized forms of the amino acid. As such, derivatization of the PTC-derivatized amino acid to an ATZ or PTH format may inhibit or prevent binding of the binding agent. Alternatively, the binding agent may have specificity and may bind to the ATZ-derivatized form, and derivatization of the amino acid to the PTH- or PTC-form may prevent binding of the binding agent to the cleaved, derivatized monomer. In an example, a cleaved PTC-derivatized amino acid that has already been contacted with a binding agent may be converted to the PTH-derivatized form by reacting with an amine-PEG-biotin, which will prevent further recognition of the binding agent.

Alternatively, or in addition to, the binding agent may comprise a quenchable moiety. For instance, the binding agent may comprise a quenchable fluorophore or dye (e.g., Black Hole Quencher™ dye, Iowa Black quenchers, ZEN quenchers). The fluorophore or dye may be detectable using an appropriate imaging technique (e.g., total internal reflection fluorescence microscopy, super resolution imaging), and the presence of a fluorescent signal may indicate the presence of a particular monomer (e.g., a cleaved PTC-derivatized amino acid). Subsequent to detection, the quenchable moiety may be quenched or photobleached, such that in subsequent iterations or cycles of cleaving and detection of monomer, any residual binding agent from earlier cycles are rendered undetectable. In an example, a cleaved PTH-derivatized amino acid may be detected with a binding agent via fluorescence microscopy, converted to the PTH-derivatized form by reaction with an amine-quencher (e.g., amine-PEG-quencher), which will help prevent fluorescence emission.

It will be appreciated that the binding of the binding agent to the cleaved monomers (e.g., monomer-capture moiety complexes) may also be enhanced using any of the aforementioned approaches. For instance, a PTC-derivatized amino acid may be further derivatized to another form (e.g., ATZ or PTH) that increases or enhances recognition or binding of the binding agent. In another example, an adduct or chemical moiety may be added to the cleaved monomers in order to increase or improve the interaction of the binding agent with the cleaved monomer. Such adducts or chemical moieties may be added to select monomer types (e.g., a subset of amino acids, such as those with similar characteristics or properties), or non-discriminatorily to all monomer types (e.g., all amino acid types). For example, a peptide may be contacted with 3-arylpropiolonitriles (APN), which may react with cysteine residues and optionally enable tagging of the cysteine residues. A binding agent specific to cysteine-APN may be used for improved detection of cysteine residues.

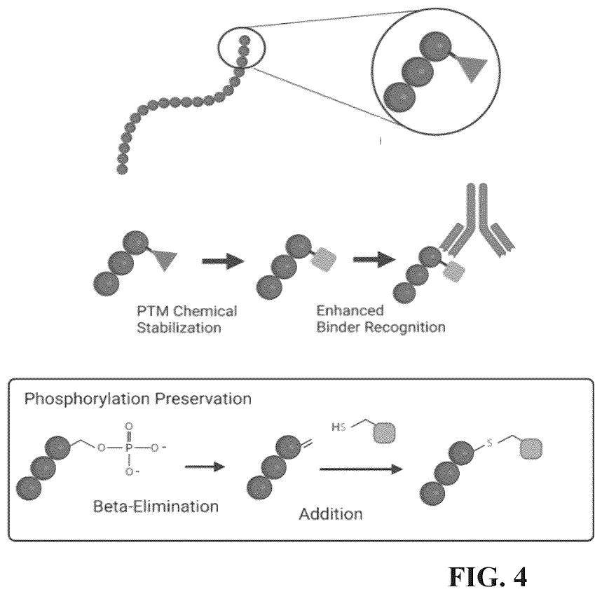

FIGS. 4-8 schematically show examples of modifications to a monomer or cleaved monomer that can influence the coupling of a binding agent thereto. FIG. 4 schematically shows an example of a post-translational modification that can be modified to improve or enhance the recognition and binding of a binding agent (e.g., antibody or antibody fragment) to the PTM. In one such example (bottom panel), a peptide may comprise a phosphorylated amino acid (naturally occurring PTM). The peptide may be subjected to a beta-elimination and then Michael addition of a thiol group, thereby generating a modified amino acid. The peptide may subsequently be subjected to conditions sufficient to tether the NTAA to a capture moiety, NTAA cleavage from the peptide, and contacting with a binding agent.

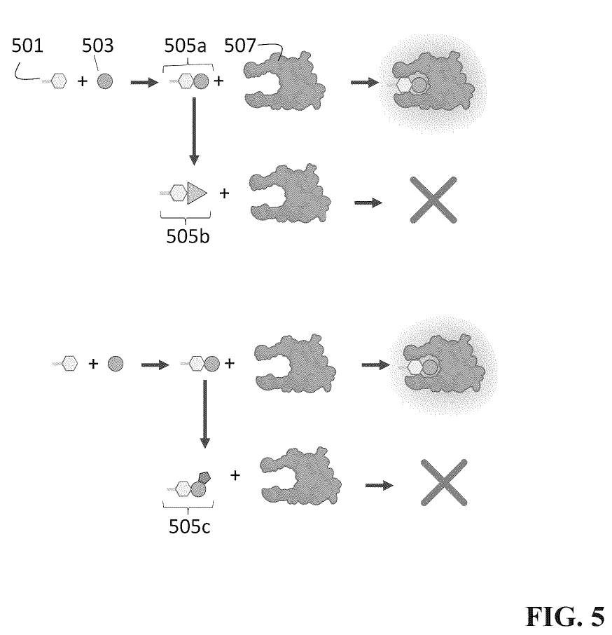

FIG. 5 schematically shows an example of a derivatization process or addition of a chemical moiety to a monomer to alter the coupling of a binding agent. A linker 501 comprising a monomer-reactive group may react with a monomer 503 to generate a linker-monomer complex 505a. The linker-monomer complex 505a may be contacted with a binding agent 507 which specifically recognizes the linker-monomer complex 505a. The linker-monomer complex 505a may then be derivatized to generate a derivatized linker-monomer complex 505b, which is not recognized by the binding agent 507. Alternatively, or in addition to, the linker-monomer complex 505a may be coupled to a chemical moiety (e.g., an adduct or blocking group) to generate a blocked linker-monomer complex 505c, which is also not recognized by the binding agent 507. In some embodiments, the linker 501 comprises an amino acid reactive group (e.g., PITC) and couples to a terminal amino acid (monomer 503) of a peptide (not shown) to generate a PTC-derivatized amino acid (linker-monomer complex 505a). The PTC-derivatized amino acid may be subjected to derivatization (e.g., using heat, acid) to generate an ATZ-derivatized amino acid or a PTH-derivatized amino acid (derivatized linker-monomer complex 505b). The binding agent 507 may specifically recognize the PTC-derivatized amino acid, but not the ATZ- or PTH-derivatized forms.

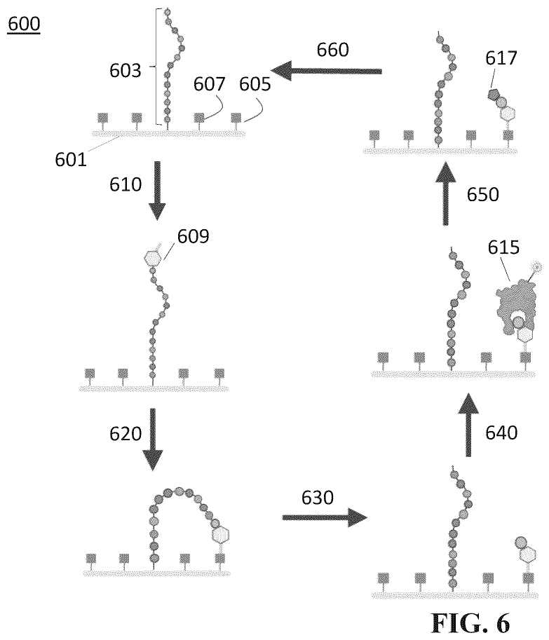

FIG. 6 schematically shows an example workflow for sequencing a polymeric analyte, including a blocking operation. In workflow 600, similar to workflow 100 in FIG. 1, a polymeric analyte 603 is provided, sequentially disassembled into individual monomers via contacting with capture moieties and cleavage, and the individual cleaved monomers are contacted with binding agents, optionally comprising polymerizable molecules that identify or encode for the binding agents. The polymerizable molecules of the binding agent may be coupled to an additional polymerizable molecule, and the monomer can subsequently be cleaved from the capture moiety to repeat the process. A substrate 601 is coupled to a polymeric analyte 603 (e.g., a peptide to be sequenced), a capture moiety 605 (e.g., a nucleic acid primer, a click chemistry moiety such as an alkyne), and an additional capture moiety 607 (e.g., an additional polymerizable molecule such as nucleic acid primer or click chemistry moiety). In some instances, the capture moiety 605 and the additional capture moiety 607 are identical molecules. The polymeric analyte 603 may be contacted with a bifunctional linker 609, which comprises an amino acid coupling group (e.g., PITC) and a capture moiety-coupling group (e.g., a nucleic acid molecule or a click chemistry moiety such as an azide). In process 610, the bifunctional linker 609 couples to a terminal monomer (e.g., terminal amino acid such as the N-terminal amino acid (NTAA)). In process 620, the bifunctional linker 609 is coupled to the capture moiety 605, which may be mediated by hybridization of the complementary nucleic acid molecules, using a splint oligonucleotide, or click chemistry (e.g., cycloaddition reaction of azide and alkyne). In process 630, the system is subjected to conditions sufficient to cleave the terminal monomer (e.g., amino acid) from the polymeric analyte 603 (e.g., peptide). The conditions may include performing an Edman degradation reaction. The cleavage of the monomer from the polymeric analyte results in a cleaved monomer coupled to the bifunctional linker 609 and the capture moiety 605, e.g., to generate a monomer-capture moiety complex. In process 640, a binding agent 615 is provided. The binding agent may comprise a detectable moiety (e.g., fluorophore), which may be detected using an appropriate detection technique (e.g., spectroscopy, microscopy), or the binding agent may comprise another polymerizable molecule (e.g., nucleic acid molecule) encoding for information regarding the identity of the binding agent (not shown). The binding agent 615 may be specific to the monomer (e.g., to a single amino acid) or to the monomer-linker complex (e.g., PITC-amino acid). In instances where the binding agent comprises a polymerizable molecule, the polymerizable molecule may comprise additional sequences, such as barcode sequences, UMI, restriction sites, transposition sites, sequencing sites (e.g., P5 or P7 sequences), a sequence to represent a cycle or iteration number, or other functional sequences. The polymerizable molecule of the binding agent may couple to the additional polymerizable molecule 607 that is coupled to the substrate 601. In some instances, an extension reaction may be performed (e.g., using a polymerase), to copy the sequence of the polymerizable molecule of the binding agent to the additional capture moiety 607 that is coupled to the substrate 601. Alternatively, the polymerizable molecule of the binding agent may be ligated to the additional polymerizable molecule 607, either chemically (e.g., via complementary click chemistry) or enzymatically (e.g., using a ligating enzyme). In process 650, the monomer-capture moiety complex may be subjected to modification, e.g., additional derivatization, or coupled to a blocking moiety 617, which may prevent coupling of binding agent 615 or other, additional binding agents. In some embodiments, the monomer may be decoupled (e.g., removed or cleaved) from the capture moiety 605, e.g., using chemical, mechanical, or enzymatic approaches. In process 660, the workflow 600 may then be iterated or repeated to sequence all or a portion of the polymeric analyte 603.

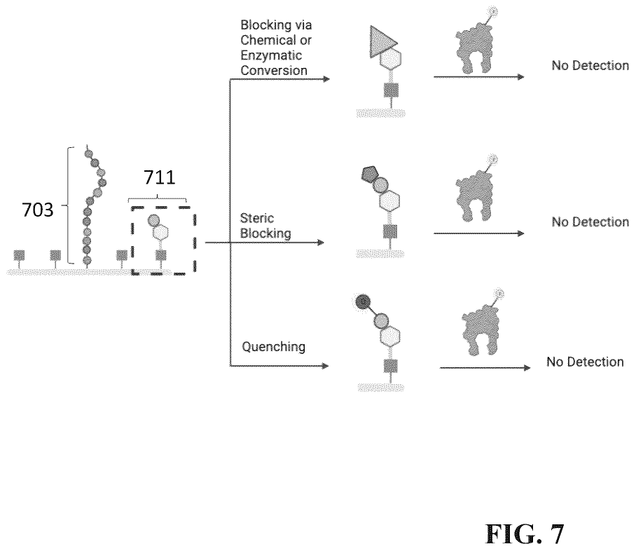

FIG. 7 schematically shows examples of processing cleaved monomers to prevent binding of binding agents. A polymeric analyte 703 (e.g., peptide) may be processed as described herein, such that a terminal monomer is linked to a capture moiety to generate a monomer-capture moiety complex 711. The monomer-capture moiety complex 711 may be contacted with a binding agent that recognizes at least a portion of the monomer-capture moiety complex (not shown). The binding agent may be detected (e.g., the binding agent may comprise a fluorophore that can be detected using imaging or microscopy, or via the polymerizable molecule approaches described herein). Subsequently, to prevent re-binding or detection of the binding agent or an additional binding agent, or to prevent off-target binding of additional binding agents with different affinities, the monomer-capture moiety complex 711 may be processed using a variety of approaches. In some examples, the monomer-capture moiety complex may be converted (e.g., derivatized) using chemical or enzymatic approaches, such that the binding agent does not recognize or bind to the converted or derivatized form. In another example, a chemical group or moiety (e.g., adduct, bulky blocking group) may be added to the monomer-capture moiety complex, which may sterically inhibit binding of the binding agent. In yet another example, the monomer-capture moiety complex may be detected using a binding agent comprising a fluorophore; subsequent to detection, a quenchable moiety may be added to the monomer-capture moiety complex (e.g., via reaction with amine-quenchable fluorophore). The quenchable moiety may quench the signal of any binding agents within the vicinity of the monomer-capture moiety complex, such that the monomer-capture moiety complex will not be detected in subsequent iterations of sequencing.

Polymeric analytes may also be modified to facilitate cleavage of a monomer therefrom. As described herein, in some instances, cleavage of the monomer from the polymeric analyte may be mediated by a cleaving enzyme or ribozyme or DNAzyme. The affinity of the cleaving enzyme (or ribozyme or DNAzyme) to the monomer (e.g., NTAA or CTAA) may be enhanced or modulated by addition of chemical group or moiety to generate a modified monomer. For example, NTAAs may be contacted with an amino-acid reactive agent, e.g., thiocyanates, SNFB, dansyl chloride, DNFB, which may alter the chemical structure of the NTAA. Alternatively, or in addition to, the monomer may be modified with a hapten, guanidinyl group, thiobenzoylation agent, thioacetylation agent, thioacylation agent, etc. The addition of the chemical group or moiety may improve or enhance the binding or recognition of the cleaving enzyme (or ribozyme or DNAzyme) to the modified monomer. The cleaving enzyme (or ribozyme or DNAzyme) may be a naturally-occurring or engineered protein, e.g., an aminopeptidase, carboxypeptidase, tRNA synthetase, or other peptide that is modified to recognize and cleave the monomer or modified monomer.

Systems, compositions, kits: Also provided herein are systems, compositions, and kits for processing polymeric analytes. A system of the present disclosure may comprise a sequencing instrument that is configured to receive a polymerizable molecule or stacked polymerizable molecule, as described herein, and to provide sequencing reads of the polymerizable molecule or stacked polymerizable molecule. Alternatively, or in addition to, a system of the present disclosure may be configured to process or sequence a polymeric analyte. The system may comprise a capture moiety and a binding agent comprising a first polymerizable molecule coupled thereto. The system may be configured to provide the polymeric analyte, the capture moiety, and the binding agent; cleave a monomer from the polymeric analyte; couple the monomer to the capture moiety to generate a monomer-capture moiety complex; and contact the monomer-capture moiety complex with the binding agent. Accordingly, systems of the present disclosure may comprise any useful apparatuses or tools, including but not limited to mixers, liquid handlers, vortexes, centrifuges, heating or cooling elements, mechanical stages, microfluidic chambers and fluidic controls.

A composition of the present disclosure may comprise any useful items or reagents for processing or sequencing a polymeric analyte. A composition may comprise a capture moiety configured to couple to a monomer of a polymeric analyte and a binding agent comprising a polymerizable molecule coupled thereto. The composition may further comprise a substrate, a linker (e.g., to couple a polymeric analyte, polymerizable molecule, or monomer to the substrate), a cleaving agent, a ligating agent (e.g., ligase), an additional polymerizable molecule, reagents for conducting a reaction, or a combination thereof. In some instances, a composition comprises a substrate that comprises the additional polymerizable molecule, and the capture moiety coupled thereto.

Kits of the present disclosure may comprise any useful reagents for processing, analyzing, or sequencing a polymeric analyte. A kit may comprise a reagent for providing a polymeric analyte and a capture moiety, a reagent for coupling the polymeric analyte and capture moiety to a substrate, a reagent for cleaving a monomer of the polymeric analyte, a reagent for coupling the monomer to the capture moiety to generate a monomer-capture moiety complex, a reagent for providing a binding agent comprising a polymerizable molecule, a reagent for coupling the polymerizable molecule to a second polymerizable molecule, or a combination thereof. The kit may further comprise reagents for removing or decoupling the monomer from the monomer-capture moiety complex. In some instances, the kit may further comprise reagents for removing or decoupling a polymerizable molecule or a stacked polymerizable molecule from a substrate, or the kit may comprise reagents for conducting a nucleic acid extension reaction. The kit may include any relevant reagents, e.g., buffers, detergents, chelating agents, cofactors, enzymes, ribozymes or DNAzymes, acids, bases, salts, metal ions, primers, nucleic acid molecules, nucleotides, proteins, polynucleotides, binding agents (e.g., antibodies, aptamers, nanobodies, antibody fragments), lipids, carbohydrates, ribozymes, riboswitches, probes, fluorophores, oxidizing agents, reducing agents, nuclease or protease inhibitors, dyes, organic molecules, inorganic molecules, emulsifiers, surfactants, stabilizers, polymers, water, small molecules, therapeutics, radioactive materials, preservatives, or other useful reagent. The kits of the present disclosure may also provide instructions for the use of the contents of the kit.

### Terminal Monomer Cleavage

Also provided herein are methods for processing polymeric analytes, e.g., peptides, including sequential degradation of individual amino acids from a terminal end of a peptide, including standard and modifications to Edman degradation. In standard Edman degradation, an N-terminal amino acid is reacted with phenyl isothiocyanate (PITC) under mildly basic conditions to form a phenylthiocarbamoyl (PTC) derivative. The PTC-derivatized amino acid is treated with acid (e.g., TFA) to generate a cleaved cyclic 2-anilino-5(4)-thiazolinone (ATZ)-derivatized amino acid, leaving a new N-terminus on the remaining peptide. The ATZ-derivatized amino acid may be converted to a phenylthiohydantoin (PTH) derivative and analyzed, e.g., by reverse phase HPLC or mass spectrometry. The process can be iterated until all or a partial number of the amino acids comprising a peptide sequence has been removed from the N-terminal end and identified.

Given the harsh reaction conditions of standard Edman degradation, the polymerizable molecules described herein (e.g., nucleic acid molecules, peptides, lipids) etc. may comprise alterations or modifications to render them more resistant to the reaction conditions. For example, nucleic acid molecules may comprise predominantly pyrimidines (e.g., thymines, cytosines, uracils) which are more resistant to acid degradation and heat as compared to purines (e.g., adenine and guanine). For example, a nucleic acid molecule may comprise at least 50%, at least 60%, at least 70%, at least 80%, at least 90%, or 100% thymines or cytosines. Alternatively or in addition to, canonical nucleotides may be substituted or may comprise acid-resistant nucleotide analogs, e.g., hexitol nucleic acids.

Alternative degradation chemistries may also be employed. Milder degradation under basic conditions for N-terminal amino acid removal can include the use of triethylamine acetate in acetonitrile or other solvent such as water, N, N-dimethylformamide (DMF), or a mixture of solvents. Alternatively, degradation may be achieved using a thioacylation approach, the use of milder acid reagents, e.g., trichloroacetic acid (pKa of 0.66) or dichloroacetic acid (pKa of 1.35), or alternative basic reaction conditions, e.g., using acid-base pairs such as N, N-Diisopropylethylamine (DIPEA), pyridine, acetic acid derivatives, etc.

C-terminal degradation strategies are also provided herein. C-terminal degradation may comprise Edman-like degradation approaches. C-terminal degradation may employ the use of activating reagents that react with the C-terminal carboxyl group of a peptide, and a derivatizing agent (e.g., a thiocyanate to generate a peptide-thiocyanate or peptide-thiohydantoin). Non-limiting examples of activating reagents include acetyl chloride and acetic anhydride. Alternatively, or in addition to, single-step C-terminal derivatization of a peptide to a peptidyl-thiohydantoin may be performed, e.g., using Schlack-Kumpf approach, in which a peptide is reacted with thiocyanic acid (e.g., in acetone) to generate a peptidyl-thiohydantoin. The peptide-thiohydantoin may be cleaved, e.g., using basic conditions, to generate an amino acid thiohydantoin and remaining peptide.

Cleavage of amino acids may also be achieved using enzymatic or enzyme-analog (e.g., ribozyme or DNAzyme) approaches. Example enzymatic cleavage may include the use of Edmanases (e.g., modified cruzain), aminopeptidases (e.g., Pfu aminopeptidase I, PhTET aminopeptidases, P. horikoshii aminopeptidases), metalloenzymatic aminopeptidases, acylpeptide hydrolases, tRNA synthetases, endopeptidases, carboxypeptidases, and the like. The enzymes or ribozymes or DNAzymes may be modified or engineered to recognize a modified amino acid, e.g., an amino acid that has a chemical moiety attached thereto (e.g., PITC, NITC, dansyl chloride, SNFB, DNP, SNP, biotin, streptavidin, nucleic acid molecules, lipids, carbohydrates, acetyl groups, acyl groups, guandinylation agents, etc.).

One or more reactions may be accelerated by application of energy or radiation, e.g., electromagnetic radiation. For example, degradation or cleavage of the terminal amino acid of a peptide may be facilitated by applying microwave energy to accelerate the reaction kinetics. For example, hydrolysis of proteins may be facilitated by application of microwave energy, e.g., as described in Margolis et al., 1991, Journal of Automatic Chemistry. Vol 13, No. 3, pp 93-95, which is incorporated by reference herein.

### Substrate Conjugation

The present disclosure provides methods for coupling molecules (e.g., polymeric analytes, e.g., biomolecules such as nucleic acid molecules, peptides, lipids, carbohydrates, etc.) to a substrate. The substrate may be functionalized to allow for covalent or noncovalent coupling of the molecules to a substrate. The substrate may comprise any useful functional moiety, e.g., a reactive moiety, that can couple or conjugate to a molecule or another reactive moiety. In a non-limiting example, a reactive moiety may comprise a click chemistry moiety, such as an azide, alkyne, nitrone, alkene (e.g., a strained alkene), tetrazine, methyltetrazine, triazole, tetrazole, phosphite, phosphine, etc. A click chemistry moiety may be reactive in copper-catalyzed Huisgen cycloaddition or the 1,3-dipolar cycloaddition between an azide and a terminal alkyne, a Diels-Alder reaction (e.g., a cycloaddition between a diene and a dienophile), or a nucleophilic substitution reaction in which one of the reactive species is an epoxy or aziridine. A molecule that is to be coupled to a substrate may comprise a complementary click chemistry moiety to that of the substrate; for example, the substrate may comprise an alkyne moiety and the molecule to be coupled may comprise an azide moiety, which can react with the alkyne moiety of the substrate to generate a covalent linkage. In one such example, the substate may comprise dibenzocyclooctyne (DBCO) moieties to which azide-comprising molecules (e.g., azide-DNA, azide-polymers, azide-peptides) can react and conjugate.

Alternatively, or in addition to, the reactive moiety may comprise a photoreactive moiety that may be activated when exposed to a photostimulus (e.g., light such as UV or visible light). Examples of photoreactive moieties include aryl (phenyl) azides (e.g., phenyl azide, ortho-hydroxyphenyl azide, meta-hydroxyphenyl azide, tetrafluorophenyl azide, ortho-nitrophenyl azide, meta-nitrophenyl azide), diazirines, azido-methyl-coumarins, benzophenones, anthraquinones, diazo compounds, diazirines, psoralen, 3-cyanovinylcarbazole phosphoramidite (CNVK), and analogs or derivatives thereof.

The reactive moiety may comprise a carboxyl-reactive crosslinker group, such as diazo compounds such as diazomethane and diazoacetyl, carbonyldiimidazole, carbodiimides (e.g., 1-ethyl-3-(3-dimethylaminopropyl)carbodiimide hydrochloride (EDC)), dicyclohexylcarbodiimide (DCC)), or an amine-reactive group (e.g., N-hydroxysulfosuccinimide (NHS), Sulfo-NHS, or NHS-esters). The reactive group may comprise a crosslinking agent, which may comprise an NHS group, an EDC group, a maleimide (e.g., for coupling with a Michael acceptor), a thiol, a cystamine, an aldehyde, a succinimidyl group, an epoxide, an acrylate. Examples of crosslinking agents include, for example, NHS (N-hydroxysuccinimide); sulfo-NHS (N-hydroxysulfosuccinimide); EDC (1-Ethyl-3-[3-dimethylaminopropyl]); carbodiimide hydrochloride; SMCC (succinimidyl 4-(N-maleimidomethyl)cyclohexane-1-carboxylate); sulfo-SMCC; DSS (disuccinimidyl suberate); DSG (disuccinimidyl glutarate); DFDNB (1,5-difluoro-2,4-dinitrobenzene); BS3 (bis(sulfosuccinimidyl)suberate); TSAT (tris-(succinimidyl)aminotriacetate); BS(PEG)5 (PEGylated bis(sulfosuccinimidyl)suberate); BS(PEG)9 (PEGylated bis(sulfosuccinimidyl)suberate); DSP(dithiobis(succinimidyl propionate)); DTSSP (3,3′-dithiobis(sulfosuccinimidyl propionate)); DST(disuccinimidyl tartrate); BSOCOES (bis(2-(succinimidooxycarbonyloxy)ethyl)sulfone); EGS (ethylene glycol bis(succinimidyl succinate)); DMA (dimethyl adipimidate); DMP (dimethyl pimelimidate); DMS (dimethyl suberimidate); DTBP (Wang and Richard's Reagent); BM(PEG)2 (1,8-bismaleimido-diethyleneglycol); BM(PEG)3 (1,11-bismaleimido-triethyleneglycol); BMB (1,4-bismaleimidobutane); DTME (dithiobismaleimidoethane); BMH (bismaleimidohexane); BMOE (bismaleimidoethane); TMEA (tris(2-maleimidoethyl)amine); SPDP (succinimidyl 3-(2-pyridyldithio)propionate); SMCC (Succinimidyl trans-4-(maleimidylmethyl)cyclohexane-1-Carboxylate); SIA (succinimidyl iodoacetate); SBAP (succinimidyl 3-(bromoacetamido)propionate); STAB (succinimidyl (4-iodoacetyl)aminobenzoate); Sulfo-SIAB (sulfosuccinimidyl (4-iodoacetyl) aminobenzoate); AMAS (N-α-maleimidoacet-oxysuccinimide ester); BMPS (N-β-maleimidopropyl-oxysuccinimide ester); GMBS (N-γ-maleimidobutyryl-oxysuccinimide ester); Sulfo-GMBS (N-γ-maleimidobutyryl-oxysulfosuccinimide ester); MBS (m-maleimidobenzoyl-N-hydroxysuccinimide ester); Sulfo-MBS (m-maleimidobenzoyl-N-hydroxysulfosuccinimide ester); SMCC (succinimidyl 4-(N-maleimidomethyl)cyclohexane-1-carboxylate); Sulfo-SMCC (sulfosuccinimidyl 4-(N-maleimidomethyl)cyclohexane-1-carboxylate); EMCS (N-ε-malemidocaproyl-oxysuccinimide ester); Sulfo-EMCS (N-ε-maleimidocaproyl-oxysulfosuccinimide ester); SMPB (succinimidyl 4-(p-maleimidophenyl)butyrate); Sulfo-SMPB (sulfosuccinimidyl 4-(N-maleimidophenyl)butyrate); SMPH (Succinimidyl 6-((beta-maleimidopropionamido)hexanoate)); LC-SMCC (succinimidyl 4-(N-maleimidomethyl) cyclohexane-1-carboxy-(6-amidocaproate)); Sulfo-KMUS (N-κ-maleimidoundecanoyl-oxysulfosuccinimide ester); SPDP (succinimidyl 3-(2-pyridyldithio)propionate); LC-SPDP (succinimidyl 6-(3(2-pyridyldithio)propionamido) hexanoate); LC-SPDP (succinimidyl 6-(3(2-pyridyldithio)propionamido)hexanoate); Sulfo-LC-SPDP (sulfosuccinimidyl 6-(3′-(2-pyridyldithio)propionamido)hexanoate); SMPT (4-succinimidyloxycarbonyl-alpha-methyl-α(2-pyridyldithio)toluene); PEG4-SPDP (PEGylated, long-chain SPDP crosslinker); PEG12-SPDP (PEGylated, long-chain SPDP crosslinker); SM(PEG)2 (PEGylated SMCC crosslinker); SM(PEG)4 (PEGylated SMCC crosslinker); SM(PEG)6 (PEGylated, long-chain SMCC crosslinker); SM(PEG)8 (PEGylated, long-chain SMCC crosslinker); SM(PEG)12 (PEGylated, long-chain SMCC crosslinker); SM(PEG)24 (PEGylated, long-chain SMCC crosslinker); BMPH (N-β-maleimidopropionic acid hydrazide); EMCH (N-ε-maleimidocaproic acid hydrazide); MPBH (4-(4-N-maleimidophenyl)butyric acid hydrazide); KMUH (N-κ-maleimidoundecanoic acid hydrazide); PDPH (3-(2-pyridyldithio)propionyl hydrazide); ATFB-SE (4-Azido-2,3,5,6-Tetrafluorobenzoic Acid, Succinimidyl Ester); ANB-NOS (N-5-azido-2-nitrobenzoyloxysuccinimide); SDA (NHS-Diazirine) (succinimidyl 4,4′-azipentanoate); LC-SDA (NHS-LC-Diazirine) (succinimidyl 6-(4,4′-azipentanamido)hexanoate); SDAD (NHS-SS-Diazirine) (succinimidyl 2-((4,4′-azipentanamido)ethyl)-1,3′-dithiopropionate); Sulfo-SDA (Sulfo-NHS-Diazirine) (sulfosuccinimidyl 4,4′-azipentanoate); Sulfo-LC-SDA (Sulfo-NHS-LC-Diazirine) (sulfosuccinimidyl 6-(4,4′-azipentanamido)hexanoate); Sulfo-SDAD (Sulfo-NHS-SS-Diazirine) (sulfosuccinimidyl 2-((4,4′-azipentanamido)ethyl)-1,3′-dithiopropionate); SPB (succinimidyl-[4-(psoralen-8-yloxy)]-butyrate); Sulfo-SANPAH (sulfosuccinimidyl 6-(4′-azido-2′-nitrophenylamino)hexanoate); DCC (dicyclohexylcarbodiimide); EDC (1-ethyl-3-(3-dimethylaminopropyl)carbodiimide hydrochloride); gluteraldehyde; formaldehyde; and combinations or derivatives thereof.

Molecules may also be attached to substrates using linkers. The linkers can have any useful number of functional groups or reactive groups and may be uni-functional (having one functional group), bi-functional, tri-functional, quadri-functional, or comprise a greater number of functional groups. In some instances, a molecule (e.g., nucleic acid molecule, peptide, or polymer) may be attached to a substrate using a heterobifunctional linker. The heterobifunctional linker may comprise any useful functional group, as described herein. Non-limiting examples of heterobifunctional linkers include: p-Azidobenzoyl hydrazide (ABH), N-5-Azido-2-nitrobenzoyloxysuccinimide (ANB-NOS), N-[4-(p-Azidosalicylamido)butyl]-3′-(2′-pyridyldithio) propionamide (APDP), p-Azidophenyl Glyoxal monohydrate (APG), Bis [B-(4-azidosalicylamido)ethyl]disulfide (BASED), Bis [2-(Succinimidooxycarbonyloxy)ethyl]Sulfone (BSOCOES), BMPS, 1,4-Di [3′-(2′-pyridyldithio)propionamido]Butane (DPDPB), Dithiobis(succinimidyl Propionate) (DSP), Disuccinimidyl Suberate (DSS), Discuccinimidyl Tartrate (DST), 3,3′-Dithiobis(sulfosuccinimidyl Propionate (DTSSP), EDC, Ethylene Glycol bis (succinimidyl succinate) (EGS), N-(E-maleimidocaproic acid hydrazide (EMCH), N-(E-maleimidocaproyloxy)-succinimide ester (EMCS), N-Maleimidobutyryloxysuccinimide ester (GMBS), Hydroxylamine-HCl, MAL-PEG-SCM, m-Maleimidobenzoyl-N-hydroxysuccinimide Ester (MBS), N-Hydroxysuccinimidyl-4-azidosalicylic acid (NHS-ASA), PDPH, N-Succinimidyl bromoacetate (SBA), SIA, Sulfo-SIA, Succinimidyl-4-(N-maleimidomethyl)cyclohexane-1-carboxylate (SMCC), Succinimidyl 4-(p-maleimidophenyl) Butyrate (SMPB), Succinimidyl-6-[ß-maleimidopropionamido]hexanoate (SMPH), N-Succinimidyl 3-[2-pyridyldithio]-propionate (SPDP), Sulfo-LC-SPDP, N-(p-Maleimidophenyl isocyanate (PMPI), N-Succinimidyl(4-iodoacetyl) Aminobenzoate (SIAB), Sulfo-MBS, Sulfo-SANPAH, Sulfo-SMCC, Sulfo-DST, Sulfo-EMCS, Sulfo-GMBS, N-Hydroxysulfosuccinimidyl-4-azidobenzoate (Sulfo-HSAB), Sulfosuccinimidyl (4-azidophenyl)-1,3 dithio propionate (Sulfo-SADP), Sulfosuccinimidyl 2-(m-azido-o-nitrobenzamido)-ethyl-1,3′-dithio propionate (Sulfo-SAND), Sulfosuccinimidyl-2-(p-azidosalicylamido)ethyl-1,3-dithiopropionate (Sulfo SASD), Sulfo-SIAB, Sulfo-SMCC, Sulfo-SMPB, and the like.

Additional examples of conjugation reactions that may be used to attach molecules to substrates include an Ullmann reaction, Heck reaction, Negishi reaction, Stille reaction, Suzuki reaction, Buchwald-Hartwig coupling, Castro-Stevens coupling, Glaser coupling, Kumada coupling, Larock indole synthesis, Miyaura borylation, Sonagashira cross-coupling, a Grubbs reaction.

More than one type of molecule may be coupled to the substrate. For example, a substrate may be coupled to nucleic acid molecules and peptides. Alternatively, a substrate may be coupled to only one type of molecule (e.g., only nucleic acid molecules, only peptides, only lipids, only carbohydrates, etc.). A substrate may be coupled to any useful combination of molecules, linkers, reactive moieties or functional groups, which may be coupled at any useful density, as described elsewhere herein. For example, a multifunctional linker may be used to attach both a nucleic acid barcode molecule and a peptide to the substrate. Alternatively, the substrate may comprise a plurality of bifunctional linkers that can conjugate to different molecules. In another example, a substrate may comprise a linker and reactive sites; the linker may be used to attach one type of molecule (e.g., peptides or nucleic acid molecules), whereas the reactive sites may be used to attach another type of molecule (e.g., nucleic acid molecules or peptides).

Linkers can comprise other functional portions, such as spacers (e.g., polymer chains, e.g., PEG, alkyl chains, etc.), cleavage sites (e.g., disulfide bridges that are cleavable upon application of a chemical stimulus, photocleavable or thermocleavable moieties, etc.), enzyme recognition sites, etc.

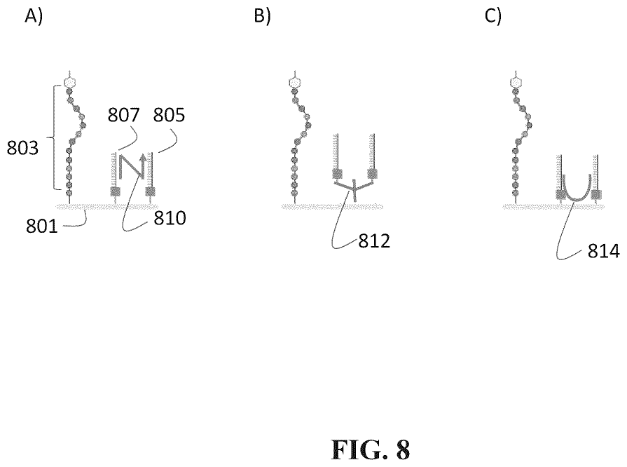

The proximity of a molecule coupled to a substrate to its nearest neighbor (e.g., another molecule) may be controlled using a variety of approaches, e.g., self-assembling monolayers, patterning approaches, linking moieties, etc. In some instances, it may be advantageous to have two molecules in close proximity (e.g., two polymerizable molecules, such as a peptide and a nucleic acid molecule, or two nucleic acid molecules). For instance, with respect to the sequencing approaches described herein, capture moieties may be used to couple a monomer of a polymeric analyte, and subsequent to monomer cleavage, an additional polymerizable molecule or plurality of polymerizable molecules may be required to be in proximity to the capture moiety to allow for transfer of information encoded by polymerizable molecules of binding agents. The proximity of the molecules (e.g., capture moiety and polymerizable molecules) may be mediated using tethering molecules, such as nucleic acid molecule “staples” or multi-functional linkers. For example, FIG. 8 shows examples of tethering approaches for coupling polymerizable molecules in close proximity to one another. In FIG. 8 Panel A, a substrate 801 is provided, coupled to polymeric analyte 803, a capture moiety 805, and an additional capture moiety 807. The capture moiety 805 and additional capture moiety 807 may be the same type of molecule (e.g., both nucleic acid molecules comprising the same sequence), or they may be different (e.g., a nucleic acid molecule and a peptide, nucleic acid molecules with different sequences, etc.). The capture moiety 805 and additional capture moiety 807 may be linked by a tethering molecule 810 comprising a first binding region that couples to the capture moiety 805 and a second binding region that couples to the additional capture moiety 807. For example, the capture moiety 805 and additional capture moiety 807 may comprise nucleic acid molecules, and the tethering molecule 810 may comprise a nucleic acid “staple” that comprises a first sequence that hybridizes to a first sequence of the capture molecule 805, and a second sequence that hybridizes to a second sequence of the additional capture molecule 807. The tethering molecule 810 may comprise additional sequences, e.g., loop sequences, spacer sequences, or other functional sequence, which may be disposed between the first sequence and the second sequence.

FIG. 8 Panel B schematically shows an example of a multi-functional linker that can be used to couple multiple molecules to a substrate. A multi-functional linker 812 may comprise a substrate-tethering group and one or more additional tethering groups, which may couple to polymerizable molecules and capture moieties 805 and 807. The multi-functional linker may be pre-coupled to the capture moieties (805 and 807) prior to coupling to the substrate, or the multi-functional linker may be provided coupled to the substrate, and the capture moieties may be introduced thereafter. The multi-functional linker 812 may comprise any number of useful functional or attachment moieties, e.g., EDC, NHS, click chemistry groups, maleimides, hydrazines, hydroxyl amines etc., as described elsewhere herein.

FIG. 8 Panel C schematically shows another example of a tethering molecule. Similar to tethering molecule 810, the tethering molecule 814 may comprise a first binding region that couples to the capture moiety 805 and a second binding region that couples to the additional capture moiety 807. For example, the capture moiety 805 and additional capture moiety 807 may comprise nucleic acid molecules, and the tethering molecule 814 may comprise a nucleic acid, U-shaped linker that comprises a first sequence that hybridizes to a first sequence of the capture molecule 805, and a second sequence that hybridizes to a second sequence of the additional capture molecule 807. The tethering molecule 810 may comprise additional sequences, e.g., loop sequences, spacer sequences, or other functional sequence, which may be disposed between the first sequence and the second sequence. The tethering group 814 may be single-stranded, double-stranded, or partially double-stranded. For instance, rather than using a U-shaped linker, the tethering group 814 may comprise two separate nucleic acid molecules comprising complementary sequences, which may be joined together to generate a U-shaped (or other morphology) linker.

Nucleic acid molecules may be coupled to a substrate by direct coupling. In such instances, the substrate or the nucleic acid molecules may comprise functional moieties that can interact. For example, the substrate and nucleic acid molecules may comprise a complementary click chemistry pair, e.g., alkyne and azide. In one such example, a substrate may comprise alkyne moieties (e.g., DBCO), which can be reacted with azide-functionalized nucleic acid molecules. The nucleic acid molecules may be reacted with the alkyne moieties in a click chemistry reaction to covalently link the substrate to the nucleic acid molecules. In another example, the substrate may comprise avidin or streptavidin moieties, to which biotinylated nucleic acid molecules may interact and bind non-covalently. Alternatively, or in addition to, the substrate may comprise a nucleic acid molecule to which additional nucleic acid molecules (e.g., nucleic acid analytes, nucleic acid linkers) are conjugated using hybridization, ligation, click chemistry, crosslinking (e.g., photocrosslinking such as CNVK).

Alternatively, or in addition to, the nucleic acid molecules may be coupled to a substrate using a linker, e.g., as described elsewhere herein. The linker may comprise at least two functional groups (e.g., a heterobifunctional linker) that can couple to both the substrate and the nucleic acid molecules. In an example, the substrate may comprise an amine group, and alkyne-functionalized DNA primers (e.g., DBCO-DNA primers) may be attached using a linker such as azidoacetic acid NHS ester. In another example, amine-functionalized substrates may be coupled to azide-functionalized DNA primers using a DBCO-NHS ester or DBCO-PEG-NHS ester linker. As described elsewhere herein, the linkers may comprise additional functional moieties (e.g., cleavage sites, spacers such as polymer or alkyl chains).

Similarly, peptides may be coupled to a substrate by direct coupling or by using a linker. A peptide may be coupled to a substrate at a terminus of the peptide (e.g., C terminus or N terminus), at an internal residue or amino acid of the peptide, or at multiple locations along the peptide. In examples of direct coupling, a peptide may be functionalized with a moiety that can interact with a moiety of the substrate (e.g., click chemistry pair, avidin-biotin). For example, the substrate and peptides may comprise a complementary click chemistry pair, e.g., alkyne and azide, or binding partners such as avidin and biotin. In one example of a click chemistry pair, a substrate may comprise alkyne moieties (e.g., DBCO), which can be reacted with azide-functionalized peptides. The peptides may be reacted with the alkyne moieties in a click chemistry reaction to covalently link the substrate to the peptides. In another example, the substrate may comprise avidin or streptavidin moieties, to which biotinylated peptides may interact and bind non-covalently.

Alternatively, or in addition to, the peptides may be coupled to a substrate using a linker, e.g., as described elsewhere herein. The linker may comprise at least two functional groups (e.g., a heterobifunctional linker) that can couple to both the substrate and the nucleic acid molecules. In an example, the substrate may comprise an amine group, and alkyne-functionalized peptides may be attached using a linker such as azidoacetic acid NHS ester. In another example, amine-functionalized substrates may be coupled to azide-functionalized peptides using a DBCO-NHS ester or DBCO-PEG-NHS ester linker. In yet another example, substrates comprising an amine group may be coupled to an azide-functionalized peptide using EDC and Sulfo-NHS.

A peptide may be functionalized with a functional moiety to enable attachment or coupling of the peptide to the substrate. The functional moiety may comprise a silane, e.g., aminosilane (e.g., APTES), amino-PEG-silane, click chemistry moiety or other linking moiety and can be attached to the peptide at a peptide terminus (N-terminus or C-terminus), at an internal amino acid, or at multiple locations (e.g., multiple internal amino acids, one or both termini, etc.). Chemical approaches to functionalize peptides can include C-terminal-specific conjugation (e.g., via C-terminal decarboxylative alkylation) using photoredox catalysis, e.g., as described by Bloom et al, Nature Chemistry 10, 205-211. 2018. and Zhang et al, ACS Chem. Biol. 2021, 16, 11, 2595-2603, each of which is incorporated by reference herein in its entirety, or amide coupling to an amine-functionalized surface. N-terminal attachment may comprise amide coupling of the N-terminus amine group to a carboxylic group functionalized surface or using 2-pyridinecarboxaldehyde variants. Alternatively, or in addition to, functionalization of terminal ends of peptides may be achieved enzymatically or using enzyme analogs such as ribozymes or DNAzymes. In an example of enzymatic functionalization and attachment, carboxypeptidases or amidases are used for C-terminal functionalization (e.g., as described in Xu et al, ACS Chem Biol. 2011 Oct. 21; 6(10): 1015-1020; Zhu et al, Chinese Chemical Letters. 2018, Vol 29 Issue 7, Pages 1116-1118; and Zhu et al, ACS Catal. 2022, 12, 13, 8019-8026, each of which is incorporated by reference herein in its entirety), which can allow for the addition of a click chemistry moiety to the peptide. The click chemistry-functionalized peptides may then be directly attached to the substrate via another clickable group (e.g., BCN-azide or DBCO-azide coupling), or, in other instances, may be reacted with another linker or polymerizable molecule (e.g., a bait nucleic acid molecule with a clickable group) that can then link to the substrate directly or indirectly (e.g., using a capture nucleic acid molecule and hybridizing the bait nucleic acid molecule). Additional examples of enzymes that can be used for functionalization or attachment include Sortase A, subtiligase, Butelase I, or trypsiligase. In some examples, ubiquitin ligase can be used to attach ubiquitin proteins with linker moieties to substrates. These linker moieties can then be used to chemically attach proteins to ubiquitin-coupled substrates. In some examples, glycosylating enzymes may be used to conjugate functionalized sugar groups (e.g., click chemistry functionalized sugars, polymer-conjugated sugars, biotinylated sugars) to amino acid residues, which can allow for attachment to a substrate (e.g., via click chemistry, polymer crosslinking or nucleic acid hybridization, avidin-biotin interactions), etc. Internal amino acid residues or post-translationally modified residues may be coupled to substrates using, for example, thiol labeling, amide coupling using EDC/NHS chemistry or DMT-MM to glutamate or aspartate residues, esterifying glutamate or aspartate residues, alkylation or disulfide bridge labeling of cysteines, or amide coupling to lysine residues.

A peptide may be treated prior to, during, or subsequent to coupling of the peptide to a substrate. In some instances, a peptide is conjugated with a tag that enables attachment to the substrate, e.g., using His tags, SNAP-tags, CLIP-tags, SpyCatcher, SpyTag, nucleic acid tags (e.g., bait oligos which can attach to capture oligos of the substrate).

In some examples, it may be advantageous to block or protect primary amines or carboxyl groups and optionally, de-block or de-protect the N-terminus primary amine or C-terminus carboxy group in order to facilitate attachment of the N-terminus or C-terminus to a substrate. In an example, single-point (e.g., C-terminal) selective attachment of peptides can be achieved by reacting the peptide with a linker comprising an amine-reactive group (e.g., isothiocyanates such as PITC) and a reactive group (e.g., click chemistry group). The linker can be, for example, PITC-conjugated click chemistry moieties such as PITC-azide, PITC-alkyne, optionally with spacer moieties in between, e.g., PITC-alkyl-azide, PITC-PEG-azide, PITC-alkyl-alkyne, PITC-PEG-azide). The linker reacts with and “blocks” the primary amines (e.g., modifies lysines), including the N-terminus. Subsequent cleavage of the N-terminal amino acid (e.g., using an Edman reagent, such as acid), can be performed, and one of the remaining modified lysines may be attached to a substrate (e.g., using the click chemistry moiety coupled to the amine-reactive group). Optionally, the peptide may be treated with a protease, e.g., LysC, which cleaves peptides such that a remaining peptide has a C-terminal lysine and such that the remaining peptide comprises a primary amine only at the C-terminal lysine residue and the N-terminus; such a cleavage may be performed prior to reacting the amine-reactive group, e.g., as shown by Xie et al. Langmuir 2022, 38, 30, 9119-9128, which is incorporated by reference herein in its entirety.

Similarly, carboxylic groups can be reacted in a way to enable C-terminal or internal residue attachment. In an example of C-terminal conjugation, carboxyl groups may be labeled with a C-terminal sequencing reagent, such as isothiocyanate, when treated with an activating reagent (e.g., acetic anhydride) to generate a peptide-thiohydantoin (at the C-terminus) and “blocked” carboxyl groups on the aspartic acid and glutamic acid residues. The thiohydantoin may then be reacted to couple to a substrate. Alternatively, cleavage of the C-terminal amino acid via a single round of C-terminal sequencing degradation, or via a protease, exposes only a single reactive carboxylic group at the C-terminal amino acid. The single reactive C-terminal carboxylic group can then be used as a reactive moiety for a single attachment site.

In another approach, a peptide or protein can be attached via the N-terminus using the specific reactivities of the N-terminus amine group. Amine-based reactions, such as amide coupling, can be carried out at low pH where only the N-terminal amine group is active. In addition, 2-pyridinecarboxyaldehyde and variants can be used to react to the N-terminal amine group.

In some instances, a peptide may be conjugated to a substrate using a polymerization reaction, e.g., a free radical polymerization, such as using PEGylated peptides, methacrylamide-modified peptides, Michael-type addition of maleimide-terminated oligo-NIPAAM-conjugated peptides; photocrosslinking of azophenyl-conjugated peptides, or other polymerization reactions with monomer-conjugated peptides, e.g., as described by Krishna et al. Biopolymers. 2010; 94(1): 32-48, which is incorporated by reference herein in its entirety.

Multiple types of molecules may be attached to a substrate. The substrate may comprise, coupled thereto, any combination of molecules, including but not limited to peptides, proteins (e.g., enzymes, ribozymes, DNAzymes, antibodies, nanobodies, antibody fragments), nucleic acid molecules, lipids, carbohydrates or sugars, metabolites, small molecules, polymers, metals, viral particles, biotin, avidin, streptavidin, neutravidin, etc. The multiple types of molecules may be attached simultaneously to the substrate or in a sequential manner. For example, a substrate may be treated to conjugate nucleic acid molecules and subsequently treated to conjugate peptides, or alternatively, the substrate may be treated to conjugate peptides prior to the nucleic acid molecules. Any number of conjugation or attachment chemistries may be used. For example, in instances where multiple types of molecules are attached to the substrate, any number of conjugation chemistries may be used for each type of molecule.

A substrate, or portion thereof, may be subjected to conditions sufficient to passivate the substrate or portion thereof. Passivation of a substrate may be useful for a variety of purposes, such as preventing nonspecific binding of binding agents, altering the surface density of a molecule (e.g., increasing the density of nucleic acid molecules or peptides), blocking reactive sites (e.g., blocking available click chemistry moieties subsequent to conjugation of the molecules on the substrate), etc. Passivation may be achieved using chemical approaches, e.g., deposition of blocking agents such as proteins (e.g., albumin), Tween-20, polymers, metals or metal oxides, or biochemical approaches, e.g., using metal microbes. Substrates comprising reactive moieties may also be passivated following molecule conjugation (e.g., coupling of nucleic acid molecules, peptides, etc.) by reacting any unreacted sites with an appropriate molecule. For example, a substrate comprising click chemistry moieties, e.g., DBCO beads, may be coupled to molecules of interest (e.g., polymerizable molecules, such as nucleic acid molecules, peptides) at a useful density using click chemistry (e.g., azide-nucleic acid molecules, azide-peptides). Unreacted sites may be passivated by providing and reacting complementary click-chemistry molecules, e.g., azide-polymers (e.g., PEG-azide), which may reduce downstream nonspecific interactions.

Substrate passivation may occur at any useful time or step. For instance, passivation to block unreacted DBCO sites may be performed prior to, during, or subsequent to conjugation of analytes or other molecules of interest (e.g., peptides and nucleic acid molecules). The passivation may be controlled by stoichiometry or densities of the passivating agent relative to the molecules of interest, or by physical approaches, e.g., photopatterning, self-assembling monolayers, etc.

### Sample Processing

The present disclosure also provides for methods of processing samples. One or more methods for processing samples may comprise preparation of biological samples for analysis, which, in some instances, includes partitioning of cells for conducting single-cell analysis. A method for processing a biological sample may comprise extraction or isolation of one or more peptides or proteins from the biological sample for further processing and analysis, as is described elsewhere herein.

Preparation of Cell Suspensions for Single-Cell Analysis: The methods described herein may involve preparation of single cell suspensions from a biological sample. Single cell suspensions may be prepared from biological samples by dissociating cells and optionally, culturing them in a liquid medium. In some instances, biological samples comprise a liquid sample. For example, a biological sample may comprise a bacterial liquid culture, a mammalian liquid culture, a blood, plasma, or serum sample. Processing of such liquid samples may include centrifugation (e.g., to isolate cells), resuspension of cells in a suitable medium, such as Dulbecco's Phosphate Buffered Saline (DPBS), and optional culturing of the isolated cells.

A biological sample may comprise cultured cells, e.g., cell cultured in suspension, or cells adhered to a solid surface, such as petri dishes or tissue culture dishes. Cultured adherent cells samples may be treated to generate a cell suspension, e.g., via a protease such as trypsin, to detach the cells from the surface. A biological sample may comprise a tissue or biopsy sample. A tissue or biopsy sample may be processed mechanically or enzymatically to generate a cell suspension. Such processing may include sonication (mechanical treatment) or enzymatic treatment, such as the use of pronase, collagenase, hyaluronidase, metalloproteinases, trypsin, or other enzymes that digest extracellular matrix components. The dissociated cells can then be stored in a suitable buffer, such as DPBS.

Cell Sorting: A biological sample or a cell suspension may be subjected to sorting to isolate a cell of interest. Sorting may be performed to select or isolate a cell based on a quality or characteristic of the cell, e.g., expression of a protein target, size, deformability, fluorescence or other optical property, or other physical property of the cell. Sorting may be accomplished using any number of approaches, e.g., using immunosorting (e.g., fluorescence activated cell sorting (FACS) or magnetic activated cell sorting (MACS)), electrophoretic approaches, chromatography, microfluidic approaches (e.g., using inertial focusing, cell traps, electrophoresis, isoelectric focusing), acoustic sorting, optical sorting (e.g., optoelectronic tweezers), mechanical cell picking (e.g., using manual or robotic pipettes) or passive approaches (e.g., gravitational settling).

Partitioning: Cells of a biological sample or cell suspension may be partitioned into individual partitions such that at least a subset of the individual partitions comprises a single cell. The individual partitions may comprise a barcode molecule (e.g., fluorophore or set of fluorophores, nucleic acid barcode molecules, etc.). Barcode molecules may be unique to the partition, such that each individual partition comprises a different barcode sequence than other partitions. The barcode molecules may be loaded into the individual partitions at any useful ratio of barcode molecules to sample species (e.g., cells, proteins, nucleic acid molecules). The barcode molecules may be loaded into partitions such that about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded into partitions such that more than about 0.0001, 0.001, 0.1, 1, 2, 3, 4, 5, 6, 7, 8, 9, 10, 20, 50, 100, 500, 1000, 5000, 10000, or 200000 barcodes are loaded per sample species. In some cases, the barcodes are loaded in the partitions so that less than about 0.0001, 0.001, 0.1, 1, 2, 3, 4, 5, 6, 7, 8, 9, 10, 20, 50, 100, 500, 1000, 5000, 10000, or 200000 barcodes are loaded per sample species.

A partition may assume any useful geometry such as a droplet, a microwell, a solid substrate, a gel (e.g., a cell encapsulated in a gel bead), a bead, a flask, a tube, a spot, a capsule, a channel, a chamber, or other compartment or vessel. A partition may be part of an array of partitions, e.g., a droplet in a microfluidic device, a microwell of a microwell plate, a spot on a multi-spot array, etc.

Lysis, Permeabilization, and Analyte Extraction: Single cells (e.g., in partitions) may be processed to obtain one or more analytes contained therein. A method for processing a single cell may comprise lysing the cell to release the contents into the individual compartment or partition. Lysis may be performed using a detergent (e.g., Triton-X 100, sodium dodecyl sulfate, sodium deoxycholate, CHAPS), RIPA buffer, a change in temperature (e.g., elevated or lower temperature, freezing, freeze-thawing), enzymes, ribozymes, DNAzymes, mechanical lysis (e.g., sonication, application of mechanical force), electrical lysis, or a combination thereof. Lysis may be performed in the presence of protease inhibitors to prevent degradation or digestion of the proteins from the cell. The contents may optionally be further processed, e.g., subjected to purification or extraction, denaturation of proteins or peptides, enzyme or chemical digestion, etc. In some instances, the contents may be subjected to enzymatic digestion to remove nucleic acid molecules, e.g. using nucleases such as DNAse or RNAse. Alternatively or in addition to, a cell may be fixed (e.g., using a fixative) and/or permeabilized. Examples of fixatives include aldehydes (e.g., glutaraldehyde, formaldehyde, paraformaldehyde), alcohols (e.g., methanol, ethanol), acetone, acids (e.g., acetic acid, Davidson's AFA), oxidizing agents (e.g., osmium tetroxide, potassium dichromate, chromic acid, permanganate salts), Zenker's fixative, picrates, Hepes-glutamic acid buffer-mediated organic solvent protection effect (HOPE), or Karnovsky fixative. Cell permeabilization may be achieved mechanically (e.g., using sonication, electroporation, shearing) or chemically (e.g., using an organic solvent such as methanol or acetone or detergents such as saponin, Tween-20, Triton X-100).

Protein Processing: The biological sample (or single cell suspensions or partitioned cells) may be further processed to enable proteomic analysis. For example, de-aggregation of proteins in the sample may be performed, e.g., using chemical or mechanical approaches. Chemical de-aggregation methods can include but are not limited to sodium dodecyl (SDS), Triton-X 100, 3-((3-cholamidopropyl) dimethylamminio)-1-proppanesulfonate (CHAPS), ethylene carbonate, or formamide. Mechanical de-aggregation methods can include but are not limited to sonication or high temperature treatment. The biological sample (or single cell suspensions or partitioned cells) may be subjected to conditions sufficient to denature one or more proteins. Denaturation may be achieved using heat, chemicals (e.g., SDS, urea, guanidine, formamide, metal organic compounds), reducing agents (e.g., dithiothreitol (DTT), beta mercaptoethanol, TCEP), urea, chaotropes, enzymes (e.g., ClpX, ClpS, unfoldases), ribozymes or DNAzymes. Similarly, the peptides or proteins may be subjected to conditions to solubilize the peptides or proteins in a solution, e.g., using detergents, organic solvents, spermidine, or tagging the peptides or proteins with polyionic tags (e.g., DNA, PEG, or other polymers). Alternatively, or in addition, the peptides or proteins may be enriched or purified; in an example, the peptides or proteins of interest may be precipitated using trichloroacetic acid, chloroform, TRIzol, or other chemical reagent. Other biological or chemical agents may be included during the protein processing, e.g., lysozymes, papain, cruzain, trypsin, protease inhibitors, nucleases or nuclease-containing proteins (e.g., DNAse, RNAse, DNA glycosylases, restriction endonucleases, transposases, micrococcal nucleases, Cas proteins).

Peptides or proteins may be fragmented prior to analysis. Fragmenting proteins may be useful in reducing the size of the proteins and allow for efficient processing of peptides, as is described elsewhere herein. Fragmentation may be performed using proteases, e.g., trypsin, chymotrypsin, pepsin, Lys-C, Glu-C, Proteinase K, furin, thrombin, endopeptidase, papain, subtilisin, elastase, enterokinase, genenanse, endoproteinase, metalloproteases, or with chemical treatment, e.g., cyanogen bromide, hydrazine, hydroxylamine, formic acid, BNPS-skatole, iodosobenzoic acid, 2-nitro-5-thiocyanobenzoic acid, etc. Alternatively or in addition to, fragmentation may be performed using mechanical methods, such as sonication, vortexing, mechanical stirring, using temperature changes (e.g., freeze/thaw, heating), or other fragmentation approach.

Enrichment of proteins or peptides in a biological sample may be performed, e.g., for separating proteins and peptides from cellular debris or other types of analytes (e.g., nucleic acids, lipids, carbohydrates, metabolites). Such enrichment may include, for example, the use of affinity columns (e.g., ion exchange), size exclusion columns, affinity precipitation (e.g., immunoprecipitation), chemical precipitation (e.g., using trichloroacetic acid, chloroform, TRIzol), chromatography (e.g., HPLC), or electrophoresis. In instances where cells are partitioned prior to enrichment, the enrichment may be performed using microbeads, affinity microcolumns, affinity beads, etc. In some instances, fractionation may be performed on the proteins or peptides, which may be used to separate the proteins by size, hydrophobicity, charge, affinity, size, mass, density, etc.

Peptides may be barcoded, in bulk or in partitions. Peptides may be barcoded with any useful type of barcode molecule, e.g., spectral or fluorescent barcodes, mass tags, nucleic acid barcode molecules, etc. The barcode molecules may allow for identification of an originating peptide, a partition, a sample, a cell, or cell compartment. For example, a cell sample may be partitioned such that a partition comprises at most one cell; the partition may comprise a unique barcode molecule (e.g., nucleic acid barcode molecule) that identifies the partition and thus the cell. Subsequent labeling of the peptides within the partition (e.g., by permeabilizing or lysing the cell) with the barcode molecules may be useful in identifying the peptides as arising or originating from the same cell or partition. In other examples, a substrate may comprise nucleic acid molecules comprising a unique barcode sequence that differs from barcode sequences of other substrates. As such, the barcode sequence may be used to identify the substrate. In some instances, barcoded substrates may be partitioned with cell samples, such that at least a subset of the partitions comprise a single cell and a single barcoded substrate. As such, the peptides arising from the single cell and transferred to the barcoded substrate may all be identifiable as originating from the single cell. Barcode molecules may comprise additional useful functional sequences, e.g., UMIs, primer sites, restriction sites, cleavage sites, transposition sites, sequencing sites, read sites, etc.

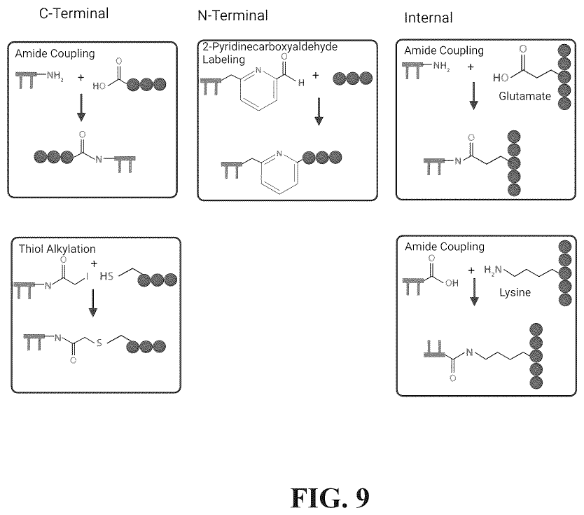

Attachment of barcode molecules to peptides may be achieved using any suitable chemistry. For example, C-terminal conjugation of nucleic acid barcode molecules may be achieved by amide coupling of amine-conjugated DNA barcode molecules to peptides or by thiol alkylation, e.g., reacting a thiolated peptide with an alkylated (e.g., iodoacetamide) DNA barcode molecule. N-terminal conjugation can be achieved, for instance, using 2-pyridinecarboxyaldehyde labeling of a DNA barcode and reacting with the N-terminus of a peptide. Internal residues, e.g., glutamate, can also be labeled with amine-conjugated DNA barcode molecules or carboxylated DNA barcodes (e.g., to react with primary amines in lysine). Examples of such conjugation approaches are schematically illustrated in FIG. 9.

Individual peptides may be barcoded at multiple locations for a given peptide. A peptide may be labeled at multiple sites with the same or different barcode sequences. For example, a peptide may be partitioned into a partition comprising a plurality of identical barcode molecules that comprise a barcode sequence that is unique to the partition. The peptide may be labeled at a single or multiple sites with the unique partition barcode sequence, optionally each comprising a unique molecular identifier (UMI), such that subsequent downstream analysis (e.g., sequencing) may be attributable to the same peptide using the barcode sequence. In some instances, a terminus of the peptide (e.g., N-terminus or C-terminus) or an internal amino acid may be labeled with a barcode. In some instances, the peptide may be fragmented prior to analysis or sequencing; accordingly, upstream attachment of multiple identical barcode molecules to the same peptide may allow for attribution of the sequence analysis back to a single peptide. Barcoding of peptides may occur prior to, during, or subsequent to fragmentation. Peptides may be labeled with barcodes (e.g., nucleic acid barcode molecules) using any suitable chemistry, e.g., as described above, or using bifunctional or trifunctional linkers comprising multiple linking moieties, e.g., as described elsewhere herein, such as click chemistry moieties, NHS-esters, EDC, etc. For example, C-terminal attachment may comprise amide coupling to C-terminus carboxylic group or photoredox tagging of C-terminus carboxylic group (e.g., to add an electrophile tag). N-terminal attachment may comprise amide coupling to N-terminus amine group, where specific attachment can occur at low pH, or using 2-pyridinecarboxaldehyde variants for specific attachment to N-terminus. Internal attachment may comprise, for example, amide coupling using EDC/NHS chemistry or DMT-MM to Glutamate or Aspartate; alkylation or disulfide bridge labeling of cysteines; or amide coupling to lysine residues (see, e.g., FIG. 9).

In some examples, a peptide may be labeled with different barcode molecules, which can be indexed by proximity to one another, e.g., using primers that can anneal to adjacent barcode molecules. In one such approach, after a protein has been labeled with a plurality of barcodes with different barcode sequences, proximity-based polymerase extension may be used to copy and associate the sequence of adjacent barcodes. For example, each barcode molecule may comprise a primer binding site, to which a dual-primer linker sequence comprising two sequences is annealed. The dual primer linker sequence can bind to the primer binding sites of two adjacent barcodes. An extension reaction, e.g., using a polymerase, may extend and copy the barcode sequences of the adjacent barcodes. Subsequently, the dual primer linker sequence, which now has copies of the two adjacent barcodes, may be removed and sequenced. From the sequencing reads, an adjacency matrix of barcode sequences may be generated (e.g., to correspond barcode sequences on a single dual primer linker as spatially adjacent). Accordingly, each of the barcode sequences may be associated with a nearby adjacent barcode sequences, and as such, peptide portions may be aligned or attributed as being adjacent. Such an approach may be useful in instances where the peptide is fragmented, such that individual fragments of a peptide may be corresponded with the nearest neighbor using the barcode sequences.

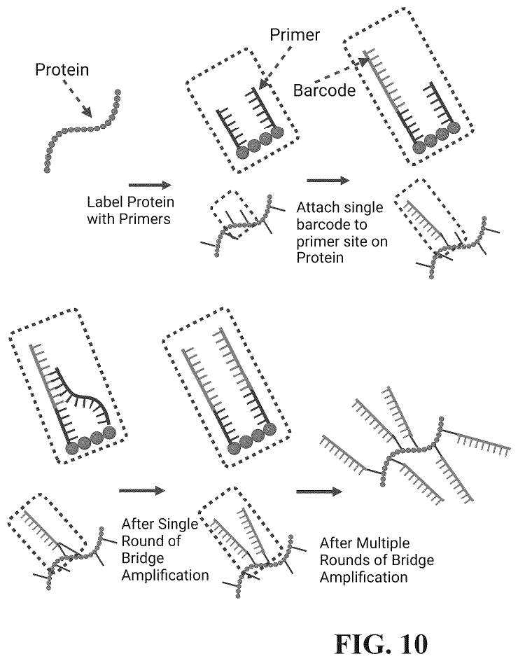

In another example, a peptide may be barcoded at multiple locations for a given peptide using bridge amplification. In such an approach, and as schematically depicted in FIG. 10, a peptide or protein may be labeled at multiple sites with a nucleic acid primer. A nucleic acid barcode molecule may be provided, which can anneal to the nucleic acid primer (not shown) or be ligated to the nucleic acid primer. Subsequent rounds of bridge amplification may be performed in order to copy the nucleic acid barcode molecule to the other primers located at other sites of the given peptide. In some examples, a peptide may be tagged with multiple copies of the nucleic acid primer, and barcode sequences may be provided sparsely, such that only one nucleic acid primer per peptide is extended by polymerase extension. Subsequent rounds of bridge amplification can result in a peptide having the same barcode sequence at each nucleic acid primer. Subsequent fragmenting of peptides may be performed, such that peptide fragments comprise on average, a single barcode. Accordingly, in some cases, the output such an amplification approach may be peptides with individual barcodes generated from fragmenting multi-labeled proteins where peptides from the same protein have the same barcodes.

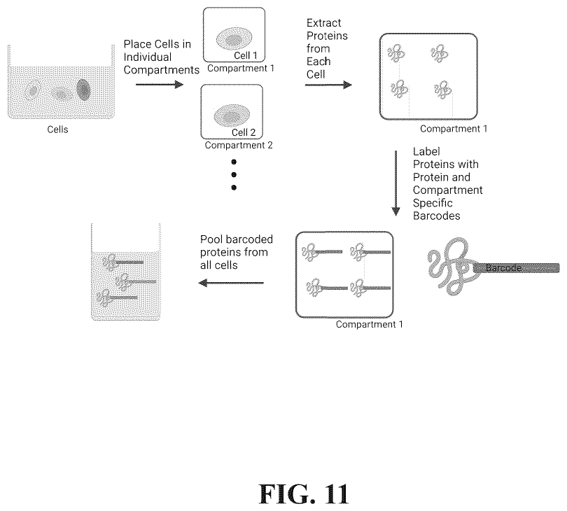

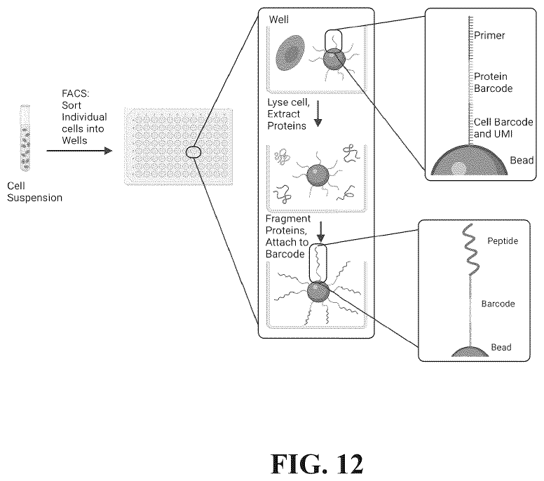

FIGS. 11-12 schematically illustrate an example workflow for processing a cell sample in partitions to obtain barcoded peptides. A sample of cells may be partitioned into individual partitions or compartments (e.g., droplets, microwells) such that at least a subset of the partitions comprise a single cell. The partitions may then be treated with a lysing agent to lyse the cells and release the proteins from the cells into the partition. The proteins may then be labeled with a partition-specific barcode (e.g., using a barcode bead, see FIG. 12), such that all peptides or proteins arising from a single compartment comprises the same barcode. In some examples, the barcodes comprise nucleic acid barcode molecules, and the barcode sequence can be used in downstream processing, e.g., via sequencing, to identify the partition or cell from which a peptide originated. The nucleic acid barcode molecule may comprise any additional useful sequences, e.g., UMIs, primer sequences, etc.

Bulk Processing: A biological sample may be processed in bulk. For example, a biological sample may be processed to obtain a suspension of cells, which may be directly lysed in the suspension, without partitioning of cells in individual compartments. Cells may be lysed in bulk using any useful approach, e.g., as described above and optionally subjected to further processing, e.g., homogenization, protease inhibition, denaturation, protein processing (e.g., chemical treatment, fragmentation), or a combination thereof. A biological sample may be subjected to pre-processing prior to cell lysis or protein extraction. Such pre-processing may include removal of debris, purification, filtration, concentration, or sorting.

Spatial barcoding: A biological sample may comprise a tissue sample comprising multiple cells. Tissue samples may be processed using an approach to retain spatial information (e.g., to identify peptides from individual cells), e.g., using spatial barcodes. For instance, a 2-D or 3-D tissue sample may be provided, and individual cells or locations within a tissue sample may be contacted with a plurality of spatial barcodes (e.g., nucleic acid barcode molecules) comprising different barcode sequences. The different barcode sequences may be attributed to a particular location in the 2-D or 3-D tissue sample, which may correspond with a location of a cell. For example, spatial barcodes may be provided using deterministic methods such as two-photon patterning, or stochastic methods such as PCR, to assign different segments of the 2-D or 3-D tissue sample with unique spatial barcodes. Accordingly, peptides that are labeled with spatial barcodes may be attributed back to a single location within a tissue sample, or back to a single cell.

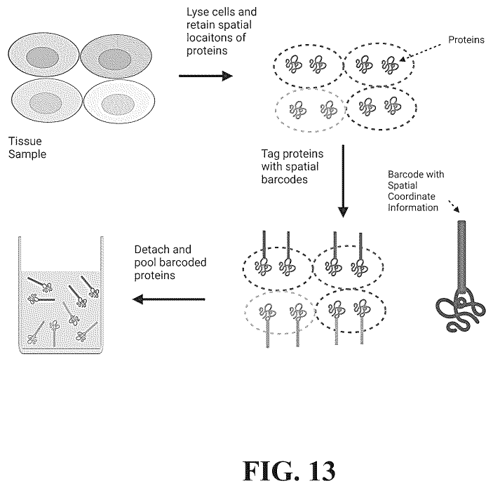

FIG. 13 schematically illustrates an example workflow of spatial barcoding of a tissue sample. A tissue sample comprising multiple cells (illustrated as a 2×2 array of cells) may be provided. The tissue sample may be subjected to lysis or fixation and permeabilization to provide access to the proteins contained within the multiple cells. Spatial barcodes, e.g., nucleic acid barcode molecules, may be provided. The spatial barcodes may comprise coordinate or location information. In an example, each cell may be contacted with a different spatial barcode, or portions of cells may be contacted with different spatial barcodes, which may optionally be pre-indexed (e.g., using imaging, or deterministic spatial barcoding approaches). Further processing of the peptides may be performed, as described elsewhere herein. As the peptides are labeled with the spatial barcodes, each peptide having a spatial barcode may be attributed back to its originating coordinate or location, which can help identify the originating cell from which a peptide arises.

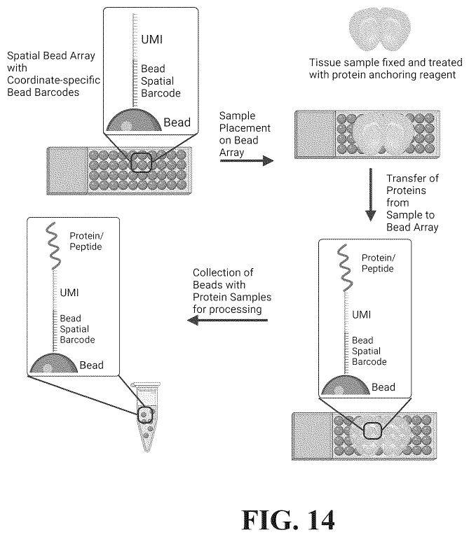

FIG. 14 schematically illustrates another example workflow of spatial barcoding of a tissue sample. A spatial barcode array may be provided on a substrate (e.g., a glass microscope slide, a hydrogel mesh). In some instances, the spatial barcodes may be directly conjugated to the substrate, or they may be provided on barcoded beads. In an example, a plurality of beads each comprising different barcode sequences may be arranged in an array on a substrate. Each bead may comprise a spatial barcode comprising a spatial barcode sequence, and optionally, a unique molecular identifier (UMI). A tissue sample (e.g., a fixed tissue sample) may be placed adjacent to (e.g., overlayed onto) the spatial barcode array. The tissue sample may then be subjected to conditions sufficient to transfer the peptides or proteins to the spatial barcode array. For example, the peptides or proteins may be transferred via passive transport, e.g., diffusion or Brownian motion, or via active transport, e.g., electrophoresis, pressure-driven flow, etc. The peptides or proteins may be attached to the spatial barcodes, e.g., using a linker (e.g., comprising amine-reactive groups, or click chemistry groups such as azide, alkyne, or other functional moieties such as aldehyde groups or NHS or carboxylic groups), conjugation chemistry, or an anchoring agent. Examples of anchoring agents include fixatives, such as formaldehyde, paraformaldehyde, glutaraldehyde, or monomers for incorporation into a hydrogel, e.g., Acryloyl-X, acrylamide, N-(3-Aminopropyl)methacrylamide, or N-(3-Aminoethyl)methacrylamide, or benzophenone. Anchoring agents may also comprise multi-functional linkers, e.g., Acryloyl-X, Biotin-NHS, Biotin-PEG-Amine, DBCO-NHS, DBCO-amine. For bead arrays, the plurality of beads may be collected from the sample for further processing.

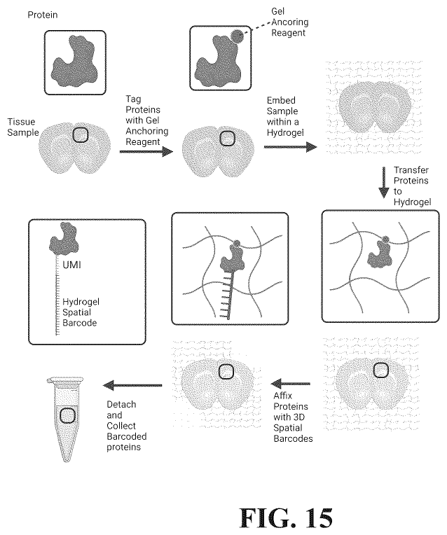

FIG. 15 schematically shows an example of tagging proteins in samples with spatial barcodes using a 3D hydrogel matrix. Intact samples can be embedded in a 3D hydrogel (e.g., polyacrylamide, polyacrylate, ExM), and proteins from samples are transferred to the hydrogel via a gel anchoring reagent (e.g., benzophenone, which can allow for photocapture). Spatial barcodes may be provided such that a pre-indexed coordinate or location comprises a unique spatial barcode. The spatial barcodes may be attached to the hydrogel matrix. The barcoded proteins may subsequently be released or detached from the hydrogel matrix for further processing. Release or detachment may be obtained using enzymatic approaches (e.g., endonuclease cleavage), chemical approaches, or mechanical approaches. The spatially barcoded proteins may then be identifiable in downstream processing operations, e.g., sequencing, to determine the barcode and the originating location of the protein.

### Computer Systems

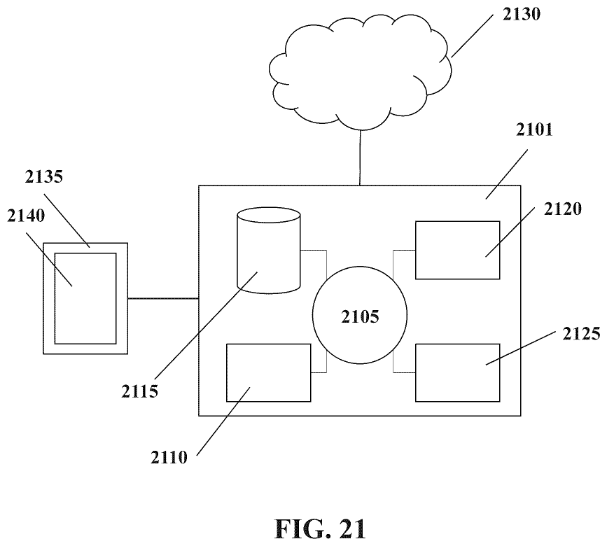

The present disclosure provides computer systems that are programmed to implement methods of the disclosure. FIG. 21 shows a computer system 2101 that is programmed or otherwise configured to sequence polymeric analytes. The computer system 2101 can regulate various aspects of sample processing of the present disclosure, such as, for example, automated handling of one or more systems, generating sequencing reads of the polymerizable molecules described herein, and outputting an identity of monomers of the polymeric analytes. The computer system 2101 can be an electronic device of a user or a computer system that is remotely located with respect to the electronic device. The electronic device can be a mobile electronic device.

The computer system 2101 includes a central processing unit (CPU, also “processor” and “computer processor” herein) 2105, which can be a single core or multi core processor, or a plurality of processors for parallel processing. The computer system 2101 also includes memory or memory location 2110 (e.g., random-access memory, read-only memory, flash memory), electronic storage unit 2115 (e.g., hard disk), communication interface 2120 (e.g., network adapter) for communicating with one or more other systems, and peripheral devices 2125, such as cache, other memory, data storage and/or electronic display adapters. The memory 2110, storage unit 2115, interface 2120 and peripheral devices 2125 are in communication with the CPU 2105 through a communication bus (solid lines), such as a motherboard. The storage unit 2115 can be a data storage unit (or data repository) for storing data. The computer system 2101 can be operatively coupled to a computer network (“network”) 2130 with the aid of the communication interface 2120. The network 2130 can be the Internet, an internet and/or extranet, or an intranet and/or extranet that is in communication with the Internet. The network 2130 in some cases is a telecommunication and/or data network. The network 2130 can include one or more computer servers, which can enable distributed computing, such as cloud computing. The network 2130, in some cases with the aid of the computer system 2101, can implement a peer-to-peer network, which may enable devices coupled to the computer system 2101 to behave as a client or a server.

The CPU 2105 can execute a sequence of machine-readable instructions, which can be embodied in a program or software. The instructions may be stored in a memory location, such as the memory 2110. The instructions can be directed to the CPU 2105, which can subsequently program or otherwise configure the CPU 2105 to implement methods of the present disclosure. Examples of operations performed by the CPU 2105 can include fetch, decode, execute, and writeback.

The CPU 2105 can be part of a circuit, such as an integrated circuit. One or more other components of the system 2101 can be included in the circuit. In some cases, the circuit is an application specific integrated circuit (ASIC).

The storage unit 2115 can store files, such as drivers, libraries and saved programs. The storage unit 2115 can store user data, e.g., user preferences and user programs. The computer system 2101 in some cases can include one or more additional data storage units that are external to the computer system 2101, such as located on a remote server that is in communication with the computer system 2101 through an intranet or the Internet.

The computer system 2101 can communicate with one or more remote computer systems through the network 2130. For instance, the computer system 2101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2101 via the network 2130.

Methods as described herein can be implemented by way of machine (e.g., computer processor) executable code stored on an electronic storage location of the computer system 2101, such as, for example, on the memory 2110 or electronic storage unit 2115. The machine executable or machine-readable code can be provided in the form of software. During use, the code can be executed by the processor 2105. In some cases, the code can be retrieved from the storage unit 2115 and stored on the memory 2110 for ready access by the processor 2105. In some situations, the electronic storage unit 2115 can be precluded, and machine-executable instructions are stored on memory 2110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 2101, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2101 can include or be in communication with an electronic display 2135 that comprises a user interface (UI) 2140 for providing, for example, sequencing reads, analysis of the sequencing reads, e.g., identities of individual monomers of a polymeric analyte. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2105. The algorithm can, for example, deconvolve a DNA sequence obtained from the polymerizable molecules and output a sequence of the polymeric analyte (e.g., amino acid sequence of a peptide).

### EXAMPLES

### Example 1—Protocol for Attachment of Nucleic Acid Molecules to Substrates

As described herein, a polymeric analyte, a capture moiety, and a polymerizable molecule may be coupled to a substrate to perform sequencing analysis of the polymeric analyte. In some examples, both the capture moiety and the polymerizable molecule are nucleic acid molecules (e.g., DNA molecules) comprising the same nucleic acid sequence. These nucleic acid molecules can be conjugated onto a substrate or surface. In one such example, the substrate is a TentaGel™ S-NH₂ (Sigma-Aldrich) bead, to which commercially-available, alkyne-functionalized (e.g., DBCO-functionalized) nucleic acid molecules (e.g., DNA primers) can attach, using a linker (e.g., azidoacetic acid NHS ester).

Materials: DBCO-functionalized DNA primers (Integrated DNA Technologies), TentaGel™ S-NH₂ beads (100 μm, Sigma-Aldrich), Azidoacetic acid NHS ester, DMSO (anhydrous), Tris (pH 8.0), NaCl, EDTA, PEG-1000 (up to 50% (w/v)). Equipment: thermomixer.

Procedure: 1. Add 10-fold molar excess of Azidoacetic acid NHS ester to the TentaGel beads in anhydrous DMSO. 2. Incubate the beads at room temperature overnight while rotating. 3. Wash the beads twice with anhydrous DMSO and twice with 50% (v/v) DMSO/water mixture (each wash for 30 min). 4. Make 1 M NaCl, 50 mM Tris (pH 8.0), and 0.5 mM EDTA (conjugation buffer) Note: up to 50% (w/v) PEG-1000 can be included in the conjugation buffer to help increase the surface density of DNA primers. 5. Add 0.2-1 nmole of DNA primers per 1 mg of beads in 40 μl conjugation buffer. 6. Incubate the beads at 37° C. overnight while shaking. 7. Wash the beads three times with conjugation buffer (each wash for 30 min).

In another example, TentaGel™ beads may be modified to have an alkyne (e.g., DBCO), to which azide-functionalized DNA primers may be attached.

Materials: Azide-functionalized DNA primers (Integrated DNA Technologies), TentaGel™ S-NH₂ beads (100 μm, Sigma-Aldrich), DBCO-PEG5-NHS ester, DMSO (anhydrous), Tris (pH 8.0), NaCl, EDTA, PEG-1000 (up to 50% (w/v)). Equipment: thermomixer.

Procedure: 1. Add 10-fold molar excess of DBCO-PEG5-NHS ester to the TentaGel beads in anhydrous DMSO. 2. Incubate the beads at room temperature overnight while rotating. 3. Wash the beads twice with anhydrous DMSO and twice with 50% (v/v) DMSO/water mixture (each wash for 30 min). 4. Make 1 M NaCl, 50 mM Tris (pH 8.0), and 0.5 mM EDTA (conjugation buffer) Note: up to 50% (w/v) PEG-1000 can be included in the conjugation buffer to help increase the surface density of DNA primers. 5. Add 0.2-1 nmole of DNA primers per 1 mg of beads in 40 μl conjugation buffer. 6. Incubate the beads at 37° C. overnight while shaking. 7. Wash the beads three times with conjugation buffer (each wash for 30 min).

In yet another example, TentaGel™ beads may be modified to generate a bilayer bead, which may be useful in generating topologically segregated beads and thereby functionalizing a portion of the bead (e.g., the exterior surface, an exterior layer). The bilayer beads may then be conjugated to azide-functionalized DNA primers. Bilayer beads can be generated as described in Ruiwu et al. QSAR & Combinatorial Science, 2005, 24(10), 1127-1140, which is incorporated by reference herein in its entirety. Briefly, TentaGel™ beads may be swollen in deionized water, then combined with Fmoc-OSu dissolved in DCM and DIEA, shaking, then washing with DMF, followed by methanol, then DMF. Acetic acid, HOBt, and DIC may be added to the resin, then washed again with DMF, methanol, DMF, then incubated with 4-methylpiperidine, followed by washing with DMF, methanol (2×), and DMF again. The resin can then be stored in DMSO.

Materials: Azide-functionalized DNA primers (Integrated DNA Technologies), bilayer TentaGel™ S-NH₂ beads, DBCO-PEG5-NHS ester, DMSO (anhydrous), Tris (pH 8.0), NaCl, EDTA, PEG-1000 (up to 50% (w/v)). Equipment: thermomixer.

Procedure: 1. Add 10-fold molar excess of DBCO-PEG5-NHS ester to the TentaGel™ bilayer beads in anhydrous DMSO. 2. Incubate the beads at room temperature overnight while rotating. 3. Wash the beads twice with anhydrous DMSO and twice with 50% (v/v) DMSO/water mixture (each wash for 30 min). 4. Make 1 M NaCl, 50 mM Tris (pH 8.0), and 0.5 mM EDTA (conjugation buffer) Note: up to 50% (w/v) PEG-1000 can be included in the conjugation buffer to help increase the surface density of DNA primers. 5. Add 0.2 nmole of DNA primers per 1 mg of beads in 40 μl conjugation buffer. 6. Incubate the beads at 37° C. overnight while shaking. 7. Wash the beads three times with conjugation buffer (each wash for 30 min).

### Example 2—Protocol for Attachment of Peptides to Substrates

Polymeric analytes, e.g., peptides may be attached to a substrate. In some instances, the peptides may be attached to the substrate after attachment of the capture moieties and polymerizable molecules (e.g., DNA primers, as described in Example 1). In some examples, the peptides may be conjugated via the C-terminus, which exposes the N-terminus of the peptides for subsequent sequencing. After peptide attachment to the substrate, the excess attachment sites on the surface can be blocked, e.g., using mPEG.

In an example, peptides may be conjugated to a substrate comprising nucleic acid molecules coupled thereto, e.g., TentaGel™ beads comprising DBCO moieties and azide-functionalized DNA primers, as described in Example 1. Peptides may be from a sample or obtained commercially (e.g., from GenScript). The peptides may be attached to the substrate using a linker, or by functionalizing the peptides to comprise a complementary click chemistry moiety (e.g., azide).

Materials: Peptides (GenScript); Azido-PEG3-amine, EDC, Sulfo-NHS, BupH™ MES buffer (pH 5.5), beta-mercapto ethanol (BME), BupH™ PBS buffer (pH 7.2), Tris (pH 8.0). Equipment: Thermomixer.

Procedure: 1. Make 0.5 mM peptides in 100 mM MES buffer (pH 5.5). 2. Add EDC and Sulfo-NHS to the peptides at a 10:1 and 25:1 molar ratio, respectively. 3. Incubate the reaction at room temperature for 30 min. 4. Quench EDC by 10-fold molar excess of BME. Make 5 mM Azido-PEG3-amine in 200 mM PBS buffer (pH 7.2). 5. Mix the peptide solution with the Azido-PEG3-amine solution at a 1:1 volume ratio. 6. Incubate the reaction at room temperature overnight. 7. Quench the reaction by adding 10 mM Tris (pH 8.0). The expected output includes peptide- and oligo-conjugated TentaGel™ beads.

In another example, azide-functionalized peptides may be conjugated to a substrate comprising nucleic acid molecules coupled thereto, e.g., TentaGel™ beads comprising DBCO moieties and azide-functionalized DNA primers, as described in Example 1.

Materials: Azide-functionalized peptides (GenScript); DBCO-functionalized TentaGel™ beads, Azide-m-PEG5, DMSO (anhydrous), Tris (pH 8.0), NaCl, EDTA. Equipment: Thermomixer.

Procedure: 1. Make 1 M NaCl, 50 mM Tris (pH 8.0), and 0.5 mM EDTA (conjugation buffer). 2. Add <1 pmole of peptides per 1 mg of Tentagel beads in 40 μl conjugation buffer. 3. Incubate the beads at 37° C. overnight while shaking. 4. Wash the beads three times with conjugation buffer (each wash for 30 min). 5. Add 10-fold molar excess of Azide-m-PEG5 to the beads in conjugation buffer. 6. Incubate the beads at 37° C. overnight while shaking. 7. Wash the beads three times with conjugation buffer (each wash for 30 min). The expected output includes peptide- and oligo-conjugated TentaGel™ beads.

### Example 3—Protocol for Coupling of the Monomer to a Capture Moiety

As described herein, a method for processing and analyzing a polymeric analyte (e.g., a peptide) may comprise coupling a monomer (e.g., N-terminal amino acid) to a capture moiety to generate a monomer-capture moiety complex. In an example, a peptide and a capture moiety (e.g., DNA primer) may be conjugated to a substrate, e.g., as described in Examples 1 and 2. A bifunctional linker comprising an amino-acid reactive group (e.g., isothiocyanate group) and a click chemistry moiety (e.g., azide) may be contacted with the monomer. A linking nucleic acid molecule comprising a complementary click chemistry moiety (e.g., alkyne) may be reacted with the bifunctional linker. The linking nucleic acid molecule may be coupled to the capture moiety, e.g., via complementary sequence hybridization, through a splint or bridge oligo, or via blunt end ligation, to generate the monomer-capture moiety complex.

In one particular example of coupling a linker to an N-terminal amino acid of a peptide that is conjugated to a substrate (at the C-terminus), 1-(2-azidoethyl)-4-isothiocyanatobenzene (also referred to herein as PITC-azide or azide-PITC) may be used as a linker and mixed with a substrate comprising the peptide and capture moieties coupled thereto. The linker comprises two reactive moieties: (i) a phenyl isothiocyanate moiety (isothiocyanatobenzene), which can react with a primary amine, e.g., the N-terminal amino acid of the peptide (e.g., at pH ˜9.5 at 50° C.), and (ii) an azide moiety, which can be reacted with an alkyne moiety in a click chemistry reaction, e.g., to couple to an alkyne-conjugated linking nucleic acid molecule.

Materials: Peptide and Oligo-conjugated beads (e.g., output from Examples 1 and 2 above), 1-(2-azidoethyl)-4-isothiocyanatobenzene, Acetonitrile, Water, Pyridine, Triethylamine. Equipment: Hot stirring plate or Shaking Incubator, Nitrogen or Argon Gas Compartment.

Procedure: 1. Combine solvents Acetonitrile, Pyridine, Water, and Triethylamine at a ratio of 10:5:3:2. 2. Add the solvent solution to the beads. 3. Add 1-(2-azidoethyl)-4-isothiocyanatobenzene (dissolved in acetonitrile) at a 100-1000 equivalent of the peptide amount or concentration. 4. Run the reaction at 50° C. for 30 minutes in an inert atmosphere (e.g., argon gas). 5. Remove the solvent. 6. Wash in acetonitrile 2-3 times to remove excess linker, pyridine, and triethylamine. The expected output is a substrate comprising a peptide-linker conjugate, which comprises the peptide, in which the N-terminal amino acid is in the PTC-derivatized form and comprises an azide moiety (from the linker).

Prior to, during, or subsequent to the coupling of the linker to the terminal amino acid of the peptide, the linker may be reacted with a linking nucleic acid molecule (e.g., DBCO-conjugated DNA molecule). In one example, the linker may be first coupled to the terminal amino acid, and excess linker is removed, as described above. The linker-amino acid (e.g., PTC-form of the N-terminal amino acid) may then be reacted with the linking nucleic acid molecule in a click chemistry reaction.

Procedure. 1. Combine 1-2 equivalents of alkyne-conjugated DNA to the peptide-linker conjugates. 2. Stir at 50° C. for 2-3 hours. 3. Remove the solvent. 4. Wash in DMSO or water 2-3 times to remove excess DBCO-conjugated DNA. The expected output is a substrate comprising a peptide-linker-linking nucleic acid molecule complex (e.g., as schematically shown in FIG. 1A Panel B).

The peptide-linker-linking nucleic acid molecule complex may be coupled to a capture moiety (e.g., DNA primer) of the substrate, e.g., via hybridization, ligation, or both. In one example, the linking nucleic acid molecule is coupled to the capture moiety using a splint oligonucleotide and ligated together using a ligase (e.g., T4 DNA ligase).

Materials: 10×DNA ligase buffer: Final 1× composition is Tris (50 mM, pH 7.5) with ATP (1 mM), MgCl2 (10 mM), DTT (10 mM), Splint DNA oligo (1 mM in water), T4 DNA ligase enzyme (4000 U/μL) Important: DNA ligase is not heat stable and must be stored at −20 C for long term stability and 4 C for short term stability, 50% PEG-2000 in water. Equipment: Shaker.

Procedure: 1. Wash beads (e.g., comprising peptide-linker-linking nucleic acid molecule complexes) with TE (10 mM Tris pH 8, 0.1 mM EDTA). 2. For a 100 uL reaction combine in a tube on ice: 48.5 uL of water, 10 μL of 10×DNA ligase buffer, 40 μL of 50% PEG-2000, 1 μL of Splint DNA oligo, and 0.5 uL of T4 DNA ligase enzyme. 3. Incubate with shaking for 30 minutes at room temperature. Optional: Add 100 μL of 20 mM EDTA to stop the reaction. The expected output is a substrate comprising a peptide comprising a monomer that is covalently coupled to a capture moiety (DNA primer) (e.g., as shown in FIG. 1A Panel C).

In another example, a different ligase may be used.

Materials: 10× Buffer: Final 1× compositions is Bis-Tris-Propane HCl (10 mM, pH 7) with MgCl2 (10 mM), and DTT (1 mM), ATP (1 mM), Mth RNA ligase (50 uM), Thermostable 5′App DNA/RNA ligase (20 uM), and 50% PEG-2000 in water. Equipment: Shaker or Shaker Incubator.

Procedure: 1. Wash beads with TE (10 mM Tris pH 8, 0.1 mM EDTA). 2. For a 100 uL reaction combine in a tube on ice: 32.5 uL of water, 10 μL of 10× Buffer, 10 μL of ATP (1 mm), 40 μL of 50% PEG-2000, 2.5 uL of Mth RNA ligase (50 uM), and 5 μL of Thermostable 5′App DNA/RNA ligase (20 uM). 3. Incubate with shaking for 2 hours minutes at 65° C. Optional: Add 100 μL of 20 mM EDTA to stop the reaction. The expected output is a substrate comprising a peptide comprising a monomer that is covalently coupled to a capture moiety (DNA primer) (e.g., as shown in FIG. 1A Panel C).

### Example 4—Protocol for Cleaving the Monomer from the Polymeric Analyte

As described herein, a method for processing and analyzing a polymeric analyte (e.g., a peptide) may comprise cleaving a monomer (e.g., NTAA) from a polymeric analyte (e.g., peptide). The cleavage may occur during any convenient step or process. In some examples, the cleavage is performed subsequent to coupling of the monomer to the capture moiety (e.g., as described in Example 3). Cleavage may be mediated by a variety of approaches, as described elsewhere herein. In some instances, cleavage is performed under acidic conditions, e.g., using trifluoroacetic acid.

Materials: Beads comprising peptide linker complexes—optionally with the NTAA covalently coupled to a capture moiety (e.g., resulting from Example 3), Anhydrous trifluoroacetic acid (TFA). Equipment: Shaker Incubator, Inert gas compartment.

Procedure: 1. Add TFA to the beads. 2. Heat the reaction mixture to 50° C. for 10 min in an inert atmosphere. 3. Remove the TFA. 4. Wash in water 2-3 times to remove excess TFA. The expected output is a cleaved NTAA and remaining peptide. If the NTAA is covalently linked to the capture moiety beforehand, the expected output is a monomer-capture moiety complex and a remaining peptide (e.g., as shown schematically in FIG. 1A Panel D).

Alternatively, the cleavage may be performed under more mild conditions, e.g., to be more compatible with all nucleic acid bases. One such approach is to perform the cleavage under basic (alkaline) conditions.

Materials: Beads comprising peptide linker complexes—optionally with the NTAA covalently coupled to a capture moiety (e.g., resulting from Example 3), Triethylamine, Acetic Acid, Acetonitrile. Equipment: Shaker Incubator, Inert gas compartment.

Procedure: 1. Prepare a 1:1:1 ratio mixture of Triethylamine, acetonitrile, and acetic acid. 2. Add the solvent to the bead mixture and heat the reaction mixture to 70° C. for 1 hour in an inert atmosphere. 3. Remove the solvent. 4. Wash in water 2-3 times to remove excess solvent. The expected output is a cleaved NTAA and remaining peptide. If the NTAA is covalently linked to the capture moiety beforehand, the expected output is a monomer-capture moiety complex and a remaining peptide (e.g., as shown schematically in FIG. 1A Panel D).

### Example 5—Derivatization of Anilinothiazolinone (ATZ) Amino Acid to Phenylthiocarbamoyl (PTC) Form

As described herein, a monomer (e.g., amino acid) may be derivatized. For example, during cleavage of a terminal amino acid from a peptide, e.g., using TFA, as described above, the amino acid may be converted to an ATZ derivative. Further derivatization, e.g., to the PTC form may be performed. In one example, triethylamine may be used for the conversion.

Materials: Triethylamine solution (10%), DTT solution (0.01%), Water. Equipment: Shaker Incubator. Inert gas compartment.

Procedure: 1. Combine Triethylamine solution: DTT solution (1:3) (aq. solution) to the sample (e.g., substrate comprising the monomer-capture moiety complex and remaining peptide resulting from Example 4). 2. Heat the reaction mixture to 60° C. for 10 min in an inert atmosphere. 3. Remove the solvent. 4. Wash 2-3 times in water to remove excess triethylamine and DTT.

In another example, ammonium hydroxide may be used for the conversion.

Materials: Ammonium Hydroxide (3%) solution, DTT (0.1%) solution, Water. Equipment: Shaker Incubator, Inert gas compartment.

Procedure: 1. Combine Ammonium hydroxide: DTT (1:3) (aq. solution) to the sample. 2. Heat the reaction to 60° C. for 10 min in an inert atmosphere. 3. Remove the solvent. 4. Wash 2-3 times in water to remove excess ammonium hydroxide and DTT.

### Example 6—Coupling of Polymerizable Molecules

As described herein, a method for processing polymeric analytes, such as peptides, may comprise coupling a monomer to a capture moiety to generate a monomer-capture moiety complex, and contacting the monomer-capture moiety complex (e.g., subsequent to cleavage of the monomer from the polymeric analyte) with a binding agent comprising a first polymerizable molecule, and coupling the first polymerizable molecule to a second polymerizable molecule. The second polymerizable molecule may be a copy of the capture moiety (e.g., two identical nucleic acid molecules).

In an example, a substrate, e.g., a bead comprising a peptide and a plurality of capture moieties may be treated as outlined in Examples 1-5 to generate a substrate comprising a remaining peptide and a monomer-capture moiety complex. The monomer-capture moiety complex can be contacted with a binding agent (e.g., antibody) or library of binding agents comprising a binding agent that couples to the monomer (e.g., a PTC-form amino acid). The binding agent may comprise a polymerizable molecule (e.g., antibody oligonucleotide) that can be ligated to an additional capture moiety. The polymerizable molecule may comprise identifying information of the binding agent.

Materials: Antibody or Antibody pool with a unique DNA barcodes in PBST; May include other agents (poly-adenosine, Pluronic F127, etc.) to help with specificity; Phosphate Buffered Saline with 0.1% Tween-20 (PBST); 10×DNA ligase buffer: Final 1× composition is Tris (50 mM, pH 7.5) with ATP (1 mM), MgCl2 (10 mM), and DTT (10 mM); T4 DNA ligase enzyme (4000 U/uL) Important: DNA ligase is not heat stable and must be stored at −20 C for long term stability and 4 C for short term stability; 50% PEG-2000 in water; 0.1M NaOH. Equipment: Shaker.

Procedure: 1. Wash beads with PBST. 2. Add the Antibody or Antibody pool comprising antibody oligonucleotides comprising unique DNA barcodes in PBST. Incubate at room temperature with mixing for 1 hour. 3. Wash beads with PBST and wait 2-5 minutes. 4. Resuspend beads in ligase mix—for a 100 uL reaction combine in a tube on ice: 49.5 uL of water, 10 μL of 10×DNA ligase buffer, 40 μL of 50% PEG-2000, and 0.5 uL of T4 DNA ligase enzyme. 5. Incubate at room temperature for 30 minutes. Optional: Add 100 μL of 20 mM EDTA to stop the reaction. 6. Wash the beads with 0.1M NaOH to remove the Antibodies and complementary barcode strands. 7. Wash the beads with water or 1×TE to remove the NaOH. The expected output is a bead comprising a peptide, monomer-capture moiety complex, and coupled polymerizable molecules (an additional capture moiety coupled to an antibody oligonucleotide).

In another example, the polymerizable molecule (e.g., antibody oligonucleotide) of the binding agent may be coupled to an additional capture moiety, and a nucleic acid extension reaction may be performed, e.g., to transfer the information from the polymerizable molecule to the additional capture moiety.

Materials: Antibody or Antibody pool with a unique DNA barcodes in PBST; May include other agents (poly-adenosine, Pluronic F127, etc.) to help with specificity; Phosphate Buffered Saline with 0.1% Tween-20 (PBST); 10×DNA polymerase buffer: Final 1× composition is Tris (10 mM, pH 7.9) with NaCl (50 mM), MgCl2 (10 mM), and DTT (1 mM), 10 mM dNTP mix; Bsu DNA polymerase, Large Fragment enzyme (5 U/uL) Important: Bsu is not heat stable and must be stored at −20 C for long term stability and 4 C for short term stability; 50% PEG-2000 in water; 0.1M NaOH. Equipment: Shaker incubator.

Procedure: 1. Wash beads with PBST. 2. Add the Antibody or Antibody pool with unique DNA barcodes in PBST. 3. Incubate at room temperature with mixing for 1 hour. 4. Wash beads with PBST and wait 2-5 minutes. 5. Resuspend beads in polymerase mix—for a 100 uL reaction combine in a tube on ice: 46 uL of water; 10 uL of 10×DNA polymerase buffer; 40 uL of 50% PEG-2000; 2 uL of 10 mM dNTP mix; 2 uL of Bsu DNA polymerase, Large Fragment. 6. Incubate at 37° C. for 30 minutes. Optional: Add 100 uL of 20 mM EDTA to stop the reaction. 7. Wash the beads with 0.1M NaOH to remove the Antibodies and complementary barcode strands. 8. Wash the beads with 1×TE to remove the NaOH.

### Example 7—Cleavage of Monomers from Monomer-Capture Moiety Complexes

In some instances, it may be advantageous after detection (e.g., via binding of a binding agent and transferring of encoded information to a capture moiety or polymerizable molecule) to cleave or release the monomer from the monomer-capture moiety complex. For example, releasing the monomer subsequent to contacting with a binding agent may help prevent downstream nonspecific binding of additional binding agents, or double-counting of the same monomer, during subsequent iterations of monomer analysis.

In an example, a linker and a linking nucleic acid molecule may be used to couple to a terminal amino acid and to a capture moiety, as described herein. The linking nucleic acid molecule may comprise a cleavage site, e.g., a restriction enzyme site, a uracil for subsequent UDG cleavage, etc., such that the linking nucleic acid molecule may be cleavable to allow decoupling or removal of the monomer from the capture moiety.

Materials: 10× Cleavage buffer (1× composition: 20 mM Tris-acetate pH 7.9, 10 mM Magnesium acetate, 50 mM Potassium acetate, 100 ug/mL recombinant albumin); Splint DNA oligo (100 uM in water); 50% PEG-2000 in water; USER enzyme (1 U/uL); Optional: 10×3′ phosphate removal buffer (1× composition: 500 mM imidazole-HCl pH 6.4, 180 mM MgCl2, 50 mM DTT, 1 mM spermidine, and 1 mM ADP); Optional: T4 PNK (10 U/uL). Equipment: Shaker incubator.

Procedure: 1. Resuspend beads in 50 uL of annealing mix: 24 uL of water; 20 uL of 50% PEG-2000; 5 uL of 10× Cleavage buffer; and 1 uL of Splint DNA oligo (100 uM in water). 2. Incubate with shaking at 60° C. for 10 minutes. 3. Add 50 uL of enzyme mix for a total of 100 uL reaction: 24.5 uL of water; 20 uL of 50% PEG-2000; 5 uL of 10× Cleavage buffer; 0.5 uL of USER enzyme. 4. Incubate with shaking at 37° C. for 30 minutes. Optional: Add 100 uL of 20 mM EDTA to stop the reaction. 5. Wash the beads with 0.1M NaOH to remove any complementary DNA strands. 6. Wash the beads with 1×TE to remove the NaOH. Optional: Resuspend the beads in 3′phosphate removal mix: water, 10× phosphate removal buffer; T4 PNK. Incubate for 30 minutes at 37° C. 7. Wash the beads with 1×TE. The expected output is a bead comprising a peptide with one fewer amino acid, a capture moiety comprising a portion of the linking nucleic acid molecule, and an additional capture moiety comprising the unique barcode of the binding agent (e.g., as shown schematically in FIG. 1A Panel F).

In another example, the linking nucleic acid molecule comprises a restriction enzyme site to allow for cleavage of the monomer and a portion of the linking nucleic acid molecule.

Materials: 10× Cleavage buffer (1× composition: 20 mM Tris-acetate pH 7.9, 10 mM Magnesium acetate, 50 mM Potassium acetate, 100 ug/mL recombinant albumin); Splint DNA oligo (100 uM in water); 50% PEG-2000 in water; Earl restriction enzyme (20 U/uL). Equipment: Shaker incubator.

Procedure: 1. Resuspend beads in 50 uL of annealing mix: 24 uL of water; 20 uL of 50% PEG-2000; 5 uL of 10× Cleavage buffer; 1 uL of Splint DNA oligo (100 uM in water). 2. Incubate with shaking at 60° C. for 10 minutes. 3. Add 50 uL of enzyme mix for a total of 100 uL reaction: 23 uL of water; 20 uL of 50% PEG-2000; 5 uL of 10× Cleavage buffer; 2 uL of Earl restriction enzyme. 4. Incubate with shaking at 37° C. for 30 minutes. Optional: Add 100 uL of 20 mM EDTA to stop the reaction. 5. Wash the beads with 0.1M NaOH to remove any complementary DNA strands. Wash the beads with 1×TE to remove the NaOH.

Subsequent to cleavage of the monomer from the monomer-capture moiety complex, the entire process, e.g., as outlined in Examples 1-7, may be reiterated until all or a portion of the entire polymeric analyte is deconstructed and encoded for using polymerizable molecules.

### Example 8—Processing of Assembled Polymerizable Molecules Comprising DNA Barcodes for Next-Generation Sequencing

Following multiple iterations of processing a polymeric analyte (e.g., cleaving of monomers, contacting with binding agents, and transfer of information from binding agents to one or more capture moieties or polymerizable molecules of a substrate), the capture moieties or polymerizable molecules may be prepared for sequence identification, e.g., via next-generation DNA sequencing. For example, the substrate may comprise a capture moiety comprising a stacked polymerizable molecule that comprises individual polymerizable molecules transferred (e.g., via ligation or an extension reaction) from binding agents that bound to the monomer-capture moiety complexes. The stacked polymerizable molecules may be removed from the substrate, or they may be amplified, using primers complementary to a sequence of the capture moiety.

Materials: Reverse DNA primer (10 uM in water); 10×DNA polymerase buffer: Final 1× composition is Tris (10 mM, pH 7.9) with NaCl (50 mM), MgCl2 (10 mM), and DTT (1 mM); 10 mM dNTP mix; Bsu DNA polymerase, Large Fragment enzyme (5 U/uL) Important: Bsu is not heat stable and must be stored at −20 C for long term stability and 4 C for short term stability. Alternative 1: Taq DNA polymerase with 68 C extension. Alternative 2: DNA polymerase 1 or Klenow or Klenow(exo-) with room temperature extension in 50% PEG-2000 in water; 0.1M NaOH; 0.4M Tris-HCl (pH 8.0) with 0.1 mM EDTA. Equipment: Shaker incubator.

Procedure: 1. Resuspend beads in 50 uL of annealing mix: 20 uL of water; 20 uL of 50% PEG-2000; 5 uL of 10×DNA polymerase buffer; 5 uL of Reverse DNA primer (10 uM in water). 2. Incubate with shaking at 60° C. for 10 minutes. 3. Add 50 uL of enzyme mix for a total of 100 uL reaction: 21 uL of water; 20 uL of 50% PEG-2000; 5 uL of 10×DNA polymerase buffer; 2 uL of 10 mM dNTP mix; 2 uL of Bsu DNA polymerase, Large Fragment. 4. Incubate with shaking at 37° C. for 30 minutes. Optional: Add 100 uL of 20 mM EDTA to stop the reaction. 5. Wash the beads with 1×TE to remove any excess primer and polymerase. 6. Add 50 uL of 0.1M NaOH to remove complementary DNA barcode strands into solution. 7. Collect the 50 uL of 0.1M NaOH supernatant and combine into a tube containing 50 μL of 0.4M Tris-HCl (pH 8.0) with 0.1 mM EDTA. This supernatant will be used for downstream processes into NGS.

In another example, the stacked polymerizable molecules may be removed from the substrate. For example, the capture moieties or polymerizable molecules may comprise a restriction site or chemically releasable moiety.

Materials: 10× Cleavage buffer (1× composition: 20 mM Tris-acetate pH 7.9, 10 mM Magnesium acetate, 50 mM Potassium acetate, 100 ug/mL recombinant albumin); Splint DNA oligo 3 (100 uM in water); 50% PEG-2000 in water; Earl restriction enzyme (20 U/uL). Equipment: Shaker incubator.

Procedure: 1. Resuspend beads in 50 uL of annealing mix: 24 uL of water; 20 uL of 50% PEG-2000; 5 uL of 10× Cleavage buffer; 1 uL of Splint DNA oligo 3 (100 uM in water). 2. Incubate with shaking at 60° C. for 10 minutes. 3. Add 50 uL of enzyme mix for a total of 50 uL reaction: 23 uL of water; 20 uL of 50% PEG-2000; 5 uL of 10× Cleavage buffer; 2 uL of Earl restriction enzyme 4. Incubate with shaking at 37° C. for 30 minutes. 5. Heat-inactivate the enzyme at 65° C. for 20 minutes. Alternative: Add 50 uL of 20 mM EDTA to stop the reaction. 6. Collect the supernatant. This supernatant will be used for downstream processes into NGS.

### Example 9—NGS Read Reconstruction of Peptides

Next Generation Sequencing may be used to identify and read the polymerizable molecules to determine the identity and sequence of amino acids in a peptide. As described herein, the polymerizable molecules may comprise stacks of polymerizable molecules coupled to binding agents and captured to the polymerizable molecule (or capture moiety) of a substrate. Optionally, the polymerizable molecules may comprise cycle information (e.g., cycle number). The polymerizable molecules may be sequenced to generate sequencing reads, which can be assembled to determine the identity of all or a portion of a peptide or protein and the sequence in which the constituent amino acids occur.

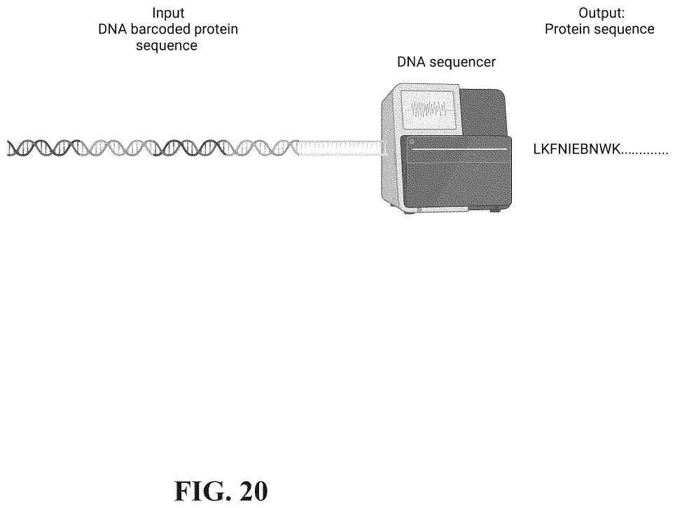

FIG. 20 schematically shows a diagram of inputting, into a sequencing instrument, barcoded DNA sequences (e.g., a stacked polymerizable molecule resulting from multiple iterations of workflow 100 of FIG. 1) and outputting a peptide or protein sequence. The barcoded DNA sequences comprise stacks of barcodes sequences obtained from individual binding agents that bind to their target monomer or monomer-capture moiety complex; each barcode sequence encodes for the identity of the monomer (e.g., amino acid).

In some examples, low quality reads are removed using a quality score filter. Reads are then grouped based on peptide barcode sequence; peptide barcode sequences are deemed identical if they are within a Hamming distance of two. Once grouped, the amino acid and cycle number are used to construct a putative sequence for each peptide. Undetected cycle numbers are recorded as gaps for unidentified amino acids in peptide sequences.

Alternatively, peptide sequences are reconstructed by matching NGS reads to simulated NGS patterns expected from a given proteomic database. For example, the human proteome database from UniprotKB can be used for predicted sequences. Digestion of proteins to peptides can be simulated to yield a library of peptides. For each peptide, a simulation may be carried out modeling amino acid capture (e.g., to a capture moiety), binding of a binding agent, barcode (from the polymerizable molecule of a binding agent) transfer, cleavage, and NGS readout assuming a range of efficiencies for each chemical and enzymatic operation. This process yields a range of peptide sequencing patterns for each peptide, which is then stored as a database.

Experimental NGS reads can be first pre-processed by removing low quality score reads, and then grouped into NGS reads from individual peptides via peptide barcode sequences. For each peptide barcode sequence, a sequencing pattern is generated denoting the identified amino acids as well as the respective cycle numbers. This sequencing pattern is compared to the simulated database of peptide sequencing patterns to find matches. Unambiguous matches result in the peptide identity being assigned directly. If a pattern matches multiple simulated peptides, a graph is generated assigning putative peptides to each pattern, which will be resolved during the protein inference/assembly stage.

### Example 10—Assembly of Putative Peptide Sequences to Proteins

Full length protein sequences along with isoforms and abundance may be obtained using a reference-based approach or by de novo assembly. In the reference-based approach, a putative peptide sequence may be compared to a proteomic database to infer the presence of target proteins in the sample, e.g., as is performed by the state-of-the-art in Mass Spectrometry proteomics. Alternatively, with a large enough data set, protein sequences can be assembled de novo by adapting assembly approaches used in transcriptomics.

Reference Based Assembly of Protein Sequences from Putative Peptide Sequences: Putative peptide sequences are filtered based on completeness and length. Sequences with more than 80% gaps are removed. In addition, sequences less than three amino acids are removed. Then, the filtered sequences used to probabilistically infer the presence of proteins by comparing them against the human proteome using Mass Spectrometry proteomics inference algorithms, such as ProteinProphet (Nesvizhskii, 2003).

De Novo Assembly of Protein Sequences from Putative Peptide Sequences: While de novo assembly of transcripts is well established in the field of transcriptomics (Martin et al, 2011), analogous methods for proteomics are lacking. Here, the de novo assembly methods Trinity (Haas et al, 2013) and Plass (Steinegger et al, 2019) are adapted to enable de novo assembly of proteins from putative peptide sequences. Briefly, peptide sequences are first broken up into overlapping k-mer sequences, where k is less than the sequence length of the peptide. Then, overlapping k-mer sequences are concatenated to form long contiguous reads (i.e. contigs). Unique contigs and contigs representing protein isoforms are represented as a De Bruijn graph where the nodes are the contigs and edges the connection between them. For each protein graph, the graph is traversed iteratively between all possible connected nodes to yield fully assembled sequences of protein isoforms. Based on the abundance of the putative peptide sequences, the relative abundance of each protein isoform can be assigned.

### Example 11—Model System of Monomer Coupling to a Capture Moiety Via Ligation and Monomer De-Coupling from the Capture Moiety

In some instances, the polymeric analyte of interest is a peptide comprising amino acid monomers. A substrate (e.g., bead) may be provided that comprises the polymeric analyte, and a plurality of identical capture moieties (e.g., nucleic acid molecules) that can be used to couple, e.g., via a linker, to a terminal amino acid, as described elsewhere herein. As a proof of concept of such a coupling approach, a model system may be used, in which a first DNA molecule (“chain oligo”) may be used to simulate a peptide-linker complex (e.g., a peptide that has been contacted with a bifunctional linker comprising a PITC moiety and linking nucleic acid molecule, as shown schematically in FIG. 1A Panel B), and a second DNA molecule (“anchor oligo”) may be used as a capture moiety. The chain oligo may be coupled to the anchor oligo via a splint oligonucleotide and ligation (e.g., using a ligase).

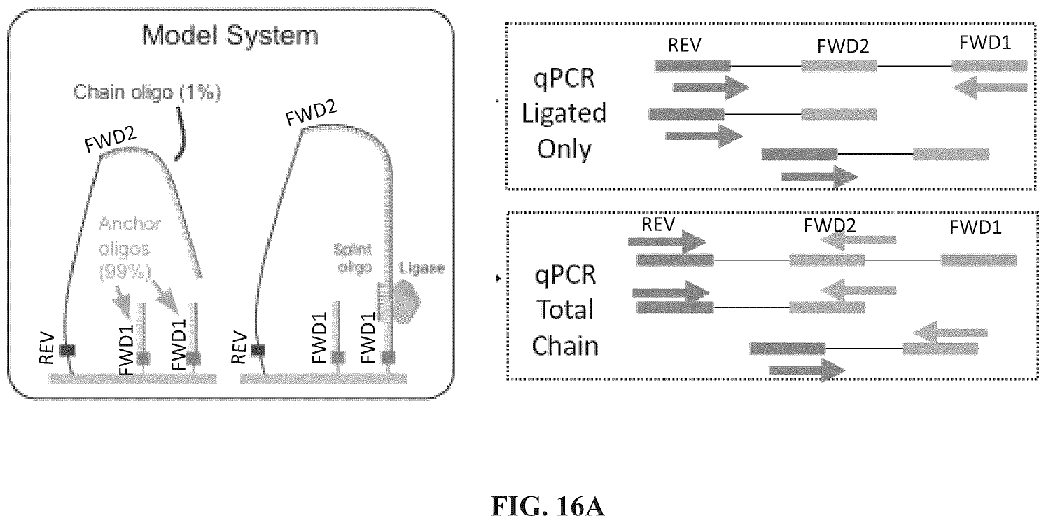

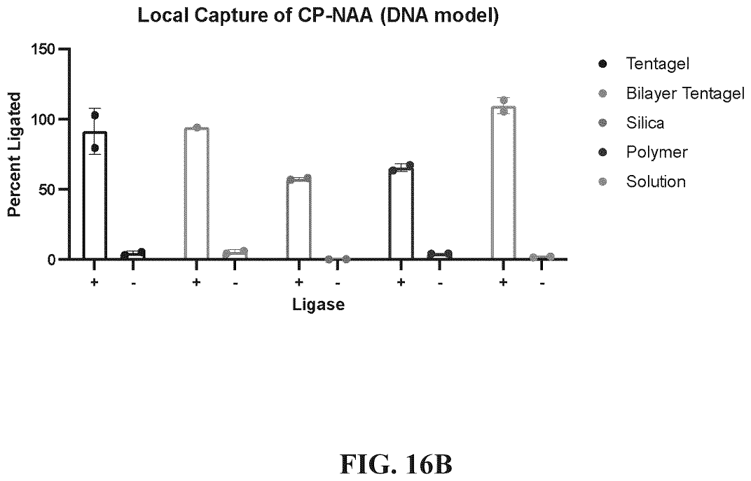

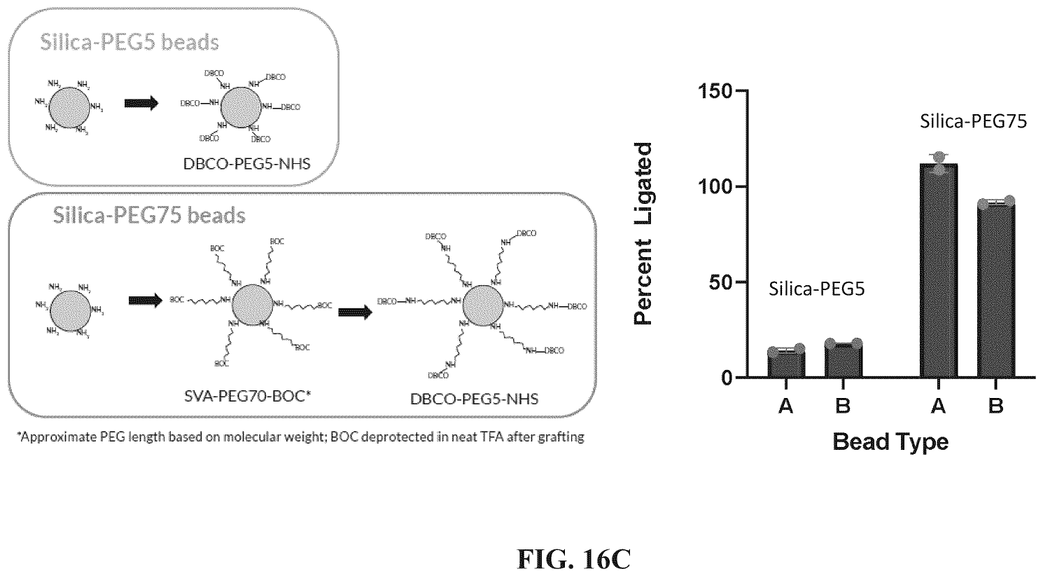

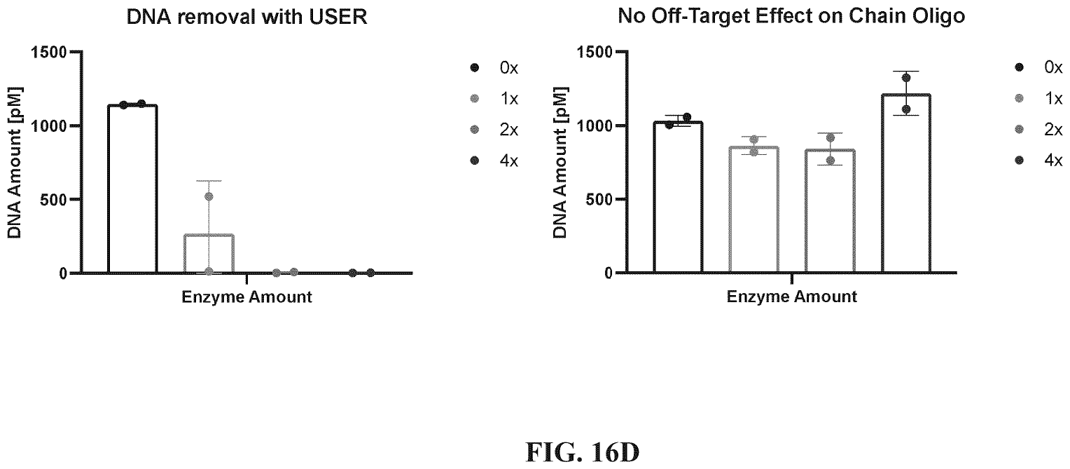

FIG. 16A schematically shows such an example model system. A bead substrate may be coupled to chain oligos and anchor oligos. As the chain oligo is a DNA model for a peptide analyte, the chain oligo is provided at a lower ratio of the anchor oligo (used to capture the chain oligo), in order to ensure that all chain oligos are coupled to an anchor oligo. For example, the chain oligo (/5Phos//ideoxyU/CTCCCTCTCTTTCTCTCTTTCCCTCTCTCTCCCTTTCTCCCTCCCTCTCCC/3AzideN/(SEQ ID NO: 1)) and the anchor oligo (/5AzideN/TTCTTCTTCTTCTCCCTCCTCTCTTCT (SEQ ID NO: 2)) are provided at a ratio of 1:99. The chain oligo comprises a forward primer sequence (“FWD2”) and a reverse primer sequence (“REV”), and the anchor oligo comprises an additional forward primer sequence (“FWD1”).

Four different types of substrates are tested: 1) TentaGel™ beads (Sigma-Aldrich) 2) Bilayer TentaGel™ beads (Sigma-Aldrich, modified to have only ˜10% surface active), 3) Silica magnetic beads (6-micron diameter DBCO beads from CD Bioparticles), 4) Polymer beads (1 micron diameter DBCO beads from Click Chemistry Tools). Beads are labeled with Cy5 for visualization. Beads are washed in 500 microliters of PBST for 15-30 mins at 37° C. three times, then 500 microliters of TE buffer for 15-30 mins at 37° C. The beads are then pelleted and resuspended in 22.5% glucose at 25 mg/mL. A ligation reaction is performed by adding 1:99 ratio of chain oligo to anchor oligo, and 10000 nM of splint oligo (5′-GGGAGAAGAAGA-3′ (SEQ ID NO: 3)) and annealing in an annealing solution comprising 10× Ligase Buffer, 50% Glucose, PEG, and water and incubating at 80° C. for 5 minutes on a Vortemp, then incubating for 30 minutes after turning off the temperature at 900 RPM. A ligation buffer comprising 10× ligase buffer, 75% PEG, glucose, t4 DNA ligase, and water is added and incubated at room temperature for 30 minutes. The ligation reaction is then stopped by heat-killing the ligase by incubating at 65° C. for 10 minutes on a thermal cycler.

In order to quantify the efficiency of the ligation, quantitative PCR (qPCR) may be used to assess (1) the number of ligated molecules, which can be assessed by the number of DNA molecules that can be amplified using the FWD1 and REV primers, and (2) the number of chain oligos, which can be assessed by the number of DNA molecules that can be amplified using FWD2 and REV primers.

FIG. 16B shows example data representing the ligation of the chain oligo to the anchor oligo for all four bead types, as compared to a positive control (solution-only, no substrate). The bar graph represents the percentage of ligated molecules in the presence of ligase (“+”) as compared to a negative control of no ligase (“−”). All of the negative control examples where no ligase was added resulted in no ligation. All of the beads with ligase added had varying degrees of successful ligation of the anchor oligo to the chain oligo. Altogether, these results indicate that, using a chain oligo to represent a peptide-linker complex, the chain oligo can be successfully coupled and ligated to a capture moiety (anchor oligo) at an efficiency of greater than 50%. These results help demonstrate feasibility of coupling a peptide-linker complex to a capture moiety (anchor oligo).

Further improvements to ligation efficiency may be improved by increasing the spacing of the chain or anchor oligos relative to the surface of a bead. For example, FIG. 16C shows schematically (left panel) that the silica magnetic beads (6-micron diameter DBCO beads from CD Bioparticles) can be modified with a shorter PEG chain (“PEG5”) or a longer PEG linker to generate Silica-PEG75 beads (“DBCO-PEG5-NHS”), e.g., via a BOC protection group, deprotection in TFA, to which the chain and anchor oligos can be conjugated. The ligations may be performed as described above (“A”) or with an additional passivation step (“B”). The right panel of FIG. 16C shows a bar graph of the qPCR data representing the ligation of the chain oligo to the anchor oligo for the Silica magnetic beads (“Silica-PEG5”) and the modified, longer-PEG silica magnetic beads (“Silica-PEG75”). As can be observed from the bar graph, the addition of the PEG linker increases or improves the ligation efficiency of the chain oligo to the anchor oligo, regardless of a passivation event.

The methods described herein may also comprise removing or decoupling of the monomer from the capture moiety, e.g., subsequent to contacting the monomer-capture moiety complex with a binding agent. The decoupling may be achieved by cleavage of the monomer-capture moiety complex, e.g., at a cleavage site such as a restriction site using a restriction enzyme, or at a uracil site using uracil DNA glycosylase. The cleavage site may be located on the capture moiety (e.g., anchor oligo) or on the peptide-linker complex (e.g., as modeled by a chain oligo).

To demonstrate cleavage of the monomer-capture moiety complex, the chain oligo is designed to include a uracil (e.g., at a 5′ end, as described above) or elsewhere in the chain oligo (not shown). The chain oligo is ligated to the anchor oligo, as described above, using a splint oligo. Subsequent to ligation, the monomer-capture moiety complex (chain-anchor oligo complex) is treated with USER® (New England Biolabs) enzyme to cleave the chain oligo, and qPCR is used to quantify the number of monomer-capture moiety complexes (chain-anchor oligo complexes) and the number of chain oligos remaining. In a successful cleavage event, it is expected to have few or no monomer-capture moiety complexes (chain-anchor oligo complexes). Similarly, the chain oligo itself is expected to remain relatively unaffected (i.e., no off-target cleavage events).

FIG. 16D shows data from such a cleavage experiment, in which a uracil is present at the 5′ end of the chain oligo (or between the FWD1 and FWD2 sequences when the chain oligo is ligated to the anchor oligo). In the left-hand panel, the number of chain-anchor oligo complexes is measured using qPCR as a function of varying enzyme amount added. The chain-anchor oligo complex is amplified by adding in FWD1 and REV primers and measuring the amount of amplicons obtained. As can be observed, the no-enzyme (“0x”) negative control results in no cleavage and high levels of chain-anchor oligo complexes (“DNA Amount”). As the enzyme amount is increased (1×, 2×, 4×), the number of chain-anchor oligo complexes decreases until no chain-anchor oligos are detected, which indicates a complete cleavage event (>98% cleavage for 2× and 4× conditions) of the chain-anchor oligo complexes. On the right-hand panel, the amount of chain oligo is measured by qPCR using FWD2 and REV primers for varying enzyme amounts. As can be seen from the bar graph, the amount of measured chain oligo does not change significantly across the experimental conditions, indicating that the USER enzyme does not have off-target effects and does not cleave the chain oligo in other locations other than where the uracil is present.

Altogether, FIGS. 16A-16D demonstrate a model system in which a chain oligo is used to represent a peptide-linker complex and that (i) the chain oligo can successfully couple and be ligated to a capture moiety (represented by an anchor oligo) and, (ii) upon application of USER, the chain oligo can be specifically cleaved.

### Example 12—Edman Degradation Alternatives and Compatibility with Nucleic Acid Molecules

As described herein, Edman degradation approaches may be used to cleave monomers (e.g., an amino acid) from a polymeric analyte (e.g., peptide). Standard Edman degradation approaches may comprise the use of elevated temperatures and strong acids, e.g., trifluoroacetic acid (TFA). Accordingly, the polymerizable molecules described herein may be designed to be stable under standard Edman degradation, or alternatively, alternative degradation strategies may be employed.

In the first approach, standard Edman degradation approaches may be used, and in order to ensure polymerizable molecules, e.g., nucleic acid molecules, are more resistant to Edman degradation procedures, the nucleic acid molecules are generated using pyrimidine bases (e.g., thymine and cytosine) rather than purines. In one particular example, all nucleic acid molecules provided on a substrate (e.g., capture moieties and polymerizable molecules) may be constructed of solely pyrimidines comprising predominantly thymines (“T”) and cytosines (“C”), also referred to herein as TC-encoded nucleic acid molecules. In one such example, a substrate (e.g., bead) may be provided that comprises the polymeric analyte, and a plurality of identical capture moieties (e.g., nucleic acid molecules) that comprise only Ts and Cs. The capture moieties can be used to couple, e.g., via a linker, to a terminal amino acid, as described elsewhere herein.

To determine whether or not the TC-encoded nucleic acid molecules would be stable under standard Edman degradation, a model system is used, as described in Example 11 and as shown in FIG. 16A, in which a first DNA molecule (“chain oligo”) may be used to simulate a peptide-linker complex (e.g., a peptide that has been contacted with a bifunctional linker comprising a PITC moiety and linking nucleic acid molecule, as shown schematically in FIG. 1A Panel B), and a second DNA molecule (“anchor oligo”) may be used as a capture moiety. The chain oligo may be coupled to the anchor oligo via a splint oligonucleotide and ligation (e.g., using a ligase). The substrate may then be exposed to TFA (Edman's reagent) or to TE Buffer (negative control). Subsequent cleavage of the chain-anchor oligo complex may be performed, using USER enzyme, as described in Example 11. An example nucleic acid sequence of TC-encoded chain oligos and anchor oligos is also provided in Example 11.

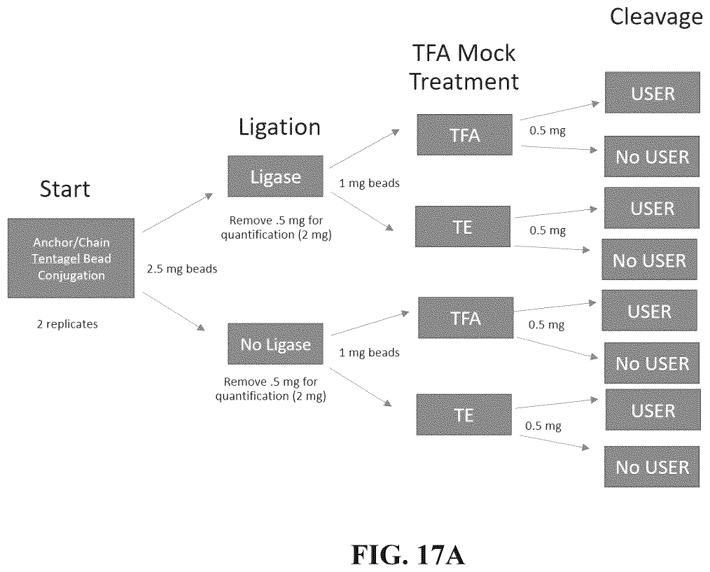

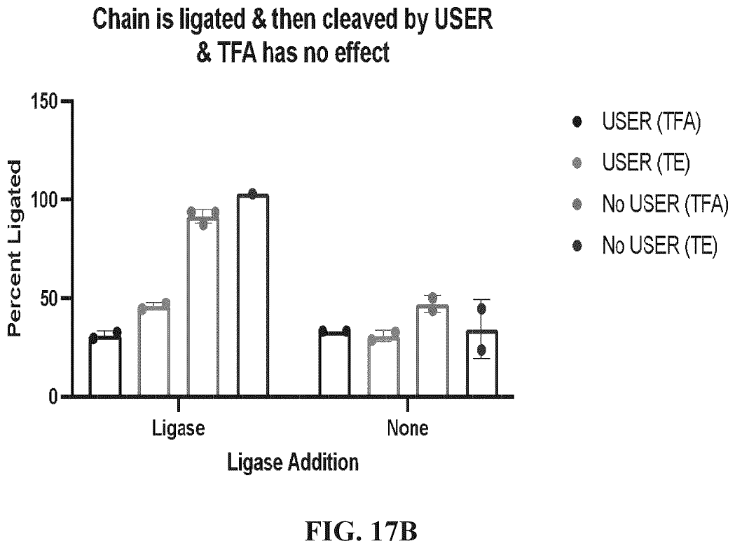

FIG. 17A shows an example experimental workflow for determining (i) the stability of TC-encoded chain and anchor oligos under Edman degradation (TFA treatment) or TE Buffer (negative control) and (ii) whether Edman degradation adversely affects downstream enzymatic cleavage to release the chain oligo from the anchor oligo. The substrate, e.g., TentaGel™ beads that have been functionalized with a 1:99 ratio of chain to anchor oligos, as described above, is either subjected to ligation or no ligation. The two populations of beads are then subjected to treatment with either Edman's reagent (TFA) or a negative control (TE Buffer). Subsequently, the four populations of beads are treated with USER or no USER (negative control). The chain-anchor complex is measured for all eight populations of beads by using qPCR on the primer handles of the chain oligo and anchor oligo (using FWD1 and REV primers).

FIG. 17B shows the result of the experiment outlined in FIG. 17A. The bar graph shows the percentage of ligated molecules (i.e., chain-anchor complexes, as measured by qPCR using the FWD1 and REV primers) as a function of treatment type. As can be observed, all of the negative control (no ligase) conditions (four right-hand bars) result in some background signal of ˜35% of chain-anchor complexes. The ligated conditions (left-hand bars) indicate that ˜35% of chain-anchor complexes are detectable when the USER is added, which is consistent with the background signal. The ligated conditions without USER result in an appreciably higher signal for the chain-anchor complexes. Overall, these results suggest that ligated oligos (chain-anchor complexes) can still be cleaved by USER regardless of Edman degradation reaction conditions, and that the Edman degradation reaction does not adversely affect downstream ligation operations, as the chain-anchor complexes are still detectable when ligase is present and when no cleaving enzyme is added.

Alternative strategies to Edman degradation for monomer cleavage may also be used. In one example, milder acidic conditions as compared to classic Edman degradation reagents (trifluoroacetic acid) may be used to remove a PITC-conjugated terminal amino acid; such conditions may comprise, for example, use of a milder acid such as dichloroacetic acid (DCA), e.g., using 2% DCA in DCM at room temperature for 30 minutes. In some instances, a Lewis acid may be used to perform the cleavage, e.g., boron triflate (BF₃), boron trifluoride etherate, boron trichloride, boron tribromide, boron triiodide, scandium triflate, or other Lewis acids. In another example, acetonitrile in triethylamine in acetic acid (1:1:1 ratio) at 70° C. for 1 hour may be used as a reaction condition.

To test the viability of these approaches in removing a linker-conjugated terminal amino acid, as described herein, a set of 17 tripeptides of Formula X-G-F is synthesized, where X represents a variable amino acid (all 20 proteinogenic amino acids excluding Cys, Gln, Lys), G represents Glycine, and F represents phenylalanine. The tripeptides are reacted with a linker, 1-(2-azidoethyl)-4-isothiocyanatobenzene, which comprises a PITC moiety and a click chemistry moiety (azide), to generate linker-tripeptide complexes. The linker-tripeptide complexes are purified using HPLC.

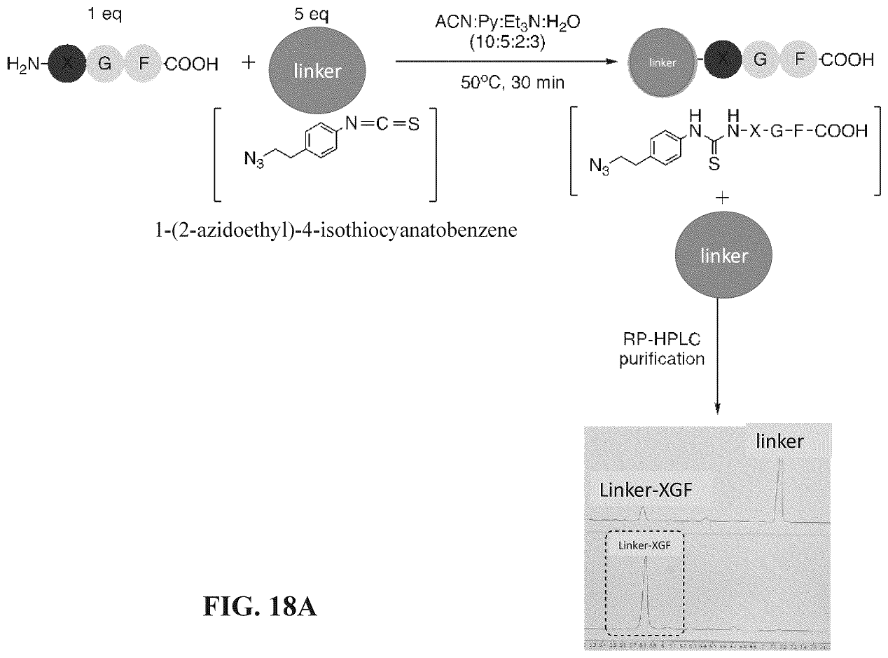

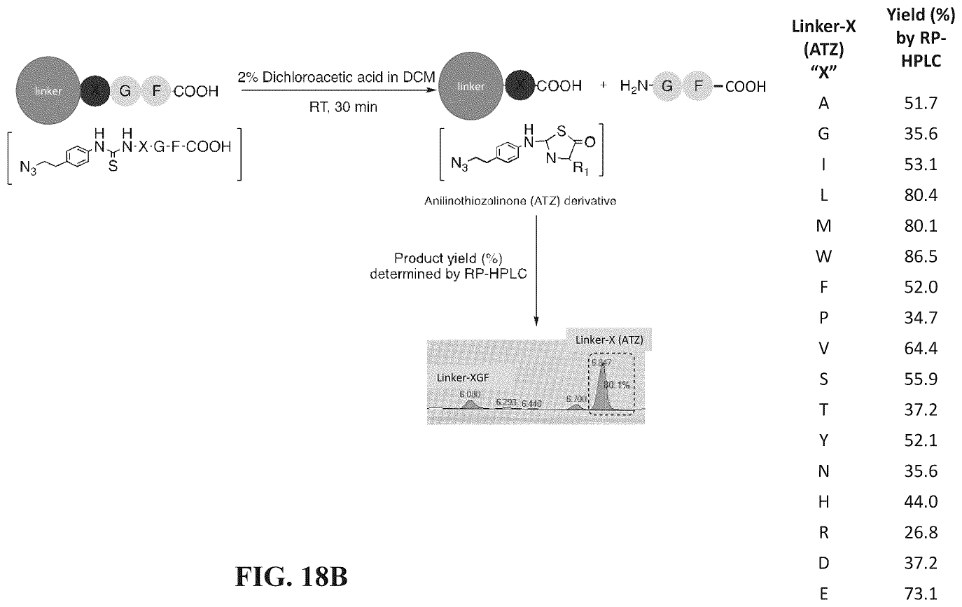

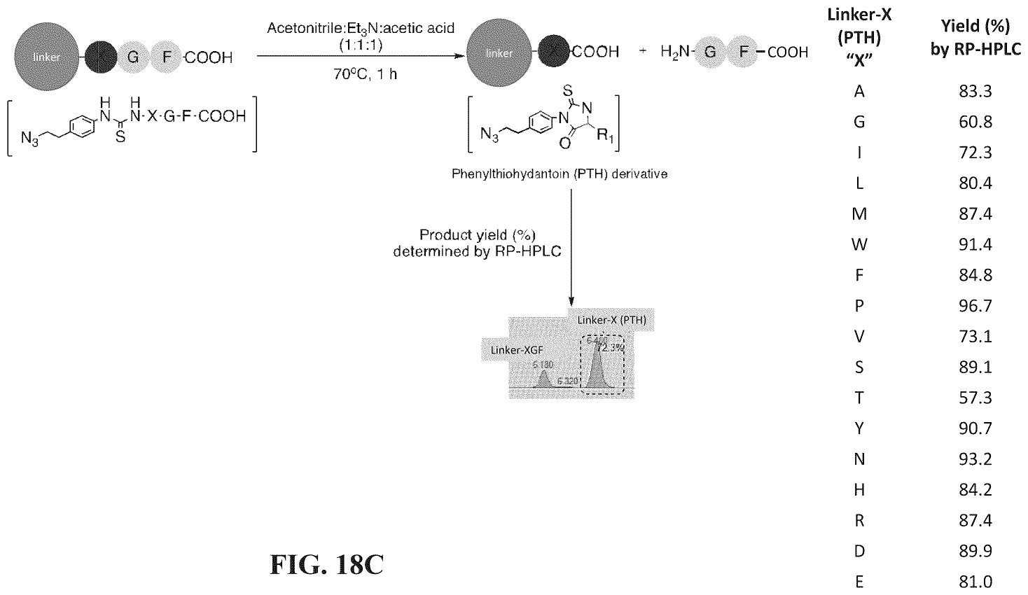

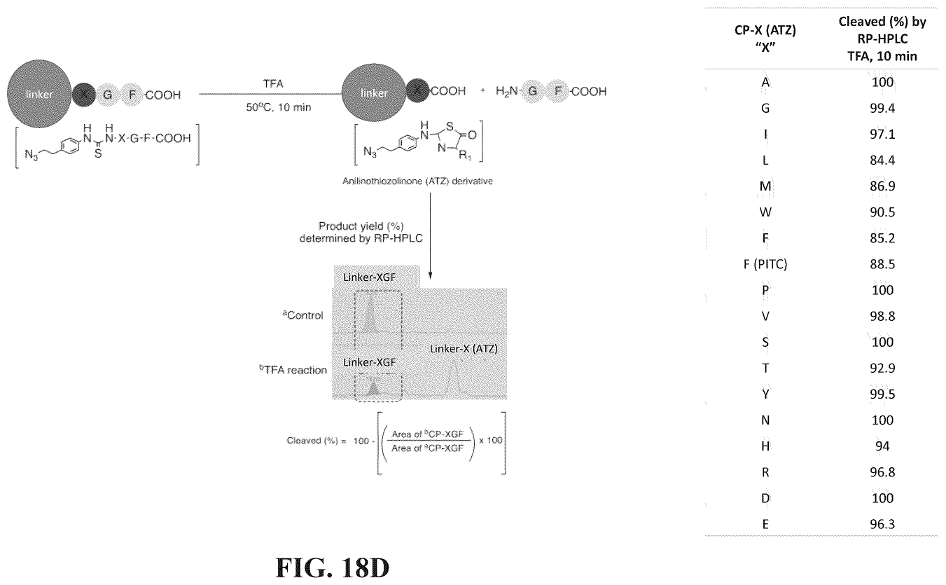

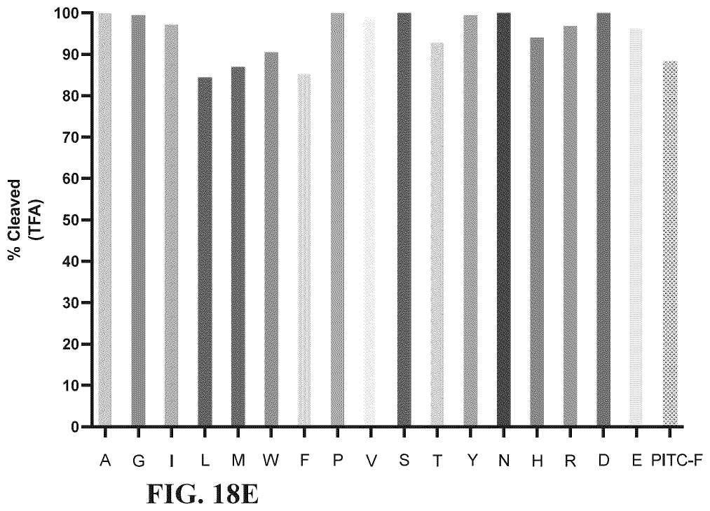

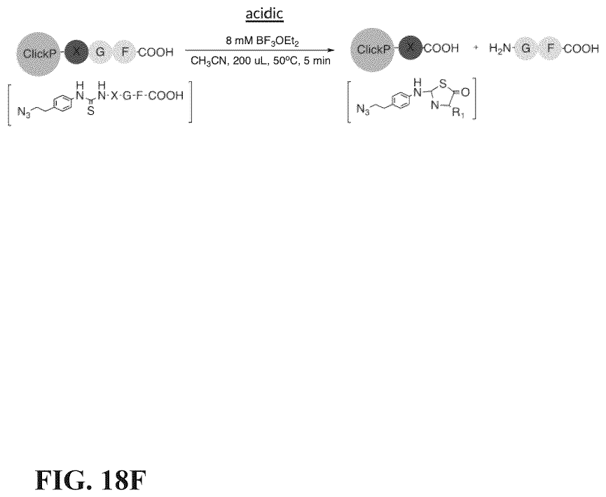

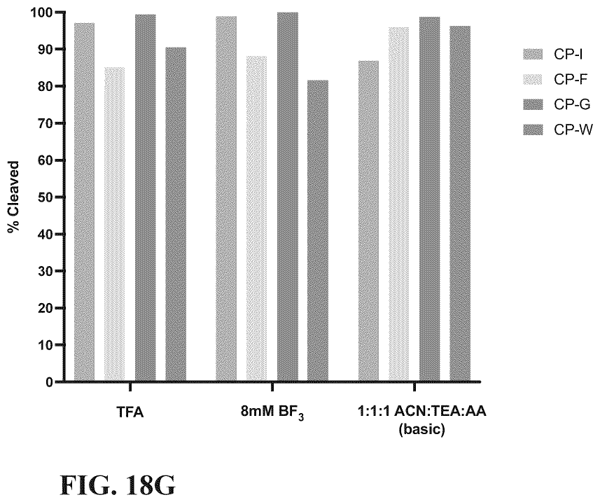

FIG. 18A shows an example synthesis approach to generate the linker-tripeptide complexes. Each tripeptide is reacted with 5 parts of linker (1-(2-azidoethyl)-4-isothiocyanatobenzene) in acetonitrile, pyrimidine, triethylamine, and water, at a ratio of 10:5:2:3 at 50° C. for 30 minutes to generate a linker-tripeptide complex. Reverse phase-HPLC is used to purify, obtain, and quantify the linker-tripeptide complexes.

Next, each of the linker-tripeptide complexes are subjected to cleavage using an alternative Edman degradation strategy. FIG. 18B shows an alternative approach in which, instead of TFA treatment, the linker-tripeptide complexes are treated with 2% DCA in DCM at room temperature for 30 minutes. The DCA treatment results in cleavage and ATZ derivatization of the X amino acid for some of the linker-tripeptide complexes (“Linker-X (ATZ)”). A subset of the linker-tripeptide complexes (“Linker-XGF”) remain unreacted under the given reaction conditions. The efficiency of the cleavage reaction can be calculated by taking the area-under-the-curve (AUC) of the derivatized, cleaved X amino acid (“Linker-X (ATZ)”), divided by the sum of the AUC of the uncleaved linker-tripeptide complex (“Linker-XGF”) and the AUC of the derivatized, cleaved X amino acid (“Linker-X (ATZ)”). The table on the right-panel shows the efficiency (“yield”) of cleavage for 17 different X amino acids.

FIG. 18C shows an alternative approach in which, instead of TFA treatment, the linker-tripeptide complexes are treated with acetonitrile in triethylamine and acetic acid (1:1:1 ratio) at 70° C. for 1 hour. The acetonitrile treatment results in cleavage and PTH derivatization of the X amino acid for some of the linker-tripeptide complexes. A subset of the linker-tripeptide complexes (“Linker-XGF”) remain unreacted under the given reaction conditions. The efficiency of the cleavage reaction can be calculated by taking the area-under-the-curve (AUC) of the derivatized, cleaved X amino acid (“Linker-X (PTH)”), divided by the sum of the AUC of the uncleaved linker-tripeptide complex (“Linker-XGF”) and the AUC of the derivatized, cleaved X amino acid (“Linker-X (PTH)”). The table on the right-panel shows the efficiency (“yield”) of cleavage for 17 different X amino acids.

FIG. 18D shows an alternative quantitation approach to calculate the efficiency of cleavage using a traditional Edman degradation agent, trifluoroacetic acid (TFA). The linker-tripeptide complexes are treated with TFA at 50° C. for 10 minutes. The TFA treatment results in cleavage and ATZ derivatization of the X amino acid for some of the linker-tripeptide complexes. The efficiency of the cleavage reaction can be calculated by subtracting out the unreacted fraction; e.g., by first taking the area-under-the-curve (AUC) of the unreacted portion of the samples treated with TFA (“Linker-XGF”) over the AUC of the unreacted portion of the untreated samples and subtracting that from 1, then multiplying by 100 to obtain the percentage of reacted or cleaved molecules. The table on the right-panel shows the efficiency (“yield”) of cleavage for 17 different X amino acids, one of which (Phe, F) was also tested using PITC as the linker. FIG. 18E visually represents the same data in the table.

FIG. 18F shows an alternative approach in which, instead of TFA treatment, the linker-tripeptide complexes are treated with boron trifluoride (BF₃) etherate (8 mM) in acetonitrile at 50° C. for 5 minutes. The BF₃ treatment results in cleavage and ATZ derivatization of the X amino acid for some of the linker-tripeptide complexes. The efficiency of the cleavage reaction is calculated by subtracting out the unreacted fraction, as described in FIG. 18D; e.g., by first taking the area-under-the-curve (AUC) of the unreacted portion of the samples treated with TFA over the AUC of the unreacted portion of the untreated samples and subtracting that from 1, then multiplying by 100 to obtain the percentage of reacted or cleaved molecules. The results of the cleavage using TFA (standard Edman), BF₃, and acetonitrile in triethylamine and acetic acid with different “X” amino acid residues (isoleucine (I), phenylalanine (F), glycine (G), and tryptophan (W)) are compared in FIG. 18G.

Overall, all the linker-tripeptide complexes show higher cleavage efficiencies under the acetonitrile cleavage conditions as compared to the DCA cleavage conditions. The non-polar amino acids Gly, Val, and Ile demonstrate the lowest cleavage efficiency, whereas Ala, Leu, Met, Trp, Phe, and Pro show higher cleavage efficiencies. Among the polar amino acids, Thr has the lowest cleavage efficiency, whereas Ser, Tyr, and Asn show much higher efficiencies. Among the charged amino acids, all of them demonstrate high cleavage efficiency. Using a different quantitation metric to calculate the cleavage efficiency, e.g., by subtracting out the uncleaved fractions, cleavage efficiency for all tripeptides using TFA, BF₃, and acetonitrile/triethylamine/acetic acid are high. Overall, these results demonstrate the feasibility of using alternative cleavage strategies to standard Edman degradation to improve compatibility with the nucleic acid encoding approaches described herein.

The acetonitrile cleaving condition is also tested for compatibility with nucleic acid molecules, for use in the methods described herein. Silica-PEG5 beads are conjugated with either TC-encoded amplicons (nucleic acid molecules comprising only C and T), or TCAG-encoded amplicons (nucleic acid molecules comprising all four bases) using click chemistry (DBCO beads reacted with azide-conjugated nucleic acid molecules). The TC-encoded amplicon (Azide-TTCTTCTTCTTCTCCCTCCTCCCTCCCTCTTTCTTTCTCTCTCTCTCTCTCTCTCTCTCCCTT TCTCCCTCCCTCTCCC (SEQ ID NO: 4)) and the TCAG-encoded amplicon (Azide-TTCTTCTTCTTCTCCCTCCTCCCTCCCTCTTTCTTTCGCACTGTATCGCACTGTATCTCCCT TTCTCCCTCCCTCTCCC (SEQ ID NO: 5)) both comprise a forward primer sequence (TTCTTCTTCTTCTCCCTCCT (SEQ ID NO: 6)) and a reverse primer sequence (CCTTTCTCCCTCCCTCTCCC (SEQ ID NO: 7)) at the ends. The beads are then treated with either acetonitrile in triethylamine and acetic acid (1:1:1 ratio), or in TE buffer (negative control) at 70° C. for 1 hour. The beads are then subjected to qPCR using the forward and reverse primers to determine the quantity of DNA present on the beads.

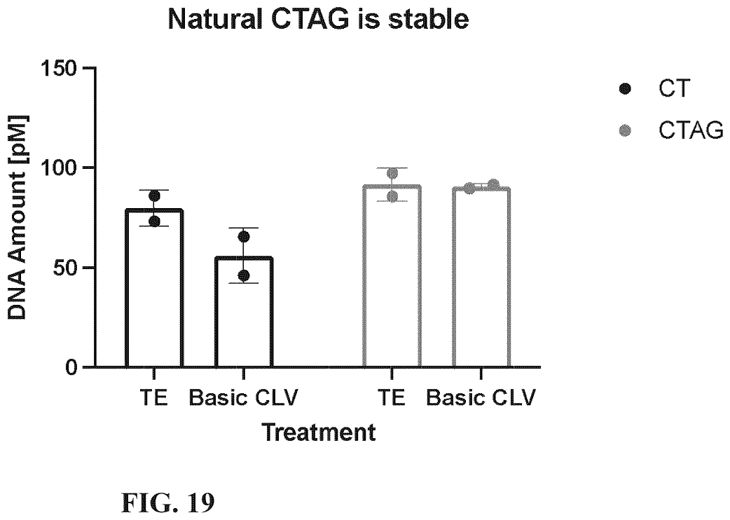

FIG. 19 shows data from the experiment. The left-hand bars represent the TC-encoded beads, treated with either TE negative control (“TE”) or in the acetonitrile mixture (“Basic CLV”), and the right-hand bars represent the TCAG-encoded beads, treated with either TE negative control (“TE”) or in the acetonitrile mixture (“Basic CLV”). As can be observed from the plot, no significant degradation or destruction of the nucleic acid molecules is observed in the acetonitrile cleavage condition as compared to the negative control, indicating the viability of this approach as an alternative to standard Edman degradation for cleaving terminal amino acids.

### Example 13—Identification of a N-Terminal Amino Acid of a Peptide Via Local Tethering

The methods described herein enable processing and analysis of polymeric analytes such as peptides. In some instances example, a peptide is coupled to a substrate comprising a plurality of capture moieties (e.g., nucleic acid molecules). The capture moieties of the substrate can be used to couple a terminal amino acid to the substrate (local tethering), e.g., via a linker and linking nucleic acid molecule, as shown in FIG. 1. Subsequent cleavage of the terminal amino acid from the peptide may be performed, followed by detection using a binding agent and a detectable label (e.g., fluorophore or a nucleic acid barcode molecule comprising identifying information of the binding agent).

As proof of concept of the local tethering, cleavage of the monomer, e.g., terminal amino acid, and detection using a binding agent, a model system is used. The model system comprises a substrate (e.g., bead such as a magnetic bead) that comprises a plurality of DNA capture moieties (also referred to herein as “anchor oligos”). The bead is attached to a model peptide via one of the DNA capture moieties. The model peptide comprises a C-terminal oligonucleotide that can couple to one of the capture moieties, e.g., via hybridization with the capture moiety or using a splint oligonucleotide. The model peptide is additionally conjugated at the N-terminus to a linking nucleic acid molecule (also referred to herein as “chain oligo”) using a linker, e.g., (1-(2-azidoethyl)-4-isothiocyanatobenzene) that is reacted with a 3′ DBCO-conjugated linking nucleic acid molecule, thereby generating a bead-peptide-linking nucleic acid molecule complex.

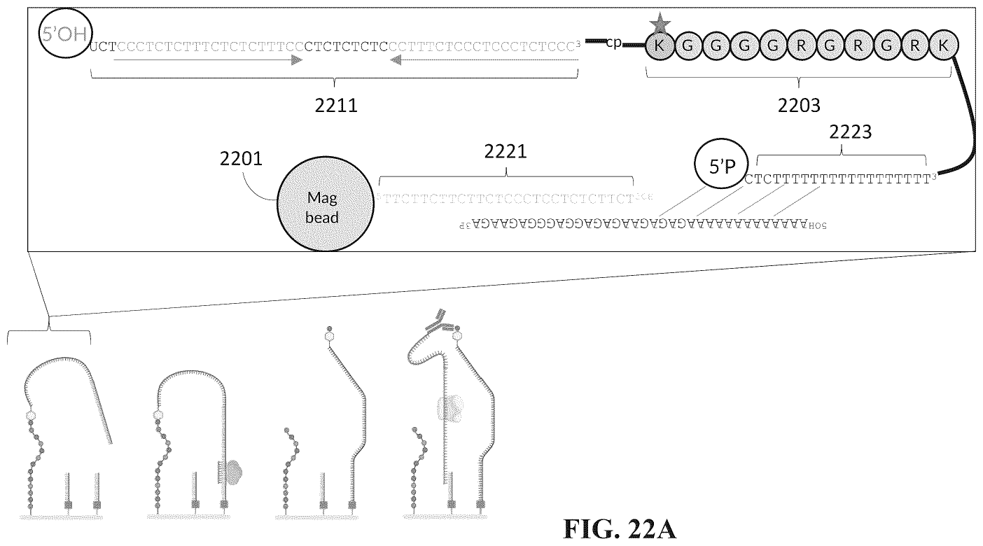

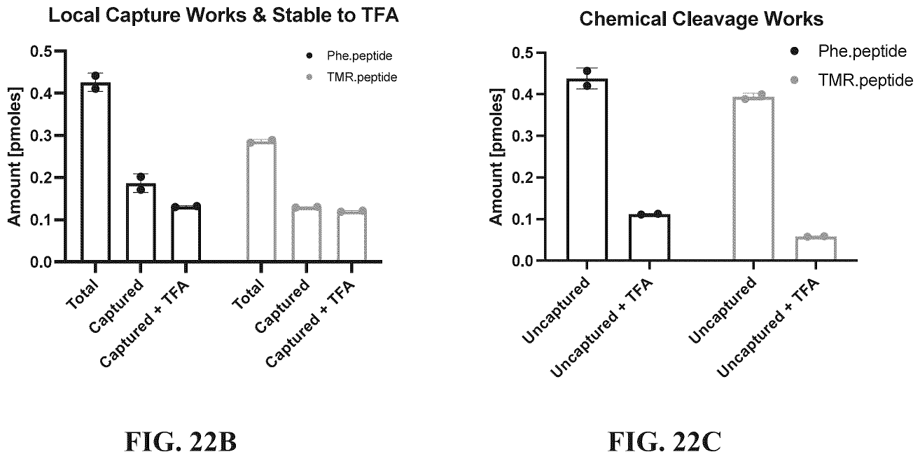

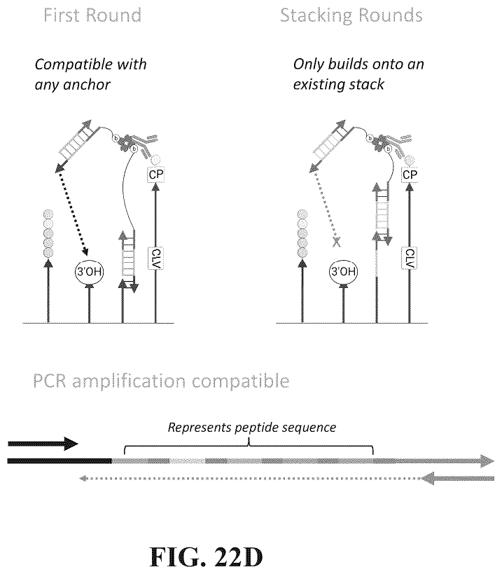

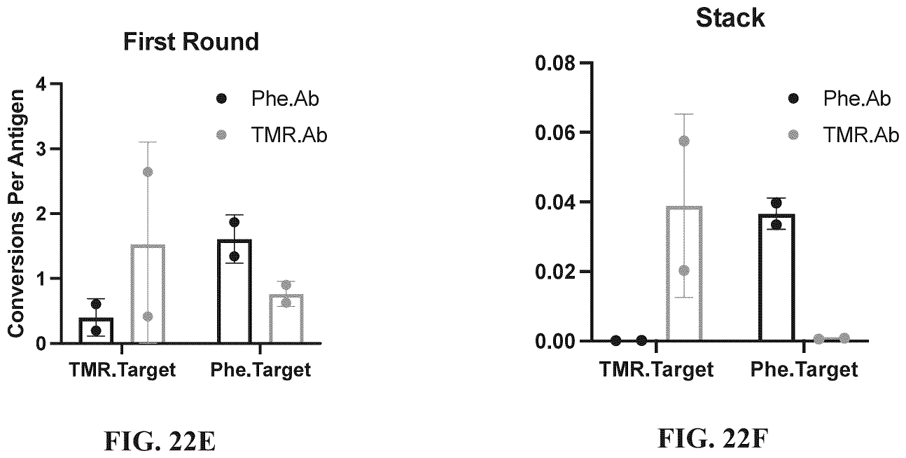

FIG. 22A schematically shows an example of such a bead-peptide-linking nucleic acid molecule complex, in relation to the workflow schematized in FIG. 1. A magnetic bead 2201 is provided as the substrate, onto which a plurality of nucleic acid capture moieties 2221 are conjugated. A model peptide 2203 comprising a C-terminal oligonucleotide 2223 is coupled to the bead via a splint molecule, which partially hybridizes to one of the nucleic acid capture moieties 2221 and to the C-terminal oligonucleotide 2223. The model peptide 2203 also comprises, at the N-terminus, a linking nucleic acid molecule 2211. The N-terminal linking nucleic acid molecule 2211 is attached to the peptide using click chemistry of a linking nucleic acid precursor comprising a 3′DBCO moiety to the azide group of a bifunctional linker (1-(2-azidoethyl)-4-isothiocyanatobenzene). The bifunctional linker also couples to the N-terminal amino acid of the model peptide 2203 via the phenylisothiocyanate (isothiocyanatobenzene) moiety. The complex may then be subject to conditions sufficient to couple the linking nucleic acid molecule 2211 to another nucleic acid capture moiety 2221 (local tethering), cleavage of the N-terminal amino acid, and detection of the cleaved N-terminal amino acid via a binding agent.

Model Peptides: Two different bead-peptide-linking nucleic acid molecule complexes are used, each type comprising a different peptide. The first type of complex comprises a first model peptide that comprises an N-terminal phenylalanine (Phe), and the second type of complex comprises a second model peptide that comprises an N-terminal fluorophore, tetramethylrhodamine (TMR), which is conjugated via an N-terminal lysine residue. Each of the model peptides comprise a C-terminal oligonucleotide (e.g., as shown in 2223, sequence /5Phos/CTCTTTTTTTTTTTTTTTTT (SEQ ID NO: 8)) and an N-terminal linking nucleic acid molecule (e.g., 2211, sequence (/5deoxyU/CTCCCTCTCTTTCTCTCTTTCCCTCTCTCTCCCTTTCTCCCTCCCTCTCCC (SEQ ID NO: 9))) that is conjugated to the N-terminus using a click chemistry reaction of a DBCO-containing linking nucleic acid molecule precursor ((/5deoxyU/CTCCCTCTCTTTCTCTCTTTCCCTCTCTCTCCCTTTCTCCCTCCCTCTCCC/3DBC ON/ (SEQ ID NO: 10)) and azide group of the bifunctional linker, 1-(2-azidoethyl)-4-isothiocyanatobenzene).

Bead preparation: The bead-peptide-linking nucleic acid molecule complexes are prepared by first preparing the beads comprising the DNA capture moieties. The beads comprise magnetic silica particles (SMP-UM16 from CD Bioparticles) that contain amine functional groups. The beads are PEGylated using tBOC-NH-PEG-SVA-3400 (Laysan Bio) followed by deprotection of the tBOC with TFA, then functionalized with DBCO using DBCO-PEG5-NHS (BroadPharm). These DBCO-functionalized beads are then attached to azide-functionalized DNA capture molecules (/5AzideN/TTCTTCTTCTTCTCCCTCCTCTCTTCT (SEQ ID NO: 2)) using click chemistry to generate DNA-functionalized beads comprising DNA capture molecules. Unreacted DBCO sites are passivated by using mPEG5-azide (Broadpharm).

The DNA-functionalized (and passivated) beads are processed to couple, via splinted hybridization and ligation, the model peptides (the first model peptide and the second model peptide) via the C-terminal oligonucleotides 2223, thereby generating the bead-peptide-linking nucleic acid molecule complexes. For the first model peptide (comprising an N-terminal Phe), a mixture is made comprising 15 picomoles of first model peptide provided in a mixture containing 15 microliters of water, and mixed with 30 microliters of 1 micromolar splint oligonucleotide (sequence 5′ AAAAAAAAAAAAAGAGAGAAGAGAGGAGGGAGAAGA (SEQ ID NO: 11)), 8 microliters of 10×T4 DNA ligase buffer (New England Biolabs), and 19 microliters of water. For the second model peptide (comprising an N-terminal TMR), 15 picomoles of the second model peptide provided in a mixture containing 15 microliters of water is combined with 30 microliters of 1 micromolar splint oligonucleotide, 8 microliters of 10×T4 DNA ligase buffer, and 19 microliters of water. The splint oligonucleotide is allowed to anneal with the model peptides at 50 degrees Celsius with a slow ramp cool at 0.1 Celsius/second down to 16 degrees Celsius for 5 minutes. The DNA-functionalized beads are aliquoted into two tubes, pelleted, the supernatant removed, and then resuspended with either the first model peptide mixture or the second peptide mixture. 2 microliters of T4 DNA ligase and 2 microliters of T4 PNK are added to each tube, and the tubes are incubated while rotating at room temperature for 30 minutes. The tubes are then washed with 500 microliters of 0.1M NaOH with 0.1% Tween-20 twice, then washed with PBST twice, then with 0.1% Tween-20. In some examples, an additional T4 PNK incubation may be performed. The resultant bead-peptide-linking nucleic acid molecule complexes may be stored for future use or used immediately.

To test the local tethering efficiency of the linking nucleic acid molecule (e.g., as shown in 2211) to an additional nucleic acid capture moiety (DNA capture molecule) of the bead, an additional splint oligonucleotide (5′ GGGAGAAGAAGA (SEQ ID NO: 3)) is provided that has sequences complementary to a portion of the linking nucleic acid molecule 2211 and the additional capture nucleic acid molecule 2221. The beads are then tested for ligation efficiency by splitting each tube into two groups, a negative control (+splint oligo but no ligase) and a ligase+splint test condition. The beads are resuspended in a mixture containing the splint oligo, ligase buffer, PEG, and water, and for the ligase test condition, T4 DNA ligase and T4 PNK. The beads are then slowly annealed on VorTemp for 5 minutes at 80 degrees Celsius, then the temperature is turned off for 30 minutes while shaking. The tubes are then spun, and cooled on ice for 1 minute. The ligase (in the test condition) is then added with the T4 PNK, or water (negative control condition) is added. The tubes are then incubated at room temperature while rotating for 30 minutes. The tubes are then subjected to heat killing of the ligase by incubating at 65 degrees Celsius for 10 minutes. The tubes are then washed with 100 microliters of 0.1M NaOH with 0.1% Tween-20 twice, then washed with PBST twice, then with 0.1% Tween-20 twice.

The stability of the oligos under Edman degradation conditions can also be tested subsequent to local tethering. For such test conditions, the beads are twice pelleted and resuspended in 100 microliters of acetonitrile, then resuspended in 50 microliters of neat trifluoroacetic acid or 50 microliters of TE buffer+0.10% Tween-20 as a negative control. The tubes are vortex and incubated at 50 degrees Celsius for 10 minutes. The beads are then washed in 0.1% Tween-20, then 1M sodium bicarbonate+0.1% Tween, and then twice in 0.1% Tween-20. The tubes are then additionally washed with 0.1M NaOH with 0.1% Tween-20 twice, then washed with PBST twice, then with 0.10% Tween-20.

Table 1 summarizes the test conditions.

| Condition | Peptide | Ligase | TFA | Replicate no. |
|---|---|---|---|---|
| 1 | TMR | Yes | Yes | 1 |
| 2 | TMR | Yes | Yes | 2 |
| 3 | TMR | No | Yes | 1 |
| 4 | TMR | No | Yes | 2 |
| 5 | TMR | Yes | No | 1 |
| 6 | TMR | Yes | No | 2 |
| 7 | TMR | No | No | 1 |
| 8 | TMR | No | No | 2 |
| 9 | Phe | Yes | Yes | 1 |
| 10 | Phe | Yes | Yes | 2 |
| 11 | Phe | No | Yes | 1 |
| 12 | Phe | No | Yes | 2 |
| 13 | Phe | Yes | No | 1 |
| 14 | Phe | Yes | No | 2 |
| 15 | Phe | No | No | 1 |
| 16 | Phe | No | No | 2 |

TMR = tetramethylrhodamine.

To assess the efficiency of the local tethering (successful ligation) and the stability of the oligos under Edman degradation, qPCR is performed to assess (1) the number of ligated molecules, which can be assessed by the number of DNA molecules that can be amplified using the FWD1 and REV primers described above, which amplicons comprise at least a portion of the DNA capture molecule and the linking nucleic acid (chain) molecule, and (2) the number of linking nucleic acid (chain oligos), which can be assessed by the number of DNA molecules that can be amplified using FWD2 and REV primers, as described above.

FIG. 22B shows example qPCR data of the above-outlined experiment. The y-axis shows the amount of DNA quantified for the varying conditions of 1) the total number of linking nucleic acid (chain) molecules (“Total”); 2) the number of ligated oligos (“Captured”), indicating successful local tethering and ligation of the linking nucleic acid (chain) molecule to the DNA capture (anchor) molecules with the addition of a splint and ligase, and 3) the number of ligated oligos following treatment with an Edman degradation reagent (“Captured and TFA”). The left-hand columns indicate the first model peptide (N-terminal Phe) and the right-hand columns indicate the second model peptide (TMR-labeled peptide). For both model peptides, the ligation or local tethering efficiency is estimated at ˜45%, as calculated by the ratio of the Captured (ligated) oligos over the total number of linking nucleic acid (chain) molecules. The capture is also stable to Edman degradation conditions (approximately 70-90% of locally tethered oligos remain after TFA treatment).

FIG. 22C shows example qPCR data of the above-outlined experiment for the non-ligated (non-tethered) negative control conditions, where no ligase was added. The y-axis shows the amount of DNA quantified for the varying conditions of 1) the number of linking nucleic acid (chain) molecules (“Uncaptured”), and 2) the number of linking nucleic acid (chain) molecules after cleavage via treatment with TFA. A decrease in the amount of linking nucleic acid (chain) oligonucleotides is observed with the TFA treatment, indicating that cleavage does successfully remove ˜80% of the N-terminal linking oligonucleotides (via N-terminal amino acid cleavage).

Subsequent to cleavage of the N-terminal amino acid from the peptide, the cleaved amino acid-capture molecule complex can be detected using a binding agent that specifically or semi-specifically binds to the amino acid residue type over other types of amino acid residues. In some embodiments, the binding agent comprises an antibody or antibody fragment (e.g., scFv).

FIG. 22D schematically shows multiple rounds of detection of cleaved terminal amino acids using binding agents. The peptide is coupled with or conjugated to a bifunctional linker (“CP”, e.g., 1-1-(2-azidoethyl)-4-isothiocyanatobenzene) that comprises a PITC group that couples to the terminal amino acid. The bifunctional linker is also conjugated to a linking nucleic acid molecule via azide-DBCO click chemistry, which then is attached to the substrate via splinted ligation with a capture moiety (e.g., DNA molecule) on the substrate (local tethering). The terminal amino acid is then cleaved from the peptide and detected using a binding agent. The schematic illustrates the peptide, which is adjacent to the cleaved terminal amino acid, the bifunctional linker, and the linking nucleic acid molecule that is tethered or ligated to a capture moiety on the substrate. A binding agent (depicted as an antibody) that comprises a nucleic acid barcode molecule that identifies the binding agent is provided. In the first round of peptide sequencing, the cleaved N-terminal amino acid is tethered to the substrate, and the binding agent couples to the cleaved N-terminal amino acid. The nucleic acid barcode molecule of the binding agent can couple to any of the additional capture moieties on the substrate. The nucleic acid barcode molecule of the binding agent may be transferred or copied onto a capture moiety, e.g., via ligation or extension, to generate a capture moiety-barcode complex. In subsequent rounds, the n-1, n-2, n-3, etc. terminal amino acid is removed, contacted with one or more binding agents or a library of binding agents that are specific or semi-specific to the amino acid target, and the nucleic acid barcode molecule of the binding agent (“binding agent barcode”) that binds to the cleaved N-terminal amino acid is transferred or copied (e.g., ligated or extended) to either an additional capture moiety of the substrate or to the capture moiety-barcode complex. In the latter scenario, the binding agent barcode of that round may be configured to only bind to the capture moiety-barcode complex. For example, the nucleic acid barcode molecule of the second cycle (to analyze the n-1 terminal amino acid) may comprise a sequence that can only couple to a nucleic acid molecule having the first cycle barcode sequence. Similarly, the nucleic acid barcode molecule of the third cycle (to analyze the n-2 terminal amino acid) may comprise a sequence that can only couple to a nucleic acid molecule having the second cycle barcode sequence. Subsequent to multiple cycles or rounds, the capture moiety-barcode complex may comprise multiple barcode sequences from multiple binding agents that are provided in multiple rounds. In some instances, the capture moiety comprises a first primer sequence, and the nucleic acid barcode molecule provided in the last round or cycle comprises a second primer sequence. Accordingly, after multiple rounds of sequencing, the first primer sequence and the second primer sequence can be used to extend or amplify the capture moiety-barcode complex to determine the sequence of the amino acids in the peptide. In other examples (not shown), the individual nucleic acid barcode molecules from each round or cycle may be included on the same, separate, or both the same and separate capture moieties. In some examples, the individual nucleic acid barcode molecules may comprise cycle barcode sequences, indicating the round or cycle in which the nucleic acid barcode molecules are provided and which can be used to deconvolve the sequence (order) or position in which an identified amino acid occurs in the peptide.

Referring again to the experiments outlined in FIGS. 22A-22C, to test the viability of the detection methods outlined herein, binding agents (e.g., antibodies) that are specific to the antigen (e.g., linker-amino acid complex) and that comprise a binding agent barcode are used to determine 1) the transfer or copy efficiency of the binding agent barcode to a capture moiety to generate a capture moiety-barcode complex and 2) the stacking efficiency of a second binding agent barcode to the capture moiety-barcode complex. The outputs of the experiment outlined in FIG. 22C, which is depicted schematically in FIG. 1A Panel D, comprise beads that comprise (i) either the first model peptide or the second model peptide, as described above; both peptide types have the N-terminal amino acids cleaved, and (ii) a capture moiety-linking nucleic acid-linker-cleaved amino acid complex.

To test the efficiency of generation of a first-round capture moiety-barcode complex, two antibody binding agents are provided: (1) a TMR-binding antibody and (2) a Phe-binding antibody at a 50 nM concentration. The Phe-binding antibody selectively recognizes a linker-Phe complex, and not Phe alone. The antibodies are conjugated to one or more streptavidin molecules (on average, between 1-5 streptavidin molecules per antibody, conjugated using BroadPharm) to which biotin-conjugated binding agent nucleic acid barcodes (also referred to herein as “binding agent barcodes”) are attached. The biotin-binding agent barcodes are provided at a 2× ratio (100 nM) to the antibody for the first cycle and 16× (800 nM) for the second cycle, thus yielding four populations of antibodies: 1) a TMR-binding antibody with 2× binding agent barcodes, 2) a linker-Phe binding antibody with 2× binding agent barcodes, 3) a TMR-binding antibody with 16× binding agent barcodes, and 4) a linker-Phe binding antibody with 16× binding agent barcodes.

All bead types are aliquoted as shown in Table 2, for the first cycle.

TABLE 2
Bead populations for testing binding and efficiency of generating
a first-round capture moiety-barcode complex.

| TMR Antibody 2x | Phe Antibody 2x |
|---|---|
| TMR-pep | TMR-pep |
| TMR-pep | TMR-pep |
| Phe-pep | Phe-pep |
| Phe-pep | Phe-pep |

The beads for each sample are pelleted, supernatant removed, and washed with TE Buffer. The supernatant is then removed and the appropriate antibody population is added (as shown in Table 2) and allowed to incubate on a rotator for ˜2 hours. The binding agent barcodes are ligated to a capture moiety by the addition of ligase in T4 ligase buffer for 30 minutes on a rotator, then washed with 20 mM EDTA, pelleted and resuspended in 0.1M NaOH, then PBST, then TE buffer.

For the second cycle, to assess the stacking efficiency of a second cycle barcode to the first cycle barcode, the 16×-conjugated antibodies are used. The beads are first pelleted, supernatant removed, and then washed in TE buffer. The 16×-conjugated antibodies (populations 3 and 4 above) are then added and incubated for one hour, and a ligation reaction is performed by addition of ligase in T4 ligase buffer for 30 minutes on a rotator, washed with 20 mM EDTA, pelleted and resuspended in 0.1M NaOH, then PBST, then TE buffer.

Next, qPCR is performed to assess the 1) the transfer or copy efficiency of the binding agent barcode to a capture moiety to generate a capture moiety-barcode complex (i.e., nucleic acid molecules comprising the capture molecule sequence and the first cycle binding agent barcode) and 2) the stacking efficiency of a second binding agent barcode to the capture moiety-barcode complex (i.e., nucleic acid molecules comprising the capture molecule sequence, the first cycle barcode, and the second cycle barcode).

FIG. 22E shows qPCR data of the first cycle transfer or copy efficiency of the binding agent barcode to a capture moiety for each of the model peptide types. The y-axis (“conversions per antigen”) represents the normalized value of the number of ligated molecules (comprising the first binding agent barcode and the capture molecule sequences) for four conditions (from left to right): 1) the TMR beads contacted with a Phe antibody; 2) the TMR beads contacted with a TMR antibody; 3) the Phe beads contacted with a Phe antibody; and 4) the Phe beads contacted with a TMR antibody. From the data, it can be observed that the Phe antibody specifically recognizes Phe over TMR, and similarly, the TMR more specifically binds to TMR over Phe. Moreover, the first-round cycle barcode of the binding agents is present on the capture moieties, as detected by qPCR at an estimated 50-70% efficiency, indicating the feasibility of attaching an antibody barcode to the capture moiety of the bead via ligation. The antibodies are removed using 0.1M sodium hydroxide for 5 minutes, prior to repeating the experiment for the second round.

FIG. 22F shows qPCR data of the second cycle transfer of the binding agent barcode to the first cycle barcode. The y-axis (“conversions per antigen”) represents the normalized value of the number of ligated molecules (comprising the capture molecule sequence, the first cycle barcode, and the second cycle barcode) for four conditions (from left to right): 1) the TMR beads contacted with a Phe antibody; 2) the TMR beads contacted with a TMR antibody; 3) the Phe beads contacted with a Phe antibody; and 4) the Phe beads contacted with a TMR antibody. From the data, it can be observed that again, the Phe antibody specifically recognizes Phe over TMR, and similarly, the TMR more specifically binds to TMR over Phe. Moreover, the stacking is very specific, albeit with reduced efficiency as compared to the first cycle barcode ligating to the capture molecule. Altogether, these results indicate the feasibility of attaching a first antibody barcode to a capture moiety of a bead, and further stacking a second antibody barcode onto the first antibody barcode.

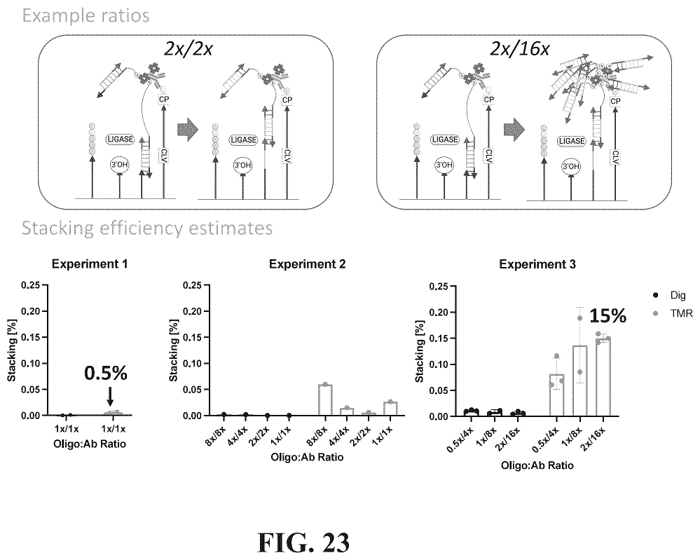

FIG. 23 shows example data regarding the stacking efficiency, which can be improved by conjugating additional barcode oligos onto the antibodies. The y-axis indicates the stacking efficiency (percentage of capture molecules that comprise a first cycle barcode and a second cycle barcode) for varying ratios of binding agent barcodes conjugated to the antibodies. The first number in the ratio represents the average number of binding agent barcodes conjugated to an antibody for the first cycle, and the second number in the ratio represents the average number of binding agent barcodes conjugated to an antibody for the second cycle. For example 1×/1× refers to 1 binding agent barcodes per antibody in the first cycle and 1 binding agent barcode per antibody in the second cycle; 2×/16× represents 2 binding agent barcodes per antibody in the first cycle and 16 binding agent barcodes per antibody in the second cycle. Two model antigens are shown: a digoxin-3′-conjugated chain oligo, and a TMR-conjugated chain oligo; for both antigens, the 3′-conjugated antigen is attached to the capture molecule without a connected peptide backbone, representing antigens that have been cleaved from the rest of the peptide. As exhibited in the plots, the highest stacking efficiency is observed at the 2×/16× condition. Further improvements may be possible for higher concentrations of binding agent barcodes conjugated to antibodies.

### Example 14—Self-Splinting Oligonucleotide for Local Tethering of an N-Terminal Amino Acid

FIG. 24 schematically shows an example of local tethering of an N-terminal amino acid using a linking nucleic acid molecule comprising a self-splinting region. A peptide may be contacted with a linker (“CP”) that comprises an amino acid coupling group and a linking nucleic acid molecule. The linking nucleic acid molecule may comprise a partially double-stranded region (e.g., hairpin) and a sequence that can hybridize to at least a portion of a capture moiety. Subsequent ligation may be used to covalently link the linking nucleic acid molecule to the capture moiety. Subsequent cleavage of the N-terminal amino acid may be performed, along with detection, as described elsewhere herein. The use of a self-splinting linking nucleic acid molecule may reduce inefficiencies associated with splinted ligation.

### Example 15—Coupling of First Polymerizable Molecules to Activating Agents Via Hybridization Chain Reaction and Coupling of First Polymerizable Molecules to Substrates

As described herein, detection of a monomeric analyte may comprise coupling a binding agent that binds specifically or semi-specifically to the monomeric analyte, providing a first polymerizable molecule (e.g., nucleic acid barcode molecule) comprising encoded information (e.g., the identity of the binding agent or the monomeric analyte), and coupling that first polymerizable molecule to a second polymerizable molecule (e.g., a substrate-bound DNA capture moiety).

In one example, the first polymerizable molecule comprises a nucleic acid barcode molecule that encodes for the identity of a binding agent (e.g., antibody or antibody fragment) that is specific for a particular amino acid type, or for the cognate molecule of the binding agent (e.g., the particular amino acid type). The nucleic acid barcode molecule may be generated using hybridization chain reaction (HCR), using an activating agent (e.g., activating nucleic acid molecule), as depicted in FIG. 1C. The activating agent may initiate hybridization of a first hairpin molecule, which then provides a template for hybridization of a second hairpin molecule which comprises a barcode sequence. The second hairpin molecule may also comprise a flap sequence that, subsequent to hybridization to the first hairpin molecule, comprises a single-stranded overhang which can be used to couple to a substrate-bound capture moiety, e.g., a DNA capture molecule.

To test the feasibility of HCR in coupling to an activating nucleic acid molecule in solution, two sets of oligonucleotides are used, a first set to demonstrate feasibility of HCR, e.g., as disclosed in Ang and Yung. Chemical Communications. Issue 22, 2016, which is incorporated by reference herein in its entirety, and a second set to demonstrate feasibility of HCR with a flap sequence that can subsequently anneal to a substrate-bound capture moiety.

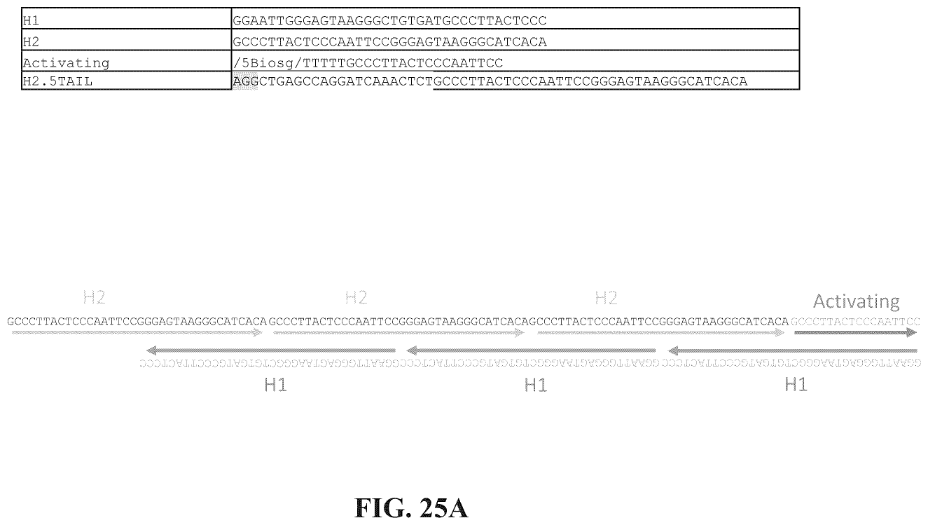

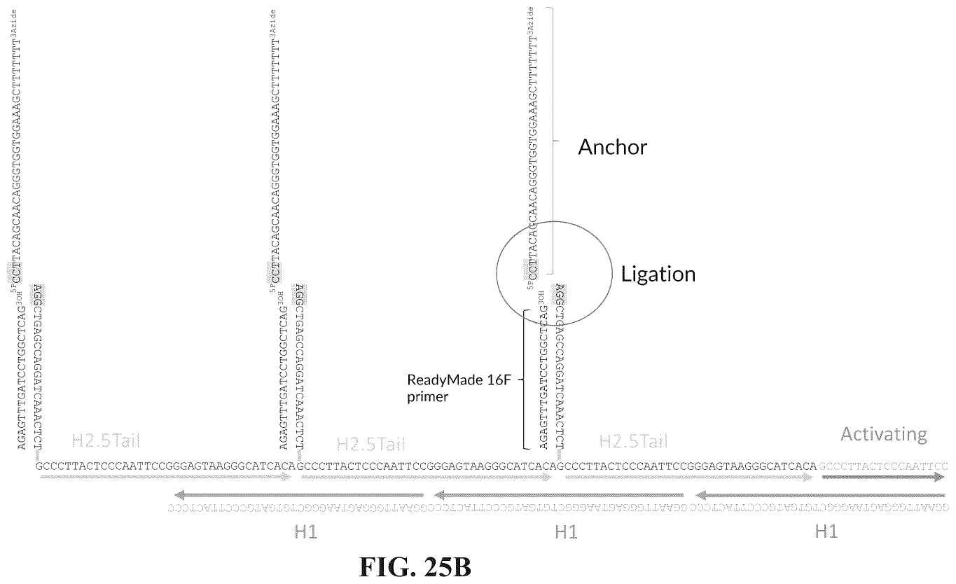

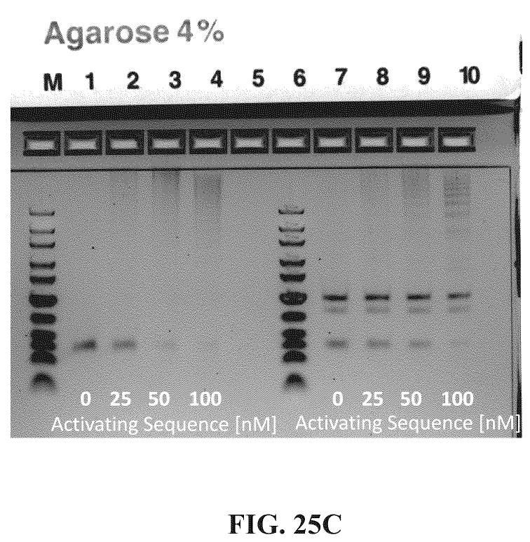

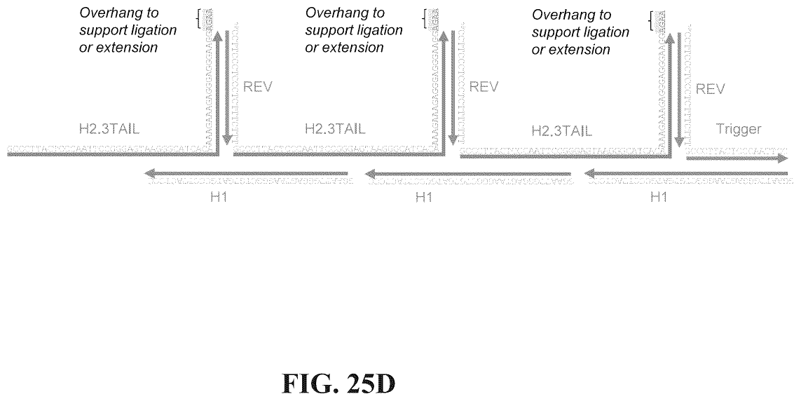

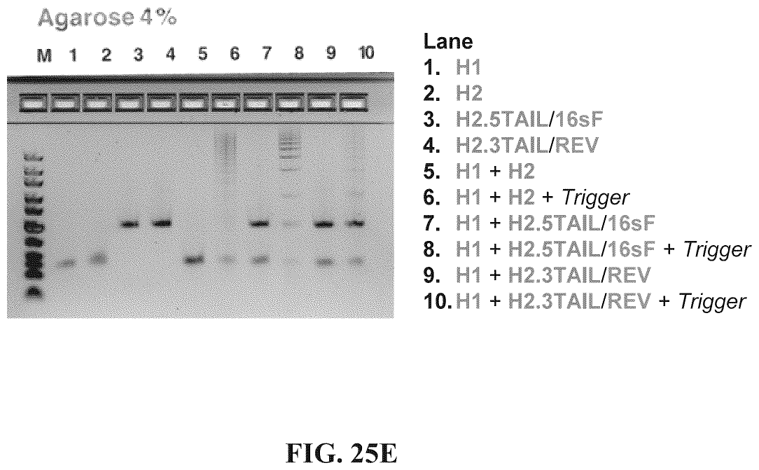

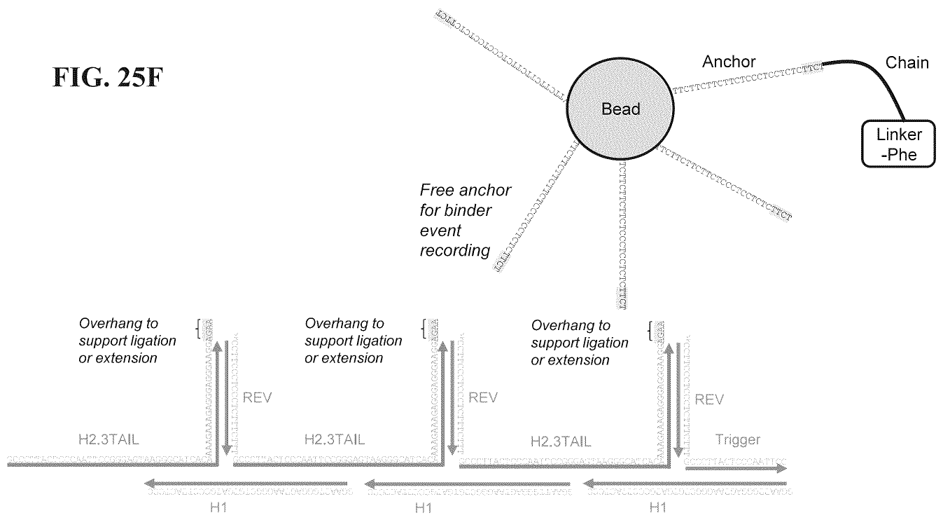

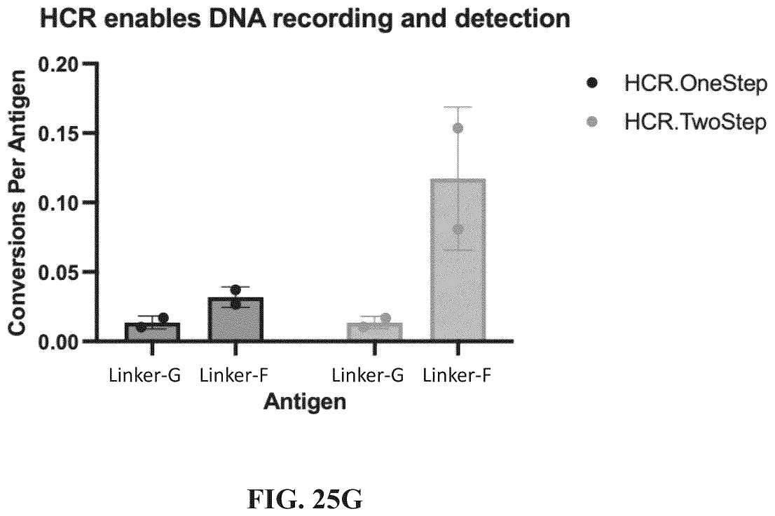

The first set comprises a first hairpin (“H1”) molecule (GGAATTGGGAGTAAGGGCTGTGATGCCCTTACTCCC (SEQ ID NO: 12)) at 500 nanomolar concentration, a second hairpin (“H2”) molecule (GCCCTTACTCCCAATTCCGGGAGTAAGGGCATCACA (SEQ ID NO: 13)) at 500 nanomolar concentration, and an activating nucleic acid sequence (/5Biosg/TTTTTGCCCTTACTCCCAATTCC (SEQ ID NO: 14)) provided at varying concentrations. The first hairpin (H1) molecule is partially complementary to the second hairpin (H2) molecule, and the first hairpin (H1) molecule is also partially complementary to the activating sequence, as shown in FIG. 25A.

The second set comprises the same first hairpin (H1) molecule, the same activating sequence, and a longer-length second hairpin (“H2.5TAIL”) molecule (AGGCTGAGCCAGGATCAAACTCTGCCCTTACTCCCAATTCCGGGAGTAAGGGCATCAC A (SEQ ID NO: 15)) that comprises a 5′ flap sequence that, when hybridized to the first hairpin molecule, comprises a single-stranded overhang that can be used to couple to a DNA capture (“anchor”) molecule, optionally which can be incubated with a primer (ReadyMade 16F primer), as shown in FIG. 25B.

For each set, the hairpin oligos were provided at 500 nM in PBST and incubated with varying concentrations of the activating sequence: 0 (control), 25, 50, or 100 nM, for one hour at room temperature. The products were then run in a DNA gel (4% agarose).

FIG. 25C shows DNA gel electrophoresis results of the HCR reaction. Lanes M and 6 indicate a DNA ladder. Lanes 1 through 4 are the results of the HCR reaction using the first set at varying concentrations of activating sequence (0, 25, 50, or 100 nM). Lanes 6-10 are the results of the HCR reaction using the second set at varying concentrations of activating sequence (0, 25, 50, or 100 nM). As indicated in the gel, the assembly of the HCR products are specific to the presence of the activating sequence (i.e., no HCR products are observed in the 0 nM condition). In lanes 1-4, one main band is present at 0 nM, representing both the first hairpin and the second hairpin molecules (same size); the signal of the one main band diminishes with increasing concentration of the activating sequence (indicating depletion of the hairpin molecules), and larger molecular weight species are observed. In lanes 6 through 10, three bands are observed initially (0 nM activating sequence), indicating presence of the first hairpin molecule, the second hairpin molecule, and a third species, possibly the second hairpin molecule that is hybridized to the primer. As the concentration of the activating sequence increases, the signal of the first hairpin molecule and second hairpin molecule decreases, with increasing signal of other larger species. Altogether, these results indicate that HCR in solution occurs in the presence of an activating nucleic acid molecule.

Next, to test the viability of HCR with a 3′ flap sequence, a third set of oligonucleotides is provided. The third set comprises the same first hairpin (H1) molecule, the same activating sequence, and a longer-length second hairpin (“H2.3Tail”) molecule (GCCCTTACTCCCAATTCCGGGAGTAAGGGCATCACAAAAGAAAGAGGGAGGGAAGGA GAA (SEQ ID NO: 16)) that comprises a 3′ flap sequence that, when hybridized to the first hairpin molecule, comprises a single-stranded overhang that can be used to couple to a DNA capture (“anchor”) molecule, optionally which can be incubated with a primer (“REV”), as shown in FIG. 25D.

FIG. 25E shows DNA gel electrophoresis results of the HCR reaction using the varying second hairpin types. Lane M indicates a DNA ladder. Lane 1 represents the first hairpin (H1) molecule, lane 2 represents the second hairpin (H2) molecule, lane 3 represents the longer-length second hairpin (H2.5Tail) with 5′ flap sequence that is pre-annealed to a 16sF primer, lane 4 represents the longer-length second (H2.3Tail) with 3′ flap sequence that is preannealed to a REV primer, lane 5 represents H1 and H2 added together, lane 6 represents an HCR reaction using H1 and H2 and the activating sequence, lane 7 represents H1 and H2.5Tail that is preannealed to 16sF, lane 8 represents the HCR reaction between H1 and H2.5Tail preannealed to 16sF, in which the activating sequence is added, lane 9 represents H1 and H2.3Tail preannealed to a REV primer, and lane 10 represents the HCR reaction between H1 and H2.3Tail preannealed to a REV primer, in which the activating sequence is added. All hairpin complexes are provided at 500 nM, and hairpin flap sequences (of the H2.5Tail and H2.3Tail) are pre-annealed at a 1:1.5 ratio with the primer (either 16sF or REV). The activating sequence, when present, is provided at 100 nM.

As can be observed in FIG. 25E, the HCR reactions occur only in the presence of an activating sequence (lanes 6, 8, and 10), and no HCR products are observed in the conditions in which both hairpin molecules are provided but no activating sequence is provided. In lanes 1-4, one main band is present, representing the first hairpin and the second hairpin molecules (either H2, H2.5tail, or H2.3tail); the signal of the main bands diminishes with addition of the activating sequence (indicating depletion of the hairpin molecules), and larger molecular weight species are observed. The presence of the flap sequence on the second hairpin molecules also resulted in HCR products. Altogether, these results indicate that HCR in solution occurs in the presence of an activating nucleic acid molecule, even with differing hairpin molecule types or sequences.

Next, to evaluate the performance of HCR in the context of a bead-based substrate system used for peptide sequencing, a magnetic bead comprising a model antigen is provided. FIG. 25F schematically depicts coupling of one of the hairpin molecules to a DNA capture molecule on a bead comprising a model antigen. As described elsewhere herein, the bead may comprise a plurality of polymerizable molecules coupled thereto, which polymerizable molecules include DNA capture molecules. The polymerizable molecules may be identical or different and can serve multiple functions, e.g., for coupling of a polymeric analyte (e.g., peptide) to the substrate, for coupling of a terminal amino acid via a linker and linking nucleic acid molecule, or for coupling of additional polymerizable molecules, e.g., encoding barcode molecules in solution or coupled to binding agents, as shown in FIG. 1A.

In one example, the bead comprises a plurality of DNA capture (“anchor”) molecules (sequence TTCTTCTTCTTCTCCCTCCTCTCTTCT (SEQ ID NO: 17)), as described elsewhere herein. A model antigen comprising a linking (chain) nucleic acid molecule and a linker-amino acid complex (1-(2-azidoethyl)-4-isothiocyanatobenzene linker that has reacted with either a Phe amino acid or a Gly amino acid) is ligated to a subset of the anchor molecules by addition of ligase, incubation at room temperature for 20 minutes, resuspension in 0.1M NaOH twice, resuspension in PBST two times, and resuspension in 0.1% Tween-20. The resultant bead, as depicted schematically in FIG. 25F, comprises a plurality of free anchor molecules, and a subset of anchor molecules ligated to the model antigen, which represents a monomeric analyte (e.g., as would result from workflow 100 of FIG. 1A Panels A-D). The model antigen comprises a linker-Phe or a linker-Gly moiety that can be recognized or bound by a binding agent (e.g., antibody).

Next, the prepared beads are combined with binding agents (antibodies) coupled to the activating sequence. The first and second hairpin molecules are also provided for conducting HCR. Two different protocols are tested, as follows:

Protocol 1 (One-step): the beads comprising the model antigens (linker-Gly or linker-Phe) are incubated with 100 nM streptavidin-conjugated mouse anti-linker-Phe-complex antibodies and 800 nM of biotinylated activating sequence. Next, the first hairpin (H1) molecule and the second hairpin molecule (H2.3Tail) with 3′ flap sequence are added to achieve a 50 nM antibody concentration, 400 nM biotinylated activating sequence concentration, 500 nM H1 concentration and 500 nM H2.3Tail concentration. The reaction is incubated for 1 hour at room temperature in PBST, washed three times in PBST, then subjected to ligation conditions (using 45 microliters of 10× T4 DNA ligase buffer, 382.5 microliters of water, and 22.5 microliters of T4 DNA ligase) for 30 minutes at room temperature, followed by heat killing at 65 degrees Celsius for 20 minutes.

Protocol 2 (Two-step): the beads comprising the model antigens (linker-Gly or linker-Phe) are incubated with 50 nM streptavidin-conjugated mouse anti-linker-Phe-complex antibodies and 400 nM of biotinylated activating sequence for one hour at room temperature, followed by three washes in PBST, then incubated with the hairpin molecules: 500 nM H1 and 500 nM H2.3Tail for 1 hour at room temperature, followed by three washes in PBST and ligation, as described above.

FIG. 25G shows the data from the above-outlined experiments. The y-axis represents the normalized value of the number of ligated molecules (comprising the H2.3Tail sequence and the anchor molecule sequence) for the model antigen types (linker Gly control or linker-Phe) as quantified by qPCR. The left-hand bars represent the results from the one-step protocol and the right-hand bars represent the result from the two-step protocol. As can be observed, both protocols yield specific signal (specific binding of the anti-linker-Phe-complex antibody to its target). The two-step process yields higher conversion events with little change to the background signal (the linker Gly control). Further experiments to investigate the effect of hairpin concentration, buffer conditions, and oligo design on HCR performance can be used to improve the signal. Overall, the results indicate that HCR is a feasible approach for generating barcode molecules that can be used for coupling to additional polymerizable molecules (e.g., DNA capture molecules), thereby specifically encoding a binding event of a binding agent to an antigen target (e.g., amino acid type).

### Example 16—Enzymatic Activating Agents for Coupling of Click Chemistry-Functionalized Tyramide to a Substrate

As described herein, detection of a monomeric analyte may comprise coupling the monomeric analyte to a binding agent comprising an activating agent. The binding agent can bind specifically or semi-specifically to the monomeric analyte. Prior to, during, or subsequent to the binding event, a first polymerizable molecule (e.g., nucleic acid barcode molecule) comprising encoded information (e.g., the identity of the binding agent or the monomeric analyte) may be provided. Detection may comprise using the activating agent to couple the first polymerizable molecule to a second polymerizable molecule, such as a protein or peptide.

In some instances, the activating agent comprises an enzyme, e.g., peroxidase, that can facilitate coupling of the first polymerizable molecule to a second polymerizable molecule (e.g., protein or peptide) that is bound to a substrate. In one such example, the activating agent comprises a peroxidase that can be used to couple a click chemistry-functionalized tyramide to a second polymerizable molecule, e.g., tyrosine-containing protein or peptide, that is bound to the substrate. The peroxidase can attach the click chemistry-functionalized tyramide to the second polymerizable molecule (e.g., protein or peptide), thereby exposing a click chemistry functional handle (e.g., azide) that can subsequently be coupled to a click chemistry-conjugated first polymerizable molecule (e.g., a DBCO-conjugated DNA barcode molecule).

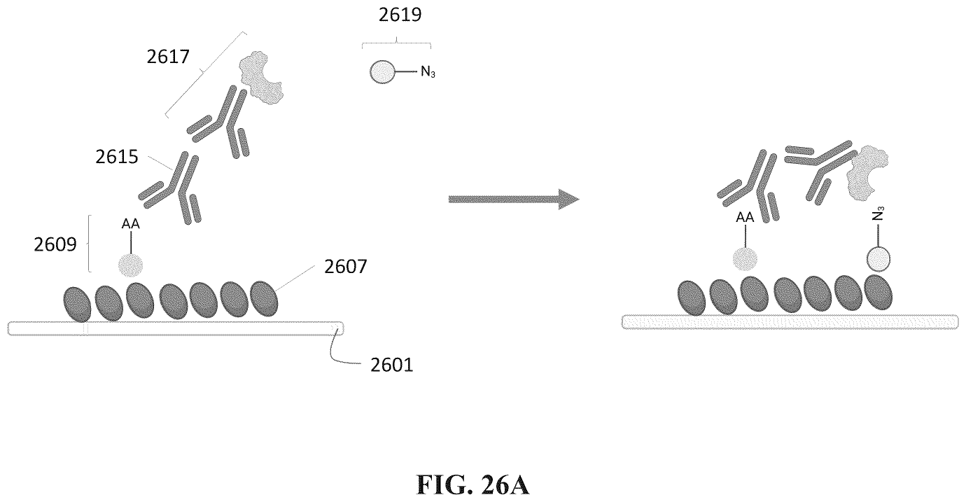

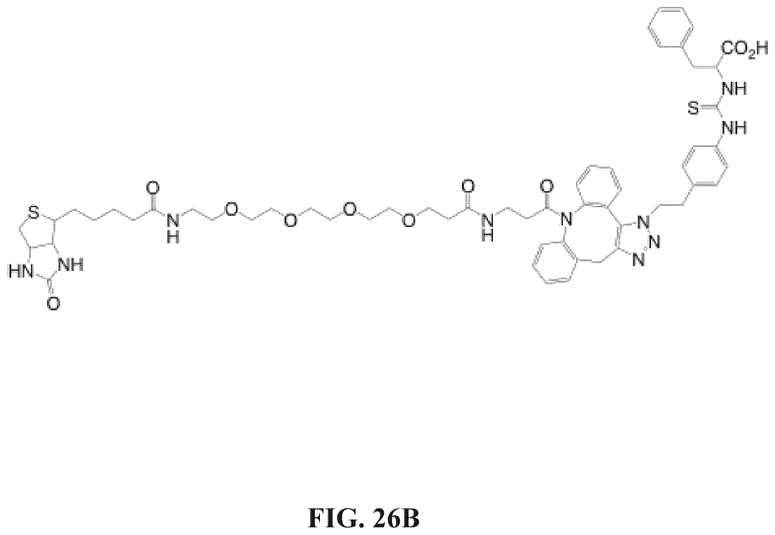

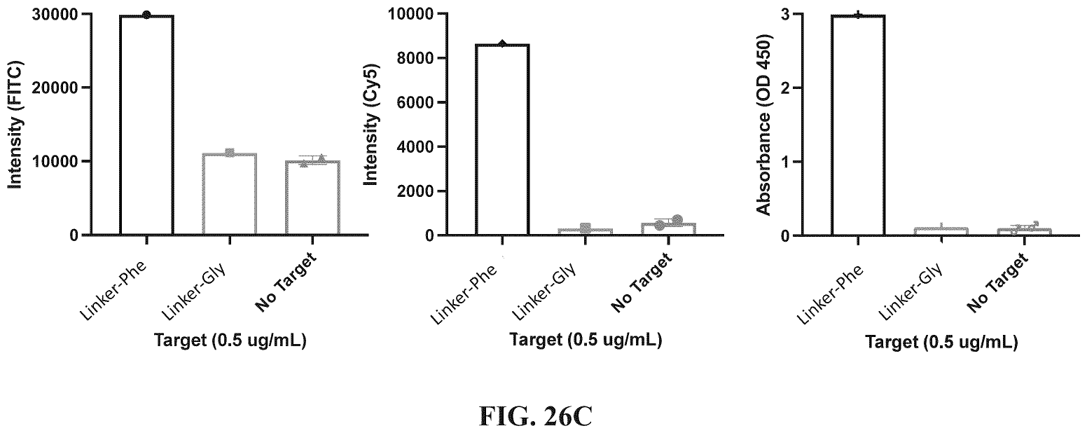

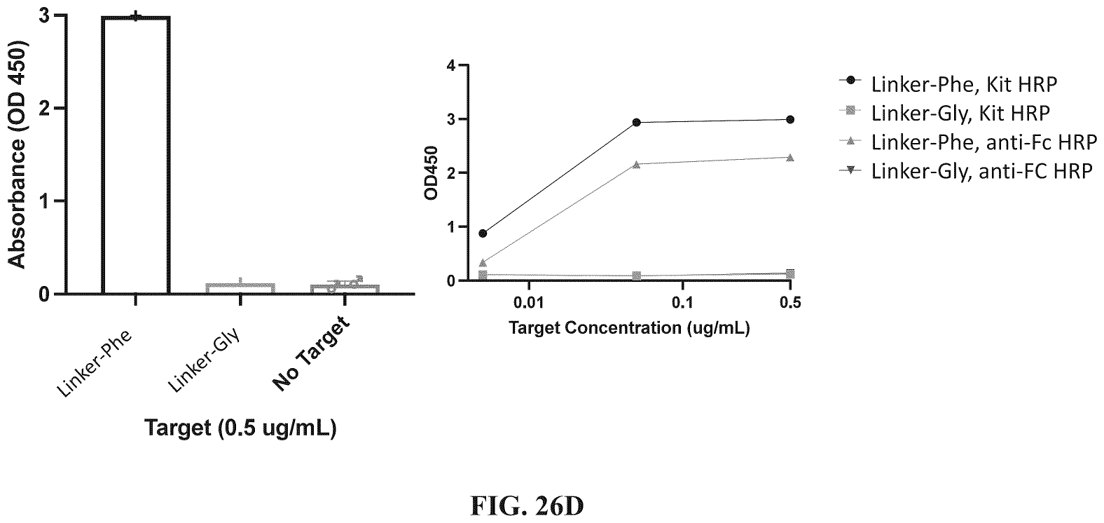

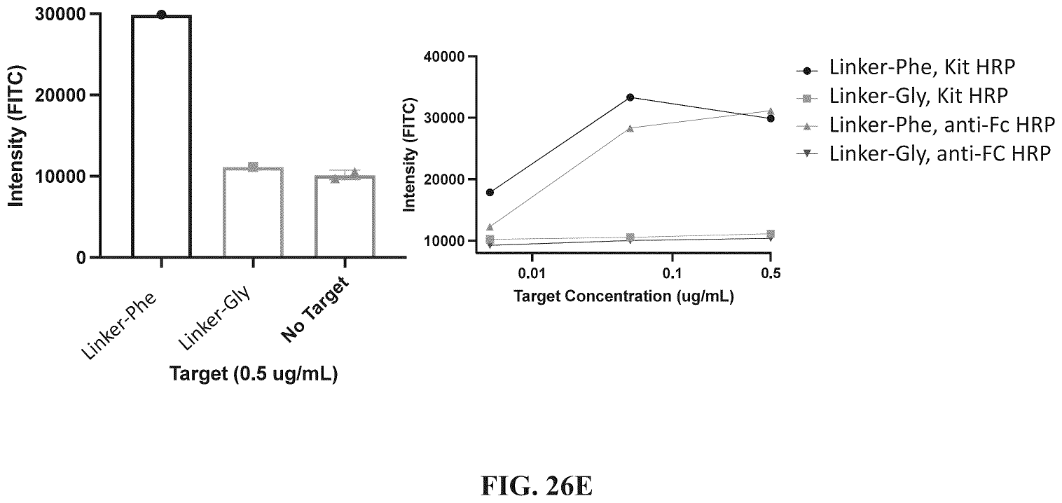

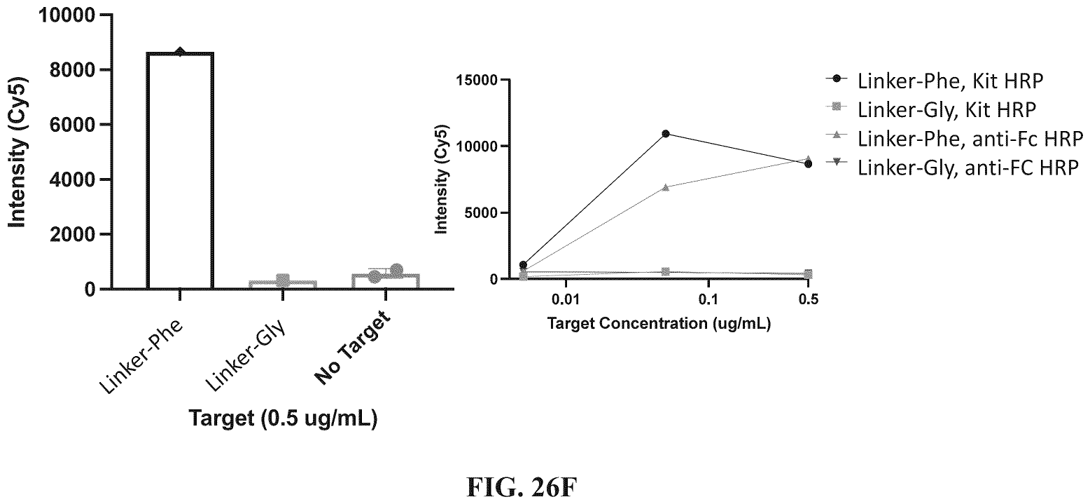

FIG. 26A schematically shows an example proof-of-concept ELISA experiment that shows local activation of a substrate to facilitate coupling of polymerizable molecules. A multi-well ELISA plate (used as a substrate 2601), is coated with a plurality of streptavidin molecules (in which the streptavidin molecules represent capture moieties 2607 that comprise peptidic polymerizable molecules). A monomeric analyte (antigen) 2609 is provided. The monomeric analyte 2609 comprises biotin, which can couple to the streptavidin on the plate, and also comprises a linker and an amino acid (in this example, phenylalanine), as described elsewhere herein. The chemical formula of the monomeric analyte is shown in FIG. 26B. The monomeric analyte 2609 is detected using a primary antibody (binding agent 2615), which specifically recognizes the linker-Phe complex. A secondary antibody (binding agent 2617) comprising an HRP or APEX enzyme is provided. A fluorescently labelled molecular substrate 2619, optionally comprising a click chemistry-functionalized tyramide (e.g., azide-tyramide-cy5 or tyramide-AlexaFluor 488) is provided, which is processed by the HRP or APEX enzyme to couple the molecular substrate 2619 to the surface via tyrosine residues in the streptavidin molecules, thereby providing click chemistry (azide)-functionalized capture moieties that can subsequently be covalently coupled to click chemistry (e.g., DBCO) functionalized polymerizable molecules (e.g., DNA barcodes (not shown)). The fluorescence of the tyramide molecules can be detected to measure the local activation.

To demonstrate the local activation empirically, a 96-well ELISA plate (Nunc 96 well Immuno Plate Maxisorb, ThermoFisher) is coated with recombinant streptavidin (A9275, Sigma Aldrich) diluted in PBS (Teknova) at 4 degrees Celsius overnight. The plate is then washed with PBS and blocked with blocking buffer (3% BSA) for 1 hour at room temperature. The plate is then washed twice with PBS, thereby providing a streptavidin-coated surface. Monomeric analytes (antigen) are then added to the plate at varying concentrations (0.01, 0.1, and 0.5 micrograms per milliliter) in 1% BSA buffer and incubated for 1 hour at room temperature. The monomeric analyte comprises a biotinylated-linker-amino acid complex comprising Phe, as shown in FIG. 26B, or Gly (negative control). The plates are then washed with PBS-T and PBS and then incubated with primary antibody (mouse IgG anti-linker-Phe-complex) in 1% BSA buffer for 1 hour. The plates are washed 3 times with PBS-T and PBS, and then HRP-conjugated polyclonal goat anti-mouse secondary antibodies (B40941 Invitrogen) or anti-mouse Fc HRP secondary antibodies (Jackson Immuno at a 1:5000 dilution) are added and incubated for 45 minutes to 1 hour. The plates are washed with PBS-T three times and then PBS two times, and then azido-cy5-tyramide or A488-tyramide (AAT Bioquest) is added for 10 minutes. The reaction is quenched with stop solution (ThermoFisher), washed three times with PBS-T and two times with PBS. PBS is added to the tyramide wells, and 3,3′,5,5′-Tetramethylbenzidine (TMB) is added to the non-tyramide wells as a positive control for the HRP reaction. The reaction is stopped with H2SO4, and the absorbance and fluorescence are measured.

FIG. 26C shows example data from the experiment outlined above. Each plot represents the measured intensity for FITC (Alexa Fluor 488), Cy5, or absorbance for each of the antigen (monomeric analyte) types (linker-Phe, linker-Gly, or no target) at 0.5 microgram/milliliter concentration when probed with an anti-Phe primary antibody and the HRP-conjugated polyclonal goat anti-mouse secondary antibody (Invitrogen). As can be observed from the plots, the signal is higher for the linker-Phe condition as compared to the controls (linker-Gly and no antigen), indicating that the primary antibody is specific, and that the use of a secondary antibody with an activating HRP leads to tyramide signal in the plate.

FIG. 26D shows example data from the experimental controls, in which TMB is used as the molecular substrate and absorbance is used as a readout for the HRP activity. The left-hand plot, the same as the absorbance plot shown in FIG. 26C, indicates the absorbance as measured for each of the antigen (monomeric analyte) types (linker-Phe, linker-Gly, or no target) at 0.5 microgram/milliliter concentration when probed with an anti-Phe primary antibody and the HRP-conjugated polyclonal goat anti-mouse secondary antibodies (B40941 Invitrogen). As can be observed from the plot, the signal is higher for the linker-Phe condition as compared to the controls (linker-Gly and no antigen), also suggesting that the primary antibody is specific, and that the use of a secondary antibody with an activating HRP leads to tyramide signal in the plate. The right-hand plot indicates the measured absorbance for the different secondary antibodies (Invitrogen “Kit” HRP or anti-Fc HRP from Jackson Immuno) for varying concentrations of monomeric analyte. As indicated in the plot, the signal is higher for the correct antigen (linker-Phe), irrespective of the type of secondary antibody used, thus indicating specificity of the primary antibody to its target.

FIG. 26E shows example data from the measurement of tyramide signal when tyramide-AlexaFluor 488 is used as the molecular substrate. The left-hand plot, the same as the FITC intensity plot shown in FIG. 26C, indicates the fluorescence as measured for each of the antigen (monomeric analyte) types (linker-Phe, linker-Gly, or no target) at 0.5 microgram/milliliter concentration when probed with an anti-Phe primary antibody and then HRP-conjugated polyclonal goat anti-mouse secondary antibodies (B40941 Invitrogen). As can be observed from the plot, the signal is higher for the linker-Phe condition as compared to the controls (linker-Gly and no antigen), also suggesting that the primary antibody is specific, and that the use of a secondary antibody with an activating HRP leads to tyramide signal in the plate. The right-hand plot indicates the measured fluorescence for the different secondary antibodies (Invitrogen “Kit” HRP or anti-Fc HRP from Jackson Immuno) for varying concentrations of monomeric analyte. As indicated in the plot, the signal is higher for the correct antigen (linker-Phe), irrespective of the type of secondary antibody used, thus indicating specificity of the primary antibody to its target.

FIG. 26F shows example data from the measurement of tyramide signal when azide-tyramide-Cy5 is used as the molecular substrate. The left-hand plot, the same as the Cy5 intensity plot shown in FIG. 26C, indicates the fluorescence as measured for each of the antigen (monomeric analyte) types (linker-Phe, linker-Gly, or no target) at 0.5 microgram/milliliter concentration when probed with an anti-Phe primary antibody and then HRP-conjugated polyclonal goat anti-mouse secondary antibodies (B40941 Invitrogen). As can be observed from the plot, the signal is higher for the linker-Phe condition as compared to the controls (linker-Gly and no antigen), also suggesting that the primary antibody is specific, and that the use of a secondary antibody with an activating HRP leads to tyramide signal in the plate. The right-hand plot indicates the measured fluorescence for the different secondary antibodies (Invitrogen “Kit” HRP or anti-Fc HRP from Jackson Immuno) for varying concentrations of monomeric analyte. As indicated in the plot, the signal is higher for the correct antigen (linker-Phe), irrespective of the type of secondary antibody used, thus indicating specificity of the primary antibody to its target.

Altogether, the results from FIG. 26C-26F indicate that using HRP as an activating agent may be an effective way to immobilize functional groups (e.g., click chemistry-functionalized tyramide) to polymerizable molecules (e.g., tyrosine-containing peptides, fluorescein-labeled DNA molecules), which can enable downstream coupling of polymerizable molecules comprising encoded information (e.g., DNA barcode molecules) that are functionalized with a complementary functional group (e.g., click chemistry). Overall, the use of HRP as an activating agent may be a feasible approach to couple polymerizable molecules and enable detection of monomeric analytes.

### Example Embodiments

The present disclosure provides example embodiments. Among the provided embodiments are:

1. A method for sequencing a peptide with single-molecule sensitivity, comprising:
(a) providing said peptide, wherein said peptide comprises a plurality of amino acids; and
(b) sequencing said peptide to determine an identity and order of at least a subset of said plurality of amino acids,
wherein an individual read accuracy of an amino acid is greater than 50% for at least 5 different amino acids.

2. The method of embodiment 1, wherein at least one amino acid of said plurality of amino acids comprises a post-translational modification.

3. The method of embodiment 1 or 2, wherein (a) comprises providing a plurality of peptides including said peptide.

4. The method of any one of embodiments 1-3, wherein said individual read accuracy of said amino acid is greater than 50%, wherein the individual read accuracy is not affected by a side chain of an adjacent amino acid.

5. The method of any one of embodiments 1-4, wherein said amino acid comprises one or more amino acids.

6. The method of embodiment 5, wherein said one or more amino acids is fewer than 6 amino acids.

7. The method of embodiment 5, wherein said amino acid comprises only one amino acid.

8. The method of any one of embodiments 1-7, wherein (b) comprises: (i) cleaving said amino acid from said peptide; (ii) coupling said amino acid to a capture moiety to generate an amino acid-capture moiety complex; (iii) contacting said amino acid-capture moiety complex with a binding agent; (iv) providing a first polymerizable molecule; and (v) coupling said first polymerizable molecule to a second polymerizable molecule.

9. The method of embodiment 8, wherein (ii) occurs prior to (i).

10. The method of embodiment 8, wherein (ii) occurs during (i).

11. The method of embodiment 8, wherein (ii) occurs subsequent to (i).

12. The method of embodiment 8, wherein (iv) occurs prior to (iii).

13. The method of embodiment 8, wherein (iv) occurs during (iii).

14. The method of embodiment 8, wherein (iv) occurs subsequent to (iii).

15. The method of any one of embodiments 8-14, wherein, during (iv), said binding agent is not coupled to said first polymerizable molecule.

16. The method of embodiment 15, wherein said binding agent comprises an activating agent, wherein said activating agent facilitates coupling of said first polymerizable molecule to said second polymerizable molecule.

17. The method of embodiment 16, wherein said activating agent comprises an activating polymerizable molecule, and further comprising, subsequent to (iv), coupling said first polymerizable molecule to said activating polymerizable molecule.

18. The method of embodiment 17, wherein said coupling is mediated using a splint molecule comprising a sequence complementary to at least a portion of (i) said first polymerizable molecule, (ii) said second polymerizable molecule and (iii) said activating polymerizable molecule.

19. The method of embodiment 17, wherein said first polymerizable molecule and said activating polymerizable molecules are nucleic acid molecules and wherein said coupling of said first polymerizable molecule to said activating polymerizable molecule occurs via hybridization chain reaction (HCR).

20. The method of embodiment 16, wherein said activating agent comprises an enzyme.

21. The method of embodiment 20, wherein said enzyme comprises a peroxidase, a Cas protein, a ligase, a kinase, or a restriction enzyme.

22. The method of embodiment 21, wherein said enzyme comprises a kinase, wherein said kinase is configured to phosphorylate said second polymerizable molecule, thereby allowing attachment of said first polymerizable molecule to said second polymerizable molecule.

23. The method of embodiment 8, wherein said binding agent is coupled indirectly to said first polymerizable molecule.

24. The method of embodiment 23, wherein said binding agent comprises an activating polymerizable molecule, and wherein said first polymerizable molecule is coupled to said activating polymerizable molecule.

25. The method of embodiment 8, wherein said binding agent comprises an activating agent, wherein said activating agent is configured to facilitate coupling of said first polymerizable molecule to said second polymerizable molecule.

26. The method of embodiment 25, wherein said activating agent comprises an enzyme or a nucleic acid molecule.

27. The method of embodiment 26, wherein said activating agent comprises an anchoring nucleic acid molecule, wherein said anchoring nucleic acid molecule is configured to couple to an additional second polymerizable molecule adjacent to said second polymerizable molecule.

28. The method of embodiment 26, wherein said activating agent comprises an enzyme selected from the group consisting of: a peroxidase, a Cas protein, a ligase, a kinase, and a restriction enzyme.

29. The method of embodiment 28, wherein said enzyme is a kinase, and further comprising, using said kinase to phosphorylate said second polymerizable molecule, thereby allowing coupling of said first polymerizable to said second polymerizable molecule.

30. The method of embodiment 28, wherein said enzyme is a horseradish peroxidase (HRP) or an ascorbate peroxidase (APEX), wherein said HRP or said APEX activates said first polymerizable molecule or said second polymerizable molecule, thereby coupling said first polymerizable molecule to said second polymerizable molecule.

31. The method of embodiment 28, wherein said enzyme is a restriction enzyme and wherein said second polymerizable molecule comprises a restriction recognition site, and further comprising, cleaving said second polymerizable molecule with said restriction enzyme prior to said coupling of said first polymerizable molecule to said second polymerizable molecule.

32. The method of embodiment 8, wherein said binding agent comprises an activating nucleic acid molecule and wherein said first polymerizable molecule is a nucleic acid molecule, and further comprising, prior to (iii), coupling said first polymerizable molecule to said activating nucleic acid sequence.

33. The method of embodiment 32, further comprising, generating said first polymerizable molecule via hybridization chain reaction (HCR).

34. The method of embodiment 33, wherein said generating comprises (A) providing a first hairpin molecule and a second hairpin molecule; (B) coupling said first hairpin molecule to said activating nucleic acid sequence; and (C) coupling said second hairpin molecule to said first hairpin molecule, thereby generating said first polymerizable molecule coupled to said activating nucleic acid molecule.

35. The method of embodiment 34, wherein subsequent to (C), said first polymerizable molecule comprises a flap sequence, and further comprising, using said flap sequence to couple said first polymerizable molecule to said second polymerizable molecule.

36. The method of any one of embodiments 8-35, further comprising, repeating (i)-(v) at least once.

37. The method of any one of embodiments 8-36, wherein said capture moiety comprises a third polymerizable molecule.

38. The method of embodiment 37, wherein said first polymerizable molecule, said second polymerizable molecule or said third polymerizable molecule comprises a nucleic acid molecule.

39. The method of embodiment 38, wherein said nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof.

40. The method of embodiment 38 or 39, wherein said nucleic acid molecule comprises a hexonucleic acid (HNA).

41. The method of any one of embodiments 38-40, wherein greater than 70% of nucleotides of said nucleic acid molecule are thymines or cytosines.

42. The method of embodiment 38, wherein said third polymerizable comprises a DNA molecule comprising a priming site, a sequencing site, a unique molecular identifier, a barcode sequence, a cleavage site, or a combination thereof.

43. The method of embodiment 38, wherein said first polymerizable molecule comprises a first nucleic acid molecule and wherein said third polymerizable molecule comprises a second nucleic acid molecule.

44. The method of embodiment 43, wherein said coupling in (iv) comprises hybridization of at least a portion of said first nucleic acid molecule to at least a portion of said second nucleic acid molecule.

45. The method of embodiment 44, further comprising, performing a nucleic acid extension reaction.

46. The method of any one of embodiments 8-45, wherein said cleaving in (i) comprises cleaving one or more amino acids from said peptide.

47. The method of embodiment 8, wherein said first polymerizable molecule comprises a first nucleic acid molecule and said second polymerizable molecule comprises a second nucleic acid molecule.

48. The method of embodiment 47, wherein said coupling in (ii) comprises ligation or hybridization of said first nucleic acid molecule to said second nucleic acid molecule.

49. The method of embodiment 47, wherein said coupling in (ii) is mediated by a splint molecule.

50. The method of any one of embodiments 8-49, wherein said first polymerizable molecule comprises a first reactive moiety and said second polymerizable molecule comprises a second reactive moiety capable of reacting with said first reactive moiety.

51. The method of embodiment 50, wherein said coupling in (ii) comprises reacting said first moiety and said second moiety.

52. The method of embodiment 50 or 51, wherein said first reactive moiety or said second reactive moiety comprises a click chemistry moiety.

53. The method of any one of embodiments 8-52, further comprising, decoupling said amino acid from said capture moiety.

54. The method of embodiment 53, further comprising, repeating (b).

55. The method of embodiment 53, further comprising, identifying at least a portion of said first polymerizable molecule or derivative thereof.

56. The method of embodiment 55, wherein said identifying comprises sequencing said first polymerizable molecule or derivative thereof.

57. The method of embodiment 56, wherein said sequencing is performed using next generation DNA sequencing.

58. The method of embodiment 55, wherein said identifying comprises contacting said first polymerizable molecule or derivative thereof with a probe.

59. The method of any one of embodiments 8-58, wherein said coupling of (ii) is mediated using a linker.

60. The method of embodiment 59, wherein said linker is a bifunctional linker.

61. The method of embodiment 59 or 60, wherein said linker comprises a first reactive group and a second reactive group, wherein said first reactive group is capable of coupling to said amino acid and wherein said second reactive group is capable of coupling to said capture moiety.

62. The method of embodiment 61, wherein said amino acid is a terminal amino acid.

63. The method of embodiment 61 or 62, wherein said first reactive group comprises a thiocyanate, aldehyde group, dansyl chloride, or fluorobenzene.

64. The method of embodiment 63, wherein said fluorobenzene is dinitrofluorobenzene.

65. The method of embodiment 63, wherein said thiocyanate comprises a phenylisothiocyanate (PITC) moiety and wherein said coupling of (ii) generates a phenylthiocarbamoyl (PTC) derivative of said amino acid.

66. The method of embodiment 65, further comprising, subsequent to (e), derivatizing said phenylthiocarbamoyl (PTC) derivative to generate a thiazolinone (ATZ) derivative or a phenylthiohydantoin (PTH) derivative.

67. The method of embodiment 66, further comprising, derivatizing said ATZ derivative or said PTH derivative to another PTC derivative.

68. The method of embodiment 61, wherein said cleaving in (i) is performed by applying a stimulus.

69. The method of embodiment 68, wherein said stimulus comprises a change in pH.

70. The method of embodiment 69, wherein said change in pH comprises the use of an acid.

71. The method of embodiment 69, wherein said change in pH comprises the use of a base.

72. The method of embodiment 68, wherein said stimulus comprises the use of a Lewis acid.

73. The method of embodiment 72, wherein said Lewis acid comprises boron triflate (BF₃), boron trifluoride etherate, boron trichloride, boron tribromide, boron triiodide, or scandium triflate.

74. The method of any one of embodiments 68-73, wherein said stimulus comprises electromagnetic radiation.

75. The method of embodiment 74, wherein said electromagnetic radiation is applied using a microwave.

76. The method of embodiment 61, wherein said capture moiety comprises a third reactive group that is capable of reacting with said second reactive group.

77. The method of embodiment 76, wherein said first reactive group, said second reactive group, or said third reactive group comprises a click chemistry moiety.

78. The method of embodiment 77, wherein said second reactive group and said third reactive group comprise a click chemistry pair.

79. The method of embodiment 59, wherein said linker comprises an amino acid reactive group and an additional polymerizable molecule, wherein said additional polymerizable molecule is configured to couple to said capture moiety.

80. The method of embodiment 79, wherein said amino acid reactive group is capable of cleaving said amino acid.

81. The method of embodiment 79 or 80, wherein said additional polymerizable molecule comprises an enzyme recognition site.

82. The method of embodiment 81, wherein said enzyme recognition site is recognized by a nuclease.

83. The method of embodiment 82, further comprising, subsequent to (iv), cleaving said additional polymerizable molecule, thereby releasing said amino acid from said capture moiety.

84. The method of embodiment 82, wherein said nuclease is a restriction enzyme, a Cas protein, or an Argonaut nuclease.

85. The method of embodiment 8, wherein said cleaving in (i) comprises applying a stimulus.

86. The method of embodiment 85, wherein said stimulus comprises a change in pH.

87. The method of embodiment 85, wherein said stimulus comprises the use of a Lewis acid.

88. The method of embodiment 87, wherein said Lewis acid comprises boron triflate (BF₃), boron trifluoride etherate, boron trichloride, boron tribromide, boron triiodide, or scandium triflate.

89. The method of any one of embodiments 85-88, wherein said stimulus comprises electromagnetic radiation.

90. The method of embodiment 89, wherein said electromagnetic radiation is applied using a microwave.

91. The method of any one of embodiments 85-90, wherein said stimulus comprises a biological stimulus.

92. The method of embodiment 91, wherein said biological stimulus comprises use of an enzyme.

93. The method of embodiment 92, wherein said enzyme is a metalloprotease, aminopeptidase, or exopeptidase.

94. The method of embodiment 93, further comprising, providing a metal catalyst.

95. The method of embodiment 92, wherein said amino acid comprises a modification, and wherein said enzyme recognizes said modification.

96. The method of any one of embodiments 8-95, wherein said first polymerizable molecule comprises a moiety that identifies said binding agent or said amino acid.

97. The method of embodiment 96, wherein said first polymerizable molecule comprises a nucleic acid molecule comprising a barcode sequence that identifies said binding agent or said amino acid.

98. The method of any one of embodiments 8-97, further comprising, subjecting said amino acid-capture moiety complex to conditions sufficient to inhibit binding of an additional binding agent to said amino acid-capture moiety complex.

99. The method of embodiment 98, wherein said conditions comprise a chemical or enzymatic treatment.

100. The method of any one of embodiments 8-99, further comprising, blocking a carboxyl group or an amine group of said peptide.

101. The method of any one of embodiments 8-100, wherein said binding agent comprises an antibody, antibody fragment, aptamer, scFv, nanobody, anticalin, tRNA-acyl synthetase, peptide, peptide mimetic, a polysaccharide, or derivative thereof.

102. The method of any one of embodiments 8-101, wherein said binding agent comprises an enzyme.

103. The method of embodiment 102, wherein said enzyme comprises a metalloprotease, aminopeptidase, or exopeptidase.

104. The method of any one of embodiments 8-103, wherein said peptide, said capture moiety, or said second polymerizable molecule is coupled to a substrate.

105. The method of embodiment 104, wherein said substrate is a solid support.

106. The method of embodiment 104 or 105, wherein said substrate is substantially planar.

107. The method of embodiment 104 or 105, wherein said substrate is a bead or particle.

108. The method of any one of embodiments 104-107, wherein said peptide, said capture moiety, or said second polymerizable molecule is coupled to said substrate via a click chemistry moiety.

109. The method of any one of embodiments 104-107, wherein said peptide, said capture moiety, or said second polymerizable molecule is coupled to said support via a functional group.

110. The method of embodiment 109, wherein said functional group is added to said peptide, said capture moiety, or said second polymerizable molecule using an enzyme.

111. The method of embodiment 110, wherein said enzyme comprises an amidase.

112. The method of embodiment 104, wherein said peptide, said capture moiety, or said second polymerizable molecule is coupled to said substrate using a linker molecule.

113. The method of embodiment 104, wherein said substrate is functionalized with a functional group, and further comprising, attaching said peptide, said capture moiety, or said second polymerizable molecule to said functional group.

114. The method of embodiment 113, wherein said peptide, said capture moiety, and said second polymerizable molecule are coupled to said substrate.

115. The method of any one of embodiments 104-114, further comprising, passivating said substrate.

116. The method of embodiment 115, wherein said passivating decreases nonspecific binding of said binding agent.

117. The method of any one of embodiments 1-116, wherein at least one amino acid of said plurality of amino acids comprises a non-naturally occurring modification.

118. The method of embodiment 117, further comprising, generating said peptide comprising said non-naturally occurring modification.

119. The method of embodiment 118, wherein said generating comprises alkylating or acetylating said at least one amino acid, beta-elimination of a phosphate group, or use of PITC or acetic anhydride.

120. The method of embodiment 118, wherein said generating comprises converting a cysteine residue to cysteic acid.

121. The method of embodiment 118, wherein said generating comprises use of an oxidizing or reducing agent.

122. The method of any one of embodiments 1-121, wherein said peptide is derived from a biological sample.

123. The method of embodiment 1, wherein (b) comprises: (i) cleaving said amino acid from said peptide; (ii) coupling said amino acid to a capture moiety to generate an amino acid-capture moiety complex; (iii) contacting said amino acid-capture moiety complex with a binding agent, wherein said binding agent comprises an activating agent; and (iv) detecting a product from said activating agent.

124. The method of embodiment 123, wherein said activating agent comprises an enzyme that generates said product.

125. The method of embodiment 124, wherein said enzyme is a horseradish peroxidase or ascorbate peroxidase.

126. A method for processing a polymeric analyte, comprising:
(a) providing said polymeric analyte and a capture moiety, wherein said polymeric analyte comprises a plurality of monomers;
(b) cleaving a monomer of said plurality of monomers from said polymeric analyte;
(c) coupling said monomer to said capture moiety to generate a monomer-capture moiety complex;
(d) subsequent to (b) and (c), contacting said monomer-capture moiety complex with a binding agent comprising an activating agent;
(e) providing a first polymerizable molecule; and
(f) using said activating agent to couple said first polymerizable molecule to a second polymerizable molecule.

127. The method of embodiment 126, wherein (e) occurs prior to (d).

128. The method of embodiment 126, wherein (e) occurs during (d).

129. The method of embodiment 126, wherein (e) occurs subsequent to (d).

130. The method of embodiment 126, wherein, during (e), said binding agent is not coupled to said first polymerizable molecule.

131. The method of embodiment 126, wherein said binding agent is coupled indirectly to said first polymerizable molecule.

132. The method of any one of embodiments 126-131, wherein said activating agent comprises an activating polymerizable molecule, and further comprising, coupling said first polymerizable molecule to said activating polymerizable molecule.

133. The method of embodiment 132, wherein said first polymerizable molecule, said second polymerizable molecule, and said activating polymerizable molecule are nucleic acid molecules, and wherein said coupling is mediated using a splint molecule comprising a sequence complementary to at least a portion of (i) said first polymerizable molecule, (ii) said second polymerizable molecule and (iii) said activating polymerizable molecule.

134. The method of any one of embodiments 126-133, wherein said first polymerizable molecule comprises a nucleic acid molecule, and further comprising, generating said first polymerizable molecule via hybridization chain reaction (HCR).

135. The method of embodiment 134, wherein said generating comprises (A) providing a first hairpin molecule and a second hairpin molecule; (B) coupling said first hairpin molecule to said activating polymerizable molecule; and (C) coupling said second hairpin molecule to said first hairpin molecule, thereby generating said first polymerizable molecule coupled to said activating polymerizable molecule.

136. The method of embodiment 135, wherein subsequent to (C), said first polymerizable molecule comprises a flap sequence, and further comprising, using said flap sequence to couple said first polymerizable molecule to said second polymerizable molecule.

137. The method of embodiment 135, further comprising, coupling an additional first hairpin molecule to said first polymerizable molecule and an additional second hairpin molecule to said additional first hairpin molecule, thereby generating an additional first polymerizable molecule that is coupled to said first polymerizable molecule.

138. The method of embodiment 137, further comprising, coupling said additional first polymerizable molecule to an additional second polymerizable molecule.

139. The method of embodiment 135, further comprising, repeating (b)-(f), wherein said repeating comprises generating additional first polymerizable molecules by repeating (A)-(C).

140. The method of embodiment 139, wherein said repeating of (A)-(C) comprises providing additional hairpin molecules, wherein at least a subset of said additional hairpin molecules comprise a different sequence than said first hairpin molecule or said second hairpin molecule.

141. The method of embodiment 140, wherein said subset of said additional hairpin molecules comprise a sequence that is configured to couple to at least a portion of said first polymerizable molecule.

142. The method of embodiment 134, further comprising, cleaving a portion of said first polymerizable molecule.

143. The method of embodiment 126, wherein said activating agent comprises an anchoring nucleic acid molecule, wherein said anchoring nucleic acid molecule is configured to couple to an additional second polymerizable molecule adjacent to said second polymerizable molecule.

144. The method of embodiment 143, wherein said first polymerizable molecule is coupled to said binding agent.

145. The method of embodiment 126, wherein said activating agent comprises an enzyme selected from the group consisting of: a peroxidase, a Cas protein, a ligase, a kinase, and a restriction enzyme.

146. The method of embodiment 145, wherein said enzyme is a kinase, and further comprising, using said kinase to phosphorylate said second polymerizable molecule, thereby allowing coupling of said first polymerizable to said second polymerizable molecule.

147. The method of embodiment 145, wherein said enzyme is a horseradish peroxidase (HRP) or an ascorbate peroxidase (APEX), wherein said HRP or said APEX activates said first polymerizable molecule or said second polymerizable molecule, thereby coupling said first polymerizable molecule to said second polymerizable molecule.

148. The method of embodiment 145, wherein said enzyme is a restriction enzyme and wherein said second polymerizable molecule comprises a restriction recognition site, and further comprising, cleaving said second polymerizable molecule with said restriction enzyme prior to said coupling of said first polymerizable molecule to said second polymerizable molecule.

149. The method of any one of embodiments 126-148, wherein (c) is performed prior to (b).

150. The method of any one of embodiments 126-148, wherein (c) is performed during (b).

151. The method of any one of embodiments 126-148, wherein (c) is performed subsequent to (b).

152. The method of any one of embodiments 126-151, wherein said cleaving in (b) comprises cleaving one or more monomers of said plurality of monomers.

153. The method of any one of embodiments 126-152, wherein said capture moiety comprises a third polymerizable molecule.

154. The method of embodiment 153, wherein said first polymerizable molecule, said second polymerizable molecule or said third polymerizable molecule comprises a nucleic acid molecule.

155. The method of embodiment 154, wherein said nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof.

156. The method of embodiment 154 or 155, wherein said nucleic acid molecule comprises a hexonucleic acid (HNA).

157. The method of any one of embodiments 154-156, wherein greater than 70% of nucleotides of said nucleic acid molecule are thymines or cytosines.

158. The method of any one of embodiments 153-157, wherein said third polymerizable comprises a DNA molecule comprising a priming site, a sequencing site, a unique molecular identifier, a barcode sequence, a cleavage site, or a combination thereof.

159. The method of any one of embodiments 153-158, wherein said first polymerizable molecule comprises a first nucleic acid molecule and wherein said third polymerizable molecule comprises a second nucleic acid molecule.

160. The method of embodiment 159, wherein said coupling in (e) comprises hybridization of at least a portion of said first nucleic acid molecule to at least a portion of said second nucleic acid molecule.

161. The method of embodiment 160, further comprising, performing a nucleic acid extension reaction.

162. The method of embodiment 126, wherein said first polymerizable molecule comprises a first nucleic acid molecule and said second polymerizable molecule comprises a second nucleic acid molecule.

163. The method of embodiment 162, wherein said coupling in (c) comprises ligation or hybridization of said first nucleic acid molecule to said second nucleic acid molecule.

164. The method of embodiment 162, wherein said coupling in (c) is mediated by a splint molecule.

165. The method of any one of embodiments 126-164, wherein said first polymerizable molecule comprises a first reactive moiety and said second polymerizable molecule comprises a reactive moiety capable of reacting with said first reactive moiety.

166. The method of embodiment 165, wherein said coupling in (c) comprises reacting said first moiety and said second moiety.

167. The method of embodiment 165 or 166, wherein said first reactive moiety or said second reactive moiety comprises a click chemistry moiety.

168. The method of any one of embodiments 126-167, further comprising, decoupling said monomer from said capture moiety.

169. The method of embodiment 168, further comprising, repeating (b)-(e).

170. The method of embodiment 169, further comprising, identifying at least a portion of said first polymerizable molecule or derivative thereof.

171. The method of embodiment 170, wherein said identifying comprises sequencing said first polymerizable molecule or derivative thereof.

172. The method of embodiment 171, wherein said sequencing is performed using next generation DNA sequencing.

173. The method of embodiment 171, wherein said identifying comprises using hybridization of probes.

174. The method of any one of embodiments 126-173, wherein said coupling of (c) is mediated using a linker.

175. The method of embodiment 174, wherein said linker is a bifunctional linker.

176. The method of embodiment 174 or 175, wherein said linker comprises a first reactive group and a second reactive group, wherein said first reactive group is capable of coupling to said monomer and wherein said second reactive group is capable of coupling to said capture moiety.

177. The method of embodiment 176, wherein said polymeric analyte is a peptide comprising a plurality of amino acids and said first reactive group is capable of coupling to an amino acid.

178. The method of embodiment 177, wherein said amino acid is a terminal amino acid.

179. The method of embodiment 177, wherein at least one amino acid of said plurality of amino acids comprises a post-translational modification.

180. The method of embodiment 177, wherein said first reactive group comprises a thiocyanate, aldehyde group, dansyl chloride, or fluorobenzene.

181. The method of embodiment 180, wherein said thiocyanate comprises a phenylisothiocyanate (PITC) moiety and wherein said coupling of (c) generates a phenylthiocarbamoyl derivative of said amino acid.

182. The method of embodiment 181, further comprising, subsequent to (e), derivatizing said phenylthiocarbamoyl derivative to generate a thiazolinone (ATZ) derivative or a phenylthiohydantoin (PTH)-amino acid derivative.

183. The method of embodiment 180, wherein said cleaving in (b) is performed by applying a stimulus.

184. The method of embodiment 183, wherein said stimulus comprises a change in pH.

185. The method of embodiment 184, wherein said change in pH comprises the use of an acid.

186. The method of embodiment 184, wherein said change in pH comprises the use of a base.

187. The method of embodiment 183, wherein said stimulus comprises the use of a Lewis acid.

188. The method of embodiment 187, wherein said Lewis acid comprises boron triflate (BF₃), boron trifluoride etherate, boron trichloride, boron tribromide, boron triiodide, or scandium triflate.

189. The method of any one of embodiments 183-188, wherein said stimulus comprises electromagnetic radiation.

190. The method of embodiment 189, wherein said electromagnetic radiation is applied using a microwave.

191. The method of embodiment 176, wherein said capture moiety comprises a third reactive group that is capable of reacting with said second reactive group.

192. The method of embodiment 191, wherein said first reactive group, said second reactive group, or said third reactive group comprises a click chemistry moiety.

193. The method of embodiment 192, wherein said second reactive group and said third reactive group comprise a click chemistry pair.

194. The method of embodiment 174, wherein said linker comprises an amino acid reactive group and an additional polymerizable molecule, wherein said additional polymerizable molecule is configured to couple to said capture moiety.

195. The method of embodiment 194, wherein said additional polymerizable molecule comprises a nucleic acid molecule comprising an enzyme recognition site.

196. The method of embodiment 195, wherein said enzyme recognition site is recognized by a nuclease.

197. The method of embodiment 196, further comprising, subsequent to (e), cleaving said additional polymerizable molecule, thereby releasing said one or more monomers from said capture moiety.

198. The method of embodiment 196, wherein said nuclease is a restriction enzyme, a Cas protein, or an Argonaut nuclease.

199. The method of any one of embodiments 126-198, wherein said polymeric analyte comprises a macromolecule.

200. The method of embodiment 199, wherein said macromolecule comprises a peptide comprising a plurality of amino acids, wherein said monomer is an amino acid of said plurality of amino acids.

201. The method of embodiment 200, wherein said amino acid is a terminal amino acid.

202. The method of embodiment 200 or 201, wherein at least one amino acid of said plurality of amino acids comprises a post-translational modification.

203. The method of any one of embodiments 200-201, wherein at least one amino acid of said plurality of amino acids comprises a non-naturally occurring modification.

204. The method of embodiment 203, further comprising, generating said peptide comprising said non-naturally occurring modification.

205. The method of embodiment 204, wherein said generating comprises alkylating or acetylating said at least one amino acid, beta-elimination of a phosphate group, or use of PITC or acetic anhydride.

206. The method of embodiment 204 or 205, wherein said generating comprises converting a cysteine residue to cysteic acid.

207. The method of any one of embodiments 204-206, wherein said generating comprises use of an oxidizing or reducing agent.

208. The method of embodiment 203, wherein said non-naturally occurring modification is located on said amino acid.

209. The method of any one of embodiments 199-208, wherein said macromolecule is derived from a biological sample.

210. The method of embodiment 126, wherein said polymeric analyte comprises a peptoid.

211. The method of any one of embodiments 126-210, wherein said binding agent comprises an antibody, antibody fragment, aptamer, scFv, nanobody, anticalin, tRNA-acyl synthetase, peptide, peptide mimetic, a polysaccharide, or derivative thereof.

212. The method of any one of embodiments 126-211, wherein said binding agent comprises an enzyme.

213. The method of embodiment 212, wherein said enzyme comprises a metalloprotease, aminopeptidase, or exopeptidase.

214. The method of any one of embodiments 126-213, wherein said polymeric analyte, said capture moiety, or said second polymerizable molecule is coupled to a substrate.

215. The method of embodiment 214, wherein said substrate is a solid support.

216. The method of embodiment 214 or 215, wherein said substrate is substantially planar.

217. The method of embodiment 214 or 215, wherein said substrate is a bead or particle.

218. The method of any one of embodiments 214-217, wherein said polymeric analyte, said capture moiety, or said second polymerizable molecules is coupled to said substrate via a click chemistry moiety.

219. The method of any one of embodiments 214-217, wherein said polymeric analyte, said capture moiety, or said second polymerizable molecules is coupled to said substrate via a functional group.

220. The method of embodiment 219, wherein said functional group is added to said polymeric analyte, said capture moiety, or said second polymerizable molecule using an enzyme.

221. The method of embodiment 220, wherein said enzyme comprises an amidase.

222. The method of any one of embodiments 214-221, wherein said polymeric analyte, said capture moiety, or said second polymerizable molecules is coupled to said substrate using a linker molecule.

223. The method of any one of embodiments 214-222, wherein said substrate is functionalized with a functional group, and further comprising, attaching said polymeric analyte, said capture moiety, or said second polymerizable molecule to said functional group.

224. The method of any one of embodiments 214-223, wherein said polymeric analyte, said capture moiety, and said second polymerizable molecule are coupled to said substrate.

225. The method of any one of embodiments 214-224, further comprising, passivating said substrate.

226. The method of embodiment 225, wherein said passivating decreases nonspecific binding of said binding agent.

227. The method of embodiment 126, wherein said cleaving in (b) comprises applying a stimulus.

228. The method of embodiment 227, wherein said stimulus comprises a change in pH.

229. The method of embodiment 227, wherein said stimulus comprises the use of a Lewis acid.

230. The method of embodiment 229, wherein said Lewis acid comprises boron triflate (BF₃), boron trifluoride etherate, boron trichloride, boron tribromide, boron triiodide, or scandium triflate.

231. The method of any one of embodiments 227-230, wherein said stimulus comprises a biological stimulus.

232. The method of embodiment 231, wherein said biological stimulus comprises use of an enzyme.

233. The method of embodiment 232, wherein said enzyme is a metalloprotease, aminopeptidase, or exopeptidase.

234. The method of embodiment 233, further comprising, providing a metal catalyst.

235. The method of embodiment 232, wherein said monomer comprises a modification, and wherein said enzyme recognizes said modification.

236. The method of any one of embodiments 126-235, wherein said first polymerizable molecule comprises a moiety that identifies said binding agent or said monomer.

237. The method of embodiment 236, wherein said first polymerizable molecule comprises a nucleic acid molecule comprising a sequence that identifies said binding agent or said monomer.

238. The method of any one of embodiments 126-237, further comprising, subjecting said monomer-capture moiety complex to conditions sufficient to inhibit binding of an additional binding agent to said monomer-capture moiety complex.

239. The method of embodiment 238, wherein said conditions comprise a chemical or enzymatic treatment.

240. The method of any one of embodiments 126-239, wherein said monomer is <10 nm in size.

241. The method of any one of embodiments 126-240, wherein said monomer has a molecular mass of less than 210 daltons.

242. A method for processing an analyte, comprising:
(a) providing (i) said analyte, (ii) a binding agent comprising an activating agent, (iii) a first polymerizable molecule, and (iv) a second polymerizable molecule, wherein said analyte is <10 nm in size;
(b) contacting said analyte with said binding agent; and
(c) using said activating agent to couple said first polymerizable molecule to said second polymerizable molecule.

243. The method of embodiment 242, wherein said binding agent is not coupled to said first polymerizable molecule.

244. The method of embodiment 242, wherein said binding agent is coupled indirectly to said first polymerizable molecule.

245. The method of embodiment 242, wherein said activating agent comprises an activating polymerizable molecule, and further comprising, coupling said first polymerizable molecule to said activating polymerizable molecule.

246. The method of embodiment 245, wherein said first polymerizable molecule, said second polymerizable molecule, and said activating polymerizable molecule are nucleic acid molecules, and wherein said coupling is mediated using a splint molecule comprising a sequence complementary to at least a portion of (i) said first polymerizable molecule, (ii) said second polymerizable molecule and (iii) said activating polymerizable molecule.

247. The method of any one of embodiments 242-246, wherein said first polymerizable molecule comprises a nucleic acid molecule, and further comprising, generating said first polymerizable molecule via hybridization chain reaction (HCR).

248. The method of embodiment 247, wherein said generating comprises (A) providing a first hairpin molecule and a second hairpin molecule; (B) coupling said first hairpin molecule to said activating polymerizable molecule; and (C) coupling said second hairpin molecule to said first hairpin molecule, thereby generating said first polymerizable molecule coupled to said activating polymerizable molecule.

249. The method of embodiment 248, wherein subsequent to (C), said first polymerizable molecule comprises a flap sequence, and further comprising, using said flap sequence to couple said first polymerizable molecule to said second polymerizable molecule.

250. The method of any one of embodiments 242-249, wherein said activating agent comprises an anchoring nucleic acid molecule, wherein said anchoring nucleic acid molecule is configured to couple to an additional second polymerizable molecule adjacent to said second polymerizable molecule.

251. The method of embodiment 250, wherein said first polymerizable molecule is coupled to said binding agent.

252. The method of any one of embodiments 242-251, wherein said activating agent comprises an enzyme selected from the group consisting of: a peroxidase, a Cas protein, a ligase, a kinase, and a restriction enzyme.

253. The method of embodiment 252, wherein said enzyme is a kinase, and further comprising, using said kinase to phosphorylate said second polymerizable molecule, thereby allowing coupling of said first polymerizable to said second polymerizable molecule.

254. The method of embodiment 253, wherein said enzyme is a horseradish peroxidase (HRP) or an ascorbate peroxidase (APEX), wherein said HRP or said APEX activates said first polymerizable molecule or said second polymerizable molecule, thereby coupling said first polymerizable molecule to said second polymerizable molecule.

255. The method of embodiment 253, wherein said enzyme is a restriction enzyme and wherein said second polymerizable molecule comprises a restriction recognition site, and further comprising, cleaving said second polymerizable molecule with said restriction enzyme prior to said coupling of said first polymerizable molecule to said second polymerizable molecule.

256. The method of any one of embodiments 242-255, wherein said analyte comprises a monomeric unit of a polymer.

257. The method of embodiment 256, wherein said polymer is a peptide and said monomeric unit comprises an amino acid.

258. The method of embodiment 257, wherein at least one amino acid of said peptide comprises a post-translational modification.

259. The method of any one of embodiments 242-258, further comprising, prior to (a), providing a polymer comprising a plurality of monomers, coupling a monomer of said plurality of monomers to a substrate, and cleaving said monomer from said polymer, thereby yielding said analyte coupled to said substrate.

260. The method of embodiment 259, wherein said polymer comprises a peptide and said monomer comprises an amino acid or modified amino acid.

261. The method of embodiment 259, wherein said coupling is mediated by a linker.

262. The method of embodiment 261, wherein said linker comprises a first reactive moiety that is capable of coupling to said monomer and a second reactive moiety that is capable of coupling to said substrate.

263. A method for processing an analyte, comprising:
(a) providing (i) said analyte, (ii) a binding agent, and (iii) a first polymerizable molecule, wherein said first polymerizable molecule is not coupled to said binding agent; and (iv) a second polymerizable molecule, wherein said analyte is <10 nm in size;
(b) contacting said analyte with said binding agent; and
(c) coupling said first polymerizable molecule to said second polymerizable molecule.

264. The method of embodiment 263, wherein said binding agent comprises an activating polymerizable molecule, and further comprising, coupling said first polymerizable molecule to said activating polymerizable molecule.

265. The method of embodiment 264, wherein said first polymerizable molecule, said second polymerizable molecule, and said activating polymerizable molecule are nucleic acid molecules, and wherein said coupling is mediated using a splint molecule comprising a sequence complementary to at least a portion of (i) said first polymerizable molecule, (ii) said second polymerizable molecule and (iii) said activating polymerizable molecule.

266. The method of any one of embodiments 263-265, wherein said first polymerizable molecule comprises a nucleic acid molecule, and further comprising, generating said first polymerizable molecule via hybridization chain reaction (HCR).

267. The method of embodiment 266, wherein said generating comprises (A) providing a first hairpin molecule and a second hairpin molecule; (B) coupling said first hairpin molecule to said activating polymerizable molecule; and (C) coupling said second hairpin molecule to said first hairpin molecule, thereby generating said first polymerizable molecule coupled to said activating polymerizable molecule.

268. The method of embodiment 267, wherein subsequent to (C), said first polymerizable molecule comprises a flap sequence, and further comprising, using said flap sequence to couple said first polymerizable molecule to said second polymerizable molecule.

269. The method of any one of embodiments 263-268, wherein said binding agent comprises an anchoring nucleic acid molecule, wherein said anchoring nucleic acid molecule is configured to couple to an additional second polymerizable molecule adjacent to said second polymerizable molecule.

270. The method of any one of embodiments 263-269, wherein said binding agent comprises an enzyme coupled thereto, wherein said enzyme is selected from the group consisting of: a peroxidase, a Cas protein, a ligase, a kinase, and a restriction enzyme.

271. The method of embodiment 270, wherein said enzyme is a kinase, and further comprising, using said kinase to phosphorylate said second polymerizable molecule, thereby allowing coupling of said first polymerizable to said second polymerizable molecule.

272. The method of embodiment 270, wherein said enzyme is a horseradish peroxidase (HRP) or an ascorbate peroxidase (APEX), wherein said HRP or said APEX activates said first polymerizable molecule or said second polymerizable molecule, thereby coupling said first polymerizable molecule to said second polymerizable molecule.

273. The method of embodiment 270, wherein said enzyme is a restriction enzyme and wherein said second polymerizable molecule comprises a restriction recognition site, and further comprising, cleaving said second polymerizable molecule with said restriction enzyme prior to said coupling of said first polymerizable molecule to said second polymerizable molecule.

274. The method of any one of embodiments 263-273, wherein said analyte comprises a monomeric unit of a polymer.

275. The method of embodiment 274, wherein said polymer is a peptide and said monomeric unit comprises an amino acid.

276. The method of embodiment 275, wherein at least one amino acid of said peptide comprises a post-translational modification.

277. The method of any one of embodiments 263-276, further comprising, prior to (a), providing a polymer comprising a plurality of monomers, coupling a monomer of said plurality of monomers to a substrate, and cleaving said monomer from said polymer, thereby yielding said analyte coupled to said substrate.

278. The method of embodiment 277, wherein said polymer comprises a peptide and said monomer comprises an amino acid or modified amino acid.

279. The method of embodiment 277, wherein said coupling is mediated by a linker.

280. The method of embodiment 279, wherein said linker comprises a first reactive moiety that is capable of coupling to said monomer and a second reactive moiety that is capable of coupling to said substrate.

281. A method, comprising:
(a) providing (i) a peptide comprising a plurality of amino acids, wherein an amino acid of said plurality of amino acids comprises a non-naturally occurring modification of said plurality of amino acids, (ii) a substrate coupled to said peptide, and (iii) a linker;
(b) using said linker to couple said amino acid or an additional amino acid of said plurality of amino acids to said substrate to yield a substrate-coupled amino acid; and
(c) subsequent to (b), cleaving said substrate-coupled amino acid from said peptide, thereby yielding (i) a removed substrate-coupled amino acid and (ii) a remainder of said peptide.

282. The method of embodiment 281, further comprising, subsequent to (c), contacting said removed substrate-coupled amino acid with a binding agent.

283. The method of embodiment 282, wherein said binding agent comprises a first polymerizable molecule, and wherein said substrate comprises a second polymerizable molecule.

284. The method of embodiment 283, wherein said first polymerizable molecule comprises a moiety that identifies said binding agent or said amino acid.

285. The method of embodiment 284, wherein said first polymerizable molecule comprises a nucleic acid molecule comprising a barcode sequence that identifies said binding agent or said amino acid.

286. The method of any one of embodiments 283-285, further comprising, coupling said first polymerizable molecule to said second polymerizable molecule.

287. The method of embodiment 286, further comprising, sequencing said first polymerizable molecule.

288. The method of embodiment 287, wherein said sequencing is performed using next generation DNA sequencing.

289. The method of any one of embodiments 283-288, wherein said first polymerizable molecule or said second polymerizable molecule comprises a nucleic acid molecule.

290. The method of embodiment 289, wherein said nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof.

291. The method of embodiment 290, wherein said nucleic acid molecule comprises a hexonucleic acid (HNA).

292. The method of any one of embodiments 289-291, wherein greater than 70% of nucleotides of said nucleic acid molecule are thymines or cytosines.

293. The method of any one of embodiments 289-292, wherein said first polymerizable molecule comprises a first nucleic acid molecule and said second polymerizable molecule comprises a second nucleic acid molecule.

294. The method of embodiment 293, wherein said coupling of said first polymerizable molecule and said second polymerizable molecule comprises ligation or hybridization of said first nucleic acid molecule to said second nucleic acid molecule.

295. The method of embodiment 293, wherein said coupling of said first polymerizable molecule and said second polymerizable molecule is mediated by a splint molecule.

296. The method of embodiment 286, wherein said coupling comprises hybridization of at least a portion of said first polymerizable molecule to at least a portion of said second polymerizable molecule.

297. The method of embodiment 296, further comprising, performing a nucleic acid extension reaction.

298. The method of any one of embodiments 283-297, wherein said first polymerizable molecule comprises a first reactive moiety and said second polymerizable molecule comprises a second reactive moiety capable of reacting with said first reactive moiety.

299. The method of embodiment 298, wherein said coupling in (ii) comprises reacting said first moiety and said second moiety.

300. The method of embodiment 298 or 299, wherein said first reactive moiety or said second reactive moiety comprises a click chemistry moiety.

301. The method of embodiment 282, wherein said binding agent comprises an antibody, antibody fragment, aptamer, nanobody, peptide, peptide mimetic, a polysaccharide, or derivative thereof.

302. The method of any one of embodiments 282-301, wherein said binding agent comprises an enzyme.

303. The method of embodiment 302, wherein said enzyme comprises a metalloprotease, aminopeptidase, or exopeptidase.

304. The method of any one of embodiments 281-303, wherein said cleaving in (c) comprises applying a stimulus.

305. The method of embodiment 304, wherein said stimulus comprises a change in pH.

306. The method of embodiment 304, wherein said stimulus comprises the use of a Lewis acid.

307. The method of embodiment 306, wherein said Lewis acid comprises boron triflate (BF₃), boron trifluoride etherate, boron trichloride, boron tribromide, boron triiodide, or scandium triflate.

308. The method of embodiment 304, wherein said stimulus comprises electromagnetic radiation.

309. The method of embodiment 308, wherein said electromagnetic radiation is applied using a microwave.

310. The method of embodiment 304, wherein said stimulus is a biological stimulus.

311. The method of embodiment 310, wherein said biological stimulus comprises use of an enzyme.

312. The method of embodiment 311, wherein said enzyme is a metalloprotease, aminopeptidase, or exopeptidase.

313. The method of embodiment 312, further comprising, providing a metal catalyst.

314. The method of embodiment 311, wherein said amino acid comprises a modification, and wherein said enzyme recognizes said modification.

315. The method of embodiment 282, further comprising, subjecting said removed substrate-coupled amino acid to conditions sufficient to inhibit binding of an additional binding agent to said removed substrate-coupled amino acid.

316. The method of embodiment 315, wherein said conditions comprise a chemical or enzymatic treatment.

317. The method of any one of embodiments 282-316, further comprising, blocking a carboxyl group or an amine group of said peptide.

318. The method of any one of embodiments 281-317, wherein said cleaving in (c) comprises cleaving one or more amino acids from said peptide.

319. The method of any one of embodiments 281-318, further comprising, removing said amino acid of said substrate-coupled amino acid from said substrate.

320. The method of any one of embodiments 281-319, wherein said substrate comprises a capture moiety, wherein said capture moiety is coupled to said amino acid or said additional amino acid via said linker.

321. The method of embodiment 320, wherein said capture moiety comprises a polymerizable molecule.

322. The method of embodiment 321, wherein said polymerizable molecule is a nucleic acid molecule.

323. The method of embodiment 322, wherein said nucleic acid molecule comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof.

324. The method of embodiment 322 or 323, wherein said nucleic acid molecule comprises a hexonucleic acid (HNA).

325. The method of any one of embodiments 322-324, wherein greater than 70% of nucleotides of said nucleic acid molecule are thymines or cytosines.

326. The method of any one of embodiments 322-325, wherein said nucleic acid molecule comprises a priming site, a sequencing site, a unique molecular identifier, a barcode sequence, a cleavage site, or a combination thereof.

327. The method of any one of embodiments 281-326, further comprising, prior to (a), generating said peptide comprising said non-naturally occurring modification.

328. The method of embodiment 327, wherein said generating comprises alkylating or acetylating an amino acid residue of said peptide.

329. The method of embodiment 327 or 328, wherein said generating comprises beta-elimination of a phosphate group and addition of a thiol group.

330. The method of any one of embodiments 327-329, wherein said generating comprises using phenylisothiocyanate or acetic anhydride.

331. The method of any one of embodiments 281-330, wherein said non-naturally occurring modification is on an N-terminal or C-terminal amino acid of said peptide.

332. The method of any one of embodiments 281-331, wherein (c) is performed using a protease that recognizes said non-naturally occurring modification.

333. The method of any one of embodiments 281-332, wherein said coupling in (b) comprises coupling a C-terminal amino acid to said substrate.

334. The method of embodiment 333, further comprising, generating said peptide comprising said non-naturally occurring modification.

335. The method of embodiment 334, wherein said generating comprises subjecting said peptide to conditions sufficient to block all carboxyl groups.

336. The method of embodiment 333, wherein said coupling of said C-terminal amino acid is performed using a photoredox reaction or an enzymatic reaction.

337. The method of embodiment 336, wherein said enzymatic reaction comprises carboxypeptidase or amidase.

338. The method of any one of embodiments 281-332, wherein said coupling in (b) comprises coupling a N-terminal amino acid to said substrate.

339. The method of any one of embodiments 281-338, wherein said linker is a bifunctional linker.

340. The method of embodiment 339, wherein said linker comprises a first reactive group and a second reactive group, wherein said first reactive group is capable of coupling to said amino acid and wherein said second reactive group is capable of coupling to said substrate.

341. The method of embodiment 340, wherein said amino acid is a terminal amino acid.

342. The method of embodiment 340 or 341, wherein said first reactive group comprises a thiocyanate, aldehyde group, dansyl chloride, or fluorobenzene.

343. The method of embodiment 342, wherein said thiocyanate comprises a phenylisothiocyanate (PITC) moiety and wherein said coupling of (b) comprises coupling said PITC moiety to said amino acid to generate a phenylthiocarbamoyl (PTC) derivative of said amino acid.

344. The method of embodiment 343, further comprising, subsequent to (c), derivatizing said PTC derivative to generate a thiazolinone (ATZ) derivative or a phenylthiohydantoin (PTH)-amino acid derivative.

345. The method of any one of embodiments 281-344, wherein said cleaving in (c) is performed by applying a stimulus.

346. The method of embodiment 345, wherein said stimulus comprises a change in pH.

347. The method of embodiment 346, wherein said change in pH comprises the use of an acid.

348. The method of embodiment 346, wherein said change in pH comprises the use of a base.

349. The method of embodiment 345, wherein said stimulus comprises the use of a Lewis acid.

350. The method of embodiment 349, wherein said Lewis acid comprises boron triflate (BF₃), boron trifluoride etherate, boron trichloride, boron tribromide, boron triiodide, or scandium triflate.

351. The method of embodiment 345, wherein said stimulus comprises electromagnetic radiation.

352. The method of embodiment 351, wherein said electromagnetic radiation is applied using a microwave.

353. The method of any one of embodiments 340-352, wherein said substrate comprises a capture moiety that comprises a third reactive group that is capable of reacting with said second reactive group.

354. The method of embodiment 353, wherein said first reactive group, said second reactive group, or said third reactive group comprises a click chemistry moiety.

355. The method of embodiment 354, wherein said second reactive group and said third reactive group comprise a click chemistry pair.

356. The method of any one of embodiments 281-355, wherein said linker comprises an amino acid reactive group and a polymerizable molecule, wherein said polymerizable molecule is configured to couple to said substrate.

357. The method of embodiment 356, wherein said polymerizable molecule comprises an enzyme recognition site.

358. The method of embodiment 357, wherein said enzyme recognition site is recognized by a nuclease.

359. The method of embodiment 358, wherein said nuclease is a restriction enzyme, a Cas protein, or an Argonaut nuclease.

360. The method of any one of embodiments 281-359, further comprising, contacting said removed substrate-coupled amino acid with a binding agent.

361. The method of embodiment 360, wherein said binding agent comprises an antibody, antibody fragment, aptamer, scFv, nanobody, anticalin, tRNA-acyl synthetase, peptide, peptide mimetic, a polysaccharide, or derivative thereof.

362. The method of embodiment 361, wherein said binding agent comprises an enzyme.

363. The method of embodiment 362, wherein said enzyme comprises a metalloprotease, aminopeptidase, or exopeptidase.

364. The method of any one of embodiments 281-363, wherein said substrate is a solid support.

365. The method of embodiment 364, wherein said substrate is substantially planar.

366. The method of embodiment 364, wherein said substrate is a bead or particle.

367. The method of embodiment 364, wherein said peptide is coupled to said support via a click chemistry moiety.

368. The method of embodiment 364, wherein said peptide is coupled to said support via a functional group.

369. The method of embodiment 368, wherein said functional group is added to said peptide, said capture moiety, or said second polymerizable molecule using an enzyme.

370. The method of embodiment 369, wherein said enzyme comprises an amidase.

371. The method of any one of embodiments 281-370, wherein said peptide is coupled to said support via a linker molecule.

372. The method of any one of embodiments 281-371, wherein said substrate is functionalized with a functional group, and further comprising, attaching said peptide to said functional group.

373. The method of any one of embodiments 281-372, further comprising, passivating said substrate.

374. The method of any one of embodiments 281-373, wherein said cleaving in (c) comprises applying a stimulus.

375. The method of embodiment 374, wherein said stimulus comprises a change in pH.

376. The method of embodiment 374, wherein said stimulus comprises the use of a Lewis acid.

377. The method of embodiment 376, wherein said Lewis acid comprises boron triflate (BF₃), boron trifluoride etherate, boron trichloride, boron tribromide, boron triiodide, or scandium triflate.

378. The method of embodiment 374, wherein said stimulus comprises electromagnetic radiation.

379. The method of embodiment 378, wherein said electromagnetic radiation is applied using a microwave.

380. The method of embodiment 374, wherein said stimulus is a biological stimulus.

381. The method of embodiment 380, wherein said biological stimulus comprises use of an enzyme.

382. The method of embodiment 381, wherein said enzyme is a metalloprotease, aminopeptidase, or exopeptidase.

383. The method of embodiment 382, further comprising, providing a metal catalyst.

384. The method of embodiment 381, wherein said amino acid comprises a modification, and wherein said enzyme recognizes said modification.

385. The method of any one of embodiments 281-384, further comprising, blocking a carboxyl group or an amine group of said peptide.

386. The method of any one of embodiments 281-385, further comprising, generating said peptide comprising said non-naturally occurring modification.

387. The method of embodiment 386, wherein said generating comprises alkylating or acetylating said at least one amino acid, beta-elimination of a phosphate group, or use of PITC or acetic anhydride.

388. The method of embodiment 386 or 387, wherein said generating comprises converting a cysteine residue to cysteic acid.

389. The method of any one of embodiments 386-388, wherein said generating comprises use of an oxidizing or reducing agent.

390. The method of any one of embodiments 281-389, wherein said peptide is derived from a biological sample.

391. A method for sequencing a peptide, comprising:
(a) providing a substrate comprising said peptide and a nucleic acid molecule;
(b) providing a linker, wherein said linker comprises an amino acid reactive group and a substrate-tethering moiety;
(c) coupling said linker to a terminal amino acid of said peptide and to said substrate;
(d) cleaving said terminal amino acid, thereby providing a cleaved amino acid-linker complex coupled to said substrate;
(e) contacting said cleaved amino acid-linker complex with a binding agent comprising an activating agent;
(f) providing a nucleic acid barcode molecule; and
(g) using said activating agent to couple said nucleic acid barcode molecule to said nucleic acid molecule.

392. The method of embodiment 391, wherein said substrate comprises a capture moiety, wherein said substrate-tethering moiety is configured to couple to said capture moiety.

393. The method of embodiment 392, wherein said capture moiety comprises an additional nucleic acid molecule, wherein said linker comprises a linking nucleic acid molecule, and wherein, in (c), said linking nucleic acid molecule couples to said additional nucleic acid molecule.

394. The method of embodiment 392, wherein said capture moiety comprises a first click chemistry moiety and wherein said linker comprises a second click chemistry moiety, and wherein said coupling in (c) comprises reacting said first click chemistry moiety with said second click chemistry moiety.

395. The method of any one of embodiments 391-394, wherein said activating agent comprises an activating nucleic acid molecule, and further comprising, coupling said nucleic acid barcode molecule to said activating polymerizable molecule.

396. The method of embodiment 395, wherein said coupling is mediated using a splint molecule comprising a sequence complementary to at least a portion of (i) said nucleic acid barcode molecule, (ii) said nucleic acid molecule and (iii) said activating nucleic acid molecule.

397. The method of any one of embodiments 391-396, further comprising, generating said nucleic acid barcode molecule via hybridization chain reaction (HCR).

398. The method of embodiment 397, wherein said generating comprises (A) providing a first hairpin molecule and a second hairpin molecule; (B) coupling said first hairpin molecule to said activating agent; and (C) coupling said second hairpin molecule to said first hairpin molecule, thereby generating said nucleic acid barcode molecule.

399. The method of embodiment 398, wherein said activating agent comprises an activating nucleic acid molecule.

400. The method of embodiment 398 or 399, wherein subsequent to (C), said nucleic acid barcode molecule comprises a flap sequence, and wherein (g) is mediated using said flap sequence to couple said nucleic acid barcode molecule to said nucleic acid molecule.

401. The method of embodiment 398, wherein said nucleic acid barcode molecule comprises said first hairpin molecule or said second hairpin molecule.

402. The method of any one of embodiments 391-401, wherein said activating agent comprises an anchoring nucleic acid molecule, wherein said anchoring nucleic acid molecule is configured to couple to an additional nucleic acid molecule adjacent to said nucleic acid molecule.

403. The method of embodiment 391, wherein said nucleic acid barcode molecule is coupled to said binding agent.

404. The method of any one of embodiments 391-403, wherein said activating agent comprises an enzyme selected from the group consisting of: a peroxidase, a Cas protein, a ligase, a kinase, and a restriction enzyme.

405. The method of embodiment 404, wherein said enzyme is a kinase, and further comprising, using said kinase to phosphorylate said nucleic acid molecule, thereby allowing coupling of said nucleic acid barcode molecule to said nucleic acid molecule.

406. The method of embodiment 404, wherein said enzyme is a horseradish peroxidase (HRP) or an ascorbate peroxidase (APEX), wherein said HRP or said APEX activates said nucleic acid barcode molecule or said nucleic acid molecule, thereby coupling said nucleic acid barcode molecule to said nucleic acid molecule.

407. The method of embodiment 404, wherein said enzyme is a restriction enzyme and wherein said nucleic acid molecule comprises a restriction recognition site, and further comprising, cleaving said nucleic acid molecule with said restriction enzyme prior to said coupling of said nucleic acid barcode molecule to said nucleic acid molecule.

408. The method of any one of embodiments 391-407, wherein at least one amino acid of said peptide comprises a post-translational modification.

409. The method of any one of embodiments 391-408, wherein said coupling in (c) is mediated by a linker.

410. A method for characterizing a plurality of analytes, comprising:
(a) providing a first analyte, a second analyte, a first binding agent capable of coupling to said first analyte and not said second analyte, and a second binding agent capable of coupling to both said first analyte and said second analyte;
(b) contacting said first analyte and said second analyte with said first binding agent;
(c) subsequent to (b), contacting said first analyte and said second analyte with said second binding agent;
(d) identifying said first binding agent and said second binding agent; and
(e) using said first binding agent and said second binding agent identified in (d) to identify said first analyte and said second analyte.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-99. (canceled)

100. A method for sequencing a peptide comprising a plurality of amino acids, comprising:
(a) providing said peptide and a linker, wherein said linker is coupled to a linking nucleic acid molecule;
(b) coupling said linker to an amino acid of said plurality of amino acids;
(c) coupling said linking nucleic acid molecule to an oligonucleotide capture moiety;
(d) cleaving said amino acid from said peptide, thereby yielding a cleaved amino acid coupled to said oligonucleotide capture moiety;
(e) contacting said cleaved amino acid with a binding agent, wherein said binding agent comprises a detectable label; and
(f) detecting said detectable label, thereby identifying said amino acid.

101. The method of claim 100, wherein said linker comprises an amino acid-reactive moiety.

102. The method of claim 100, wherein said oligonucleotide capture moiety is coupled to a substrate.

103. The method of claim 102, wherein said peptide is coupled to said substrate.

104. The method of claim 102, wherein said substrate comprises a bead, a flow cell, a glass surface, or a silicon surface.

105. The method of claim 100, wherein said detectable label is a fluorophore, radioisotope, mass tag, or chromogenic enzyme.

106. The method of claim 105, wherein said detectable label comprises a fluorophore and wherein said detecting comprises imaging.

107. The method of claim 106, wherein said imaging comprises super resolution microscopy.

108. The method of claim 100, wherein said binding agent comprises an antibody, antibody fragment, aptamer, scFv, nanobody, anticalin, tRNA-acyl synthetase, peptide, peptide mimetic, a polysaccharide, or derivative thereof.

109. The method of claim 100, wherein (c) comprises providing a plurality of binding agents, where said plurality of binding agents have specificity to different amino acids or derivatives thereof.

110. The method of claim 100, further comprising, repeating (a)-(e).

111. The method of claim 100, wherein said linking nucleic acid molecule or said oligonucleotide capture moiety comprises a barcode sequence.

112. The method of claim 110, wherein said barcode sequence comprises temporal information.

113. The method of claim 112, wherein said temporal information comprises a cycle number.

114. The method of claim 100, wherein said binding agent comprises a cycle barcode that indicates a round or iteration in which said binding agent is provided.

115. The method of claim 100, wherein said plurality of amino acids comprises a non-naturally occurring modification.

116. The method of claim 100, wherein said amino acid is a terminal amino acid.

117. The method of claim 100, wherein said oligonucleotide capture moiety comprises a polynucleotide analog.

118. The method of claim 117, wherein said oligonucleotide capture moiety comprises a DNA molecule with a pseudo-complementary base, a bridged nucleic acid (BNA), a xenonucleic acid (XNA), a locked nucleic acid (LNA), a peptide nucleic acid (PNA), a gamma-PNA molecule, a morpholino, or a combination thereof.

119. The method of claim 100, wherein said linking nucleic acid molecule or said oligonucleotide capture moiety comprises a unique molecular identifier (UMI).


SEQUENCE LISTING (30 sequences)

SEQ ID NO: 1 - DNA, 52 bp, synthetic construct
  source 1..52: mol_type = other DNA
  modified_base 1: mod_base = OTHER; note = 5' Phosphate modified uracil
  modified_base 52: mod_base = OTHER; note = 3' Azide (N-hydroxysuccinimide Ester) modified nucleotide
tctccctctctttctctctttccctctctctccctttctccctccctctccc

SEQ ID NO: 2 - DNA, 27 bp, synthetic construct
  source 1..27: mol_type = other DNA
  modified_base 1: mod_base = OTHER; note = 5' Azide (N-hydroxysuccinimide Ester) modified nucleotide
ttcttcttcttctccctcctctcttct

SEQ ID NO: 3 - DNA, 12 bp, synthetic construct
  source 1..12: mol_type = other DNA
gggagaagaaga

SEQ ID NO: 4 - DNA, 79 bp, synthetic construct
  source 1..79: mol_type = other DNA
  modified_base 1: mod_base = OTHER; note = Azide modified nucleotide
ttcttcttcttctccctcctccctccctctttctttctctctctctctctctctctctccctttctccctccctctccc

SEQ ID NO: 5 - DNA, 79 bp, synthetic construct
  source 1..79: mol_type = other DNA
  modified_base 1: mod_base = OTHER; note = Azide modified nucleotide
ttcttcttcttctccctcctccctccctctttctttcgcactgtatcgcactgtatctccctttctccctccctctccc

SEQ ID NO: 6 - DNA, 20 bp, synthetic construct
  source 1..20: mol_type = other DNA
ttcttcttcttctccctcct

SEQ ID NO: 7 - DNA, 20 bp, synthetic construct
  source 1..20: mol_type = other DNA
cctttctccctccctctccc

SEQ ID NO: 8 - DNA, 20 bp, synthetic construct
  source 1..20: mol_type = other DNA
  modified_base 1: mod_base = OTHER; note = 5' Phosphate modified nucleotide
ctcttttttttttttttttt

SEQ ID NO: 9 - DNA, 52 bp, synthetic construct; identical to SEQ ID NO: 1
  source 1..52: mol_type = other DNA
  modified_base 1: mod_base = OTHER; note = Uracil

SEQ ID NO: 10 - DNA, 52 bp, synthetic construct; identical to SEQ ID NO: 1
  source 1..52: mol_type = other DNA
  modified_base 1: mod_base = OTHER; note = Uracil
  modified_base 52: mod_base = OTHER; note = Dibenzocyclooctyne modified nucleotide

SEQ ID NO: 11 - DNA, 36 bp, synthetic construct
  source 1..36: mol_type = other DNA
aaaaaaaaaaaaagagagaagagaggagggagaaga

SEQ ID NO: 12 - DNA, 36 bp, synthetic construct
  source 1..36: mol_type = other DNA
ggaattgggagtaagggctgtgatgcccttactccc

SEQ ID NO: 13 - DNA, 36 bp, synthetic construct
  source 1..36: mol_type = other DNA
gcccttactcccaattccgggagtaagggcatcaca

SEQ ID NO: 14 - DNA, 23 bp, synthetic construct
  source 1..23: mol_type = other DNA
  modified_base 1: mod_base = OTHER; note = Biotin modified nucleotide
tttttgcccttactcccaattcc

SEQ ID NO: 15 - DNA, 59 bp, synthetic construct
  source 1..59: mol_type = other DNA
aggctgagccaggatcaaactctgcccttactcccaattccgggagtaagggcatcaca

SEQ ID NO: 16 - DNA, 60 bp, synthetic construct
  source 1..60: mol_type = other DNA
gcccttactcccaattccgggagtaagggcatcacaaaagaaagagggagggaaggagaa

SEQ ID NO: 17 - DNA, 27 bp, synthetic construct
  source 1..27: mol_type = other DNA
ttcttcttcttctccctcctctcttct

SEQ ID NO: 18 - DNA, 20 bp, synthetic construct
  source 1..20: mol_type = other DNA
  modified_base 1: mod_base = OTHER; note = 5' Phosphate modified nucleotide
ccttccctccctctttcttt

SEQ ID NO: 19 - protein, 10 aa, synthetic construct
  VARIANT 7: note = A, R, N, D, C, Q, E, G, H, I, L, K, M, F, P, S, T, W, Y or V
LKFNIEXNWK

SEQ ID NO: 20 - DNA, 52 bp, synthetic construct; identical to SEQ ID NO: 1
  source 1..52: mol_type = other DNA
  modified_base 1: mod_base = OTHER; note = Uracil

SEQ ID NO: 21 - protein, 11 aa, synthetic construct
KGGGGRGRGRK

SEQ ID NO: 22 - DNA, 20 bp, synthetic construct
  source 1..20: mol_type = other DNA
  modified_base 1: mod_base = OTHER; note = 5' Phosphate modified nucleotide
ctcttttttttttttttttt

SEQ ID NO: 23 - DNA, 18 bp, synthetic construct
  source 1..18: mol_type = other DNA
gcccttactcccaattcc

SEQ ID NO: 24 - DNA, 34 bp, synthetic construct
  source 1..34: mol_type = other DNA
  modified_base 1: mod_base = OTHER; note = 5' Phosphate modified nucleotide
  modified_base 34: mod_base = OTHER; note = Azide modified nucleotide
ccttacagcaacagggtggtggaaagcttttttt

SEQ ID NO: 25 - DNA, 20 bp, synthetic construct
  source 1..20: mol_type = other DNA
agagtttgatcctggctcag

SEQ ID NO: 26 - DNA, 59 bp, synthetic construct; identical to SEQ ID NO: 15
  source 1..59: mol_type = other DNA

SEQ ID NO: 27 - DNA, 126 bp, synthetic construct
  source 1..126: mol_type = other DNA
gcccttactcccaattccgggagtaagggcatcacagcccttactcccaattccgggagtaagggcatcacagcccttactcccaattccgggagtaagggcatcacagcccttactcccaattcc

SEQ ID NO: 28 - DNA, 108 bp, synthetic construct
  source 1..108: mol_type = other DNA
ggaattgggagtaagggctgtgatgcccttactcccggaattgggagtaagggctgtgatgcccttactcccggaattgggagtaagggctgtgatgcccttactccc

SEQ ID NO: 29 - DNA, 36 bp, synthetic construct; identical to SEQ ID NO: 11
  source 1..36: mol_type = other DNA
  modified_base 36: mod_base = OTHER; note = 3' Phosphate modified nucleotide

SEQ ID NO: 30 - DNA, 26 bp, synthetic construct
  source 1..26: mol_type = other DNA
tcttcttcttctccctcctctcttct